(19) United States
(12) Patent Application Publication
(10) Pub. No.: US 2010/0144700 A1
(43) Pub. Date: Jun. 10, 2010

(54) HETEROCYCLIC-CARBONYL-DIAZABICYCLOALKANES AS MODULATORS OF THE NEURONAL NICOTINIC ACETYLCHOLINE ALPHA 4 BETA 2, SUBTYPE RECEPTOR FOR THE TREATMENT OF CNS RELATED DISORDERS

(75) Inventors: Philip S. Hammond, Pinnacle, NC (US); Anatoly A. Mazurov, Greensboro, NC (US); Lan Miao, Advance, NC (US); Yun-De Xiao, Clemmons, NC (US); Balwinder Singh Bhatti, Winston-Salem, NC (US); Jon-Paul Strachan, Burlington, NC (US); V. Srinivasa Murthy, Winston-Salem, NC (US); David C. Kombo, Winston-Salem, NC (US); Srinivisa Rao Akireddy, Winston-Salem, NC (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: TARGACEPT, INC., Winston-Salem, NC (US)

(21) Appl. No.: 12/530,997

(22) PCT Filed: Mar. 12, 2008
(86) PCT No.: PCT/US2008/056607

§ 371 Date: Feb. 26, 2010

Related U.S. Application Data
(60) Provisional application No. 60/906,762, filed on Mar. 13, 2007.

(51) Int. Cl. A61K 31/439; C07D 471/08; C07D 487/04; C07D 471/04; A61K 31/407; A61K 31/422; A61K 31/437; A61K 31/4353; A61P 25/00; A61P 25/28; A61P 25/18

(52) U.S. Cl. 514/210.16; 546/113; 548/453; 548/248; 514/300; 514/414; 514/378

(57) ABSTRACT

A compound of Formula 1: A-C(O)-Cy, wherein A is a diazabicyclic core, containing 7, 8, or 9 ring atoms, and selected from the following: 2,6-diazabicyclo[3.2.0]heptane; 3,6-diazabicyclo[3.ZO]heptane; 2,7-diazabicyclo[4.2.0]octane; 3,7-diazabicyclo[4.2.0]octane; 3,8-diazabicyclo[4.2.0]octane; 2,7-diazabicyclo[3.3.0]octane; 2,7-diazbicyclo[4.3.0]nonane; 2,8-diazbicyclo[4.3.0]nonane; 3,7-diazabicyclo[4.3.0]nonane; 3,8-diazabicyclo[4.3.0]nonane; 3,9-diazabicyclo[4.3.0]nonane; 2,6-diazabicyclo[3.2.1]octane; 3,6-diazabicyclo[3.2.1]octane; wherein the diazabicycle is attached as a radical to the depicted carbonyl via either one of the two ring nitrogen atoms, such that the carbonyl forms an amide bond with the ring nitrogen; Cy is a heteroaryl group; The compounds exhibit selectivity for, and bind with high affinity to, neuronal nicotinic receptors of the α402 subtype in the central nervous system (CNS). The compounds and compositions can be used to treat and/or prevent a wide variety of conditions or disorders, particularly CNS disorders. The compounds are believed to: (i) alter the number of nicotinic cholinergic receptors of the brain of the patient, (ii) exhibit neuroprotective effects, and (iii) when employed in effective amounts, not result in appreciable adverse side effects, namely side effects such as significant Increases in blood pressure and heart rate, significant negative effects upon the gastrointestinal tract, and significant effects upon skeletal muscle.

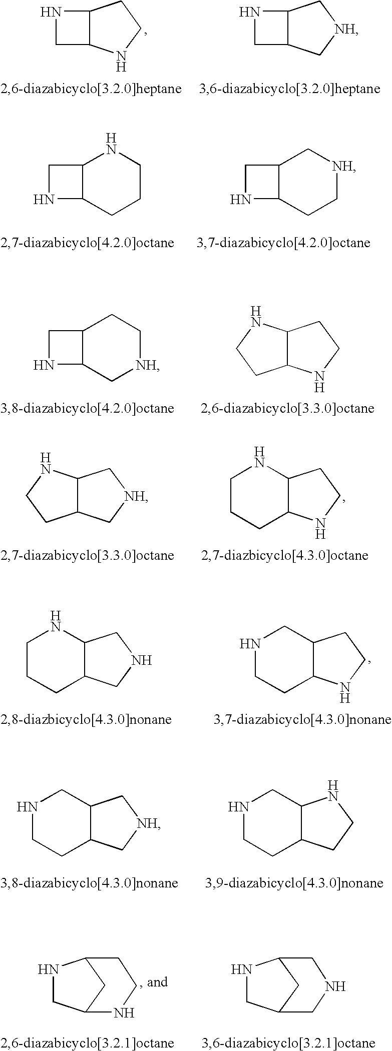

### FIELD OF THE INVENTION

The present invention relates to compounds that bind to and modulate the activity of neuronal nicotinic acetylcholine receptors, to processes for preparing these compounds, to pharmaceutical compositions containing these compounds and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several recent reviews. See, Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002), each of which is incorporated by reference with regard to such teaching. Among the kinds of indications for which NNR ligands have been proposed as therapies are cognitive disorders and dysfunctions, including Alzheimer's disease, attention deficit disorder and schizophrenia. See, Newhouse et al., Curr. Opin. Pharmacol. 4: 36 (2004), Levin and Rezvani, Curr. Drug Targets: CNS Neurol. Disord. 1: 423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Curr. Drug Targets: CNS Neurol. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry. 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001); each of which is herein incorporated by reference with regard to such teaching.

A limitation of some nicotinic compounds is that they are associated with various undesirable side effects, for example, by stimulating muscle and ganglionic receptors. It would be desirable to have compounds, compositions and methods for preventing and/or treating various conditions or disorders (e.g., CNS disorders), including alleviating the symptoms of these disorders, where the compounds exhibit nicotinic pharmacology with a beneficial effect (e.g., upon the functioning of the CNS), but without significant associated side effects. It would further be highly desirable to provide compounds, compositions and methods that affect CNS function without significantly affecting those receptor subtypes which have the potential to induce undesirable side effects (e.g., appreciable activity at cardiovascular and skeletal muscle sites). The present invention provides such compounds, compositions and methods.

### SUMMARY OF THE INVENTION

The present invention includes a compound of Formula 1:

A-C(O)-Cy Formula 1

or a pharmaceutically acceptable salt thereof,
wherein A is a diazabicyclic core, containing 7, 8, or 9 ring atoms and chosen from the following:

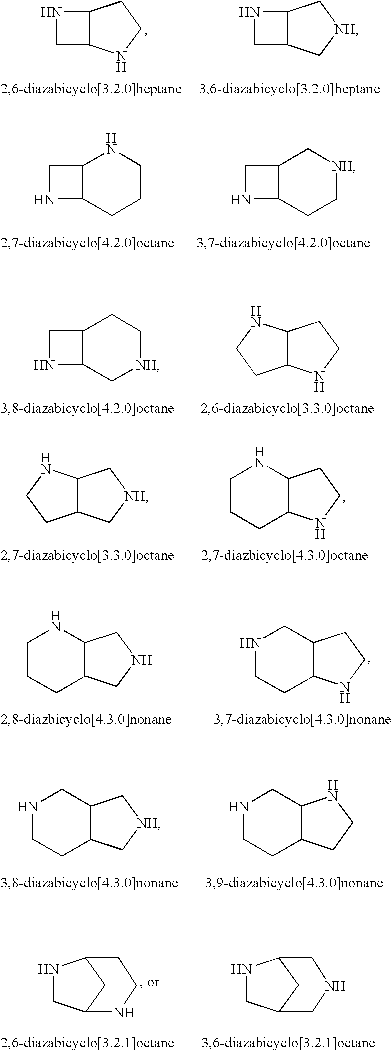

wherein the diazabicycle is attached as a radical to the depicted carbonyl via either one of the two ring nitrogen atoms, such that the carbonyl forms an amide bond with the ring nitrogen;
Cy is a heteroaryl group chosen from the group of 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 1,3,4-oxadiazol-2-yl, 1,2,4-oxadiazol-3-yl, 1,2,4-oxadiazol-5-yl, 2-thiazolyl, 4-thiazolyl, 5-thiazolyl, 3-isothiazolyl, 4-isothiazolyl, 5-isothiazolyl, 1,3,4-thiadiazol-2-yl, 1,2,4-thiadiazol-3-yl, 1,2,4-thiadiazol-5-yl, 3-pyridinyl, and 4-pyridinyl, each of which may be optionally substituted with up to three non-hydrogen substituents selected from alkyl, alkenyl, heterocyclyl, cycloalkyl, aryl, heteroaryl, alkylaryl, arylalkyl, halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)O R″, —SO₂R′, —SO₂NR′R″, and —NR′SO₂R″;
wherein each of alkyl, alkenyl, heterocyclyl, cycloalkyl, aryl, heteroaryls, alkylaryl, or arylalkyl may be substituted with one or more substituents selected from halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)O R″, —SO₂R′, —SO₂NR′R″, and —NR′SO₂R″ where R′ and R″ are individually hydrogen, alkyl, cycloalkyl, heterocyclyl, aryl, heteroaryl, or arylalkyl, or R′ and R″ can combine with the atoms to which they are attached to form a 3- to 8-membered cyclic functionality.

In one embodiment, the compound of the present invention is in isolated form.

In one embodiment, A is selected from 3,7-diazabicyclo[4.2.0]octane, 2,7-diazabicyclo[4.2.0]octane, 3,8-diazabicyclo[4.2.0]octane, or 3,6-diazabicyclo[3.2.1]octane.

In one embodiment, A is 3,6-diazabicyclo[3.2.1]octane.

In one embodiment, Cy is 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 3-pyridinyl, and 4-pyridinyl, each optionally substituted. In one embodiment, Cy is substituted with one or more of alkyl, aryl, heteroaryl, alkylaryl, arylalkyl, halogen, —CN, or —OR′, where R′ is alkyl, aryl, or arylalkyl.

One embodiment of the invention relates to compounds of Formula 1 wherein A is 3,6-diazabicyclo[3.2.1]octane and Cy is a heteroaromatic ring chosen from the group of 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 1,3,4-oxadiazol-2-yl, 1,2,4-oxadiazol-3-yl, 1,2,4-oxadiazol-5-yl, 2-thiazolyl, 4-thiazolyl, 5-thiazolyl, 3-isothiazolyl, 4-isothiazolyl, 5-isothiazolyl, 1,3,4-thiadiazol-2-yl, 1,2,4-thiadiazol-3-yl, 1,2,4-thiadiazol-5-yl, 3-pyridinyl and 4-pyridinyl. In another embodiment, the attachment of the heteroarylcarbonyl group is to the 3-position of the 3,6-diazabicyclo[3.2.1]octane ring system. In another embodiment, Cy is substituted by halogen. In another embodiment Cy 2-furanyl. In yet a further embodiment Cy is 2-furanyl optionally substituted with halo.

For the avoidance of doubt, the present invention relates to any compound falling within the scope of compounds of formula 1 as defined above.

One aspect of the present invention includes the use of a compound according to the present invention in the manufacture of a medicament for treatment or prevention of central nervous system disorders.

One aspect of the present invention includes a method for treatment or prevention of central nervous system disorders, comprising administering a compound of the present invention. In one embodiment, the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder and schizoaffective disorder.

One aspect of the present invention includes a pharmaceutical composition comprising a compound of the present invention and one or more pharmaceutically acceptable diluent, excipient, or inert carrier. In one embodiment, the pharmaceutical composition is useful for the treatment of central nervous system disorders. One aspect of the present invention includes a compound selected from the group consisting of:
2-(furan-2-ylcarbonyl)-2,6-diazabicyclo[3.2.0]heptane,
6-(furan-2-ylcarbonyl)-2,6-diazabicyclo[3.2.0]heptane,
2-(3-methylfuran-2-ylcarbonyl)-2,6-diazabicyclo[3.2.0]heptane,
6-(3-methylfuran-2-ylcarbonyl)-2,6-diazabicyclo[3.2.0]heptane,
2-(5-methylfuran-2-ylcarbonyl)-2,6-diazabicyclo[3.2.0]heptane,
6-(5-methylfuran-2-ylcarbonyl)-2,6-diazabicyclo[3.2.0]heptane,
2-(3-chlorofuran-2-ylcarbonyl)-2,6-diazabicyclo[3.2.0]heptane,
6-(3-chlorofuran-2-ylcarbonyl)-2,6-diazabicyclo[3.2.0]heptane,
2-(5-chlorofuran-2-ylcarbonyl)-2,6-diazabicyclo[3.2.0]heptane,
6-(5-chlorofuran-2-ylcarbonyl)-2,6-diazabicyclo[3.2.0]heptane,
2-(3-bromofuran-2-ylcarbonyl)-2,6-diazabicyclo[3.2.0]heptane,
6-(3-bromofuran-2-ylcarbonyl)-2,6-diazabicyclo[3.2.0]heptane,
2-(5-bromofuran-2-ylcarbonyl)-2,6-diazabicyclo[3.2.0]heptane,
6-(5-bromofuran-2-ylcarbonyl)-2,6-diazabicyclo[3.2.0]heptane,
2-(furan-3-ylcarbonyl)-2,6-diazabicyclo[3.2.0]heptane,
6-(furan-3-ylcarbonyl)-2,6-diazabicyclo[3.2.0]heptane,
2-(2-methylfuran-3-ylcarbonyl)-2,6-diazabicyclo[3.2.0]heptane,
6-(2-methylfuran-3-ylcarbonyl)-2,6-diazabicyclo[3.2.0]heptane,
2-(oxazol-2-ylcarbonyl)-2,6-diazabicyclo[3.2.0]heptane,
6-(oxazol-2-ylcarbonyl)-2,6-diazabicyclo[3.2.0]heptane,
2-(oxazol-5-ylcarbonyl)-2,6-diazabicyclo[3.2.0]heptane,
6-(oxazol-5-ylcarbonyl)-2,6-diazabicyclo[3.2.0]heptane,
2-(4-methyloxazol-5-ylcarbonyl)-2,6-diazabicyclo[3.2.0]heptane,
6-(4-methyloxazol-5-ylcarbonyl)-2,6-diazabicyclo[3.2.0]heptane,
2-(isoxazol-3-ylcarbonyl)-2,6-diazabicyclo[3.2.0]heptane,
6-(isoxazol-3-ylcarbonyl)-2,6-diazabicyclo[3.2.0]heptane,
2-(5-methylisoxazol-3-ylcarbonyl)-2,6-diazabicyclo[3.2.0]heptane,
6-(5-methylisoxazol-3-ylcarbonyl)-2,6-diazabicyclo[3.2.0]heptane,
2-(isoxazol-4-ylcarbonyl)-2,6-diazabicyclo[3.2.0]heptane,
6-(isoxazol-4-ylcarbonyl)-2,6-diazabicyclo[3.2.0]heptane,
2-(3-methylisoxazol-4-ylcarbonyl)-2,6-diazabicyclo[3.2.0]heptane,
6-(3-methylisoxazol-4-ylcarbonyl)-2,6-diazabicyclo[3.2.0]heptane,
2-(5-methylisoxazol-4-ylcarbonyl)-2,6-diazabicyclo[3.2.0]heptane,
6-(5-methylisoxazol-4-ylcarbonyl)-2,6-diazabicyclo[3.2.0]heptane,
2-(isoxazol-5-ylcarbonyl)-2,6-diazabicyclo[3.2.0]heptane,
6-(isoxazol-5-ylcarbonyl)-2,6-diazabicyclo[3.2.0]heptane,
2-(3-methylisoxazol-5-ylcarbonyl)-2,6-diazabicyclo[3.2.0]heptane,
6-(3-methylisoxazol-5-ylcarbonyl)-2,6-diazabicyclo[3.2.0]heptane,
2-(3-bromoisoxazol-5-ylcarbonyl)-2,6-diazabicyclo[3.2.0]heptane,
6-(3-bromoisoxazol-5-ylcarbonyl)-2,6-diazabicyclo[3.2.0]heptane,
2-(3-methoxyisoxazol-5-ylcarbonyl)-2,6-diazabicyclo[3.2.0]heptane,
6-(3-methoxyisoxazol-5-ylcarbonyl)-2,6-diazabicyclo[3.2.0]heptane,
2-(pyridin-4-ylcarbonyl)-2,6-diazabicyclo[3.2.0]heptane,
6-(pyridin-4-ylcarbonyl)-2,6-diazabicyclo[3.2.0]heptane,
3-(furan-2-ylcarbonyl)-3,6-diazabicyclo[3.2.0]heptane,
6-(furan-2-ylcarbonyl)-3,6-diazabicyclo[3.2.0]heptane,
3-(3-methylfuran-2-ylcarbonyl)-3,6-diazabicyclo[3.2.0]heptane,
6-(3-methylfuran-2-ylcarbonyl)-3,6-diazabicyclo[3.2.0]heptane,
3-(5-methylfuran-2-ylcarbonyl)-3,6-diazabicyclo[3.2.0]heptane,
6-(5-methylfuran-2-ylcarbonyl)-3,6-diazabicyclo[3.2.0]heptane,
3-(3-chlorofuran-2-ylcarbonyl)-3,6-diazabicyclo[3.2.0]heptane,
6-(3-chlorofuran-2-ylcarbonyl)-3,6-diazabicyclo[3.2.0]heptane,
3-(5-chlorofuran-2-ylcarbonyl)-3,6-diazabicyclo[3.2.0]heptane,
6-(5-chlorofuran-2-ylcarbonyl)-3,6-diazabicyclo[3.2.0]heptane,
3-(3-bromofuran-2-ylcarbonyl)-3,6-diazabicyclo[3.2.0]heptane,
6-(3-bromofuran-2-ylcarbonyl)-3,6-diazabicyclo[3.2.0]heptane,
3-(5-bromofuran-2-ylcarbonyl)-3,6-diazabicyclo[3.2.0]heptane,
6-(5-bromofuran-2-ylcarbonyl)-3,6-diazabicyclo[3.2.0]heptane,
3-(furan-3-ylcarbonyl)-3,6-diazabicyclo[3.2.0]heptane,
6-(furan-3-ylcarbonyl)-3,6-diazabicyclo[3.2.0]heptane,
3-(2-methylfuran-3-ylcarbonyl)-3,6-diazabicyclo[3.2.0]heptane,
6-(2-methylfuran-3-ylcarbonyl)-3,6-diazabicyclo[3.2.0]heptane,
3-(oxazol-2-ylcarbonyl)-3,6-diazabicyclo[3.2.0]heptane,
6-(oxazol-2-ylcarbonyl)-3,6-diazabicyclo[3.2.0]heptane,
3-(oxazol-5-ylcarbonyl)-3,6-diazabicyclo[3.2.0]heptane,
6-(oxazol-5-ylcarbonyl)-3,6-diazabicyclo[3.2.0]heptane,
3-(4-methyloxazol-5-ylcarbonyl)-3,6-diazabicyclo[3.2.0]heptane,
6-(4-methyloxazol-5-ylcarbonyl)-3,6-diazabicyclo[3.2.0]heptane,
3-(isoxazol-3-ylcarbonyl)-3,6-diazabicyclo[3.2.0]heptane,
6-(isoxazol-3-ylcarbonyl)-3,6-diazabicyclo[3.2.0]heptane,
3-(5-methylisoxazol-3-ylcarbonyl)-3,6-diazabicyclo[3.2.0]heptane,
6-(5-methylisoxazol-3-ylcarbonyl)-3,6-diazabicyclo[3.2.0]heptane,
3-(isoxazol-4-ylcarbonyl)-3,6-diazabicyclo[3.2.0]heptane,
6-(isoxazol-4-ylcarbonyl)-3,6-diazabicyclo[3.2.0]heptane,
3-(3-methylisoxazol-4-ylcarbonyl)-3,6-diazabicyclo[3.2.0]heptane,
6-(3-methylisoxazol-4-ylcarbonyl)-3,6-diazabicyclo[3.2.0]heptane,
3-(5-methylisoxazol-4-ylcarbonyl)-3,6-diazabicyclo[3.2.0]heptane,
6-(5-methylisoxazol-4-ylcarbonyl)-3,6-diazabicyclo[3.2.0]heptane,
3-(isoxazol-5-ylcarbonyl)-3,6-diazabicyclo[3.2.0]heptane,
6-(isoxazol-5-ylcarbonyl)-3,6-diazabicyclo[3.2.0]heptane,
3-(3-methylisoxazol-5-ylcarbonyl)-3,6-diazabicyclo[3.2.0]heptane,
6-(3-methylisoxazol-5-ylcarbonyl)-3,6-diazabicyclo[3.2.0]heptane,
3-(3-bromoisoxazol-5-ylcarbonyl)-3,6-diazabicyclo[3.2.0]heptane,
6-(3-bromoisoxazol-5-ylcarbonyl)-3,6-diazabicyclo[3.2.0]heptane,
3-(3-methoxyisoxazol-5-ylcarbonyl)-3,6-diazabicyclo[3.2.0]heptane,
6-(3-methoxyisoxazol-5-ylcarbonyl)-3,6-diazabicyclo[3.2.0]heptane,
3-(pyridin-4-ylcarbonyl)-3,6-diazabicyclo[3.2.0]heptane,
6-(pyridin-4-ylcarbonyl)-3,6-diazabicyclo[3.2.0]heptane,
2-(furan-2-ylcarbonyl)-2,7-diazabicyclo[4.2.0]octane,
7-(furan-2-ylcarbonyl)-2,7-diazabicyclo[4.2.0]octane,
2-(3-methylfuran-2-ylcarbonyl)-2,7-diazabicyclo[4.2.0]octane,
7-(3-methylfuran-2-ylcarbonyl)-2,7-diazabicyclo[4.2.0]octane,
2-(5-methylfuran-2-ylcarbonyl)-2,7-diazabicyclo[4.2.0]octane,
7-(5-methylfuran-2-ylcarbonyl)-2,7-diazabicyclo[4.2.0]octane,
2-(3-chlorofuran-2-ylcarbonyl)-2,7-diazabicyclo[4.2.0]octane,
7-(3-chlorofuran-2-ylcarbonyl)-2,7-diazabicyclo[4.2.0]octane,
2-(5-chlorofuran-2-ylcarbonyl)-2,7-diazabicyclo[4.2.0]octane,
7-(5-chlorofuran-2-ylcarbonyl)-2,7-diazabicyclo[4.2.0]octane,
2-(3-bromofuran-2-ylcarbonyl)-2,7-diazabicyclo[4.2.0]octane,
7-(3-bromofuran-2-ylcarbonyl)-2,7-diazabicyclo[4.2.0]octane,
2-(5-bromofuran-2-ylcarbonyl)-2,7-diazabicyclo[4.2.0]octane,
7-(5-bromofuran-2-ylcarbonyl)-2,7-diazabicyclo[4.2.0]octane,
2-(furan-3-ylcarbonyl)-2,7-diazabicyclo[4.2.0]octane,
7-(furan-3-ylcarbonyl)-2,7-diazabicyclo[4.2.0]octane,
2-(2-methylfuran-3-ylcarbonyl)-2,7-diazabicyclo[4.2.0]octane,
7-(2-methylfuran-3-ylcarbonyl)-2,7-diazabicyclo[4.2.0]octane,
2-(oxazol-2-ylcarbonyl)-2,7-diazabicyclo[4.2.0]octane,
7-(oxazol-2-ylcarbonyl)-2,7-diazabicyclo[4.2.0]octane,
2-(oxazol-5-ylcarbonyl)-2,7-diazabicyclo[4.2.0]octane,
7-(oxazol-5-ylcarbonyl)-2,7-diazabicyclo[4.2.0]octane,
2-(4-methyloxazol-5-ylcarbonyl)-2,7-diazabicyclo[4.2.0]octane,
7-(4-methyloxazol-5-ylcarbonyl)-2,7-diazabicyclo[4.2.0]octane,
2-(isoxazol-3-ylcarbonyl)-2,7-diazabicyclo[4.2.0]octane,
7-(isoxazol-3-ylcarbonyl)-2,7-diazabicyclo[4.2.0]octane,
2-(5-methylisoxazol-3-ylcarbonyl)-2,7-diazabicyclo[4.2.0]octane,
7-(5-methylisoxazol-3-ylcarbonyl)-2,7-diazabicyclo[4.2.0]octane,
2-(isoxazol-4-ylcarbonyl)-2,7-diazabicyclo[4.2.0]octane,
7-(isoxazol-4-ylcarbonyl)-2,7-diazabicyclo[4.2.0]octane,
2-(3-methylisoxazol-4-ylcarbonyl)-2,7-diazabicyclo[4.2.0]octane,
7-(3-methylisoxazol-4-ylcarbonyl)-2,7-diazabicyclo[4.2.0]octane,
2-(5-methylisoxazol-4-ylcarbonyl)-2,7-diazabicyclo[4.2.0]octane,
7-(5-methylisoxazol-4-ylcarbonyl)-2,7-diazabicyclo[4.2.0]octane,
2-(isoxazol-5-ylcarbonyl)-2,7-diazabicyclo[4.2.0]octane,
7-(isoxazol-5-ylcarbonyl)-2,7-diazabicyclo[4.2.0]octane,
2-(3-methylisoxazol-5-ylcarbonyl)-2,7-diazabicyclo[4.2.0]octane,
7-(3-methylisoxazol-5-ylcarbonyl)-2,7-diazabicyclo[4.2.0]octane,
2-(3-bromoisoxazol-5-ylcarbonyl)-2,7-diazabicyclo[4.2.0]octane,
7-(3-bromoisoxazol-5-ylcarbonyl)-2,7-diazabicyclo[4.2.0]octane,
2-(3-methoxyisoxazol-5-ylcarbonyl)-2,7-diazabicyclo[4.2.0]octane,
7-(3-methoxyisoxazol-5-ylcarbonyl)-2,7-diazabicyclo[4.2.0]octane,
2-(pyridin-4-ylcarbonyl)-2,7-diazabicyclo[4.2.0]octane,
7-(pyridin-4-ylcarbonyl)-2,7-diazabicyclo[4.2.0]octane,
3-(furan-2-ylcarbonyl)-3,7-diazabicyclo[4.2.0]octane,
7-(furan-2-ylcarbonyl)-3,7-diazabicyclo[4.2.0]octane,
3-(3-methylfuran-2-ylcarbonyl)-3,7-diazabicyclo[4.2.0]octane,
7-(3-methylfuran-2-ylcarbonyl)-3,7-diazabicyclo[4.2.0]octane,
3-(5-methylfuran-2-ylcarbonyl)-3,7-diazabicyclo[4.2.0]octane,
7-(5-methylfuran-2-ylcarbonyl)-3,7-diazabicyclo[4.2.0]octane,
3-(3-chlorofuran-2-ylcarbonyl)-3,7-diazabicyclo[4.2.0]octane,
7-(3-chlorofuran-2-ylcarbonyl)-3,7-diazabicyclo[4.2.0]octane,
3-(5-chlorofuran-2-ylcarbonyl)-3,7-diazabicyclo[4.2.0]octane,
7-(5-chlorofuran-2-ylcarbonyl)-3,7-diazabicyclo[4.2.0]octane,
3-(3-bromofuran-2-ylcarbonyl)-3,7-diazabicyclo[4.2.0]octane,
7-(3-bromofuran-2-ylcarbonyl)-3,7-diazabicyclo[4.2.0]octane,
3-(5-bromofuran-2-ylcarbonyl)-3,7-diazabicyclo[4.2.0]octane,
7-(5-bromofuran-2-ylcarbonyl)-3,7-diazabicyclo[4.2.0]octane,
3-(furan-3-ylcarbonyl)-3,7-diazabicyclo[4.2.0]octane,
7-(furan-3-ylcarbonyl)-3,7-diazabicyclo[4.2.0]octane,
3-(2-methylfuran-3-ylcarbonyl)-3,7-diazabicyclo[4.2.0]octane,
7-(2-methylfuran-3-ylcarbonyl)-3,7-diazabicyclo[4.2.0]octane,
3-(oxazol-2-ylcarbonyl)-3,7-diazabicyclo[4.2.0]octane,
7-(oxazol-2-ylcarbonyl)-3,7-diazabicyclo[4.2.0]octane,
3-(oxazol-5-ylcarbonyl)-3,7-diazabicyclo[4.2.0]octane,
7-(oxazol-5-ylcarbonyl)-3,7-diazabicyclo[4.2.0]octane,
3-(4-methyloxazol-5-ylcarbonyl)-3,7-diazabicyclo[4.2.0]octane,
7-(4-methyloxazol-5-ylcarbonyl)-3,7-diazabicyclo[4.2.0]octane,
3-(isoxazol-3-ylcarbonyl)-3,7-diazabicyclo[4.2.0]octane,
7-(isoxazol-3-ylcarbonyl)-3,7-diazabicyclo[4.2.0]octane,
3-(5-methylisoxazol-3-ylcarbonyl)-3,7-diazabicyclo[4.2.0]octane,
7-(5-methylisoxazol-3-ylcarbonyl)-3,7-diazabicyclo[4.2.0]octane,
3-(isoxazol-4-ylcarbonyl)-3,7-diazabicyclo[4.2.0]octane,
7-(isoxazol-4-ylcarbonyl)-3,7-diazabicyclo[4.2.0]octane,
3-(3-methylisoxazol-4-ylcarbonyl)-3,7-diazabicyclo[4.2.0]octane,
7-(3-methylisoxazol-4-ylcarbonyl)-3,7-diazabicyclo[4.2.0]octane,
3-(5-methylisoxazol-4-ylcarbonyl)-3,7-diazabicyclo[4.2.0]octane,
7-(5-methylisoxazol-4-ylcarbonyl)-3,7-diazabicyclo[4.2.0]octane,
3-(isoxazol-5-ylcarbonyl)-3,7-diazabicyclo[4.2.0]octane,
7-(isoxazol-5-ylcarbonyl)-3,7-diazabicyclo[4.2.0]octane,
3-(3-methylisoxazol-5-ylcarbonyl)-3,7-diazabicyclo[4.2.0]octane,
7-(3-methylisoxazol-5-ylcarbonyl)-3,7-diazabicyclo[4.2.0]octane,
3-(3-bromoisoxazol-5-ylcarbonyl)-3,7-diazabicyclo[4.2.0]octane,
7-(3-bromoisoxazol-5-ylcarbonyl)-3,7-diazabicyclo[4.2.0]octane,
3-(3-methoxyisoxazol-5-ylcarbonyl)-3,7-diazabicyclo[4.2.0]octane,
7-(3-methoxyisoxazol-5-ylcarbonyl)-3,7-diazabicyclo[4.2.0]octane,
3-(pyridin-4-ylcarbonyl)-3,7-diazabicyclo[4.2.0]octane,
7-(pyridin-4-ylcarbonyl)-3,7-diazabicyclo[4.2.0]octane,
3-(furan-2-ylcarbonyl)-3,8-diazabicyclo[4.2.0]octane,
8-(furan-2-ylcarbonyl)-3,8-diazabicyclo[4.2.0]octane,
3-(3-methylfuran-2-ylcarbonyl)-3,8-diazabicyclo[4.2.0]octane,
8-(3-methylfuran-2-ylcarbonyl)-3,8-diazabicyclo[4.2.0]octane,
3-(5-methylfuran-2-ylcarbonyl)-3,8-diazabicyclo[4.2.0]octane,
8-(5-methylfuran-2-ylcarbonyl)-3,8-diazabicyclo[4.2.0]octane,
3-(3-chlorofuran-2-ylcarbonyl)-3,8-diazabicyclo[4.2.0]octane,
8-(3-chlorofuran-2-ylcarbonyl)-3,8-diazabicyclo[4.2.0]octane,
3-(5-chlorofuran-2-ylcarbonyl)-3,8-diazabicyclo[4.2.0]octane,
8-(5-chlorofuran-2-ylcarbonyl)-3,8-diazabicyclo[4.2.0]octane,
3-(3-bromofuran-2-ylcarbonyl)-3,8-diazabicyclo[4.2.0]octane,
8-(3-bromofuran-2-ylcarbonyl)-3,8-diazabicyclo[4.2.0]octane,
3-(5-bromofuran-2-ylcarbonyl)-3,8-diazabicyclo[4.2.0]octane,
8-(5-bromofuran-2-ylcarbonyl)-3,8-diazabicyclo[4.2.0]octane,
3-(furan-3-ylcarbonyl)-3,8-diazabicyclo[4.2.0]octane,
8-(furan-3-ylcarbonyl)-3,8-diazabicyclo[4.2.0]octane,
3-(2-methylfuran-3-ylcarbonyl)-3,8-diazabicyclo[4.2.0]octane,
8-(2-methylfuran-3-ylcarbonyl)-3,8-diazabicyclo[4.2.0]octane,
3-(oxazol-2-ylcarbonyl)-3,8-diazabicyclo[4.2.0]octane,
8-(oxazol-2-ylcarbonyl)-3,8-diazabicyclo[4.2.0]octane,
3-(oxazol-5-ylcarbonyl)-3,8-diazabicyclo[4.2.0]octane,
8-(oxazol-5-ylcarbonyl)-3,8-diazabicyclo[4.2.0]octane,
3-(4-methyloxazol-5-ylcarbonyl)-3,8-diazabicyclo[4.2.0]octane,
8-(4-methyloxazol-5-ylcarbonyl)-3,8-diazabicyclo[4.2.0]octane,
3-(isoxazol-3-ylcarbonyl)-3,8-diazabicyclo[4.2.0]octane,
8-(isoxazol-3-ylcarbonyl)-3,8-diazabicyclo[4.2.0]octane,
3-(5-methylisoxazol-3-ylcarbonyl)-3,8-diazabicyclo[4.2.0]octane,
8-(5-methylisoxazol-3-ylcarbonyl)-3,8-diazabicyclo[4.2.0]octane,
3-(isoxazol-4-ylcarbonyl)-3,8-diazabicyclo[4.2.0]octane,
8-(isoxazol-4-ylcarbonyl)-3,8-diazabicyclo[4.2.0]octane,
3-(3-methylisoxazol-4-ylcarbonyl)-3,8-diazabicyclo[4.2.0]octane,
8-(3-methylisoxazol-4-ylcarbonyl)-3,8-diazabicyclo[4.2.0]octane,
3-(5-methylisoxazol-4-ylcarbonyl)-3,8-diazabicyclo[4.2.0]octane,
8-(5-methylisoxazol-4-ylcarbonyl)-3,8-diazabicyclo[4.2.0]octane,
3-(isoxazol-5-ylcarbonyl)-3,8-diazabicyclo[4.2.0]octane,
8-(isoxazol-5-ylcarbonyl)-3,8-diazabicyclo[4.2.0]octane,
3-(3-methylisoxazol-5-ylcarbonyl)-3,8-diazabicyclo[4.2.0]octane,
8-(3-methylisoxazol-5-ylcarbonyl)-3,8-diazabicyclo[4.2.0]octane,
3-(3-bromoisoxazol-5-ylcarbonyl)-3,8-diazabicyclo[4.2.0]octane,
8-(3-bromoisoxazol-5-ylcarbonyl)-3,8-diazabicyclo[4.2.0]octane,
3-(3-methoxyisoxazol-5-ylcarbonyl)-3,8-diazabicyclo[4.2.0]octane,
8-(3-methoxyisoxazol-5-ylcarbonyl)-3,8-diazabicyclo[4.2.0]octane,
3-(pyridin-4-ylcarbonyl)-3,8-diazabicyclo[4.2.0]octane,
8-(pyridin-4-ylcarbonyl)-3,8-diazabicyclo[4.2.0]octane,
2-(furan-2-ylcarbonyl)-2,6-diazabicyclo[3.3.0]octane,
2-(3-methylfuran-2-ylcarbonyl)-2,6-diazabicyclo[3.3.0]octane,
2-(5-methylfuran-2-ylcarbonyl)-2,6-diazabicyclo[3.3.0]octane,
2-(3-chlorofuran-2-ylcarbonyl)-2,6-diazabicyclo[3.3.0]octane,
2-(5-chlorofuran-2-ylcarbonyl)-2,6-diazabicyclo[3.3.0]octane,
2-(3-bromofuran-2-ylcarbonyl)-2,6-diazabicyclo[3.3.0]octane,
2-(5-bromofuran-2-ylcarbonyl)-2,6-diazabicyclo[3.3.0]octane,
2-(furan-3-ylcarbonyl)-2,6-diazabicyclo[3.3.0]octane,
2-(2-methylfuran-3-ylcarbonyl)-2,6-diazabicyclo[3.3.0]octane,
2-(oxazol-2-ylcarbonyl)-2,6-diazabicyclo[3.3.0]octane,
2-(oxazol-5-ylcarbonyl)-2,6-diazabicyclo[3.3.0]octane,
2-(4-methyloxazol-5-ylcarbonyl)-2,6-diazabicyclo[3.3.0]octane,
2-(isoxazol-3-ylcarbonyl)-2,6-diazabicyclo[3.3.0]octane,
2-(5-methylisoxazol-3-ylcarbonyl)-2,6-diazabicyclo[3.3.0]octane,
2-(isoxazol-4-ylcarbonyl)-2,6-diazabicyclo[3.3.0]octane,
2-(3-methylisoxazol-4-ylcarbonyl)-2,6-diazabicyclo[3.3.0]octane,
2-(5-methylisoxazol-4-ylcarbonyl)-2,6-diazabicyclo[3.3.0]octane,
2-(isoxazol-5-ylcarbonyl)-2,6-diazabicyclo[3.3.0]octane,
2-(3-methylisoxazol-5-ylcarbonyl)-2,6-diazabicyclo[3.3.0]octane,
2-(3-bromoisoxazol-5-ylcarbonyl)-2,6-diazabicyclo[3.3.0]octane,
2-(3-methoxyisoxazol-5-ylcarbonyl)-2,6-diazabicyclo[3.3.0]octane,
2-(pyridin-4-ylcarbonyl)-2,6-diazabicyclo[3.3.0]octane,
2-(furan-2-ylcarbonyl)-2,7-diazabicyclo[3.3.0]octane,
7-(furan-2-ylcarbonyl)-2,7-diazabicyclo[3.3.0]octane,
2-(3-methylfuran-2-ylcarbonyl)-2,7-diazabicyclo[3.3.0]octane,
7-(3-methylfuran-2-ylcarbonyl)-2,7-diazabicyclo[3.3.0]octane,
2-(5-methylfuran-2-ylcarbonyl)-2,7-diazabicyclo[3.3.0]octane,
7-(5-methylfuran-2-ylcarbonyl)-2,7-diazabicyclo[3.3.0]octane,
2-(3-chlorofuran-2-ylcarbonyl)-2,7-diazabicyclo[3.3.0]octane,
7-(3-chlorofuran-2-ylcarbonyl)-2,7-diazabicyclo[3.3.0]octane,
2-(5-chlorofuran-2-ylcarbonyl)-2,7-diazabicyclo[3.3.0]octane,
7-(5-chlorofuran-2-ylcarbonyl)-2,7-diazabicyclo[3.3.0]octane,
2-(3-bromofuran-2-ylcarbonyl)-2,7-diazabicyclo[3.3.0]octane,
7-(3-bromofuran-2-ylcarbonyl)-2,7-diazabicyclo[3.3.0]octane,
2-(5-bromofuran-2-ylcarbonyl)-2,7-diazabicyclo[3.3.0]octane,
7-(5-bromofuran-2-ylcarbonyl)-2,7-diazabicyclo[3.3.0]octane,
2-(furan-3-ylcarbonyl)-2,7-diazabicyclo[3.3.0]octane,
7-(furan-3-ylcarbonyl)-2,7-diazabicyclo[3.3.0]octane,
2-(2-methylfuran-3-ylcarbonyl)-2,7-diazabicyclo[3.3.0]octane,
7-(2-methylfuran-3-ylcarbonyl)-2,7-diazabicyclo[3.3.0]octane,
2-(oxazol-2-ylcarbonyl)-2,7-diazabicyclo[3.3.0]octane,
7-(oxazol-2-ylcarbonyl)-2,7-diazabicyclo[3.3.0]octane,
2-(oxazol-5-ylcarbonyl)-2,7-diazabicyclo[3.3.0]octane,
7-(oxazol-5-ylcarbonyl)-2,7-diazabicyclo[3.3.0]octane,
2-(4-methyloxazol-5-ylcarbonyl)-2,7-diazabicyclo[3.3.0]octane,
7-(4-methyloxazol-5-ylcarbonyl)-2,7-diazabicyclo[3.3.0]octane,
2-(isoxazol-3-ylcarbonyl)-2,7-diazabicyclo[3.3.0]octane,
7-(isoxazol-3-ylcarbonyl)-2,7-diazabicyclo[3.3.0]octane,
2-(5-methylisoxazol-3-ylcarbonyl)-2,7-diazabicyclo[3.3.0]octane,
7-(5-methylisoxazol-3-ylcarbonyl)-2,7-diazabicyclo[3.3.0]octane,
2-(isoxazol-4-ylcarbonyl)-2,7-diazabicyclo[3.3.0]octane,
7-(isoxazol-4-ylcarbonyl)-2,7-diazabicyclo[3.3.0]octane,
2-(3-methylisoxazol-4-ylcarbonyl)-2,7-diazabicyclo[3.3.0]octane,
7-(3-methylisoxazol-4-ylcarbonyl)-2,7-diazabicyclo[3.3.0]octane,
2-(5-methylisoxazol-4-ylcarbonyl)-2,7-diazabicyclo[3.3.0]octane,
7-(5-methylisoxazol-4-ylcarbonyl)-2,7-diazabicyclo[3.3.0]octane,
2-(isoxazol-5-ylcarbonyl)-2,7-diazabicyclo[3.3.0]octane,
7-(isoxazol-5-ylcarbonyl)-2,7-diazabicyclo[3.3.0]octane,
2-(3-methylisoxazol-5-ylcarbonyl)-2,7-diazabicyclo[3.3.0]octane,
7-(3-methylisoxazol-5-ylcarbonyl)-2,7-diazabicyclo[3.3.0]octane,
2-(3-bromoisoxazol-5-ylcarbonyl)-2,7-diazabicyclo[3.3.0]octane,
7-(3-bromoisoxazol-5-ylcarbonyl)-2,7-diazabicyclo[3.3.0]octane,
2-(3-methoxyisoxazol-5-ylcarbonyl)-2,7-diazabicyclo[3.3.0]octane,
7-(3-methoxyisoxazol-5-ylcarbonyl)-2,7-diazabicyclo[3.3.0]octane,
2-(pyridin-4-ylcarbonyl)-2,7-diazabicyclo[3.3.0]octane,
7-(pyridin-4-ylcarbonyl)-2,7-diazabicyclo[3.3.0]octane,
2-(furan-2-ylcarbonyl)-2,7-diazabicyclo[4.3.0]nonane,
7-(furan-2-ylcarbonyl)-2,7-diazabicyclo[4.3.0]nonane,
2-(3-methylfuran-2-ylcarbonyl)-2,7-diazabicyclo[4.3.0]nonane,
7-(3-methylfuran-2-ylcarbonyl)-2,7-diazabicyclo[4.3.0]nonane,
2-(5-methylfuran-2-ylcarbonyl)-2,7-diazabicyclo[4.3.0]nonane,
7-(5-methylfuran-2-ylcarbonyl)-2,7-diazabicyclo[4.3.0]nonane,
2-(3-chlorofuran-2-ylcarbonyl)-2,7-diazabicyclo[4.3.0]nonane,
7-(3-chlorofuran-2-ylcarbonyl)-2,7-diazabicyclo[4.3.0]nonane,
2-(5-chlorofuran-2-ylcarbonyl)-2,7-diazabicyclo[4.3.0]nonane,
7-(5-chlorofuran-2-ylcarbonyl)-2,7-diazabicyclo[4.3.0]nonane,
2-(3-bromofuran-2-ylcarbonyl)-2,7-diazabicyclo[4.3.0]nonane,
7-(3-bromofuran-2-ylcarbonyl)-2,7-diazabicyclo[4.3.0]nonane,
2-(5-bromofuran-2-ylcarbonyl)-2,7-diazabicyclo[4.3.0]nonane,
7-(5-bromofuran-2-ylcarbonyl)-2,7-diazabicyclo[4.3.0]nonane,
2-(furan-3-ylcarbonyl)-2,7-diazabicyclo[4.3.0]nonane,
7-(furan-3-ylcarbonyl)-2,7-diazabicyclo[4.3.0]nonane,
2-(2-methylfuran-3-ylcarbonyl)-2,7-diazabicyclo[4.3.0]nonane,
7-(2-methylfuran-3-ylcarbonyl)-2,7-diazabicyclo[4.3.0]nonane,
2-(oxazol-2-ylcarbonyl)-2,7-diazabicyclo[4.3.0]nonane,
7-(oxazol-2-ylcarbonyl)-2,7-diazabicyclo[4.3.0]nonane,
2-(oxazol-5-ylcarbonyl)-2,7-diazabicyclo[4.3.0]nonane,
7-(oxazol-5-ylcarbonyl)-2,7-diazabicyclo[4.3.0]nonane,
2-(4-methyloxazol-5-ylcarbonyl)-2,7-diazabicyclo[4.3.0]nonane,
7-(4-methyloxazol-5-ylcarbonyl)-2,7-diazabicyclo[4.3.0]nonane,
2-(isoxazol-3-ylcarbonyl)-2,7-diazabicyclo[4.3.0]nonane,
7-(isoxazol-3-ylcarbonyl)-2,7-diazabicyclo[4.3.0]nonane,
2-(5-methylisoxazol-3-ylcarbonyl)-2,7-diazabicyclo[4.3.0]nonane,
7-(5-methylisoxazol-3-ylcarbonyl)-2,7-diazabicyclo[4.3.0]nonane,
2-(isoxazol-4-ylcarbonyl)-2,7-diazabicyclo[4.3.0]nonane,
7-(isoxazol-4-ylcarbonyl)-2,7-diazabicyclo[4.3.0]nonane,
2-(3-methylisoxazol-4-ylcarbonyl)-2,7-diazabicyclo[4.3.0]nonane,
7-(3-methylisoxazol-4-ylcarbonyl)-2,7-diazabicyclo[4.3.0]nonane,
2-(5-methylisoxazol-4-ylcarbonyl)-2,7-diazabicyclo[4.3.0]nonane,
7-(5-methylisoxazol-4-ylcarbonyl)-2,7-diazabicyclo[4.3.0]nonane,
2-(isoxazol-5-ylcarbonyl)-2,7-diazabicyclo[4.3.0]nonane,
7-(isoxazol-5-ylcarbonyl)-2,7-diazabicyclo[4.3.0]nonane,
2-(3-methylisoxazol-5-ylcarbonyl)-2,7-diazabicyclo[4.3.0]nonane,
7-(3-methylisoxazol-5-ylcarbonyl)-2,7-diazabicyclo[4.3.0]nonane,
2-(3-bromoisoxazol-5-ylcarbonyl)-2,7-diazabicyclo[4.3.0]nonane,
7-(3-bromoisoxazol-5-ylcarbonyl)-2,7-diazabicyclo[4.3.0]nonane,
2-(3-methoxyisoxazol-5-ylcarbonyl)-2,7-diazabicyclo[4.3.0]nonane,
7-(3-methoxyisoxazol-5-ylcarbonyl)-2,7-diazabicyclo[4.3.0]nonane,
2-(pyridin-4-ylcarbonyl)-2,7-diazabicyclo[4.3.0]nonane,
7-(pyridin-4-ylcarbonyl)-2,7-diazabicyclo[4.3.0]nonane,
2-(furan-2-ylcarbonyl)-2,8-diazabicyclo[4.3.0]nonane,
8-(furan-2-ylcarbonyl)-2,8-diazabicyclo[4.3.0]nonane,
2-(3-methylfuran-2-ylcarbonyl)-2,8-diazabicyclo[4.3.0]nonane,
8-(3-methylfuran-2-ylcarbonyl)-2,8-diazabicyclo[4.3.0]nonane,
2-(5-methylfuran-2-ylcarbonyl)-2,8-diazabicyclo[4.3.0]nonane,
8-(5-methylfuran-2-ylcarbonyl)-2,8-diazabicyclo[4.3.0]nonane,
2-(3-chlorofuran-2-ylcarbonyl)-2,8-diazabicyclo[4.3.0]nonane,
8-(3-chlorofuran-2-ylcarbonyl)-2,8-diazabicyclo[4.3.0]nonane,
2-(5-chlorofuran-2-ylcarbonyl)-2,8-diazabicyclo[4.3.0]nonane,
8-(5-chlorofuran-2-ylcarbonyl)-2,8-diazabicyclo[4.3.0]nonane,
2-(3-bromofuran-2-ylcarbonyl)-2,8-diazabicyclo[4.3.0]nonane,
8-(3-bromofuran-2-ylcarbonyl)-2,8-diazabicyclo[4.3.0]nonane,
2-(5-bromofuran-2-ylcarbonyl)-2,8-diazabicyclo[4.3.0]nonane,
8-(5-bromofuran-2-ylcarbonyl)-2,8-diazabicyclo[4.3.0]nonane,
2-(furan-3-ylcarbonyl)-2,8-diazabicyclo[4.3.0]nonane,
8-(furan-3-ylcarbonyl)-2,8-diazabicyclo[4.3.0]nonane,
2-(2-methylfuran-3-ylcarbonyl)-2,8-diazabicyclo[4.3.0]nonane,
8-(2-methylfuran-3-ylcarbonyl)-2,8-diazabicyclo[4.3.0]nonane,
2-(oxazol-2-ylcarbonyl)-2,8-diazabicyclo[4.3.0]nonane,
8-(oxazol-2-ylcarbonyl)-2,8-diazabicyclo[4.3.0]nonane,
2-(oxazol-5-ylcarbonyl)-2,8-diazabicyclo[4.3.0]nonane,
8-(oxazol-5-ylcarbonyl)-2,8-diazabicyclo[4.3.0]nonane,
2-(4-methyloxazol-5-ylcarbonyl)-2,8-diazabicyclo[4.3.0]nonane,
8-(4-methyloxazol-5-ylcarbonyl)-2,8-diazabicyclo[4.3.0]nonane,
2-(isoxazol-3-ylcarbonyl)-2,8-diazabicyclo[4.3.0]nonane,
8-(isoxazol-3-ylcarbonyl)-2,8-diazabicyclo[4.3.0]nonane,
2-(5-methylisoxazol-3-ylcarbonyl)-2,8-diazabicyclo[4.3.0]nonane,
8-(5-methylisoxazol-3-ylcarbonyl)-2,8-diazabicyclo[4.3.0]nonane,
2-(isoxazol-4-ylcarbonyl)-2,8-diazabicyclo[4.3.0]nonane,
8-(isoxazol-4-ylcarbonyl)-2,8-diazabicyclo[4.3.0]nonane,
2-(3-methylisoxazol-4-ylcarbonyl)-2,8-diazabicyclo[4.3.0]nonane,
8-(3-methylisoxazol-4-ylcarbonyl)-2,8-diazabicyclo[4.3.0]nonane,
2-(5-methylisoxazol-4-ylcarbonyl)-2,8-diazabicyclo[4.3.0]nonane,
8-(5-methylisoxazol-4-ylcarbonyl)-2,8-diazabicyclo[4.3.0]nonane,
2-(isoxazol-5-ylcarbonyl)-2,8-diazabicyclo[4.3.0]nonane,
8-(isoxazol-5-ylcarbonyl)-2,8-diazabicyclo[4.3.0]nonane,
2-(3-methylisoxazol-5-ylcarbonyl)-2,8-diazabicyclo[4.3.0]nonane,
8-(3-methylisoxazol-5-ylcarbonyl)-2,8-diazabicyclo[4.3.0]nonane,
2-(3-bromoisoxazol-5-ylcarbonyl)-2,8-diazabicyclo[4.3.0]nonane,
8-(3-bromoisoxazol-5-ylcarbonyl)-2,8-diazabicyclo[4.3.0]nonane,
2-(3-methoxyisoxazol-5-ylcarbonyl)-2,8-diazabicyclo[4.3.0]nonane,
8-(3-methoxyisoxazol-5-ylcarbonyl)-2,8-diazabicyclo[4.3.0]nonane,
2-(pyridin-4-ylcarbonyl)-2,8-diazabicyclo[4.3.0]nonane,
8-(pyridin-4-ylcarbonyl)-2,8-diazabicyclo[4.3.0]nonane,
3-(furan-2-ylcarbonyl)-3,7-diazabicyclo[4.3.0]nonane,
7-(furan-2-ylcarbonyl)-3,7-diazabicyclo[4.3.0]nonane,
3-(3-methylfuran-2-ylcarbonyl)-3,7-diazabicyclo[4.3.0]nonane,
7-(3-methylfuran-2-ylcarbonyl)-3,7-diazabicyclo[4.3.0]nonane,
3-(5-methylfuran-2-ylcarbonyl)-3,7-diazabicyclo[4.3.0]nonane,
7-(5-methylfuran-2-ylcarbonyl)-3,7-diazabicyclo[4.3.0]nonane,
3-(3-chlorofuran-2-ylcarbonyl)-3,7-diazabicyclo[4.3.0]nonane,
7-(3-chlorofuran-2-ylcarbonyl)-3,7-diazabicyclo[4.3.0]nonane,
3-(5-chlorofuran-2-ylcarbonyl)-3,7-diazabicyclo[4.3.0]nonane,
7-(5-chlorofuran-2-ylcarbonyl)-3,7-diazabicyclo[4.3.0]nonane,
3-(3-bromofuran-2-ylcarbonyl)-3,7-diazabicyclo[4.3.0]nonane,
7-(3-bromofuran-2-ylcarbonyl)-3,7-diazabicyclo[4.3.0]nonane,
3-(5-bromofuran-2-ylcarbonyl)-3,7-diazabicyclo[4.3.0]nonane,
7-(5-bromofuran-2-ylcarbonyl)-3,7-diazabicyclo[4.3.0]nonane,
3-(furan-3-ylcarbonyl)-3,7-diazabicyclo[4.3.0]nonane,
7-(furan-3-ylcarbonyl)-3,7-diazabicyclo[4.3.0]nonane,
3-(2-methylfuran-3-ylcarbonyl)-3,7-diazabicyclo[4.3.0]nonane,
7-(2-methylfuran-3-ylcarbonyl)-3,7-diazabicyclo[4.3.0]nonane,
3-(oxazol-2-ylcarbonyl)-3,7-diazabicyclo[4.3.0]nonane,
7-(oxazol-2-ylcarbonyl)-3,7-diazabicyclo[4.3.0]nonane,
3-(oxazol-5-ylcarbonyl)-3,7-diazabicyclo[4.3.0]nonane,
7-(oxazol-5-ylcarbonyl)-3,7-diazabicyclo[4.3.0]nonane,
3-(4-methyloxazol-5-ylcarbonyl)-3,7-diazabicyclo[4.3.0]nonane,
7-(4-methyloxazol-5-ylcarbonyl)-3,7-diazabicyclo[4.3.0]nonane,
3-(isoxazol-3-ylcarbonyl)-3,7-diazabicyclo[4.3.0]nonane,
7-(isoxazol-3-ylcarbonyl)-3,7-diazabicyclo[4.3.0]nonane,
3-(5-methylisoxazol-3-ylcarbonyl)-3,7-diazabicyclo[4.3.0]nonane,
7-(5-methylisoxazol-3-ylcarbonyl)-3,7-diazabicyclo[4.3.0]nonane,
3-(isoxazol-4-ylcarbonyl)-3,7-diazabicyclo[4.3.0]nonane,
7-(isoxazol-4-ylcarbonyl)-3,7-diazabicyclo[4.3.0]nonane,
3-(3-methylisoxazol-4-ylcarbonyl)-3,7-diazabicyclo[4.3.0]nonane,
7-(3-methylisoxazol-4-ylcarbonyl)-3,7-diazabicyclo[4.3.0]nonane,
3-(5-methylisoxazol-4-ylcarbonyl)-3,7-diazabicyclo[4.3.0]nonane,
7-(5-methylisoxazol-4-ylcarbonyl)-3,7-diazabicyclo[4.3.0]nonane,
3-(isoxazol-5-ylcarbonyl)-3,7-diazabicyclo[4.3.0]nonane,
7-(isoxazol-5-ylcarbonyl)-3,7-diazabicyclo[4.3.0]nonane,
3-(3-methylisoxazol-5-ylcarbonyl)-3,7-diazabicyclo[4.3.0]nonane,
7-(3-methylisoxazol-5-ylcarbonyl)-3,7-diazabicyclo[4.3.0]nonane,
3-(3-bromoisoxazol-5-ylcarbonyl)-3,7-diazabicyclo[4.3.0]nonane,
7-(3-bromoisoxazol-5-ylcarbonyl)-3,7-diazabicyclo[4.3.0]nonane,
3-(3-methoxyisoxazol-5-ylcarbonyl)-3,7-diazabicyclo[4.3.0]nonane,
7-(3-methoxyisoxazol-5-ylcarbonyl)-3,7-diazabicyclo[4.3.0]nonane,
3-(pyridin-4-ylcarbonyl)-3,7-diazabicyclo[4.3.0]nonane,
7-(pyridin-4-ylcarbonyl)-3,7-diazabicyclo[4.3.0]nonane,
3-(furan-2-ylcarbonyl)-3,8-diazabicyclo[4.3.0]nonane,
8-(furan-2-ylcarbonyl)-3,8-diazabicyclo[4.3.0]nonane,
3-(3-methylfuran-2-ylcarbonyl)-3,8-diazabicyclo[4.3.0]nonane,
8-(3-methylfuran-2-ylcarbonyl)-3,8-diazabicyclo[4.3.0]nonane,
3-(5-methylfuran-2-ylcarbonyl)-3,8-diazabicyclo[4.3.0]nonane,
8-(5-methylfuran-2-ylcarbonyl)-3,8-diazabicyclo[4.3.0]nonane,
3-(3-chlorofuran-2-ylcarbonyl)-3,8-diazabicyclo[4.3.0]nonane,
8-(3-chlorofuran-2-ylcarbonyl)-3,8-diazabicyclo[4.3.0]nonane,
3-(5-chlorofuran-2-ylcarbonyl)-3,8-diazabicyclo[4.3.0]nonane,
8-(5-chlorofuran-2-ylcarbonyl)-3,8-diazabicyclo[4.3.0]nonane,
3-(3-bromofuran-2-ylcarbonyl)-3,8-diazabicyclo[4.3.0]nonane,
8-(3-bromofuran-2-ylcarbonyl)-3,8-diazabicyclo[4.3.0]nonane,
3-(5-bromofuran-2-ylcarbonyl)-3,8-diazabicyclo[4.3.0]nonane,
8-(5-bromofuran-2-ylcarbonyl)-3,8-diazabicyclo[4.3.0]nonane,
3-(furan-3-ylcarbonyl)-3,8-diazabicyclo[4.3.0]nonane,
8-(furan-3-ylcarbonyl)-3,8-diazabicyclo[4.3.0]nonane,
3-(2-methylfuran-3-ylcarbonyl)-3,8-diazabicyclo[4.3.0]nonane,
8-(2-methylfuran-3-ylcarbonyl)-3,8-diazabicyclo[4.3.0]nonane,
3-(oxazol-2-ylcarbonyl)-3,8-diazabicyclo[4.3.0]nonane,
8-(oxazol-2-ylcarbonyl)-3,8-diazabicyclo[4.3.0]nonane,
3-(oxazol-5-ylcarbonyl)-3,8-diazabicyclo[4.3.0]nonane,
8-(oxazol-5-ylcarbonyl)-3,8-diazabicyclo[4.3.0]nonane,
3-(4-methyloxazol-5-ylcarbonyl)-3,8-diazabicyclo[4.3.0]nonane,
8-(4-methyloxazol-5-ylcarbonyl)-3,8-diazabicyclo[4.3.0]nonane,
3-(isoxazol-3-ylcarbonyl)-3,8-diazabicyclo[4.3.0]nonane,
8-(isoxazol-3-ylcarbonyl)-3,8-diazabicyclo[4.3.0]nonane,
3-(5-methylisoxazol-3-ylcarbonyl)-3,8-diazabicyclo[4.3.0]nonane,
8-(5-methylisoxazol-3-ylcarbonyl)-3,8-diazabicyclo[4.3.0]nonane,
3-(isoxazol-4-ylcarbonyl)-3,8-diazabicyclo[4.3.0]nonane,
8-(isoxazol-4-ylcarbonyl)-3,8-diazabicyclo[4.3.0]nonane,
3-(3-methylisoxazol-4-ylcarbonyl)-3,8-diazabicyclo[4.3.0]nonane,
8-(3-methylisoxazol-4-ylcarbonyl)-3,8-diazabicyclo[4.3.0]nonane,
3-(5-methylisoxazol-4-ylcarbonyl)-3,8-diazabicyclo[4.3.0]nonane,
8-(5-methylisoxazol-4-ylcarbonyl)-3,8-diazabicyclo[4.3.0]nonane,
3-(isoxazol-5-ylcarbonyl)-3,8-diazabicyclo[4.3.0]nonane,
8-(isoxazol-5-ylcarbonyl)-3,8-diazabicyclo[4.3.0]nonane,
3-(3-methylisoxazol-5-ylcarbonyl)-3,8-diazabicyclo[4.3.0]nonane,
8-(3-methylisoxazol-5-ylcarbonyl)-3,8-diazabicyclo[4.3.0]nonane,
3-(3-bromoisoxazol-5-ylcarbonyl)-3,8-diazabicyclo[4.3.0]nonane,
8-(3-bromoisoxazol-5-ylcarbonyl)-3,8-diazabicyclo[4.3.0]nonane,
3-(3-methoxyisoxazol-5-ylcarbonyl)-3,8-diazabicyclo[4.3.0]nonane,
8-(3-methoxyisoxazol-5-ylcarbonyl)-3,8-diazabicyclo[4.3.0]nonane,
3-(pyridin-4-ylcarbonyl)-3,8-diazabicyclo[4.3.0]nonane,
8-(pyridin-4-ylcarbonyl)-3,8-diazabicyclo[4.3.0]nonane,
3-(furan-2-ylcarbonyl)-3,9-diazabicyclo[4.3.0]nonane,
9-(furan-2-ylcarbonyl)-3,9-diazabicyclo[4.3.0]nonane,
3-(3-methylfuran-2-ylcarbonyl)-3,9-diazabicyclo[4.3.0]nonane,
9-(3-methylfuran-2-ylcarbonyl)-3,9-diazabicyclo[4.3.0]nonane,
3-(5-methylfuran-2-ylcarbonyl)-3,9-diazabicyclo[4.3.0]nonane,
9-(5-methylfuran-2-ylcarbonyl)-3,9-diazabicyclo[4.3.0]nonane,
3-(3-chlorofuran-2-ylcarbonyl)-3,9-diazabicyclo[4.3.0]nonane,
9-(3-chlorofuran-2-ylcarbonyl)-3,9-diazabicyclo[4.3.0]nonane,
3-(5-chlorofuran-2-ylcarbonyl)-3,9-diazabicyclo[4.3.0]nonane,
9-(5-chlorofuran-2-ylcarbonyl)-3,9-diazabicyclo[4.3.0]nonane,
3-(3-bromofuran-2-ylcarbonyl)-3,9-diazabicyclo[4.3.0]nonane,
9-(3-bromofuran-2-ylcarbonyl)-3,9-diazabicyclo[4.3.0]nonane,
3-(5-bromofuran-2-ylcarbonyl)-3,9-diazabicyclo[4.3.0]nonane,
9-(5-bromofuran-2-ylcarbonyl)-3,9-diazabicyclo[4.3.0]nonane,
3-(furan-3-ylcarbonyl)-3,9-diazabicyclo[4.3.0]nonane,
9-(furan-3-ylcarbonyl)-3,9-diazabicyclo[4.3.0]nonane,
3-(2-methylfuran-3-ylcarbonyl)-3,9-diazabicyclo[4.3.0]nonane,
9-(2-methylfuran-3-ylcarbonyl)-3,9-diazabicyclo[4.3.0]nonane,
3-(oxazol-2-ylcarbonyl)-3,9-diazabicyclo[4.3.0]nonane,
9-(oxazol-2-ylcarbonyl)-3,9-diazabicyclo[4.3.0]nonane,
3-(oxazol-5-ylcarbonyl)-3,9-diazabicyclo[4.3.0]nonane,
9-(oxazol-5-ylcarbonyl)-3,9-diazabicyclo[4.3.0]nonane,
3-(4-methyloxazol-5-ylcarbonyl)-3,9-diazabicyclo[4.3.0]nonane,
9-(4-methyloxazol-5-ylcarbonyl)-3,9-diazabicyclo[4.3.0]nonane,
3-(isoxazol-3-ylcarbonyl)-3,9-diazabicyclo[4.3.0]nonane,
9-(isoxazol-3-ylcarbonyl)-3,9-diazabicyclo[4.3.0]nonane,
3-(5-methylisoxazol-3-ylcarbonyl)-3,9-diazabicyclo[4.3.0]nonane,
9-(5-methylisoxazol-3-ylcarbonyl)-3,9-diazabicyclo[4.3.0]nonane,
3-(isoxazol-4-ylcarbonyl)-3,9-diazabicyclo[4.3.0]nonane,
9-(isoxazol-4-ylcarbonyl)-3,9-diazabicyclo[4.3.0]nonane,
3-(3-methylisoxazol-4-ylcarbonyl)-3,9-diazabicyclo[4.3.0]nonane,
9-(3-methylisoxazol-4-ylcarbonyl)-3,9-diazabicyclo[4.3.0]nonane,
3-(5-methylisoxazol-4-ylcarbonyl)-3,9-diazabicyclo[4.3.0]nonane,
9-(5-methylisoxazol-4-ylcarbonyl)-3,9-diazabicyclo[4.3.0]nonane,
3-(isoxazol-5-ylcarbonyl)-3,9-diazabicyclo[4.3.0]nonane,
9-(isoxazol-5-ylcarbonyl)-3,9-diazabicyclo[4.3.0]nonane,
3-(3-methylisoxazol-5-ylcarbonyl)-3,9-diazabicyclo[4.3.0]nonane,
9-(3-methylisoxazol-5-ylcarbonyl)-3,9-diazabicyclo[4.3.0]nonane,
3-(3-bromoisoxazol-5-ylcarbonyl)-3,9-diazabicyclo[4.3.0]nonane,
9-(3-bromoisoxazol-5-ylcarbonyl)-3,9-diazabicyclo[4.3.0]nonane,
3-(3-methoxyisoxazol-5-ylcarbonyl)-3,9-diazabicyclo[4.3.0]nonane,
9-(3-methoxyisoxazol-5-ylcarbonyl)-3,9-diazabicyclo[4.3.0]nonane,
3-(pyridin-4-ylcarbonyl)-3,9-diazabicyclo[4.3.0]nonane,
9-(pyridin-4-ylcarbonyl)-3,9-diazabicyclo[4.3.0]nonane,
2-(furan-2-ylcarbonyl)-2,6-diazabicyclo[3.2.1]octane,
6-(furan-2-ylcarbonyl)-2,6-diazabicyclo[3.2.1]octane,
2-(3-methylfuran-2-ylcarbonyl)-2,6-diazabicyclo[3.2.1]octane,
6-(3-methylfuran-2-ylcarbonyl)-2,6-diazabicyclo[3.2.1]octane,
2-(5-methylfuran-2-ylcarbonyl)-2,6-diazabicyclo[3.2.1]octane,
6-(5-methylfuran-2-ylcarbonyl)-2,6-diazabicyclo[3.2.1]octane,
2-(3-chlorofuran-2-ylcarbonyl)-2,6-diazabicyclo[3.2.1]octane,
6-(3-chlorofuran-2-ylcarbonyl)-2,6-diazabicyclo[3.2.1]octane,
2-(5-chlorofuran-2-ylcarbonyl)-2,6-diazabicyclo[3.2.1]octane,
6-(5-chlorofuran-2-ylcarbonyl)-2,6-diazabicyclo[3.2.1]octane,
2-(3-bromofuran-2-ylcarbonyl)-2,6-diazabicyclo[3.2.1]octane,
6-(3-bromofuran-2-ylcarbonyl)-2,6-diazabicyclo[3.2.1]octane,
2-(5-bromofuran-2-ylcarbonyl)-2,6-diazabicyclo[3.2.1]octane,
6-(5-bromofuran-2-ylcarbonyl)-2,6-diazabicyclo[3.2.1]octane,
2-(furan-3-ylcarbonyl)-2,6-diazabicyclo[3.2.1]octane,
6-(furan-3-ylcarbonyl)-2,6-diazabicyclo[3.2.1]octane,
2-(2-methylfuran-3-ylcarbonyl)-2,6-diazabicyclo[3.2.1]octane,
6-(2-methylfuran-3-ylcarbonyl)-2,6-diazabicyclo[3.2.1]octane,
2-(oxazol-2-ylcarbonyl)-2,6-diazabicyclo[3.2.1]octane,
6-(oxazol-2-ylcarbonyl)-2,6-diazabicyclo[3.2.1]octane,
2-(oxazol-5-ylcarbonyl)-2,6-diazabicyclo[3.2.1]octane,
6-(oxazol-5-ylcarbonyl)-2,6-diazabicyclo[3.2.1]octane,
2-(4-methyloxazol-5-ylcarbonyl)-2,6-diazabicyclo[3.2.1]octane,
6-(4-methyloxazol-5-ylcarbonyl)-2,6-diazabicyclo[3.2.1]octane,
2-(isoxazol-3-ylcarbonyl)-2,6-diazabicyclo[3.2.1]octane,
6-(isoxazol-3-ylcarbonyl)-2,6-diazabicyclo[3.2.1]octane,
2-(5-methylisoxazol-3-ylcarbonyl)-2,6-diazabicyclo[3.2.1]octane,
6-(5-methylisoxazol-3-ylcarbonyl)-2,6-diazabicyclo[3.2.1]octane,
2-(isoxazol-4-ylcarbonyl)-2,6-diazabicyclo[3.2.1]octane,
6-(isoxazol-4-ylcarbonyl)-2,6-diazabicyclo[3.2.1]octane,
2-(3-methylisoxazol-4-ylcarbonyl)-2,6-diazabicyclo[3.2.1]octane,
6-(3-methylisoxazol-4-ylcarbonyl)-2,6-diazabicyclo[3.2.1]octane,
2-(5-methylisoxazol-4-ylcarbonyl)-2,6-diazabicyclo[3.2.1]octane,
6-(5-methylisoxazol-4-ylcarbonyl)-2,6-diazabicyclo[3.2.1]octane,
2-(isoxazol-5-ylcarbonyl)-2,6-diazabicyclo[3.2.1]octane,
6-(isoxazol-5-ylcarbonyl)-2,6-diazabicyclo[3.2.1]octane,
2-(3-methylisoxazol-5-ylcarbonyl)-2,6-diazabicyclo[3.2.1]octane,
6-(3-methylisoxazol-5-ylcarbonyl)-2,6-diazabicyclo[3.2.1]octane,
2-(3-bromoisoxazol-5-ylcarbonyl)-2,6-diazabicyclo[3.2.1]octane,
6-(3-bromoisoxazol-5-ylcarbonyl)-2,6-diazabicyclo[3.2.1]octane,
2-(3-methoxyisoxazol-5-ylcarbonyl)-2,6-diazabicyclo[3.2.1]octane,
6-(3-methoxyisoxazol-5-ylcarbonyl)-2,6-diazabicyclo[3.2.1]octane,
2-(pyridin-4-ylcarbonyl)-2,6-diazabicyclo[3.2.1]octane,
6-(pyridin-4-ylcarbonyl)-2,6-diazabicyclo[3.2.1]octane,
3-(furan-2-ylcarbonyl)-3,6-diazabicyclo[3.2.1]octane,
6-(furan-2-ylcarbonyl)-3,6-diazabicyclo[3.2.1]octane,
3-(3-methylfuran-2-ylcarbonyl)-3,6-diazabicyclo[3.2.1]octane,
6-(3-methylfuran-2-ylcarbonyl)-3,6-diazabicyclo[3.2.1]octane,
3-(5-methylfuran-2-ylcarbonyl)-3,6-diazabicyclo[3.2.1]octane,
6-(5-methylfuran-2-ylcarbonyl)-3,6-diazabicyclo[3.2.1]octane,
3-(3-chlorofuran-2-ylcarbonyl)-3,6-diazabicyclo[3.2.1]octane,
6-(3-chlorofuran-2-ylcarbonyl)-3,6-diazabicyclo[3.2.1]octane,
3-(5-chlorofuran-2-ylcarbonyl)-3,6-diazabicyclo[3.2.1]octane,
6-(5-chlorofuran-2-ylcarbonyl)-3,6-diazabicyclo[3.2.1]octane,
3-(3-bromofuran-2-ylcarbonyl)-3,6-diazabicyclo[3.2.1]octane,
6-(3-bromofuran-2-ylcarbonyl)-3,6-diazabicyclo[3.2.1]octane,
3-(5-bromofuran-2-ylcarbonyl)-3,6-diazabicyclo[3.2.1]octane,
6-(5-bromofuran-2-ylcarbonyl)-3,6-diazabicyclo[3.2.1]octane,
3-(furan-3-ylcarbonyl)-3,6-diazabicyclo[3.2.1]octane,
6-(furan-3-ylcarbonyl)-3,6-diazabicyclo[3.2.1]octane,
3-(2-methylfuran-3-ylcarbonyl)-3,6-diazabicyclo[3.2.1]octane,
6-(2-methylfuran-3-ylcarbonyl)-3,6-diazabicyclo[3.2.1]octane,
3-(oxazol-2-ylcarbonyl)-3,6-diazabicyclo[3.2.1]octane,
6-(oxazol-2-ylcarbonyl)-3,6-diazabicyclo[3.2.1]octane,
3-(oxazol-5-ylcarbonyl)-3,6-diazabicyclo[3.2.1]octane,
6-(oxazol-5-ylcarbonyl)-3,6-diazabicyclo[3.2.1]octane,
3-(4-methyloxazol-5-ylcarbonyl)-3,6-diazabicyclo[3.2.1]octane,
6-(4-methyloxazol-5-ylcarbonyl)-3,6-diazabicyclo[3.2.1]octane,
3-(isoxazol-3-ylcarbonyl)-3,6-diazabicyclo[3.2.1]octane,
6-(isoxazol-3-ylcarbonyl)-3,6-diazabicyclo[3.2.1]octane,
3-(5-methylisoxazol-3-ylcarbonyl)-3,6-diazabicyclo[3.2.1]octane,
6-(5-methylisoxazol-3-ylcarbonyl)-3,6-diazabicyclo[3.2.1]octane,
3-(isoxazol-4-ylcarbonyl)-3,6-diazabicyclo[3.2.1]octane,
6-(isoxazol-4-ylcarbonyl)-3,6-diazabicyclo[3.2.1]octane,
3-(3-methylisoxazol-4-ylcarbonyl)-3,6-diazabicyclo[3.2.1]octane,
6-(3-methylisoxazol-4-ylcarbonyl)-3,6-diazabicyclo[3.2.1]octane,
3-(5-methylisoxazol-4-ylcarbonyl)-3,6-diazabicyclo[3.2.1]octane,
6-(5-methylisoxazol-4-ylcarbonyl)-3,6-diazabicyclo[3.2.1]octane,
3-(isoxazol-5-ylcarbonyl)-3,6-diazabicyclo[3.2.1]octane,
6-(isoxazol-5-ylcarbonyl)-3,6-diazabicyclo[3.2.1]octane,
3-(3-methylisoxazol-5-ylcarbonyl)-3,6-diazabicyclo[3.2.1]octane,
6-(3-methylisoxazol-5-ylcarbonyl)-3,6-diazabicyclo[3.2.1]octane,
3-(3-bromoisoxazol-5-ylcarbonyl)-3,6-diazabicyclo[3.2.1]octane,
6-(3-bromoisoxazol-5-ylcarbonyl)-3,6-diazabicyclo[3.2.1]octane,
3-(3-methoxyisoxazol-5-ylcarbonyl)-3,6-diazabicyclo[3.2.1]octane,
6-(3-methoxyisoxazol-5-ylcarbonyl)-3,6-diazabicyclo[3.2.1]octane, 3-(pyridin-4-ylcarbonyl)-3,6-diazabicyclo[3.2.1]octane, and 6-(pyridin-4-ylcarbonyl)-3,6-diazabicyclo[3.2.1]octane,
   or a pharmaceutically acceptable salt thereof.

In one embodiment, the compound is in isolated form.

One aspect of the present invention includes a method for treatment or prevention of central nervous system disorders, comprising administering a salt of such a compound.

One aspect of the present invention includes a method for treatment or prevention of central nervous system disorders, comprising administering such a compound.

In one embodiment, the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, cognitive dysfunction in schizophrenia, schizophreniform disorder and schizoaffective disorder.

In still further an embodiment, the disorder is selected from the group consisting of mild to moderate dementia of the Alzheimer's type, attention deficit disorder, mild cognitive impairment and age associated memory impairment.

One aspect of the present invention includes (1S,5S)-3-(5-bromofuran-2-ylcarbonyl)-3,6-diazabicyclo[3.2.1]octane or a pharmaceutically acceptable salt thereof.

The present invention includes all combinations of aspects and embodiments.

The present invention relates to amide compounds which can be formed from certain heteroarylcarboxylic acids and certain diazabicycloalkanes. These amide compounds (heteroarylcarboxamides) bind with high affinity to neuronal nicotinic receptors of the α4β2 subtype, found in the central nervous system (CNS), and exhibit selectivity for the α4β2 subtype over the α7 NNR subtype, also found in the CNS.

The present invention also relates to pharmaceutically acceptable salts prepared from these amide compounds and the pharmaceutical compositions thereof, which can be used for treating and/or preventing a wide variety of conditions or disorders, and particularly those disorders characterized by dysfunction of nicotinic cholinergic neurotransmission or the degeneration of the nicotinic cholinergic neurons.

The present invention also relates to methods for treating or preventing disorders, such as CNS disorders and also for treating certain conditions, namely, alleviating pain and inflammation. The methods involve administering to a subject a therapeutically effective amount of the compounds, including salts, or pharmaceutical compositions including such compounds. Further provided is a method for treatment of disorders selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, cognitive dysfunction in schizophrenia, schizophreniform disorder, and schizoaffective disorder. Even further provided is a method for treatment of disorders selected from the group consisting of the treatment of mild to moderate dementia of the Alzheimer's type, attention deficit disorder, mild cognitive impairment, age associated memory impairment, and cognitive dysfunction in schizophrenia.

The pharmaceutical compositions incorporate a compound of the present invention which, when employed in effective amounts, interacts with relevant nicotinic receptor sites of a subject, and hence acts as a therapeutic agent to treat and prevent a wide variety of conditions and disorders. The pharmaceutical compositions provide therapeutic benefit to individuals suffering from such disorders and exhibiting clinical manifestations of such disorders, in that the compounds within those compositions, when employed in effective amounts, can (i) exhibit nicotinic pharmacology and affect relevant nicotinic receptors sites (e.g., act as a pharmacological agonist to activate nicotinic receptors), and/or (ii) elicit neurotransmitter secretion, and hence prevent and suppress the symptoms associated with those diseases. In addition, the compounds have the potential to (i) increase the number of nicotinic cholinergic receptors of the brain of the patient, (ii) exhibit neuroprotective effects, and/or (iii) when employed in effective amounts, to not cause appreciable adverse side effects (e.g., significant increases in blood pressure and heart rate, significant negative effects upon the gastro-intestinal tract, and significant effects upon skeletal muscle).

The foregoing and other aspects of the present invention are explained in detail in the detailed description and examples set forth below.

### BRIEF DESCRIPTION OF THE FIGURES

FIG. 1 is a chart showing the results of a study on object recognition in rats treated orally with (1S,5S)-3-(5-bromofuran-2-ylcarbonyl)-3,6-diazabicyclo[3.2.1]octane. The results are shown as a function of recognition index (%) versus dose (mg/kg).

### DETAILED DESCRIPTION

The subtype selective compounds, pharmaceutical compositions including these compounds, methods of preparing the compounds, and methods of treatment and/or prevention using the compounds are described in detail below.

The following definitions are meant to clarify, but not limit, the terms defined. If a particular term used herein is not specifically defined, such term should not be considered indefinite. Rather, terms are used within their accepted meanings.

As used herein the term “alkyl” refers to a straight or branched chain hydrocarbon having one to twelve carbon atoms, preferably one to six, which may be optionally substituted as herein further described, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used throughout this specification, the preferred number of atoms, such as carbon atoms, will be represented by, for example, the phrase “Cₓ—C_{y} alkyl,” which refers to an alkyl group, as herein defined, containing the specified number of carbon atoms. Similar terminology will apply for other preferred terms and ranges as well. One embodiment of the present invention includes so-called ‘lower’ alkyl chains of one to six carbon atoms. Thus, C₁-C₆ alkyl represents a lower alkyl chain as hereinabove described.

As used herein the term “alkenyl” refers to a straight or branched chain aliphatic hydrocarbon having two to twelve carbon atoms, preferably two to six, and containing one or more carbon-to-carbon double bonds, which may be optionally substituted as herein further described, with multiple degrees of substitution being allowed. Examples of “alkenyl” as used herein include, but are not limited to, vinyl, and allyl.

As used herein, the term “cycloalkyl” refers to a partially or fully saturated, optionally substituted, non-aromatic, three- to twelve-membered, monocyclic, bicyclic, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, cycloheptyl, and cyclooctyl, as well as rings containing one or more degrees of unsaturation but short of aromatic, such as cyclopropenyl, cyclobutenyl, cyclopentenyl, cyclohexenyl, and cycloheptenyl.

As used herein, the term “heterocycle” or “heterocyclyl” refers to an optionally substituted mono- or polycyclic ring system, optionally containing one or more degrees of unsaturation and also containing one or more heteroatoms, which may be optionally substituted as herein further described, with multiple degrees of substitution being allowed. Exemplary heteroatoms include nitrogen, oxygen, or sulfur atoms, including N-oxides, sulfur oxides, and dioxides. Preferably, the ring is three to twelve-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to a univalent benzene ring or fused benzene ring system, which may be optionally substituted as herein further described, with multiple degrees of substitution being allowed. Examples of “aryl” groups as used include, but are not limited to, phenyl, 2-naphthyl, 1-naphthyl, anthracene, and phenanthrene. Preferably, aryl is phenyl or naphthyl.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and hexahydrocyclopenta-cyclooctene.

As used herein, the term “arylalkyl” refers to an “aryl” group as herein defined attached through a divalent alkylene linker.

As used herein, the term “heteroaryl” refers to a monocyclic five to seven membered aromatic ring, or to a fused bicyclic aromatic ring system comprising two of such aromatic rings, which may be optionally substituted as herein further described, with multiple degrees of substitution being allowed. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but should not be limited to, furanyl, thiophenyl or thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzodioxolyl, benzothiophenyl, indolyl, indolinyl, indazole, benzimidizolyl, indolizinyl, imidazopyridinyl, purinyl, pyrazolopyridinyl, and pyrazolopyrimidinyl.

In this specification, unless stated otherwise, the terms “halo” and “halogen” may be fluorine, iodine, chlorine, or bromine.

It will be appreciated that throughout the specification, the number and nature of substituents on rings in the compounds of the invention will be selected so as to avoid sterically undesirable combinations.

Certain compound names of the present invention were generated with the aid of computer software (ACDLabs 8.0/Name(IUPAC)).

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. Representative salts are provided as described in U.S. Pat. Nos. 5,597,919 to Dull et al., 5,616,716 to Dull et al. and 5,663,356 to Ruecroft et al.

The compounds of Formula 1 and pharmaceutically acceptable salts thereof may exist in solvated, for example hydrated, as well as unsolvated forms, or as cocrystals and the present invention encompasses all such forms.

For the avoidance of doubt, the present invention relates to any salts of forms as mentioned above for any compound falling within the scope of compounds of formula 1, or any one of the specific compounds mentioned below or any one of the salt mentioned above.

Additionally, the present invention includes solvate of the compounds herein described, including combinations solvates of a salt. The compounds of the present invention may exist in solvated, for example hydrated, as well as unsolvated forms, and the present invention encompasses all such forms.

As herein described, the present invention includes a compound of the present invention in isolated form. As used herein, the phrase “in isolated form” provides for the compound to be substantially free from other compounds, including by-products, impurities, and synthetic reagents. As used herein, the phrase “substantially free” should be interpreted to be approximately 95% free from such described other components.

As used herein, an “agonist” is a substance that stimulates its binding partner, typically a receptor. Stimulation is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects.

As used herein, an “antagonist” is a substance that inhibits its binding partner, typically a receptor. Inhibition is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Inhibition may be defined with respect to a decrease in a particular effect or function that is induced by interaction of the antagonist with a binding partner, and can include allosteric effects.

As used herein, a “partial agonist” is a substance that provides a level of stimulation to its binding partner that is intermediate between that of a full or complete antagonist and an agonist defined by any accepted standard for agonist activity. It will be recognized that stimulation, and hence, inhibition is defined intrinsically for any substance or category of substances to be defined as agonists, antagonists, or partial agonists.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. See Hoyer, D. and Boddeke, H., Trends Pharmacol. Sci. 14(7): 270-5 (1993). Where such contextually specific evaluations are relevant, and how they might be relevant in the context of the present invention, will be apparent to one of ordinary skill in the art.

As used herein, modulation of a receptor includes agonism, partial agonism, antagonism, partial antagonism, or inverse agonism of a receptor.

As used herein, neurotransmitters whose release is mediated by the compounds described herein include, but are not limited to, acetylcholine, dopamine, norepinephrine, serotonin and glutamate, and the compounds described herein function as modulators at the α4β2 subtype of the CNS NNRs.

As will be appreciated by those skilled in the art, compounds of the present invention are chiral. The present invention includes all stereoisomeric forms (e.g., enantiomeric or diastereomeric forms) of such compounds and mixtures thereof. Thus, the scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

Representative compounds of the present invention include the following:
2-(furan-2-ylcarbonyl)-2,6-diazabicyclo[3.2.0]heptane,
6-(furan-2-ylcarbonyl)-2,6-diazabicyclo[3.2.0]heptane,
2-(3-methylfuran-2-ylcarbonyl)-2,6-diazabicyclo[3.2.0]heptane,
6-(3-methylfuran-2-ylcarbonyl)-2,6-diazabicyclo[3.2.0]heptane,
2-(5-methylfuran-2-ylcarbonyl)-2,6-diazabicyclo[3.2.0]heptane,
6-(5-methylfuran-2-ylcarbonyl)-2,6-diazabicyclo[3.2.0]heptane,
2-(3-chlorofuran-2-ylcarbonyl)-2,6-diazabicyclo[3.2.0]heptane,
6-(3-chlorofuran-2-ylcarbonyl)-2,6-diazabicyclo[3.2.0]heptane,
2-(5-chlorofuran-2-ylcarbonyl)-2,6-diazabicyclo[3.2.0]heptane,
6-(5-chlorofuran-2-ylcarbonyl)-2,6-diazabicyclo[3.2.0]heptane,
2-(3-bromofuran-2-ylcarbonyl)-2,6-diazabicyclo[3.2.0]heptane,
6-(3-bromofuran-2-ylcarbonyl)-2,6-diazabicyclo[3.2.0]heptane,
2-(5-bromofuran-2-ylcarbonyl)-2,6-diazabicyclo[3.2.0]heptane,
6-(5-bromofuran-2-ylcarbonyl)-2,6-diazabicyclo[3.2.0]heptane,
2-(furan-3-ylcarbonyl)-2,6-diazabicyclo[3.2.0]heptane,
6-(furan-3-ylcarbonyl)-2,6-diazabicyclo[3.2.0]heptane,
2-(2-methylfuran-3-ylcarbonyl)-2,6-diazabicyclo[3.2.0]heptane,
6-(2-methylfuran-3-ylcarbonyl)-2,6-diazabicyclo[3.2.0]heptane,
2-(oxazol-2-ylcarbonyl)-2,6-diazabicyclo[3.2.0]heptane,
6-(oxazol-2-ylcarbonyl)-2,6-diazabicyclo[3.2.0]heptane,
2-(oxazol-5-ylcarbonyl)-2,6-diazabicyclo[3.2.0]heptane,
6-(oxazol-5-ylcarbonyl)-2,6-diazabicyclo[3.2.0]heptane,
2-(4-methyloxazol-5-ylcarbonyl)-2,6-diazabicyclo[3.2.0]heptane,
6-(4-methyloxazol-5-ylcarbonyl)-2,6-diazabicyclo[3.2.0]heptane,
2-(isoxazol-3-ylcarbonyl)-2,6-diazabicyclo[3.2.0]heptane,
6-(isoxazol-3-ylcarbonyl)-2,6-diazabicyclo[3.2.0]heptane,
2-(5-methylisoxazol-3-ylcarbonyl)-2,6-diazabicyclo[3.2.0]heptane,
6-(5-methylisoxazol-3-ylcarbonyl)-2,6-diazabicyclo[3.2.0]heptane,
2-(isoxazol-4-ylcarbonyl)-2,6-diazabicyclo[3.2.0]heptane,
6-(isoxazol-4-ylcarbonyl)-2,6-diazabicyclo[3.2.0]heptane,
2-(3-methylisoxazol-4-ylcarbonyl)-2,6-diazabicyclo[3.2.0]heptane,
6-(3-methylisoxazol-4-ylcarbonyl)-2,6-diazabicyclo[3.2.0]heptane,
2-(5-methylisoxazol-4-ylcarbonyl)-2,6-diazabicyclo[3.2.0]heptane,
6-(5-methylisoxazol-4-ylcarbonyl)-2,6-diazabicyclo[3.2.0]heptane,
2-(isoxazol-5-ylcarbonyl)-2,6-diazabicyclo[3.2.0]heptane,
6-(isoxazol-5-ylcarbonyl)-2,6-diazabicyclo[3.2.0]heptane,
2-(3-methylisoxazol-5-ylcarbonyl)-2,6-diazabicyclo[3.2.0]heptane,
6-(3-methylisoxazol-5-ylcarbonyl)-2,6-diazabicyclo[3.2.0]heptane,
2-(3-bromoisoxazol-5-ylcarbonyl)-2,6-diazabicyclo[3.2.0]heptane,
6-(3-bromoisoxazol-5-ylcarbonyl)-2,6-diazabicyclo[3.2.0]heptane,
2-(3-methoxyisoxazol-5-ylcarbonyl)-2,6-diazabicyclo[3.2.0]heptane,
6-(3-methoxyisoxazol-5-ylcarbonyl)-2,6-diazabicyclo[3.2.0]heptane,
2-(pyridin-4-ylcarbonyl)-2,6-diazabicyclo[3.2.0]heptane,
6-(pyridin-4-ylcarbonyl)-2,6-diazabicyclo[3.2.0]heptane,
   and pharmaceutically acceptable salts thereof.

Representative compounds of the present invention also include the following:
3-(furan-2-ylcarbonyl)-3,6-diazabicyclo[3.2.0]heptane,
6-(furan-2-ylcarbonyl)-3,6-diazabicyclo[3.2.0]heptane,
3-(3-methylfuran-2-ylcarbonyl)-3,6-diazabicyclo[3.2.0]heptane,
6-(3-methylfuran-2-ylcarbonyl)-3,6-diazabicyclo[3.2.0]heptane,
3-(5-methylfuran-2-ylcarbonyl)-3,6-diazabicyclo[3.2.0]heptane,
6-(5-methylfuran-2-ylcarbonyl)-3,6-diazabicyclo[3.2.0]heptane,
3-(3-chlorofuran-2-ylcarbonyl)-3,6-diazabicyclo[3.2.0]heptane,
6-(3-chlorofuran-2-ylcarbonyl)-3,6-diazabicyclo[3.2.0]heptane,
3-(5-chlorofuran-2-ylcarbonyl)-3,6-diazabicyclo[3.2.0]heptane,
6-(5-chlorofuran-2-ylcarbonyl)-3,6-diazabicyclo[3.2.0]heptane,
3-(3-bromofuran-2-ylcarbonyl)-3,6-diazabicyclo[3.2.0]heptane,
6-(3-bromofuran-2-ylcarbonyl)-3,6-diazabicyclo[3.2.0]heptane,
3-(5-bromofuran-2-ylcarbonyl)-3,6-diazabicyclo[3.2.0]heptane,
6-(5-bromofuran-2-ylcarbonyl)-3,6-diazabicyclo[3.2.0]heptane,
3-(furan-3-ylcarbonyl)-3,6-diazabicyclo[3.2.0]heptane,
6-(furan-3-ylcarbonyl)-3,6-diazabicyclo[3.2.0]heptane,
3-(2-methylfuran-3-ylcarbonyl)-3,6-diazabicyclo[3.2.0]heptane,
6-(2-methylfuran-3-ylcarbonyl)-3,6-diazabicyclo[3.2.0]heptane,
3-(oxazol-2-ylcarbonyl)-3,6-diazabicyclo[3.2.0]heptane,
6-(oxazol-2-ylcarbonyl)-3,6-diazabicyclo[3.2.0]heptane,
3-(oxazol-5-ylcarbonyl)-3,6-diazabicyclo[3.2.0]heptane,
6-(oxazol-5-ylcarbonyl)-3,6-diazabicyclo[3.2.0]heptane,
3-(4-methyloxazol-5-ylcarbonyl)-3,6-diazabicyclo[3.2.0]heptane,
6-(4-methyloxazol-5-ylcarbonyl)-3,6-diazabicyclo[3.2.0]heptane,
3-(isoxazol-3-ylcarbonyl)-3,6-diazabicyclo[3.2.0]heptane,
6-(isoxazol-3-ylcarbonyl)-3,6-diazabicyclo[3.2.0]heptane,
3-(5-methylisoxazol-3-ylcarbonyl)-3,6-diazabicyclo[3.2.0]heptane,
6-(5-methylisoxazol-3-ylcarbonyl)-3,6-diazabicyclo[3.2.0]heptane,
3-(isoxazol-4-ylcarbonyl)-3,6-diazabicyclo[3.2.0]heptane,
6-(isoxazol-4-ylcarbonyl)-3,6-diazabicyclo[3.2.0]heptane,
3-(3-methylisoxazol-4-ylcarbonyl)-3,6-diazabicyclo[3.2.0]heptane,
6-(3-methylisoxazol-4-ylcarbonyl)-3,6-diazabicyclo[3.2.0]heptane,
3-(5-methylisoxazol-4-ylcarbonyl)-3,6-diazabicyclo[3.2.0]heptane,
6-(5-methylisoxazol-4-ylcarbonyl)-3,6-diazabicyclo[3.2.0]heptane,
3-(isoxazol-5-ylcarbonyl)-3,6-diazabicyclo[3.2.0]heptane,
6-(isoxazol-5-ylcarbonyl)-3,6-diazabicyclo[3.2.0]heptane,
3-(3-methylisoxazol-5-ylcarbonyl)-3,6-diazabicyclo[3.2.0]heptane,
6-(3-methylisoxazol-5-ylcarbonyl)-3,6-diazabicyclo[3.2.0]heptane,
3-(3-bromoisoxazol-5-ylcarbonyl)-3,6-diazabicyclo[3.2.0]heptane,
6-(3-bromoisoxazol-5-ylcarbonyl)-3,6-diazabicyclo[3.2.0]heptane,
3-(3-methoxyisoxazol-5-ylcarbonyl)-3,6-diazabicyclo[3.2.0]heptane,
6-(3-methoxyisoxazol-5-ylcarbonyl)-3,6-diazabicyclo[3.2.0]heptane,
3-(pyridin-4-ylcarbonyl)-3,6-diazabicyclo[3.2.0]heptane,
6-(pyridin-4-ylcarbonyl)-3,6-diazabicyclo[3.2.0]heptane,
   and pharmaceutically acceptable salts thereof.

Representative compounds of the present invention also include the following:
2-(furan-2-ylcarbonyl)-2,7-diazabicyclo[4.2.0]octane,
7-(furan-2-ylcarbonyl)-2,7-diazabicyclo[4.2.0]octane,
2-(3-methylfuran-2-ylcarbonyl)-2,7-diazabicyclo[4.2.0]octane,
7-(3-methylfuran-2-ylcarbonyl)-2,7-diazabicyclo[4.2.0]octane,
2-(5-methylfuran-2-ylcarbonyl)-2,7-diazabicyclo[4.2.0]octane,
7-(5-methylfuran-2-ylcarbonyl)-2,7-diazabicyclo[4.2.0]octane,
2-(3-chlorofuran-2-ylcarbonyl)-2,7-diazabicyclo[4.2.0]octane,
7-(3-chlorofuran-2-ylcarbonyl)-2,7-diazabicyclo[4.2.0]octane,
2-(5-chlorofuran-2-ylcarbonyl)-2,7-diazabicyclo[4.2.0]octane,
7-(5-chlorofuran-2-ylcarbonyl)-2,7-diazabicyclo[4.2.0]octane,
2-(3-bromofuran-2-ylcarbonyl)-2,7-diazabicyclo[4.2.0]octane,
7-(3-bromofuran-2-ylcarbonyl)-2,7-diazabicyclo[4.2.0]octane,
2-(5-bromofuran-2-ylcarbonyl)-2,7-diazabicyclo[4.2.0]octane,
7-(5-bromofuran-2-ylcarbonyl)-2,7-diazabicyclo[4.2.0]octane,
2-(furan-3-ylcarbonyl)-2,7-diazabicyclo[4.2.0]octane,
7-(furan-3-ylcarbonyl)-2,7-diazabicyclo[4.2.0]octane,
2-(2-methylfuran-3-ylcarbonyl)-2,7-diazabicyclo[4.2.0]octane,
7-(2-methylfuran-3-ylcarbonyl)-2,7-diazabicyclo[4.2.0]octane,
2-(oxazol-2-ylcarbonyl)-2,7-diazabicyclo[4.2.0]octane,
7-(oxazol-2-ylcarbonyl)-2,7-diazabicyclo[4.2.0]octane,
2-(oxazol-5-ylcarbonyl)-2,7-diazabicyclo[4.2.0]octane,
7-(oxazol-5-ylcarbonyl)-2,7-diazabicyclo[4.2.0]octane,
2-(4-methyloxazol-5-ylcarbonyl)-2,7-diazabicyclo[4.2.0]octane,
7-(4-methyloxazol-5-ylcarbonyl)-2,7-diazabicyclo[4.2.0]octane,
2-(isoxazol-3-ylcarbonyl)-2,7-diazabicyclo[4.2.0]octane,
7-(isoxazol-3-ylcarbonyl)-2,7-diazabicyclo[4.2.0]octane,
2-(5-methylisoxazol-3-ylcarbonyl)-2,7-diazabicyclo[4.2.0]octane,
7-(5-methylisoxazol-3-ylcarbonyl)-2,7-diazabicyclo[4.2.0]octane,
2-(isoxazol-4-ylcarbonyl)-2,7-diazabicyclo[4.2.0]octane,
7-(isoxazol-4-ylcarbonyl)-2,7-diazabicyclo[4.2.0]octane,
2-(3-methylisoxazol-4-ylcarbonyl)-2,7-diazabicyclo[4.2.0]octane,
7-(3-methylisoxazol-4-ylcarbonyl)-2,7-diazabicyclo[4.2.0]octane,
2-(5-methylisoxazol-4-ylcarbonyl)-2,7-diazabicyclo[4.2.0]octane,
7-(5-methylisoxazol-4-ylcarbonyl)-2,7-diazabicyclo[4.2.0]octane,
2-(isoxazol-5-ylcarbonyl)-2,7-diazabicyclo[4.2.0]octane,
7-(isoxazol-5-ylcarbonyl)-2,7-diazabicyclo[4.2.0]octane,
2-(3-methylisoxazol-5-ylcarbonyl)-2,7-diazabicyclo[4.2.0]octane,
7-(3-methylisoxazol-5-ylcarbonyl)-2,7-diazabicyclo[4.2.0]octane,
2-(3-bromoisoxazol-5-ylcarbonyl)-2,7-diazabicyclo[4.2.0]octane,
7-(3-bromoisoxazol-5-ylcarbonyl)-2,7-diazabicyclo[4.2.0]octane,
2-(3-methoxyisoxazol-5-ylcarbonyl)-2,7-diazabicyclo[4.2.0]octane,
7-(3-methoxyisoxazol-5-ylcarbonyl)-2,7-diazabicyclo[4.2.0]octane,
2-(pyridin-4-ylcarbonyl)-2,7-diazabicyclo[4.2.0]octane,
7-(pyridin-4-ylcarbonyl)-2,7-diazabicyclo[4.2.0]octane,
   and pharmaceutically acceptable salts thereof.

Representative compounds of the present invention also include the following:
3-(furan-2-ylcarbonyl)-3,7-diazabicyclo[4.2.0]octane,
7-(furan-2-ylcarbonyl)-3,7-diazabicyclo[4.2.0]octane,
3-(3-methylfuran-2-ylcarbonyl)-3,7-diazabicyclo[4.2.0]octane,
7-(3-methylfuran-2-ylcarbonyl)-3,7-diazabicyclo[4.2.0]octane,
3-(5-methylfuran-2-ylcarbonyl)-3,7-diazabicyclo[4.2.0]octane,
7-(5-methylfuran-2-ylcarbonyl)-3,7-diazabicyclo[4.2.0]octane,
3-(3-chlorofuran-2-ylcarbonyl)-3,7-diazabicyclo[4.2.0]octane,
7-(3-chlorofuran-2-ylcarbonyl)-3,7-diazabicyclo[4.2.0]octane,
3-(5-chlorofuran-2-ylcarbonyl)-3,7-diazabicyclo[4.2.0]octane,
7-(5-chlorofuran-2-ylcarbonyl)-3,7-diazabicyclo[4.2.0]octane,
3-(3-bromofuran-2-ylcarbonyl)-3,7-diazabicyclo[4.2.0]octane,
7-(3-bromofuran-2-ylcarbonyl)-3,7-diazabicyclo[4.2.0]octane,
3-(5-bromofuran-2-ylcarbonyl)-3,7-diazabicyclo[4.2.0]octane,
7-(5-bromofuran-2-ylcarbonyl)-3,7-diazabicyclo[4.2.0]octane,
3-(furan-3-ylcarbonyl)-3,7-diazabicyclo[4.2.0]octane,
7-(furan-3-ylcarbonyl)-3,7-diazabicyclo[4.2.0]octane,
3-(2-methylfuran-3-ylcarbonyl)-3,7-diazabicyclo[4.2.0]octane,
7-(2-methylfuran-3-ylcarbonyl)-3,7-diazabicyclo[4.2.0]octane,
3-(oxazol-2-ylcarbonyl)-3,7-diazabicyclo[4.2.0]octane,
7-(oxazol-2-ylcarbonyl)-3,7-diazabicyclo[4.2.0]octane,
3-(oxazol-5-ylcarbonyl)-3,7-diazabicyclo[4.2.0]octane,
7-(oxazol-5-ylcarbonyl)-3,7-diazabicyclo[4.2.0]octane,
3-(4-methyloxazol-5-ylcarbonyl)-3,7-diazabicyclo[4.2.0]octane,
7-(4-methyloxazol-5-ylcarbonyl)-3,7-diazabicyclo[4.2.0]octane,
3-(isoxazol-3-ylcarbonyl)-3,7-diazabicyclo[4.2.0]octane,
7-(isoxazol-3-ylcarbonyl)-3,7-diazabicyclo[4.2.0]octane,
3-(5-methylisoxazol-3-ylcarbonyl)-3,7-diazabicyclo[4.2.0]octane,
7-(5-methylisoxazol-3-ylcarbonyl)-3,7-diazabicyclo[4.2.0]octane,
3-(isoxazol-4-ylcarbonyl)-3,7-diazabicyclo[4.2.0]octane,
7-(isoxazol-4-ylcarbonyl)-3,7-diazabicyclo[4.2.0]octane,
3-(3-methylisoxazol-4-ylcarbonyl)-3,7-diazabicyclo[4.2.0]octane,
7-(3-methylisoxazol-4-ylcarbonyl)-3,7-diazabicyclo[4.2.0]octane,
3-(5-methylisoxazol-4-ylcarbonyl)-3,7-diazabicyclo[4.2.0]octane,
7-(5-methylisoxazol-4-ylcarbonyl)-3,7-diazabicyclo[4.2.0]octane,
3-(isoxazol-5-ylcarbonyl)-3,7-diazabicyclo[4.2.0]octane,
7-(isoxazol-5-ylcarbonyl)-3,7-diazabicyclo[4.2.0]octane,
3-(3-methylisoxazol-5-ylcarbonyl)-3,7-diazabicyclo[4.2.0]octane,
7-(3-methylisoxazol-5-ylcarbonyl)-3,7-diazabicyclo[4.2.0]octane,
3-(3-bromoisoxazol-5-ylcarbonyl)-3,7-diazabicyclo[4.2.0]octane,
7-(3-bromoisoxazol-5-ylcarbonyl)-3,7-diazabicyclo[4.2.0]octane,
3-(3-methoxyisoxazol-5-ylcarbonyl)-3,7-diazabicyclo[4.2.0]octane,
7-(3-methoxyisoxazol-5-ylcarbonyl)-3,7-diazabicyclo[4.2.0]octane,
3-(pyridin-4-ylcarbonyl)-3,7-diazabicyclo[4.2.0]octane,
7-(pyridin-4-ylcarbonyl)-3,7-diazabicyclo[4.2.0]octane,
   and pharmaceutically acceptable salts thereof.

Representative compounds of the present invention also include the following:
3-(furan-2-ylcarbonyl)-3,8-diazabicyclo[4.2.0]octane,
8-(furan-2-ylcarbonyl)-3,8-diazabicyclo[4.2.0]octane,
3-(3-methylfuran-2-ylcarbonyl)-3,8-diazabicyclo[4.2.0]octane,
8-(3-methylfuran-2-ylcarbonyl)-3,8-diazabicyclo[4.2.0]octane,
3-(5-methylfuran-2-ylcarbonyl)-3,8-diazabicyclo[4.2.0]octane,
8-(5-methylfuran-2-ylcarbonyl)-3,8-diazabicyclo[4.2.0]octane,
3-(3-chlorofuran-2-ylcarbonyl)-3,8-diazabicyclo[4.2.0]octane,
8-(3-chlorofuran-2-ylcarbonyl)-3,8-diazabicyclo[4.2.0]octane,
3-(5-chlorofuran-2-ylcarbonyl)-3,8-diazabicyclo[4.2.0]octane,
8-(5-chlorofuran-2-ylcarbonyl)-3,8-diazabicyclo[4.2.0]octane,
3-(3-bromofuran-2-ylcarbonyl)-3,8-diazabicyclo[4.2.0]octane,
8-(3-bromofuran-2-ylcarbonyl)-3,8-diazabicyclo[4.2.0]octane,
3-(5-bromofuran-2-ylcarbonyl)-3,8-diazabicyclo[4.2.0]octane,
8-(5-bromofuran-2-ylcarbonyl)-3,8-diazabicyclo[4.2.0]octane,
3-(furan-3-ylcarbonyl)-3,8-diazabicyclo[4.2.0]octane,
8-(furan-3-ylcarbonyl)-3,8-diazabicyclo[4.2.0]octane,
3-(2-methylfuran-3-ylcarbonyl)-3,8-diazabicyclo[4.2.0]octane,
8-(2-methylfuran-3-ylcarbonyl)-3,8-diazabicyclo[4.2.0]octane,
3-(oxazol-2-ylcarbonyl)-3,8-diazabicyclo[4.2.0]octane,
8-(oxazol-2-ylcarbonyl)-3,8-diazabicyclo[4.2.0]octane,
3-(oxazol-5-ylcarbonyl)-3,8-diazabicyclo[4.2.0]octane,
8-(oxazol-5-ylcarbonyl)-3,8-diazabicyclo[4.2.0]octane,
3-(4-methyloxazol-5-ylcarbonyl)-3,8-diazabicyclo[4.2.0]octane,
8-(4-methyloxazol-5-ylcarbonyl)-3,8-diazabicyclo[4.2.0]octane,
3-(isoxazol-3-ylcarbonyl)-3,8-diazabicyclo[4.2.0]octane,
8-(isoxazol-3-ylcarbonyl)-3,8-diazabicyclo[4.2.0]octane,
3-(5-methylisoxazol-3-ylcarbonyl)-3,8-diazabicyclo[4.2.0]octane,
8-(5-methylisoxazol-3-ylcarbonyl)-3,8-diazabicyclo[4.2.0]octane,
3-(isoxazol-4-ylcarbonyl)-3,8-diazabicyclo[4.2.0]octane,
8-(isoxazol-4-ylcarbonyl)-3,8-diazabicyclo[4.2.0]octane,
3-(3-methylisoxazol-4-ylcarbonyl)-3,8-diazabicyclo[4.2.0]octane,
8-(3-methylisoxazol-4-ylcarbonyl)-3,8-diazabicyclo[4.2.0]octane,
3-(5-methylisoxazol-4-ylcarbonyl)-3,8-diazabicyclo[4.2.0]octane,
8-(5-methylisoxazol-4-ylcarbonyl)-3,8-diazabicyclo[4.2.0]octane,
3-(isoxazol-5-ylcarbonyl)-3,8-diazabicyclo[4.2.0]octane,
8-(isoxazol-5-ylcarbonyl)-3,8-diazabicyclo[4.2.0]octane,
3-(3-methylisoxazol-5-ylcarbonyl)-3,8-diazabicyclo[4.2.0]octane,
8-(3-methylisoxazol-5-ylcarbonyl)-3,8-diazabicyclo[4.2.0]octane,
3-(3-bromoisoxazol-5-ylcarbonyl)-3,8-diazabicyclo[4.2.0]octane,
8-(3-bromoisoxazol-5-ylcarbonyl)-3,8-diazabicyclo[4.2.0]octane,
3-(3-methoxyisoxazol-5-ylcarbonyl)-3,8-diazabicyclo[4.2.0]octane,
8-(3-methoxyisoxazol-5-ylcarbonyl)-3,8-diazabicyclo[4.2.0]octane,
3-(pyridin-4-ylcarbonyl)-3,8-diazabicyclo[4.2.0]octane,
8-(pyridin-4-ylcarbonyl)-3,8-diazabicyclo[4.2.0]octane,
   and pharmaceutically acceptable salts thereof.

Representative compounds of the present invention also include the following:
2-(furan-2-ylcarbonyl)-2,6-diazabicyclo[3.3.0]octane,
2-(3-methylfuran-2-ylcarbonyl)-2,6-diazabicyclo[3.3.0]octane,
2-(5-methylfuran-2-ylcarbonyl)-2,6-diazabicyclo[3.3.0]octane,
2-(3-chlorofuran-2-ylcarbonyl)-2,6-diazabicyclo[3.3.0]octane,
2-(5-chlorofuran-2-ylcarbonyl)-2,6-diazabicyclo[3.3.0]octane,
2-(3-bromofuran-2-ylcarbonyl)-2,6-diazabicyclo[3.3.0]octane,
2-(5-bromofuran-2-ylcarbonyl)-2,6-diazabicyclo[3.3.0]octane,
2-(furan-3-ylcarbonyl)-2,6-diazabicyclo[3.3.0]octane,
2-(2-methylfuran-3-ylcarbonyl)-2,6-diazabicyclo[3.3.0]octane,
2-(oxazol-2-ylcarbonyl)-2,6-diazabicyclo[3.3.0]octane,
2-(oxazol-5-ylcarbonyl)-2,6-diazabicyclo[3.3.0]octane,
2-(4-methyloxazol-5-ylcarbonyl)-2,6-diazabicyclo[3.3.0]octane,
2-(isoxazol-3-ylcarbonyl)-2,6-diazabicyclo[3.3.0]octane,
2-(5-methylisoxazol-3-ylcarbonyl)-2,6-diazabicyclo[3.3.0]octane,
2-(isoxazol-4-ylcarbonyl)-2,6-diazabicyclo[3.3.0]octane,
2-(3-methylisoxazol-4-ylcarbonyl)-2,6-diazabicyclo[3.3.0]octane,
2-(5-methylisoxazol-4-ylcarbonyl)-2,6-diazabicyclo[3.3.0]octane,
2-(isoxazol-5-ylcarbonyl)-2,6-diazabicyclo[3.3.0]octane,
2-(3-methylisoxazol-5-ylcarbonyl)-2,6-diazabicyclo[3.3.0]octane,
2-(3-bromoisoxazol-5-ylcarbonyl)-2,6-diazabicyclo[3.3.0]octane,
2-(3-methoxyisoxazol-5-ylcarbonyl)-2,6-diazabicyclo[3.3.0]octane,
2-(pyridin-4-ylcarbonyl)-2,6-diazabicyclo[3.3.0]octane,
   and pharmaceutically acceptable salts thereof.

Representative compounds of the present invention also include the following:
2-(furan-2-ylcarbonyl)-2,7-diazabicyclo[3.3.0]octane,
7-(furan-2-ylcarbonyl)-2,7-diazabicyclo[3.3.0]octane,
2-(3-methylfuran-2-ylcarbonyl)-2,7-diazabicyclo[3.3.0]octane,
7-(3-methylfuran-2-ylcarbonyl)-2,7-diazabicyclo[3.3.0]octane,
2-(5-methylfuran-2-ylcarbonyl)-2,7-diazabicyclo[3.3.0]octane,
7-(5-methylfuran-2-ylcarbonyl)-2,7-diazabicyclo[3.3.0]octane,
2-(3-chlorofuran-2-ylcarbonyl)-2,7-diazabicyclo[3.3.0]octane,
7-(3-chlorofuran-2-ylcarbonyl)-2,7-diazabicyclo[3.3.0]octane,
2-(5-chlorofuran-2-ylcarbonyl)-2,7-diazabicyclo[3.3.0]octane,
7-(5-chlorofuran-2-ylcarbonyl)-2,7-diazabicyclo[3.3.0]octane,
2-(3-bromofuran-2-ylcarbonyl)-2,7-diazabicyclo[3.3.0]octane,
7-(3-bromofuran-2-ylcarbonyl)-2,7-diazabicyclo[3.3.0]octane,
2-(5-bromofuran-2-ylcarbonyl)-2,7-diazabicyclo[3.3.0]octane,
7-(5-bromofuran-2-ylcarbonyl)-2,7-diazabicyclo[3.3.0]octane,
2-(furan-3-ylcarbonyl)-2,7-diazabicyclo[3.3.0]octane,
7-(furan-3-ylcarbonyl)-2,7-diazabicyclo[3.3.0]octane,
2-(2-methylfuran-3-ylcarbonyl)-2,7-diazabicyclo[3.3.0]octane,
7-(2-methylfuran-3-ylcarbonyl)-2,7-diazabicyclo[3.3.0]octane,
2-(oxazol-2-ylcarbonyl)-2,7-diazabicyclo[3.3.0]octane,
7-(oxazol-2-ylcarbonyl)-2,7-diazabicyclo[3.3.0]octane,
2-(oxazol-5-ylcarbonyl)-2,7-diazabicyclo[3.3.0]octane,
7-(oxazol-5-ylcarbonyl)-2,7-diazabicyclo[3.3.0]octane,
2-(4-methyloxazol-5-ylcarbonyl)-2,7-diazabicyclo[3.3.0]octane,
7-(4-methyloxazol-5-ylcarbonyl)-2,7-diazabicyclo[3.3.0]octane,
2-(isoxazol-3-ylcarbonyl)-2,7-diazabicyclo[3.3.0]octane,
7-(isoxazol-3-ylcarbonyl)-2,7-diazabicyclo[3.3.0]octane,
2-(5-methylisoxazol-3-ylcarbonyl)-2,7-diazabicyclo[3.3.0]octane,
7-(5-methylisoxazol-3-ylcarbonyl)-2,7-diazabicyclo[3.3.0]octane,
2-(isoxazol-4-ylcarbonyl)-2,7-diazabicyclo[3.3.0]octane,
7-(isoxazol-4-ylcarbonyl)-2,7-diazabicyclo[3.3.0]octane,
2-(3-methylisoxazol-4-ylcarbonyl)-2,7-diazabicyclo[3.3.0]octane,
7-(3-methylisoxazol-4-ylcarbonyl)-2,7-diazabicyclo[3.3.0]octane,
2-(5-methylisoxazol-4-ylcarbonyl)-2,7-diazabicyclo[3.3.0]octane,
7-(5-methylisoxazol-4-ylcarbonyl)-2,7-diazabicyclo[3.3.0]octane,
2-(isoxazol-5-ylcarbonyl)-2,7-diazabicyclo[3.3.0]octane,
7-(isoxazol-5-ylcarbonyl)-2,7-diazabicyclo[3.3.0]octane,
2-(3-methylisoxazol-5-ylcarbonyl)-2,7-diazabicyclo[3.3.0]octane,
7-(3-methylisoxazol-5-ylcarbonyl)-2,7-diazabicyclo[3.3.0]octane,
2-(3-bromoisoxazol-5-ylcarbonyl)-2,7-diazabicyclo[3.3.0]octane,
7-(3-bromoisoxazol-5-ylcarbonyl)-2,7-diazabicyclo[3.3.0]octane,
2-(3-methoxyisoxazol-5-ylcarbonyl)-2,7-diazabicyclo[3.3.0]octane,
7-(3-methoxyisoxazol-5-ylcarbonyl)-2,7-diazabicyclo[3.3.0]octane,
2-(pyridin-4-ylcarbonyl)-2,7-diazabicyclo[3.3.0]octane,
7-(pyridin-4-ylcarbonyl)-2,7-diazabicyclo[3.3.0]octane,
   and pharmaceutically acceptable salts thereof.

Representative compounds of the present invention also include the following:
2-(furan-2-ylcarbonyl)-2,7-diazabicyclo[4.3.0]nonane,
7-(furan-2-ylcarbonyl)-2,7-diazabicyclo[4.3.0]nonane,
2-(3-methylfuran-2-ylcarbonyl)-2,7-diazabicyclo[4.3.0]nonane,
7-(3-methylfuran-2-ylcarbonyl)-2,7-diazabicyclo[4.3.0]nonane,
2-(5-methylfuran-2-ylcarbonyl)-2,7-diazabicyclo[4.3.0]nonane,
7-(5-methylfuran-2-ylcarbonyl)-2,7-diazabicyclo[4.3.0]nonane,
2-(3-chlorofuran-2-ylcarbonyl)-2,7-diazabicyclo[4.3.0]nonane,
7-(3-chlorofuran-2-ylcarbonyl)-2,7-diazabicyclo[4.3.0]nonane,
2-(5-chlorofuran-2-ylcarbonyl)-2,7-diazabicyclo[4.3.0]nonane,
7-(5-chlorofuran-2-ylcarbonyl)-2,7-diazabicyclo[4.3.0]nonane,
2-(3-bromofuran-2-ylcarbonyl)-2,7-diazabicyclo[4.3.0]nonane,
7-(3-bromofuran-2-ylcarbonyl)-2,7-diazabicyclo[4.3.0]nonane,
2-(5-bromofuran-2-ylcarbonyl)-2,7-diazabicyclo[4.3.0]nonane,
7-(5-bromofuran-2-ylcarbonyl)-2,7-diazabicyclo[4.3.0]nonane,
2-(furan-3-ylcarbonyl)-2,7-diazabicyclo[4.3.0]nonane,
7-(furan-3-ylcarbonyl)-2,7-diazabicyclo[4.3.0]nonane,
2-(2-methylfuran-3-ylcarbonyl)-2,7-diazabicyclo[4.3.0]nonane,
7-(2-methylfuran-3-ylcarbonyl)-2,7-diazabicyclo[4.3.0]nonane,
2-(oxazol-2-ylcarbonyl)-2,7-diazabicyclo[4.3.0]nonane,
7-(oxazol-2-ylcarbonyl)-2,7-diazabicyclo[4.3.0]nonane,
2-(oxazol-5-ylcarbonyl)-2,7-diazabicyclo[4.3.0]nonane,
7-(oxazol-5-ylcarbonyl)-2,7-diazabicyclo[4.3.0]nonane,
2-(4-methyloxazol-5-ylcarbonyl)-2,7-diazabicyclo[4.3.0]nonane,
7-(4-methyloxazol-5-ylcarbonyl)-2,7-diazabicyclo[4.3.0]nonane,
2-(isoxazol-3-ylcarbonyl)-2,7-diazabicyclo[4.3.0]nonane,
7-(isoxazol-3-ylcarbonyl)-2,7-diazabicyclo[4.3.0]nonane,
2-(5-methylisoxazol-3-ylcarbonyl)-2,7-diazabicyclo[4.3.0]nonane,
7-(5-methylisoxazol-3-ylcarbonyl)-2,7-diazabicyclo[4.3.0]nonane,
2-(isoxazol-4-ylcarbonyl)-2,7-diazabicyclo[4.3.0]nonane,
7-(isoxazol-4-ylcarbonyl)-2,7-diazabicyclo[4.3.0]nonane,
2-(3-methylisoxazol-4-ylcarbonyl)-2,7-diazabicyclo[4.3.0]nonane,
7-(3-methylisoxazol-4-ylcarbonyl)-2,7-diazabicyclo[4.3.0]nonane,
2-(5-methylisoxazol-4-ylcarbonyl)-2,7-diazabicyclo[4.3.0]nonane,
7-(5-methylisoxazol-4-ylcarbonyl)-2,7-diazabicyclo[4.3.0]nonane,
2-(isoxazol-5-ylcarbonyl)-2,7-diazabicyclo[4.3.0]nonane,
7-(isoxazol-5-ylcarbonyl)-2,7-diazabicyclo[4.3.0]nonane,
2-(3-methylisoxazol-5-ylcarbonyl)-2,7-diazabicyclo[4.3.0]nonane,
7-(3-methylisoxazol-5-ylcarbonyl)-2,7-diazabicyclo[4.3.0]nonane,
2-(3-bromoisoxazol-5-ylcarbonyl)-2,7-diazabicyclo[4.3.0]nonane,
7-(3-bromoisoxazol-5-ylcarbonyl)-2,7-diazabicyclo[4.3.0]nonane,
2-(3-methoxyisoxazol-5-ylcarbonyl)-2,7-diazabicyclo[4.3.0]nonane,
7-(3-methoxyisoxazol-5-ylcarbonyl)-2,7-diazabicyclo[4.3.0]nonane,
2-(pyridin-4-ylcarbonyl)-2,7-diazabicyclo[4.3.0]nonane,
7-(pyridin-4-ylcarbonyl)-2,7-diazabicyclo[4.3.0]nonane,
   and pharmaceutically acceptable salts thereof.

Representative compounds of the present invention also include the following:
2-(furan-2-ylcarbonyl)-2,8-diazabicyclo[4.3.0]nonane,
8-(furan-2-ylcarbonyl)-2,8-diazabicyclo[4.3.0]nonane,
2-(3-methylfuran-2-ylcarbonyl)-2,8-diazabicyclo[4.3.0]nonane,
8-(3-methylfuran-2-ylcarbonyl)-2,8-diazabicyclo[4.3.0]nonane,
2-(5-methylfuran-2-ylcarbonyl)-2,8-diazabicyclo[4.3.0]nonane,
8-(5-methylfuran-2-ylcarbonyl)-2,8-diazabicyclo[4.3.0]nonane,
2-(3-chlorofuran-2-ylcarbonyl)-2,8-diazabicyclo[4.3.0]nonane,
8-(3-chlorofuran-2-ylcarbonyl)-2,8-diazabicyclo[4.3.0]nonane,
2-(5-chlorofuran-2-ylcarbonyl)-2,8-diazabicyclo[4.3.0]nonane,
8-(5-chlorofuran-2-ylcarbonyl)-2,8-diazabicyclo[4.3.0]nonane,
2-(3-bromofuran-2-ylcarbonyl)-2,8-diazabicyclo[4.3.0]nonane,
8-(3-bromofuran-2-ylcarbonyl)-2,8-diazabicyclo[4.3.0]nonane,
2-(5-bromofuran-2-ylcarbonyl)-2,8-diazabicyclo[4.3.0]nonane,
8-(5-bromofuran-2-ylcarbonyl)-2,8-diazabicyclo[4.3.0]nonane,
2-(furan-3-ylcarbonyl)-2,8-diazabicyclo[4.3.0]nonane,
8-(furan-3-ylcarbonyl)-2,8-diazabicyclo[4.3.0]nonane,
2-(2-methylfuran-3-ylcarbonyl)-2,8-diazabicyclo[4.3.0]nonane,
8-(2-methylfuran-3-ylcarbonyl)-2,8-diazabicyclo[4.3.0]nonane,
2-(oxazol-2-ylcarbonyl)-2,8-diazabicyclo[4.3.0]nonane,
8-(oxazol-2-ylcarbonyl)-2,8-diazabicyclo[4.3.0]nonane,
2-(oxazol-5-ylcarbonyl)-2,8-diazabicyclo[4.3.0]nonane,
8-(oxazol-5-ylcarbonyl)-2,8-diazabicyclo[4.3.0]nonane,
2-(4-methyloxazol-5-ylcarbonyl)-2,8-diazabicyclo[4.3.0]nonane,
8-(4-methyloxazol-5-ylcarbonyl)-2,8-diazabicyclo[4.3.0]nonane,
2-(isoxazol-3-ylcarbonyl)-2,8-diazabicyclo[4.3.0]nonane,
8-(isoxazol-3-ylcarbonyl)-2,8-diazabicyclo[4.3.0]nonane,
2-(5-methylisoxazol-3-ylcarbonyl)-2,8-diazabicyclo[4.3.0]nonane,
8-(5-methylisoxazol-3-ylcarbonyl)-2,8-diazabicyclo[4.3.0]nonane,
2-(isoxazol-4-ylcarbonyl)-2,8-diazabicyclo[4.3.0]nonane,
8-(isoxazol-4-ylcarbonyl)-2,8-diazabicyclo[4.3.0]nonane,
2-(3-methylisoxazol-4-ylcarbonyl)-2,8-diazabicyclo[4.3.0]nonane,
8-(3-methylisoxazol-4-ylcarbonyl)-2,8-diazabicyclo[4.3.0]nonane,
2-(5-methylisoxazol-4-ylcarbonyl)-2,8-diazabicyclo[4.3.0]nonane,
8-(5-methylisoxazol-4-ylcarbonyl)-2,8-diazabicyclo[4.3.0]nonane,
2-(isoxazol-5-ylcarbonyl)-2,8-diazabicyclo[4.3.0]nonane,
8-(isoxazol-5-ylcarbonyl)-2,8-diazabicyclo[4.3.0]nonane,
2-(3-methylisoxazol-5-ylcarbonyl)-2,8-diazabicyclo[4.3.0]nonane,
8-(3-methylisoxazol-5-ylcarbonyl)-2,8-diazabicyclo[4.3.0]nonane,
2-(3-bromoisoxazol-5-ylcarbonyl)-2,8-diazabicyclo[4.3.0]nonane,
8-(3-bromoisoxazol-5-ylcarbonyl)-2,8-diazabicyclo[4.3.0]nonane,
2-(3-methoxyisoxazol-5-ylcarbonyl)-2,8-diazabicyclo[4.3.0]nonane,
8-(3-methoxyisoxazol-5-ylcarbonyl)-2,8-diazabicyclo[4.3.0]nonane,
2-(pyridin-4-ylcarbonyl)-2,8-diazabicyclo[4.3.0]nonane,
8-(pyridin-4-ylcarbonyl)-2,8-diazabicyclo[4.3.0]nonane,
   and pharmaceutically acceptable salts thereof.

Representative compounds of the present invention also include the following:
3-(furan-2-ylcarbonyl)-3,7-diazabicyclo[4.3.0]nonane,
7-(furan-2-ylcarbonyl)-3,7-diazabicyclo[4.3.0]nonane,
3-(3-methylfuran-2-ylcarbonyl)-3,7-diazabicyclo[4.3.0]nonane,
7-(3-methylfuran-2-ylcarbonyl)-3,7-diazabicyclo[4.3.0]nonane,
3-(5-methylfuran-2-ylcarbonyl)-3,7-diazabicyclo[4.3.0]nonane,
7-(5-methylfuran-2-ylcarbonyl)-3,7-diazabicyclo[4.3.0]nonane,
3-(3-chlorofuran-2-ylcarbonyl)-3,7-diazabicyclo[4.3.0]nonane,
7-(3-chlorofuran-2-ylcarbonyl)-3,7-diazabicyclo[4.3.0]nonane,
3-(5-chlorofuran-2-ylcarbonyl)-3,7-diazabicyclo[4.3.0]nonane,
7-(5-chlorofuran-2-ylcarbonyl)-3,7-diazabicyclo[4.3.0]nonane,
3-(3-bromofuran-2-ylcarbonyl)-3,7-diazabicyclo[4.3.0]nonane,
7-(3-bromofuran-2-ylcarbonyl)-3,7-diazabicyclo[4.3.0]nonane,
3-(5-bromofuran-2-ylcarbonyl)-3,7-diazabicyclo[4.3.0]nonane,
7-(5-bromofuran-2-ylcarbonyl)-3,7-diazabicyclo[4.3.0]nonane,
3-(furan-3-ylcarbonyl)-3,7-diazabicyclo[4.3.0]nonane,
7-(furan-3-ylcarbonyl)-3,7-diazabicyclo[4.3.0]nonane,
3-(2-methylfuran-3-ylcarbonyl)-3,7-diazabicyclo[4.3.0]nonane,
7-(2-methylfuran-3-ylcarbonyl)-3,7-diazabicyclo[4.3.0]nonane,
3-(oxazol-2-ylcarbonyl)-3,7-diazabicyclo[4.3.0]nonane,
7-(oxazol-2-ylcarbonyl)-3,7-diazabicyclo[4.3.0]nonane,
3-(oxazol-5-ylcarbonyl)-3,7-diazabicyclo[4.3.0]nonane,
7-(oxazol-5-ylcarbonyl)-3,7-diazabicyclo[4.3.0]nonane,
3-(4-methyloxazol-5-ylcarbonyl)-3,7-diazabicyclo[4.3.0]nonane,
7-(4-methyloxazol-5-ylcarbonyl)-3,7-diazabicyclo[4.3.0]nonane,
3-(isoxazol-3-ylcarbonyl)-3,7-diazabicyclo[4.3.0]nonane,
7-(isoxazol-3-ylcarbonyl)-3,7-diazabicyclo[4.3.0]nonane,
3-(5-methylisoxazol-3-ylcarbonyl)-3,7-diazabicyclo[4.3.0]nonane,
7-(5-methylisoxazol-3-ylcarbonyl)-3,7-diazabicyclo[4.3.0]nonane,
3-(isoxazol-4-ylcarbonyl)-3,7-diazabicyclo[4.3.0]nonane,
7-(isoxazol-4-ylcarbonyl)-3,7-diazabicyclo[4.3.0]nonane,
3-(3-methylisoxazol-4-ylcarbonyl)-3,7-diazabicyclo[4.3.0]nonane,
7-(3-methylisoxazol-4-ylcarbonyl)-3,7-diazabicyclo[4.3.0]nonane,
3-(5-methylisoxazol-4-ylcarbonyl)-3,7-diazabicyclo[4.3.0]nonane,
7-(5-methylisoxazol-4-ylcarbonyl)-3,7-diazabicyclo[4.3.0]nonane,
3-(isoxazol-5-ylcarbonyl)-3,7-diazabicyclo[4.3.0]nonane,
7-(isoxazol-5-ylcarbonyl)-3,7-diazabicyclo[4.3.0]nonane,
3-(3-methylisoxazol-5-ylcarbonyl)-3,7-diazabicyclo[4.3.0]nonane,
7-(3-methylisoxazol-5-ylcarbonyl)-3,7-diazabicyclo[4.3.0]nonane,
3-(3-bromoisoxazol-5-ylcarbonyl)-3,7-diazabicyclo[4.3.0]nonane,
7-(3-bromoisoxazol-5-ylcarbonyl)-3,7-diazabicyclo[4.3.0]nonane,
3-(3-methoxyisoxazol-5-ylcarbonyl)-3,7-diazabicyclo[4.3.0]nonane,
7-(3-methoxyisoxazol-5-ylcarbonyl)-3,7-diazabicyclo[4.3.0]nonane,
3-(pyridin-4-ylcarbonyl)-3,7-diazabicyclo[4.3.0]nonane,
7-(pyridin-4-ylcarbonyl)-3,7-diazabicyclo[4.3.0]nonane,
   and pharmaceutically acceptable salts thereof.

Representative compounds of the present invention also include the following:
3-(furan-2-ylcarbonyl)-3,8-diazabicyclo[4.3.0]nonane, 8-(furan-2-ylcarbonyl)-3,8-diazabicyclo[4.3.0]nonane,
3-(3-methylfuran-2-ylcarbonyl)-3,8-diazabicyclo[4.3.0]nonane,
8-(3-methylfuran-2-ylcarbonyl)-3,8-diazabicyclo[4.3.0]nonane,
3-(5-methylfuran-2-ylcarbonyl)-3,8-diazabicyclo[4.3.0]nonane,
8-(5-methylfuran-2-ylcarbonyl)-3,8-diazabicyclo[4.3.0]nonane,
3-(3-chlorofuran-2-ylcarbonyl)-3,8-diazabicyclo[4.3.0]nonane,
8-(3-chlorofuran-2-ylcarbonyl)-3,8-diazabicyclo[4.3.0]nonane,
3-(5-chlorofuran-2-ylcarbonyl)-3,8-diazabicyclo[4.3.0]nonane,
8-(5-chlorofuran-2-ylcarbonyl)-3,8-diazabicyclo[4.3.0]nonane,
3-(3-bromofuran-2-ylcarbonyl)-3,8-diazabicyclo[4.3.0]nonane,
8-(3-bromofuran-2-ylcarbonyl)-3,8-diazabicyclo[4.3.0]nonane,
3-(5-bromofuran-2-ylcarbonyl)-3,8-diazabicyclo[4.3.0]nonane,
8-(5-bromofuran-2-ylcarbonyl)-3,8-diazabicyclo[4.3.0]nonane,
3-(furan-3-ylcarbonyl)-3,8-diazabicyclo[4.3.0]nonane,
8-(furan-3-ylcarbonyl)-3,8-diazabicyclo[4.3.0]nonane,
3-(2-methylfuran-3-ylcarbonyl)-3,8-diazabicyclo[4.3.0]nonane,
8-(2-methylfuran-3-ylcarbonyl)-3,8-diazabicyclo[4.3.0]nonane,
3-(oxazol-2-ylcarbonyl)-3,8-diazabicyclo[4.3.0]nonane,
8-(oxazol-2-ylcarbonyl)-3,8-diazabicyclo[4.3.0]nonane,
3-(oxazol-5-ylcarbonyl)-3,8-diazabicyclo[4.3.0]nonane,
8-(oxazol-5-ylcarbonyl)-3,8-diazabicyclo[4.3.0]nonane,
3-(4-methyloxazol-5-ylcarbonyl)-3,8-diazabicyclo[4.3.0]nonane,
8-(4-methyloxazol-5-ylcarbonyl)-3,8-diazabicyclo[4.3.0]nonane,
3-(isoxazol-3-ylcarbonyl)-3,8-diazabicyclo[4.3.0]nonane,
8-(isoxazol-3-ylcarbonyl)-3,8-diazabicyclo[4.3.0]nonane,
3-(5-methylisoxazol-3-ylcarbonyl)-3,8-diazabicyclo[4.3.0]nonane,
8-(5-methylisoxazol-3-ylcarbonyl)-3,8-diazabicyclo[4.3.0]nonane,
3-(isoxazol-4-ylcarbonyl)-3,8-diazabicyclo[4.3.0]nonane,
8-(isoxazol-4-ylcarbonyl)-3,8-diazabicyclo[4.3.0]nonane,
3-(3-methylisoxazol-4-ylcarbonyl)-3,8-diazabicyclo[4.3.0]nonane,
8-(3-methylisoxazol-4-ylcarbonyl)-3,8-diazabicyclo[4.3.0]nonane,
3-(5-methylisoxazol-4-ylcarbonyl)-3,8-diazabicyclo[4.3.0]nonane,
8-(5-methylisoxazol-4-ylcarbonyl)-3,8-diazabicyclo[4.3.0]nonane,
3-(isoxazol-5-ylcarbonyl)-3,8-diazabicyclo[4.3.0]nonane,
8-(isoxazol-5-ylcarbonyl)-3,8-diazabicyclo[4.3.0]nonane,
3-(3-methylisoxazol-5-ylcarbonyl)-3,8-diazabicyclo[4.3.0]nonane,
8-(3-methylisoxazol-5-ylcarbonyl)-3,8-diazabicyclo[4.3.0]nonane,
3-(3-bromoisoxazol-5-ylcarbonyl)-3,8-diazabicyclo[4.3.0]nonane,
8-(3-bromoisoxazol-5-ylcarbonyl)-3,8-diazabicyclo[4.3.0]nonane,
3-(3-methoxyisoxazol-5-ylcarbonyl)-3,8-diazabicyclo[4.3.0]nonane,
8-(3-methoxyisoxazol-5-ylcarbonyl)-3,8-diazabicyclo[4.3.0]nonane,
3-(pyridin-4-ylcarbonyl)-3,8-diazabicyclo[4.3.0]nonane,
8-(pyridin-4-ylcarbonyl)-3,8-diazabicyclo[4.3.0]nonane,
   and pharmaceutically acceptable salts thereof.

Representative compounds of the present invention also include the following:
3-(furan-2-ylcarbonyl)-3,9-diazabicyclo[4.3.0]nonane,
9-(furan-2-ylcarbonyl)-3,9-diazabicyclo[4.3.0]nonane,
3-(3-methylfuran-2-ylcarbonyl)-3,9-diazabicyclo[4.3.0]nonane,
9-(3-methylfuran-2-ylcarbonyl)-3,9-diazabicyclo[4.3.0]nonane,
3-(5-methylfuran-2-ylcarbonyl)-3,9-diazabicyclo[4.3.0]nonane,
9-(5-methylfuran-2-ylcarbonyl)-3,9-diazabicyclo[4.3.0]nonane,
3-(3-chlorofuran-2-ylcarbonyl)-3,9-diazabicyclo[4.3.0]nonane,
9-(3-chlorofuran-2-ylcarbonyl)-3,9-diazabicyclo[4.3.0]nonane,
3-(5-chlorofuran-2-ylcarbonyl)-3,9-diazabicyclo[4.3.0]nonane,
9-(5-chlorofuran-2-ylcarbonyl)-3,9-diazabicyclo[4.3.0]nonane,
3-(3-bromofuran-2-ylcarbonyl)-3,9-diazabicyclo[4.3.0]nonane,
9-(3-bromofuran-2-ylcarbonyl)-3,9-diazabicyclo[4.3.0]nonane,
3-(5-bromofuran-2-ylcarbonyl)-3,9-diazabicyclo[4.3.0]nonane,
9-(5-bromofuran-2-ylcarbonyl)-3,9-diazabicyclo[4.3.0]nonane,
3-(furan-3-ylcarbonyl)-3,9-diazabicyclo[4.3.0]nonane,
9-(furan-3-ylcarbonyl)-3,9-diazabicyclo[4.3.0]nonane,
3-(2-methylfuran-3-ylcarbonyl)-3,9-diazabicyclo[4.3.0]nonane,
9-(2-methylfuran-3-ylcarbonyl)-3,9-diazabicyclo[4.3.0]nonane,
3-(oxazol-2-ylcarbonyl)-3,9-diazabicyclo[4.3.0]nonane,
9-(oxazol-2-ylcarbonyl)-3,9-diazabicyclo[4.3.0]nonane,
3-(oxazol-5-ylcarbonyl)-3,9-diazabicyclo[4.3.0]nonane,
9-(oxazol-5-ylcarbonyl)-3,9-diazabicyclo[4.3.0]nonane,
3-(4-methyloxazol-5-ylcarbonyl)-3,9-diazabicyclo[4.3.0]nonane,
9-(4-methyloxazol-5-ylcarbonyl)-3,9-diazabicyclo[4.3.0]nonane,
3-(isoxazol-3-ylcarbonyl)-3,9-diazabicyclo[4.3.0]nonane,
9-(isoxazol-3-ylcarbonyl)-3,9-diazabicyclo[4.3.0]nonane,
3-(5-methylisoxazol-3-ylcarbonyl)-3,9-diazabicyclo[4.3.0]nonane,
9-(5-methylisoxazol-3-ylcarbonyl)-3,9-diazabicyclo[4.3.0]nonane,
3-(isoxazol-4-ylcarbonyl)-3,9-diazabicyclo[4.3.0]nonane,
9-(isoxazol-4-ylcarbonyl)-3,9-diazabicyclo[4.3.0]nonane,
3-(3-methylisoxazol-4-ylcarbonyl)-3,9-diazabicyclo[4.3.0]nonane,
9-(3-methylisoxazol-4-ylcarbonyl)-3,9-diazabicyclo[4.3.0]nonane,
3-(5-methylisoxazol-4-ylcarbonyl)-3,9-diazabicyclo[4.3.0]nonane,
9-(5-methylisoxazol-4-ylcarbonyl)-3,9-diazabicyclo[4.3.0]nonane,
3-(isoxazol-5-ylcarbonyl)-3,9-diazabicyclo[4.3.0]nonane, 9-(isoxazol-5-ylcarbonyl)-3,9-diazabicyclo[4.3.0]nonane,
3-(3-methylisoxazol-5-ylcarbonyl)-3,9-diazabicyclo[4.3.0]nonane,
9-(3-methylisoxazol-5-ylcarbonyl)-3,9-diazabicyclo[4.3.0]nonane,
3-(3-bromoisoxazol-5-ylcarbonyl)-3,9-diazabicyclo[4.3.0]nonane,
9-(3-bromoisoxazol-5-ylcarbonyl)-3,9-diazabicyclo[4.3.0]nonane,
3-(3-methoxyisoxazol-5-ylcarbonyl)-3,9-diazabicyclo[4.3.0]nonane,
9-(3-methoxyisoxazol-5-ylcarbonyl)-3,9-diazabicyclo[4.3.0]nonane,
3-(pyridin-4-ylcarbonyl)-3,9-diazabicyclo[4.3.0]nonane,
9-(pyridin-4-ylcarbonyl)-3,9-diazabicyclo[4.3.0]nonane,
   and pharmaceutically acceptable salts thereof.

Representative compounds of the present invention also include the following:
2-(furan-2-ylcarbonyl)-2,6-diazabicyclo[3.2.1]octane,
6-(furan-2-ylcarbonyl)-2,6-diazabicyclo[3.2.1]octane,
2-(3-methylfuran-2-ylcarbonyl)-2,6-diazabicyclo[3.2.1]octane,
6-(3-methylfuran-2-ylcarbonyl)-2,6-diazabicyclo[3.2.1]octane,
2-(5-methylfuran-2-ylcarbonyl)-2,6-diazabicyclo[3.2.1]octane,
6-(5-methylfuran-2-ylcarbonyl)-2,6-diazabicyclo[3.2.1]octane,
2-(3-chlorofuran-2-ylcarbonyl)-2,6-diazabicyclo[3.2.1]octane,
6-(3-chlorofuran-2-ylcarbonyl)-2,6-diazabicyclo[3.2.1]octane,
2-(5-chlorofuran-2-ylcarbonyl)-2,6-diazabicyclo[3.2.1]octane,
6-(5-chlorofuran-2-ylcarbonyl)-2,6-diazabicyclo[3.2.1]octane,
2-(3-bromofuran-2-ylcarbonyl)-2,6-diazabicyclo[3.2.1]octane,
6-(3-bromofuran-2-ylcarbonyl)-2,6-diazabicyclo[3.2.1]octane,
2-(5-bromofuran-2-ylcarbonyl)-2,6-diazabicyclo[3.2.1]octane,
6-(5-bromofuran-2-ylcarbonyl)-2,6-diazabicyclo[3.2.1]octane,
2-(furan-3-ylcarbonyl)-2,6-diazabicyclo[3.2.1]octane,
6-(furan-3-ylcarbonyl)-2,6-diazabicyclo[3.2.1]octane,
2-(2-methylfuran-3-ylcarbonyl)-2,6-diazabicyclo[3.2.1]octane,
6-(2-methylfuran-3-ylcarbonyl)-2,6-diazabicyclo[3.2.1]octane,
2-(oxazol-2-ylcarbonyl)-2,6-diazabicyclo[3.2.1]octane,
6-(oxazol-2-ylcarbonyl)-2,6-diazabicyclo[3.2.1]octane,
2-(oxazol-5-ylcarbonyl)-2,6-diazabicyclo[3.2.1]octane,
6-(oxazol-5-ylcarbonyl)-2,6-diazabicyclo[3.2.1]octane,
2-(4-methyloxazol-5-ylcarbonyl)-2,6-diazabicyclo[3.2.1]octane,
6-(4-methyloxazol-5-ylcarbonyl)-2,6-diazabicyclo[3.2.1]octane,
2-(isoxazol-3-ylcarbonyl)-2,6-diazabicyclo[3.2.1]octane,
6-(isoxazol-3-ylcarbonyl)-2,6-diazabicyclo[3.2.1]octane, 2-(5-methylisoxazol-3-ylcarbonyl)-2,6-diazabicyclo[3.2.1]octane,
6-(5-methylisoxazol-3-ylcarbonyl)-2,6-diazabicyclo[3.2.1]octane,
2-(isoxazol-4-ylcarbonyl)-2,6-diazabicyclo[3.2.1]octane,
6-(isoxazol-4-ylcarbonyl)-2,6-diazabicyclo[3.2.1]octane,
2-(3-methylisoxazol-4-ylcarbonyl)-2,6-diazabicyclo[3.2.1]octane,
6-(3-methylisoxazol-4-ylcarbonyl)-2,6-diazabicyclo[3.2.1]octane,
2-(5-methylisoxazol-4-ylcarbonyl)-2,6-diazabicyclo[3.2.1]octane,
6-(5-methylisoxazol-4-ylcarbonyl)-2,6-diazabicyclo[3.2.1]octane,
2-(isoxazol-5-ylcarbonyl)-2,6-diazabicyclo[3.2.1]octane,
6-(isoxazol-5-ylcarbonyl)-2,6-diazabicyclo[3.2.1]octane,
2-(3-methylisoxazol-5-ylcarbonyl)-2,6-diazabicyclo[3.2.1]octane,
6-(3-methylisoxazol-5-ylcarbonyl)-2,6-diazabicyclo[3.2.1]octane,
2-(3-bromoisoxazol-5-ylcarbonyl)-2,6-diazabicyclo[3.2.1]octane,
6-(3-bromoisoxazol-5-ylcarbonyl)-2,6-diazabicyclo[3.2.1]octane,
2-(3-methoxyisoxazol-5-ylcarbonyl)-2,6-diazabicyclo[3.2.1]octane,
6-(3-methoxyisoxazol-5-ylcarbonyl)-2,6-diazabicyclo[3.2.1]octane,
2-(pyridin-4-ylcarbonyl)-2,6-diazabicyclo[3.2.1]octane,
6-(pyridin-4-ylcarbonyl)-2,6-diazabicyclo[3.2.1]octane,
   and pharmaceutically acceptable salts thereof.

Representative compounds of the present invention also include the following:
3-(furan-2-ylcarbonyl)-3,6-diazabicyclo[3.2.1]octane,
6-(furan-2-ylcarbonyl)-3,6-diazabicyclo[3.2.1]octane,
3-(3-methylfuran-2-ylcarbonyl)-3,6-diazabicyclo[3.2.1]octane,
6-(3-methylfuran-2-ylcarbonyl)-3,6-diazabicyclo[3.2.1]octane,
3-(5-methylfuran-2-ylcarbonyl)-3,6-diazabicyclo[3.2.1]octane,
6-(5-methylfuran-2-ylcarbonyl)-3,6-diazabicyclo[3.2.1]octane,
3-(3-chlorofuran-2-ylcarbonyl)-3,6-diazabicyclo[3.2.1]octane,
6-(3-chlorofuran-2-ylcarbonyl)-3,6-diazabicyclo[3.2.1]octane,
3-(5-chlorofuran-2-ylcarbonyl)-3,6-diazabicyclo[3.2.1]octane,
6-(5-chlorofuran-2-ylcarbonyl)-3,6-diazabicyclo[3.2.1]octane,
3-(3-bromofuran-2-ylcarbonyl)-3,6-diazabicyclo[3.2.1]octane,
6-(3-bromofuran-2-ylcarbonyl)-3,6-diazabicyclo[3.2.1]octane,
3-(5-bromofuran-2-ylcarbonyl)-3,6-diazabicyclo[3.2.1]octane,
6-(5-bromofuran-2-ylcarbonyl)-3,6-diazabicyclo[3.2.1]octane,
3-(furan-3-ylcarbonyl)-3,6-diazabicyclo[3.2.1]octane,
6-(furan-3-ylcarbonyl)-3,6-diazabicyclo[3.2.1]octane,
3-(2-methylfuran-3-ylcarbonyl)-3,6-diazabicyclo[3.2.1]octane,
6-(2-methylfuran-3-ylcarbonyl)-3,6-diazabicyclo[3.2.1]octane,
3-(oxazol-2-ylcarbonyl)-3,6-diazabicyclo[3.2.1]octane,
6-(oxazol-2-ylcarbonyl)-3,6-diazabicyclo[3.2.1]octane,
3-(oxazol-5-ylcarbonyl)-3,6-diazabicyclo[3.2.1]octane,
6-(oxazol-5-ylcarbonyl)-3,6-diazabicyclo[3.2.1]octane,
3-(4-methyloxazol-5-ylcarbonyl)-3,6-diazabicyclo[3.2.1]octane,
6-(4-methyloxazol-5-ylcarbonyl)-3,6-diazabicyclo[3.2.1]octane,
3-(isoxazol-3-ylcarbonyl)-3,6-diazabicyclo[3.2.1]octane,
6-(isoxazol-3-ylcarbonyl)-3,6-diazabicyclo[3.2.1]octane,
3-(5-methylisoxazol-3-ylcarbonyl)-3,6-diazabicyclo[3.2.1]octane,
6-(5-methylisoxazol-3-ylcarbonyl)-3,6-diazabicyclo[3.2.1]octane,
3-(isoxazol-4-ylcarbonyl)-3,6-diazabicyclo[3.2.1]octane,
6-(isoxazol-4-ylcarbonyl)-3,6-diazabicyclo[3.2.1]octane,
3-(3-methylisoxazol-4-ylcarbonyl)-3,6-diazabicyclo[3.2.1]octane,
6-(3-methylisoxazol-4-ylcarbonyl)-3,6-diazabicyclo[3.2.1]octane,
3-(5-methylisoxazol-4-ylcarbonyl)-3,6-diazabicyclo[3.2.1]octane,
6-(5-methylisoxazol-4-ylcarbonyl)-3,6-diazabicyclo[3.2.1]octane,
3-(isoxazol-5-ylcarbonyl)-3,6-diazabicyclo[3.2.1]octane,
6-(isoxazol-5-ylcarbonyl)-3,6-diazabicyclo[3.2.1]octane,
3-(3-methylisoxazol-5-ylcarbonyl)-3,6-diazabicyclo[3.2.1]octane,
6-(3-methylisoxazol-5-ylcarbonyl)-3,6-diazabicyclo[3.2.1]octane,
3-(3-bromoisoxazol-5-ylcarbonyl)-3,6-diazabicyclo[3.2.1]octane,
6-(3-bromoisoxazol-5-ylcarbonyl)-3,6-diazabicyclo[3.2.1]octane,
3-(3-methoxyisoxazol-5-ylcarbonyl)-3,6-diazabicyclo[3.2.1]octane,
6-(3-methoxyisoxazol-5-ylcarbonyl)-3,6-diazabicyclo[3.2.1]octane,
3-(pyridin-4-ylcarbonyl)-3,6-diazabicyclo[3.2.1]octane,
6-(pyridin-4-ylcarbonyl)-3,6-diazabicyclo[3.2.1]octane,
   and pharmaceutically acceptable salts thereof.

For the avoidance of doubt, the present invention relates to any one of the specific compound mentioned above.

### Compound Preparation

The compounds of the present invention can be prepared via the coupling of an unprotected or mono-protected diazabicyclic core (i.e., one in which one of the two amine functional groups is rendered un-reactive by suitable derivatization) with a suitably functionalized heteroarylcarboxylic acid, the corresponding acid chloride or other reactive heteroarylcarboxylic acid derivative.

There are numerous methods for preparing the mono-protected diazabicycles used to prepare the compounds of the present invention. For instance, methods for the synthesis of a suitably protected single enantiomer 2,6-diazabicyclo[3.2.0]heptanes are described in PCT WO 05/028477 to Basha et al. and in US application 2002/0019388 to Schrimpf et al., each of which is herein incorporated by reference with regard to such synthetic teaching, in which trans-3-hydroxy-L-proline is treated with di-tert-butyl dicarbonate to give (2S,3S)-3-hydroxypyrrolidine-1,2-dicarboxylic acid 1-tert-butyl ester. Reduction with borane, and subsequent treatment with methanesulfonyl chloride and triethylamine affords (2R,3S)-3-methanesulfonyloxy-2-methanesulfonyloxymethylpyrrolidine-1-carboxylic acid tert-butyl ester, which is converted to (1R,5R)-6-benzyl-2,6-diazabicyclo[3.2.0]heptane-2-carboxylic acid tert-butyl ester upon treatment with benzyl amine. Each of the two protecting groups can be removed selectively, to provide suitably protected intermediates for conversion to compounds of the present invention. Thus, hydrogenation gives the (1R,5R)-2,6-diazabicyclo[3.2.0]heptane-2-carboxylic acid tert-butyl ester. Alternatively, treatment of the (1R,5R)-6-benzyl-2,6-diazabicyclo[3.2.0]heptane-2-carboxylic acid tert-butyl ester with strong acid, such as trifluoroacetic acid, gives (1R,5R)-6-benzyl-2,6-diazabicyclo[3.2.0]heptane. Both the 2,6-diazabicyclo[3.2.0]heptane-2-carboxylic acid tert-butyl ester and the 6-benzyl-2,6-diazabicyclo[3.2.0]heptane are appropriately constructed for conversion into compounds of the present invention. If the same sequence is carried out, using trans-3-hydroxy-D-proline as a starting material, the corresponding intermediates of the (1S,5S) configuration are produced.

Methods for the synthesis of a suitably protected 3,6-diazabicyclo[3.2.0]heptane can vary. For instance, one synthesis is described in PCT WO 05/028477 to Basha et al., and in U.S. application 2006/0035937 to Wayne et al., each of which is herein incorporated by reference with regard to such synthetic teaching, in which 2,2-dimethoxyethylcarbamic acid benzyl ester is prepared via either the combination of benzylchloroformate or benzyloxycarbonyl oxysuccinimide with aminoacetaldehyde dimethyl acetal (or 2,2-dimethoxyethylamine). Alkylation with base and allyl bromide, followed by the conversion of the dimethoxy acetal to the corresponding oxime (using hydroxylamine) affords allyl-(2-hydroxyiminoethyl)-carbamic acid benzyl ester. Heating effects a 3+2 cycloaddition, and subsequent reduction affords benzyl cis-3-amino-4-(hydroxymethyl)pyrrolidine-1-carboxylate, an intermediate which can be resolved into its corresponding (3R,4R) and (3S, 4S) stereoisomers by reaction with (S)- or (R)-mandelic acid respectively. Subsequent protection of the amine with a tert-butoxycarbonyl group, conversion of the alcohol to an alkyl chloride, removal of the tert-butoxycarbonyl protecting group, and treatment with base gives the 3-(benzyloxycarbonyl)-3,6-diazabicyclo[3.2.0]heptane, of either the (1R,5R) or (1S,5S) configuration. Protection of the free 6-position amine with di-tert-butyl dicarbonate, followed by hydrogenation, affords an alternate mono-protected product, 6-(tert-butoxycarbonyl)-3,6-diazabicyclo[3.2.0]heptane, in single enantiomer forms. Intermediates such as 3-(benzyloxycarbonyl)-3,6-diazabicyclo[3.2.0]heptane and 6-(tert-butoxycarbonyl)-3,6-diazabicyclo[3.2.0]heptane are suitable for conversion into compounds of the present invention.

Methods for the synthesis of a suitably protected 2,6-diazabicyclo[3.3.0]octane can vary. One such synthesis is described by Cope and Shen in J. Am. Chem. Soc. 78: 5916-20 (1956) and in U.S. Pat. No. 2,932,650, each of which is herein incorporated by reference with regard to such synthetic teaching, in which isomannide dichloride (from D-mannitol) is catalytically hydrogenated to produce D-2,6-dioxabicyclo[3.3.0]octane. Treatment of D-2,6-dioxabicyclo[3.3.0]octane with dry hydrogen bromide gas gives D-1,6-dibromohexane-3,4-diol, which is subsequently converted to its corresponding ditosylate. Treatment of D-1,6-dibromohexane-3,4-diol ditosylate with benzylamine, followed by hydrogenolysis of the benzyl protecting group, gives the (1R,5R)-2,6-diazabicyclo[3.3.0]octane. The hydrogenolysis can be interrupted before completion to gain access to the mono-benzyl derivative. (1S,5S)-2,6-Diazabicyclo[3.3.0]octane can be produced similarly, from L-2,6-dioxabicyclo[3.3.0]octane, which is produced from D-1,6-dibromohexane-3,4-diol ditosylate by inversion of stereochemistry by acetate displacement, followed by cyclization with methoxide ion. Both 2,6-diazabicyclo[3.3.0]octane and its 2-benzyl derivative are suitable intermediates for conversion into compounds of the present invention.

Methods for the synthesis of a suitably protected 2,7-diazabicyclo[3.3.0]octane can vary. One such method is described in PCT WO 05/028477 to Basha et al. and in U.S. Pat. No. 5,071,999 to Schenke and Petersen, each of which is herein incorporated by reference with regard to such synthetic teaching, in which ethyl bromoacetate is reacted with α-methylbenzylamine to give ethyl (R)-(1-phenylethyl)aminoacetate, which is then hydrolyzed in water to the corresponding acetic acid. Condensation of (R)-(1-phenylethyl)aminoacetic acid with ethyl N-allyl-N-(2-oxoethyl)carbamate, at reflux in toluene, gives diastereomeric ethyl 2-((R)-1-phenylethyl)-2,7-diazabicyclo[3.3.0]octane-7-carboxylates, which can be separated chromatographically. The separated diastereomers have the (1R,5R) and (1S,5S) configurations at the ring junction and are differentially protected at the 2- and 7-positions. Thus selective deprotection by acid hydrolysis or hydrogenation gives single enantiomer 2,7-diazabicyclo[3.3.0]octanes, with a free 7-position amine or a free 2-position amine respectively. Such compounds are suitable intermediates for conversion into compounds of the present invention. Other protecting group manipulations are possible.

Methods of making suitably protected 2,8-diazabicyclo[4.3.0]nonanes can vary. One such method is reported by Takemura et al. in EP 0603887, which is herein incorporated by reference with regard to such synthetic teaching, in which pyridine-2,3-dicarboxylic acid is converted into the corresponding N-benzyl imide, and then sequentially reduced by hydrogenation over ruthenium and lithium aluminum hydride. The resulting 8-benzyl-2,8-diazabicyclo[4.3.0]nonane can be used directly as an intermediate in the synthesis of compounds of the present invention, or can be further transformed, by reaction with di-tert-butyl dicarbonate and subsequent hydrogenation, to produce tert-butyl 2,8-diazabicyclo[4.3.0]nonane-2-carboxylate (also an intermediate suitable for synthesis of compounds of the present invention). The 8-benzyl-2,8-diazabicyclo[4.3.0]nonane can be resolved into its enantiomers by selective crystallization of its D- and L-tartrate salts, to form single enantiomer intermediates suitable for conversion into compounds of the present invention.

Methods for the synthesis of a suitably protected 3,8-diazabicyclo[4.3.0]nonane can vary. One such method is described in US application 2002/0019388 to Schrimpf et al., which is herein incorporated by reference with regard to such synthetic teaching, in which commercially available 3,4-pyridinedicarboximide is sequentially alkylated on the imide nitrogen with benzyl bromide, hydrogenated over platinum, and reduced with lithium aluminum hydride. The resulting 8-benzyl-3,8-diazabicyclo[4.3.0]nonane can be use directly in forming compounds of the present invention or can be treated with di-tert-butyl dicarbonate to form 8-benzyl-3-(tert-butoxycarbonyl)-3,8-diazabicyclo[4.3.0]nonane. Hydrogenation of the 8-benzyl-3-(tert-butoxycarbonyl)-3,8-diazabicyclo[4.3.0]nonane will produce 3-(tert-butoxycarbonyl)-3,8-diazabicyclo[4.3.0]nonane, which can be used to generate compounds of the present invention.

Another method of making suitably protected 3,8-diazabicyclo[4.3.0]nonanes is reported in PCT WO 05/028477 to Basha et al., which is herein incorporated by reference with regard to such synthetic teaching, in which cyclopentenone, N-benzyl-N-(methoxymethyl)trimethylsilylmethylamine, and trifluoroacetic acid are reacted. The cycloaddition reaction results in the production of 7-benzyl-7-azabicyclo[3.3.0]octan-2-one, which is subsequently reacted with hydroxylamine hydrochloride and sodium acetate to give the corresponding oxime. Treatment with polyphosphoric acid affords the ring-expanded lactam (8-benzyl-3,8-diazabicyclo[4.3.0]nonan-2-one), which is subsequently reduced by treatment with lithium aluminum hydride to give 8-benzyl-3,8-diazabicyclo[4.3.0]nonane (which is also named 2-benzyloctahydropyrrolo[3,4-c]pyridine), an intermediate suitable for conversion to compounds of the present invention.

Alternatively, a suitably protected 3,8-diazabicyclo[4.3.0]nonane can be prepared via the conversion of tert-butyl 7-oxo-3-azabicyclo[3.3.0]octane-3-carboxylate (available as described by Becker and Flynn, Tetrahedron 49(23): 5047-5054 (1993), which is herein incorporated by reference with regard to such synthetic teaching) to its oxime derivative, followed by treatment with polyphosphoric acid to give the lactam, tert-butyl 4-oxo-3,8-diazabicyclo[4.3.0]nonane-8-carboxylate. Reduction with borane-methyl sulfide complex affords the mono-protected product, tert-butyl 3,8-diazabicyclo[4.3.0]nonane-8-carboxylate. To generate the other mono-protected amine product, protection of the free amine with 9-fluorenylmethoxycarbonyl followed by removal of the tert-butoxycarbonyl group affords 9-fluorenylmethyl 3,8-diazabicyclo[4.3.0]nonane-8-carboxylate. Methods of separating the enantiomeric forms of 3,8-diazabicyclo[4.3.0]nonanes are known to those of skill in the art of organic synthesis. Thus, resolution by formation of diastereomeric salts, using single enantiomer chiral acids, is possible, as well as resolution by formation of diastereomeric intermediates (for instance, the (R)- or (S)-1-phenylethyl derivatives at either to 3- or 8-positions) that can be separated by chromatographic means. Thus produced and suitably protected, these single enantiomer forms can be converted into compounds of the present invention.

Methods for the synthesis of a suitably protected 2,6-diazabicyclo[3.2.1]octanes can vary. One such method is described in PCT WO 05/028477 to Basha et al., which is herein incorporated by reference with regard to such synthetic teaching, in which benzyl 5-oxo-2-azabicyclo[2.2.1]heptane-2-carboxylate (prepared according to the procedure described by Carroll, et al., J. Med. Chem. 35: 2184 (1992), which is herein incorporated by reference with regard to such synthetic teaching), is converted into its oxime derivative, which is then stirred with trimethylsilylpolyphosphate to effect ring expansion, giving benzyl 3-oxo-2,6-diazabicyclo[3.2.1]octane-6-carboxylate. Sequential treatment with borane-methyl sulfide complex and n-propylamine gives the mono-protected diazabicyclic product, benzyl 2,6-diazabicyclo[3.2.1]octane-6-carboxylate. Protection of the free 2-position amine with di-tert-butyl dicarbonate, followed by hydrogenolysis of the benzyloxycarbonyl protecting group, gives another mono-protected diazabicycle, tert-butyl 2,6-diazabicyclo[3.2.1]octane-2-carboxylate. Methods of separating the enantiomeric forms of 2,6-diazabicyclo[3.2.1]octanes are known to those of skill in the art of organic synthesis. Thus, resolution by formation of diastereomeric salts, using single enantiomer chiral acids, is possible, as well as resolution by formation of diastereomeric intermediates that can be separated by chromatographic means. Thus produced and suitably protected, these single enantiomer forms can be converted into compounds of the present invention.

Methods for the synthesis of a suitably protected 3,6-diazabicyclo[3.2.1]octanes can vary. One such method is described in PCT WO 05/028477 to Basha et al., which is herein incorporated by reference with regard to such synthetic teaching, in which formalin and ammonium chloride are combined with cyclopentadiene, followed by reaction with di-tert-butyl dicarbonate, to afford tert-butyl 2-azabicyclo[2.2.1]hept-5-en-2-carboxylate. Sequential treatment with ozone and dimethylsulfide produces tert-butyl 2,4-diformylpyrrolidin-1-carboxylate. Treatment of tert-butyl 2,4-diformylpyrrolidin-1-carboxylate with benzylamine and sodium cyanoborohydride affords tert-butyl 3-benzyl-3,6-diazabicyclo[3.2.1]octane-6-carboxylate. To produce mono-protected diazabicyclic amine compounds, either the benzyl group can be removed by hydrogenation or the tert-butoxycarbonyl group can be removed by treatment with strong acid, affording tert-butyl 3,6-diazabicyclo[3.2.1]octane-6-carboxylate and 3-benzyl-3,6-diazabicyclo[3.2.1]octane respectively. Methods of separating the enantiomeric forms of 3,6-diazabicyclo[3.2.1]octanes are known to those of skill in the art of organic synthesis. Thus, resolution by formation of diastereomeric salts, using single enantiomer chiral acids, is possible, as well as resolution by formation of diastereomeric intermediates (for instance, as would be produced by the use of either (R)- or (S)-1-phenylethylamine in place of benzylamine in the reductive amination step) that can be separated by chromatographic means. Thus produced and suitably protected, these single enantiomer forms can be converted into compounds of the present invention.

Alternately suitably protected single enantiomer 3,6-diazabicyclo[3.2.1]octanes can be made from single enantiomer starting materials. Thus, sequential treatment of commercially available (1R)-2-azabicyclo[2.2.1]hept-5-en-3-one or (1S)-2-azabicyclo[2.2.1]hept-5-en-3-one with lithium aluminum hydride and di-tert-butyl dicarbonate will generate tert-butyl (1R)-2-azabicyclo[2.2.1]hept-5-en-2-carboxylate and tert-butyl (1S)-2-azabicyclo[2.2.1]hept-5-en-2-carboxylate respectively. These single enantiomer intermediates can be transformed, as described above for the corresponding racemate, into the single enantiomers of tert-butyl 3,6-diazabicyclo[3.2.1]octane-6-carboxylate and 3-benzyl-3,6-diazabicyclo[3.2.1]octane. In a slightly different approach, the single enantiomer tert-butyl 2,4-diformylpyrrolidin-1-carboxylates can be converted into the single enantiomer 3,6-diazabicyclo[3.2.1]octanes by reduction of the formyl groups to the corresponding alcohols, followed by formation of the di-mesylate derivatives and cyclization with ammonia and cuprous iodide. This produces the enantiomeric tert-butyl 3,6-diazabicyclo[3.2.1]octane-6-carboxylates directly, without having to remove a benzyl protecting group. The enantiomeric tert-butyl 3,6-diazabicyclo[3.2.1]octane-6-carboxylates are suitable intermediates for conversion into compounds of the present invention.

Methods for the synthesis of a suitably protected 3,8-diazabicyclo[4.2.0]octane can vary. One such method is described in PCT WO 05/028477 to Basha et al. and in Frost et al., J. Med. Chem. 49: 7843 (2006), each of which is herein incorporated by reference with regard to such synthetic teaching, in which ethyl N-benzyl-3-oxo-4-piperidinecarboxylate hydrochloride, di-tert-butyl dicarbonate, triethylamine, and palladium hydroxide on carbon were shaken together under hydrogen. The resulting 3-oxopiperidine-1,4-dicarboxylic acid 1-tert-butylester 4-ethyl ester is condensed with (R)-methylbenzylamine at reflux in toluene, and the product is subsequently reduced with sodium triacetoxyborohydride and acetic acid to give 3-((1R)-1-phenylethylamino)-piperidine-1,4-dicarboxylic acid 1-tert-butyl ester 4-ethyl ester. Reduction with lithium aluminum hydride gives 4-(hydroxymethyl)-3-((1R)-1-phenylethylamino)-piperidine-1-carboxylic acid tert-butyl ester. Treatment of this intermediate with triethylamine, methanesulfonylchloride and cesium carbonate gives a pair of diastereomeric 3-((1R)-1-phenylethyl)-3,8-diazabicyclo[4.2.0]octane-3-carboxylic acid tert-butyl esters, which are separable chromatographically. Once separated from one another, the two intermediates can be either hydrogenated (to remove the 1-phenylethyl group) or treated with strong acid (to remove the tert-butoxycarbonyl group), affording single enantiomer forms of 3,8-diazabicyclo[4.2.0]octane, differentially protected for conversion into compounds of the present invention.

The procedures found in PCT WO 05/028477 to Basha et al. and in Frost et al., J. Med. Chem. 49: 7843 (2006), each of which is herein incorporated by reference with regard to such synthetic teaching, can be adapted for the synthesis of tert-butyl 3,7-diazabicyclo[4.2.0]octane-7-carboxylate, by using the commercially available ethyl N-benzyl-3-oxo-4-piperidinecarboxylate hydrochloride as the starting material and carrying out the analogous transformations. The tert-butyl 3,7-diazabicyclo[4.2.0]octane-7-carboxylate, thus produced, can be sequentially treated with trifluoroacetic anhydride and trifluoroacetic acid to make 3-trifluoroacetyl-3,7-diazabicyclo[4.2.0]octane. Both tert-butyl 3,7-diazabicyclo[4.2.0]octane-7-carboxylate and 3-trifluoroacetyl-3,7-diazabicyclo[4.2.0]octane can be coupled with various heteroaryl carboxylic acids to make compounds of the present invention.

Methods for making other suitably protected diazabicycles will be apparent to those of skill in the art of organic synthesis. For instance, the synthesis of 2,7-diazabicyclo[4.3.0]nonanes, 3,7-diazabicyclo[4.3.0]nonanes and 3,9-diazabicyclo[4.3.0]nonanes, among others, are outlined in US application 2002/0019388 to Schrimpf et al., the contents of which are incorporated by reference. Such compounds can serve as intermediates for the synthesis of compounds of the present invention.

Other methods for installation and removal of the benzyl, tert-butoxycarbonyl, and other amine protecting groups are well known by those skilled in the art and are described further in T. W. Greene and P. G. M. Wuts, Protective Groups in Organic Synthesis, 3^{rd} Edition, John Wiley & Sons, New York (1999), which is herein incorporated by reference with regard to such synthetic teaching,

Methods of making heteroarylcarboxamides of the present invention vary. Generally, the suitably protected biazabicycle is reacted with either a heteroarylcarboxylic acid or an activated derivative thereof (e.g., a heteroarylcarboxylic acid chloride), in the presence of dehydrating agents and/or bases. A variety of conditions are possible. Coupling of the heteroarylcarboxylic acid to the suitably protected diazabicycle can be accomplished in a number of ways. Typically, the heteroarylcarboxylic acid is coupled to a diazabicyclic intermediate with a free amine functionality, using any one of various agents used for forming amide bonds (for instance, in the synthesis of peptides). Such reagents include N,N′-dicyclohexylcarbodiimide (DCC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), 0-(benzotriazol-1-yl)-N,N,N′,N′-bis(tetramethylene)uronium hexafluorophosphate (HBPyU), O-(benzotriazol-1-yl)-N,N,N,N′-tetramethyluronium hexafluorophosphate (HBTU), O-(benzotriazol-1-yl)-N,N,N′,N′-tetramethyluronium tetrafluoroborate (TBTU), and (1-ethyl-3-(3-dimethylaminopropyl)carbodiimide) (EDCI) with 1-hydroxybenzotriazole (HOBt). Other coupling agents are well known to those skilled in the art (for example, see Kiso and Yajima, Peptides, pp 39-91, Academic Press, San Diego, Calif. (1995), which is herein incorporated by reference with regard to such synthetic teaching). In some cases these reagents are commercially available as polymer supported modifications, which greatly facilitate isolation of coupling products. An example of such a reagent is polystyrene bound N,N′-dicyclohexylcarbodiimide (PS-DCC).

Alternatively, the amide bond, in compounds of the present invention, can be formed by coupling a suitably protected diazabicycle with a heteroarylcarboxylic acid chloride, which may be available commercially or may be prepared by reaction of a heteroarylcarboxylic acid with any of various reagents, such as thionyl chloride or oxalyl chloride. The reaction between the acid chloride and the diazabicycle is typically performed in the presence of a tertiary amine, usually a hindered one.

Typically, after amide bond formation, a protecting group (e.g., the tert-butoxycarbonyl group or a benzyl group) must be removed to generate compounds of the present invention. Means of removal of the protecting groups mentioned herein, and other suitable protecting groups, described in T. W. Greene and P. G. M. Wuts, Protective Groups in Organic Synthesis, 3^{rd} Edition, John Wiley & Sons, New York (1999), which is herein incorporated by reference with regard to such synthetic teaching.

The heteroarylcarboxylic acids used to make compounds of the present invention are often commercially available. Those that are not commercially available can be made by a variety of synthetic methodologies, related to the particular heteroaromatic ring and the particular substitution pattern desired. The variation in synthetic methodology will be readily apparent to those of skill in the art of organic synthesis.

Those skilled in the art of organic synthesis will appreciate that there exist multiple means of producing compounds of the present invention which are labeled with a radioisotope appropriate to various diagnostic uses. For example, the use of a ¹¹C- or ¹⁸F-labeled heteroaromatic carboxylic acid in condensation with one of the tert-butoxycarbonyl-protected diazabicyclic cores described herein, using the methods described above, and subsequent removal of the tert-butoxycarbonyl group will produce a compound suitable for use in positron emission tomography.

### Methods of Treatment

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition. Thus, in another aspect, the invention provides a method for treating a subject having or at risk of developing or experiencing a recurrence of a NNR or nAChR mediated disorder. The compounds and pharmaceutical compositions of the invention may be used to achieve a beneficial therapeutic or prophylactic effect, for example, in a subject with a CNS dysfunction.

As noted above, the compounds of the present invention are modulators of the α4β2 NNR subtype, characteristic of the CNS, and can be used for preventing or treating various conditions or disorders, including those of the CNS, in subjects which have or are susceptible to such conditions or disorders, by modulation of α4β2 NNRs. The compounds have the ability to selectively bind to the α4β2 NNRs and express nicotinic pharmacology, for example, to act as agonists, partial agonists, antagonists, as described. For example, compounds of the present invention, when administered in effective amounts to patients in need thereof, provide some degree of prevention of the progression of the CNS disorder, namely, providing protective effects, amelioration of the symptoms of the CNS disorder, or amelioration of the reoccurrence of the CNS disorder, or a combination thereof.

The compounds of the present invention can be used to treat or prevent those types of conditions and disorders for which other types of nicotinic compounds have been proposed or are shown to be useful as therapeutics. See, for example, the references previously listed hereinabove, as well as Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. Nos. 5,583,140 to Bencherif et al., 5,597,919 to Dull et al., 5,604,231 to Smith et al. and 5,852,041 to Cosford et al., the disclosures of which are incorporated herein by reference with regard to such therapeutic teaching.

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment, mild cognitive impairment, age-related cognitive decline, pre-senile dementia, early onset Alzheimer's disease, senile dementia, dementia of the Alzheimer's type, Lewy body dementia, HIV-dementia, vascular dementia, Alzheimer's disease, stroke, ischemia, traumatic brain injury, AIDS dementia complex, attention deficit is disorder, attention deficit hyperactivity disorder, dyslexia, schizophrenia, schizophreniform disorder, schizoaffective disorder, cognitive dysfunction in schizophrenia, Parkinsonism including Parkinson's disease, Pick's disease, Huntington's chorea, tardive dyskinesia, hyperkinesia, progressive supranuclear palsy, restless leg syndrome, Creutzfeld-Jakob disease, multiple sclerosis, amyotrophic lateral sclerosis, epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, oppositional defiant disorder, Tourette's syndrome, autism, drug and alcohol addiction, tobacco addiction, obesity, cachexia, psoriasis, lupus, acute cholangitis, aphthous stomatitis, ulcers, asthma, ulcerative colitis, inflammatory bowel disease, Crohn's disease, spastic dystonia, diarrhea, constipation, pouchitis, viral pneumonitis, arthritis, including, rheumatoid arthritis and osteoarthritis, endotoxaemia, sepsis, atherosclerosis, idiopathic pulmonary fibrosis, acute pain, chronic pain, neuropathies, urinary incontinence, diabetes and neoplasias.

Cognitive impairments or dysfunctions may be associated with psychiatric disorders or conditions, such as schizophrenia and other psychotic disorders (including but not limited to psychotic disorder, schizophreniform disorder, schizoaffective disorder, delusional disorder, brief psychotic disorder, shared psychotic disorder, and psychotic disorders due to a general medical conditions), dementias and other cognitive disorders (including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions), anxiety disorders (including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition), mood disorders (including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions), sleep disorders (including but not limited to dyssomnia disorders, primary insomnia, primary hypersomnia, narcolepsy, parasomnia disorders, nightmare disorder, sleep terror disorder and sleepwalking disorder), mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood or adults, tic disorders, elimination disorders, substance-related disorders (including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders), personality disorders (including but not limited to obsessive-compulsive personality disorder and impulse-control disorders).

The above conditions and disorders are defined for example in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence, and is herein incorporated by reference with regard to such.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, can modulate the activity of the α4β2 NNRs without appreciable interaction with the nicotinic subtypes that characterize the human ganglia, as demonstrated by their lack of the ability of to elicit nicotinic function in adrenal chromaffin tissue, or skeletal muscle, as demonstrated by their lack of ability to elicit nicotinic function in cell preparations expressing muscle-type nicotinic receptors. Thus, these compounds are capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is sufficient to provide the desired effects upon the disease, disorder or condition, but is insufficient, namely is not at a high enough level, to provide undesirable side effects.

Thus, the present invention provides the use of a compound of the present invention, or a pharmaceutically acceptable salt thereof, for use in therapy, such such as any one of the therapies described above.

In yet another aspect the present invention provides the use of a compound of the present invention, or a pharmaceutically acceptable salt thereof, in the manufacture of a medicament for the treatment of a CNS disorder, such as a disorder, disease or condition described hereinabove.

In a further aspect the invention provides the use of a compound of the present invention, or a pharmaceutically acceptable salt thereof, in the manufacture of a medicament for the treatment mild to moderate dementia of the Alzheimer's type, attention deficit disorder, mild cognitive impairment, age-associated memory impairment and cognitive dysfunction in schizophrenia.

### Diagnostic Uses

The compounds can be used in diagnostic compositions, such as probes, particularly when they are modified to include appropriate labels. The probes can be used, for example, to determine the relative number and/or function of specific receptors, particularly the α4β2 receptor subtype. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al, herein incorporated by reference with regard to such techniques. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate to techniques in order to indicate the presence, quantity, and functionality of selected nicotinic cholinergic receptor subtypes. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al. for a disclosure of representative imaging techniques; each herein incorporated by reference with regard to such teaching.

The radiolabeled compounds bind with high affinity to selective nAChR subtypes (e.g., α4β2) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions can be used in a method to diagnose disease in a subject, such as a human patient. The method involves administering to that patient a detectably labeled compound as described herein, and detecting the binding of that compound to selected nicotinic receptor subtypes (e.g., α4β2 receptor subtype). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth herein as well as in U.S. Pat. No. 5,952,339 to Bencherif et al., herein incorporated by reference in entirety.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes (e.g., the α4β2 receptor subtype).

### Pharmaceutical Compositions

The pharmaceutical compositions of the present invention incorporate a compound of the present invention which, when employed in effective amounts, interacts with relevant nicotinic receptor sites of a subject, and acts as a therapeutic agent to treat and prevent a wide variety of conditions and disorders. The pharmaceutical compositions provide therapeutic benefit to individuals suffering from affected disorders or exhibiting clinical manifestations of affected disorders, in that the compounds within those compositions, when employed in effective amounts, can: (i) exhibit nicotinic pharmacology and affect relevant nicotinic receptors sites, for example by acting as a pharmacological agonist to activate a nicotinic receptor; or (ii) elicit neurotransmitter secretion, and hence prevent and suppress the symptoms associated with those diseases.

The compounds of the present invention have the potential to (i) increase the number of nicotinic cholinergic receptors of the brain of a subject in need thereof; (ii) exhibit neuroprotective effects; and (iii) when employed in effective amounts, to not cause appreciable adverse side effects, for example, significant increases in blood pressure and heart rate, significant negative effects upon the gastro-intestinal tract, or significant effects upon skeletal muscle.

The present invention further provides pharmaceutical compositions that include effective amounts of compounds of the formulae of the present invention and salts and solvates, thereof, and one or more pharmaceutically acceptable carriers, diluents, or excipients. The compounds of the formulae of the present invention, including salts and solvates, thereof, are as herein described. The carrier(s), diluent(s), or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formulae of the present invention, including a salt, solvate, or prodrug thereof, with one or more pharmaceutically acceptable carriers, diluents or excipients.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Compositions may be formulated in unit dose form, or in multiple or subunit doses. Preferred compositions are in liquid or semisolid form.

Compositions including a liquid pharmaceutically inert carrier such as water or other pharmaceutically compatible liquids or semisolids may be used. The use of such liquids and semisolids is well known to those of skill in the art.

The compositions can also be administered via injection, i.e., intravenously, intramuscularly, subcutaneously, intraperitoneally, intraarterially, intrathecally, and intracerebroventricularly. Intravenous administration is a preferred method of injection. Suitable carriers for injection are well known to those of skill in the art, and include 5% dextrose solutions, saline, and phosphate buffered saline. The compounds can also be administered as an infusion or injection (e.g., as a suspension or as an emulsion in a pharmaceutically acceptable liquid or mixture of liquids).

The formulations may also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in is U.S. Pat. No. 4,922,901 to Brooks et al., the disclosure of which is incorporated herein in its entirety); topically (e.g., in lotion form); transdermally (e.g., using a transdermal patch, using technology that is commercially available from Novartis and Alza Corporation, or by powder injection); or by buccal or intranasal absorption. Although it is possible to administer the compounds in the form of a bulk active chemical, it is preferred to present each compound in the form of a pharmaceutical composition or formulation for efficient and effective administration.

Exemplary methods for administering such compounds will be apparent to the skilled artisan. The usefulness of these formulations may depend on the particular composition used and the particular subject receiving the treatment. For example, the compositions can be administered in the form of a tablet, a hard gelatin capsule or as a time release capsule. These formulations may contain a liquid carrier that may be oily, aqueous, emulsified or contain certain solvents suitable to the mode of administration.

The administration of the pharmaceutical compositions described herein can be intermittent, or at a gradual, continuous, constant or controlled rate to a warm-blooded animal, (e.g., a mammal such as a mouse, rat, cat, rabbit, dog, pig, cow, or monkey); but advantageously is preferably administered to a human being. In addition, the time of day and the number of times per day that the pharmaceutical composition is administered can vary.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder. Thus, when treating a CNS disorder, an effective amount of compound is an amount sufficient to pass across the blood-brain barrier of the subject, to bind to relevant receptor sites in the brain of the subject, and to modulate the activity of relevant nicotinic receptor subtypes (e.g., modulate neurotransmitter secretion, thus resulting in effective prevention or treatment of the disorder). Prevention of the disorder is manifested by delaying the onset of the symptoms of the disorder. Treatment of the disorder is manifested by a decrease in the symptoms associated with the disorder or an amelioration of the reoccurrence of the symptoms of the disorder.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to modulate disease-relevant receptors to affect neurotransmitter (e.g., dopamine) release but the amount should be insufficient to induce effects on skeletal muscles and ganglia to any significant degree. The effective dose of compounds will of course differ from patient to patient but in general includes amounts starting where CNS effects or other desired therapeutic effects occur, but below the amount where muscular and ganglionic effects are observed.

Typically, to be administered in an effective dose, compounds require administering in an amount of less than 5 mg/kg of patient weight. Often, the compounds may be administered in an amount from less than about 1 mg/kg patient weight to less than about 100 μg/kg of patient weight, and occasionally between about 10 μg/kg to less than 100 μg/kg of patient weight. The foregoing effective doses typically represent that amount administered as a single dose, or as one or more doses administered over a 24 hours period. For human patients, the effective dose of the compounds may require administering the compound in an amount of at least about 1, but not more than about 1000, and often not more than about 500 mg/24 hr/patient.

Compositions useful as diagnostics can be employed, as set forth in U.S. Pat. Nos. 5,853,696 to Elmalch et al. and 5,969,144 to London et al., the contents of which are hereby incorporated by reference. The compounds also can be administered in formulation compositions that incorporate other ingredients, such as those types of ingredients that are useful in formulating a diagnostic composition.

The present invention also encompasses combination therapy for treating or preventing a disorder mediated by a NNR or nAChR in a subject. The combination therapy comprises administering to the subject a therapeutically or prophylactically effective amount of a compound of the present invention and one or more other therapy including chemotherapy, radiation therapy, gene therapy, or immunotherapy.

In an embodiment of the present invention, the compound of the present invention may be administered in combination with other therapeutic compounds. In particular, a compound of this invention can be advantageously used in combination with other NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine olanzapine and quetiapine), immuno-suppressants (such as cyclosporin and methotrexate), neuroprotective agents, steroids (such as steroid hormones), corticosteroids (such as dexamethasone, predisone and hydrocortisone), vitamins, minerals, nutraceuticals, anti-depressants (such as imipramine, fluoxetine, paroxetine, escitalopram, sertraline, venlafaxine and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil and olmesartan), laxatives, stool softeners, diuretics (such as furosemide), anti-spasmotics (such as dicyclomine), anti-dyskinetic agents, and anti-ulcer medications (such as esomeprazole).

The compounds of the present invention may be employed alone or in combination with other therapeutic agents, including other compounds of the present invention. Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the formulae of the present invention including salts or solvates thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The compounds of the present invention may be used in the treatment of a variety of disorders and conditions and, as such, the compounds of the present invention may be used in combination with a variety of other suitable therapeutic agents useful in the treatment and/or prophylaxis of those disorders or conditions.

The following examples are provided to illustrate the present invention, and should not be construed as limiting thereof. In these examples, all parts and percentages are by weight, unless otherwise noted.

### List of Abbreviations

The following definitions for abbreviations used herein are meant to clarify, but not limit, the terms defined. If a particular abbreviation used herein is not specifically defined, the abbreviation term should not be considered indefinite. Rather, abbreviations are used within their accepted meanings in the art.

THF (tetrahydrofuran)
CMA 90 (chloroform:methanol: aqueous ammonium hydroxide (90:9:1))
DCC(N,N′-dicyclohexylcarbodiimide)
PS-DCC (polystyrene bound N,N′-dicyclohexylcarbodiimide)
HOBt (1-hydroxybenzotriazole)
TFA (trifluoroacetic acid)
HPLC (high performance liquid chromatography)
MLA (methyllycaconitine)
NOR (novel object recognition)
ND (not determined)
[³H] tritium, labeled with radioactive hydrogen
[³H]DA dopamine radiolabeled with tritium
[³H]MLA methyllycaconitine radiolabeled with tritium
[³H]QNB 3-quinuclidinyl benzilate radiolabeled with tritium
° C. degrees celcius
⁸⁶Rb⁺ radioactive rubidium

### AIDS Acquired Immune Deficiency Syndrome

CaCl₂ calcium chloride

### Ci Curie

CNS central nervous system
CO₂ carbon dioxide
DA dopamine
EC₅₀ drug concentration that provokes a half-maximal response
EDTA ethylenediaminetetraacetic acid
Eₘₐₓ maximal effect
g grams
g unit of force to which a body is subjected when undergoing acceleration
GF/B glass fiber filter, pore size B
h hours
HEPES 4-(2-hydroxyethyl)-1-piperazineethanesulfonic acid

### HIV Human Immunodeficiency Virus

HTS high throughput screening
IC₅₀ concentration that inhibits activity by 50 percent
KCl potassium chloride
KH₂PO₄ potassium phosphate, monobasic

equilibrium dissociation constant for competitor inhibited radioligand

Kᵢ binding
M molar
mg milligram
μg microgram
MgCl₂ magnesium chloride
Min minutes
mL milliliter
μl microliter
MLA methyllycaconitine
mM millimolar
μM micromolar
mmol millimol
Na₂HPO₄ sodium phosphate, dibasic
nAChR nicotinic acetylcholine receptor
nAChRs nicotinic acetylcholine receptors
NaCl sodium chloride
nM nanomolar
NNR neuronal nicotinic receptor
NNRs neuronal nicotinic receptors
NOR novel object recognition
NSAIDs nonsteroidal anti-inflammatory drugs
PBS phosphate buffered saline
PET positron emission tomography
pH negative logarithm of the effective hydrogen ion concentration
PMSF phenylmethylsulphonyl fluoride
QNB 3-quinuclidinyl benzilate
SPECT single-photon emission computed tomography

### Biological Assays

### Example 1

### Radioligand Binding at CNS nAChRs

α4β2 nAChR Subtype

Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. Brains were removed and placed on an ice-cold platform. The cerebral cortex was removed and placed in 20 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the suspension was homogenized by Polytron. The homogenate was centrifuged at 18,000×g for 20 min at 4° C. and the resulting pellet was re-suspended in 20 volumes of ice-cold water. After 60 min incubation on ice, a new pellet was collected by centrifugation at 18,000×g for 20 min at 4° C. The final pellet was re-suspended in 10 volumes of buffer and stored at −20° C. On the day of the assay, tissue was thawed, centrifuged at 18,000×g for 20 min, and then re-suspended in ice-cold PBS (Dulbecco's Phosphate Buffered Saline, 138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4) to a final concentration of approximately 4 mg protein/mL. Protein was determined by the method of Lowry et al., J. Biol. Chem. 193: 265 (1951), using bovine serum albumin as the standard.

The binding of [³H]nicotine was measured using a modification of the methods of Romano et al., Science 210: 647 (1980) and Marks et al., Mol. Pharmacol. 30: 427 (1986). The [³H]nicotine (Specific Activity=81.5 Ci/mmol) was obtained from NEN Research Products. The binding of [³H]nicotine was measured using a 3 h incubation at 4° C. Incubations were conducted in 48-well micro-titre plates and contained about 400 μg of protein per well in a final incubation volume of 300 μL. The incubation buffer was PBS and the final concentration of [³H]nicotine was 5 nM. The binding reaction was terminated by filtration of the protein containing bound ligand onto glass fiber filters (GF/B, Brandel) using a Brandel Tissue Harvester at 4° C. Filters were soaked in de-ionized water containing 0.33% polyethyleneimine to reduce non-specific binding. Each filter was washed with ice-cold buffer (3×1 mL). Non-specific binding was determined by inclusion of 10 μM non-radioactive L-nicotine (Acros Organics) in selected wells.

The inhibition of [³H]nicotine binding by test compounds was determined by including seven different concentrations of the test compound in selected wells. Each concentration was replicated in triplicate. IC₅₀ values were estimated as the concentration of compound that inhibited 50 percent of specific [³H]nicotine binding. Inhibition constants (Ki values), reported in nM, were calculated from the IC₅₀ values using the method of Cheng et al., Biochem. Pharmacol. 22: 3099 (1973).

α7 nAChR Subtype

Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. Brains were removed and placed on an ice-cold platform. The hippocampus was removed and placed in 10 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the tissue suspension was homogenized by Polytron. The homogenate was centrifuged at 18,000×g for 20 min at 4° C. and the resulting pellet was re-suspended in 10 volumes of ice-cold water. After 60 min incubation on ice, a new pellet was collected by centrifugation at 18,000×g for 20 min at 4° C. The final pellet was re-suspended in 10 volumes of buffer and stored at −20° C. On the day of the assay, tissue was thawed, centrifuged at 18,000×g for 20 min, and then re-suspended in ice-cold PBS (Dulbecco's Phosphate Buffered Saline, 138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4) to a final concentration of approximately 2 mg protein/mL. Protein was determined by the method of Lowry et al., J. Biol. Chem. 193: 265 (1951), using bovine serum albumin as the standard.

The binding of [³H]MLA was measured using a modification of the methods of Davies et al., Neuropharmacol. 38: 679 (1999). [³H]MLA (Specific Activity=25-35 Ci/mmol) was obtained from Tocris. The binding of [³H]MLA was determined using a 2 h incubation at 21° C. Incubations were conducted in 48-well micro-titre plates and contained about 200 μg of protein per well in a final incubation volume of 300 μL. The incubation buffer was PBS and the final concentration of [³H]MLA was 5 nM. The binding reaction was terminated by filtration of the protein containing bound ligand onto glass fiber filters (GF/B, Brandel) using a Brandel Tissue Harvester at room temperature. Filters were soaked in de-ionized water containing 0.33% polyethyleneimine to reduce non-specific binding. Each filter was washed with PBS (3×1 mL) at room temperature. Non-specific binding was determined by inclusion of 50 μM non-radioactive MLA in selected wells.

The inhibition of [³H]MLA binding by test compounds was determined by including seven different concentrations of the test compound in selected wells. Each concentration was replicated in triplicate. IC₅₀ values were estimated as the concentration of compound that inhibited 50 percent of specific [³H]MLA binding. Inhibition constants (Ki values), reported in nM, were calculated from the IC₅₀ values using the method of Cheng et al., Biochem. Pharmacol. 22: 3099-3108 (1973).

### Example 2

### Determination of Dopamine Release

Dopamine release was measured using striatal synaptosomes obtained from rat brain, according to the procedures set forth by Rapier et al., J. Neurochem. 54: 937 (1990). Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. The brains were quickly removed and the striata dissected. Striatal tissue from each of 2 rats was pooled and homogenized in ice-cold 0.32 M sucrose (5 mL) containing 5 mM HEPES, pH 7.4, using a glass/glass homogenizer. The tissue was then centrifuged at 1,000×g for 10 min. The pellet was discarded and the supernatant was centrifuged at 12,000×g for 20 min. The resulting pellet was re-suspended in perfusion buffer containing monoamine oxidase inhibitors (128 mM NaCl, 1.2 mM KH₂PO₄, 2.4 mM KCl, 3.2 mM CaCl₂, 1.2 mM MgSO₄, 25 mM HEPES, 1 mM ascorbic acid, 0.02 mM pargyline HCl and 10 mM glucose, pH 7.4) and centrifuged for 15 min at 25,000×g. The final pellet was resuspended in perfusion buffer (1.4 mL) for immediate use.

The synaptosomal suspension was incubated for 10 min at 37° C. to restore metabolic activity. [³H]Dopamine ([³H]DA, specific activity=28.0 Ci/mmol, NEN Research Products) was added at a final concentration of 0.1 μM and the suspension was incubated at 37° C. for another 10 min. Aliquots of tissue (50 μL) and perfusion buffer (100 μL) were loaded into the suprafusion chambers of a Brandel Suprafusion System (series 2500, Gaithersburg, Md.). Perfusion buffer (room temperature) was pumped into the chambers at a rate of 3 mL/min for a wash period of 8 min. Test compound (10 μM) or nicotine (10 μM) was then applied in the perfusion stream for 40 sec. Fractions (12 sec each) were continuously collected from each chamber throughout the experiment to capture basal release and agonist-induced peak release and to re-establish the baseline after the agonist application. The perfusate was collected directly into scintillation vials, to which scintillation fluid was added. [³H]DA released was quantified by scintillation counting. For each chamber, the integrated area of the peak was normalized to its baseline.

Release was expressed as a percentage of release obtained with an equal concentration of L-nicotine. Within each assay, each test compound was replicated using 2-3 chambers; replicates were averaged. When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also defined.

### Example 3

### Selectivity vs. Peripheral nAChRs

Interaction at the Human Muscle nAChR Subtype

Activation of muscle-type nAChRs was established on the human clonal line TE671/RD, which is derived from an embryonal rhabdomyosarcoma (Stratton et al., Carcinogen 10: 899 (1989)). These cells express receptors that have pharmacological (Lukas, J. Pharmacol. Exp. Ther. 251: 175 (1989)), electrophysiological (Oswald et al., Neurosci. Lett. 96: 207 (1989)), and molecular biological profiles (Luther et al., J. Neurosci. 9: 1082 (1989)) similar to the muscle-type nAChR.

TE671/RD cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well polystyrene plates (Costar). Experiments were conducted when the cells reached 100% confluency.

Nicotinic acetylcholine receptor (nAChR) function was assayed using ⁸⁶Rb⁺ efflux according to the method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺ was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH. 7.4), taking care not to disturb the cells. Next, cells were exposed to either 100 μM of test compound, 100 μM of L-nicotine (Acros Organics) or buffer alone for 4 min. Following the exposure period, the supernatant containing the released ⁸⁶Rb⁺ was removed and transferred to scintillation vials. Scintillation fluid was added and released radioactivity was measured by liquid scintillation counting.

Within each assay, each point had 2 replicates, which were averaged. The amount of ⁸⁶Rb⁺ release was compared to both a positive control (100 μM L-nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also determined.

Interaction at the Rat Ganglionic nAChR Subtype

Activation of rat ganglion nAChRs was established on the pheochromocytoma clonal line PC12, which is a continuous clonal cell line of neural crest origin, derived from a tumor of the rat adrenal medulla. These cells express ganglion-like nAChR s (see Whiting et al., Nature 327: 515 (1987); Lukas, J. Pharmacol. Exp. Ther. 251: 175 (1989); Whiting et al., Mol. Brain. Res. 10: 61 (1990)).

Rat PC12 cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well Nunc plates (Nunclon) and coated with 0.03% poly-L-lysine (Sigma, dissolved in 100 mM boric acid). Experiments were conducted when the cells reached 80% confluency.

Nicotinic acetylcholine receptor (nAChR) function was assayed using ⁸⁶Rb⁺ efflux according to a method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺ was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH. 7.4), taking care not to disturb the cells. Next, cells were exposed to either 100 μM of test compound, 100 μM of nicotine or buffer alone for 4 min. Following the exposure period, the supernatant containing the released ⁸⁶Rb⁺ was removed and transferred to scintillation vials. Scintillation fluid was added and released radioactivity was measured by liquid scintillation counting

Within each assay, each point had 2 replicates, which were averaged. The amount of ⁸⁶Rb⁺ release was compared to both a positive control (100 μM nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also determined.

Interaction at the Human Ganglionic nAChR Subtype

The cell line SH-SY5Y is a continuous line derived by sequential subcloning of the parental cell line, SK-N-SH, which was originally obtained from a human peripheral neuroblastoma. SH-SY5Y cells express a ganglion-like nAChR (Lukas et al., Mol. Cell. Neurosci. 4: 1 (1993)).

Human SH-SY5Y cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well polystyrene plates (Costar). Experiments were conducted when the cells reached 100% confluency.

Nicotinic acetylcholine receptor (nAChR) function was assayed using ⁸⁶Rb⁺ efflux according to a method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺ was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4), taking care not to disturb the cells. Next, cells were exposed to either 100 μM of test compound, 100 μM of nicotine, or buffer alone for 4 min. Following the exposure period, the supernatant containing the released ⁸⁶Rb⁺ was removed and transferred to scintillation vials. Scintillation fluid was added and released radioactivity was measured by liquid scintillation counting

Within each assay, each point had 2 replicates, which were averaged. The amount of ⁸⁶Rb⁺ release was compared to both a positive control (100 μM nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also defined.

### Example 4

### Determination of Binding at Non-nicotinic Receptors

Muscarinic M3 Subtype

The human clonal line TE671/RD, derived from an embryonal rhabdomyosarcoma (Stratton et al., Carcinogen 10: 899 (1989)), was used to define binding to the muscarinic M3 receptor subtype. As evidenced through pharmacological (Bencherif et al., J. Pharmacol. Exp. Ther. 257: 946 (1991) and Lukas, J. Pharmacol. Exp. Ther. 251: 175 (1989)), electrophysiological (Oswald et al., Neurosci. Lett. 96: 207 (1989)), and molecular biological studies (Luther et al., J. Neurosci. 9: 1082 (1989)) these cells express muscle-like nicotinic receptors.

TE671/RD cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). They were grown to confluency on 20-150 mm tissue culture treated plates. The media was then removed and cells scraped using 80 mL of PBS (Dulbecco's Phosphate Buffered Saline, 138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4) and then centrifuged at 1000 rpm for 10 min. The supernatant was then suctioned off and the pellet(s) stored at −20° C. until use.

On the day of the assay, the pellets were thawed, re-suspended with PBS and centrifuged at 18,000×g for 20 min, then re-suspended in PBS to a final concentration of approximately 4 mg protein/mL and homogenized by Polytron. Protein was determined by the method of Lowry et al., J. Biol. Chem. 193: 265 (1951), using bovine serum albumin as the standard.

The binding of [³H]QNB was measured using a modification of the methods of Bencherif et al., J. Pharmacol. Exp. Ther. 257: 946 (1991). [³H]QNB (Specific Activity=30-60 Ci/mmol) was obtained from NEN Research Products. The binding of [³H]QNB was measured using a 3 h incubation at 4° C. Incubations were conducted in 48-well micro-titre plates and contained about 400 μg of protein per well in a final incubation volume of 300 μL. The incubation buffer was PBS and the final concentration of [³H]QNB was 1 nM. The binding reaction was terminated by filtration of the protein containing bound ligand onto glass fiber filters (GF/B, Brandel) using a Brandel Tissue Harvester at 4° C. Filters were pre-soaked in de-ionized water containing 0.33% polyethyleneimine to reduce non-specific binding. Each filter was washed with ice-cold buffer (3×1 mL). Non-specific binding was determined by inclusion of 10 μM non-radioactive atropine in selected wells.

The inhibition of [³H]QNB binding by test compounds was determined by including seven different concentrations of the test compound in selected wells. Each concentration was replicated in triplicate. IC₅₀ values were estimated as the concentration of compound that inhibited 50 percent of specific [³H]QNB binding. Inhibition constants (Ki values), reported in nM, were calculated from the IC₅₀ values using the method of Cheng et al., Biochem. Pharmacol. 22: 3099 (1973).

### Synthetic Examples

Except as otherwise noted, all reactions were run under a nitrogen atmosphere, and reagents and solvents were used as obtained from commercial sources.

### Example 5

### Synthesis of tert-butyl 3,6-diazabicyclo[3.2.1]octane-6-carboxylate

The following general procedures can be employed using either racemic or single enantiomer starting materials, all of which are commercially available. Using these procedures tert-butyl (1R,5S)-3,6-diazabicyclo[3.2.1]octane-6-carboxylate was obtained in 35% overall yield from (1S,4R)-2-azabicyclo[2.2.1]hept-5-en-3-one (Aldrich Chemical), and tert-butyl (1S,5R)-3,6-diazabicyclo[3.2.1]octane-6-carboxylate was obtained in 45% overall yield from (1R,4S)-2-azabicyclo[2.2.1]hept-5-en-3-one (Aldrich Chemical).

A solution of azabicyclo[2.2.1]hept-5-en-3-one (5.0 g, 49 mmol) in dry tetrahydrofuran (THF) (100 mL) was added to a slurry of lithium aluminum hydride (1.8 g, 49 mmol) in dry THF (100 mL) at 0° C. The reaction mixture was heated at reflux for 3 h and then cooled to ambient temperature. Ether (100 mL) was added and the mixture was cooled and stirred at 0° C. as sodium hydroxide solution (5N, 20 mL) was slowly added to quench the reaction. The slurry was filtered through diatomaceous earth, and the filtrate was combined with di-tert-butyl dicarbonate (10.6 g, 48.6 mmol) and triethylamine (6.3 mL, 45 mmol). This mixture was stirred at ambient temperature for 12 h. The solvent was removed by rotary evaporation, and the residue was dissolved in dichloromethane (200 mL), washed with saturated aqueous ammonium chloride (200 mL), and dried over anhydrous magnesium sulfate. Evaporation of the dichloromethane left 9.4 g of tert-butyl 2-azabicyclo[2.2.1]hept-5-ene-2-carboxylate as a oil.

The tert-butyl 2-azabicyclo[2.2.1]hept-5-ene-2-carboxylate was dissolved in 200 mL of dichloromethane-methanol (2:1), and the solution was cooled to −78° C. Ozone was passed through the solution until it turned blue and then for a further 10 min. Argon was bubbled through the solution to remove excess ozone (the solution turned colorless). This process (ozone, followed by argon) was repeated one more time to ensure complete formation of the ozonide. Sodium borohydride (3.7 g, 97 mmol) was carefully added to the reaction mixture at −78° C., and the resulting mixture stirred for 16 h, as the temperature of the reaction was gradually increased to ambient. Saturated ammonium chloride solution (100 mL) was added, and the mixture was stirred for an additional 1 h. The mixture was extracted with dichloromethane (2×150 mL), and the combined organic extracts were dried over anhydrous magnesium sulfate. The solvent was removed by rotary evaporation to give tert-butyl 2,4-bis(hydroxymethyl)pyrrolidine-1-carboxylate, as a light yellow oil.

The tert-butyl 2,4-bis(hydroxymethyl)pyrrolidine-1-carboxylate was dissolved in 300 mL of dry dichloromethane and cooled to 0° C. Triethylamine (9.7 mL, 70 mmol) was added to the cooled solution, followed by a careful addition of methanesulfonyl chloride (5.4 mL, 70 mmol). The reaction was stirred at ambient temperature for 16 h. Saturated ammonium chloride solution (200 mL) was added, and the layers were separated. The aqueous layer was washed with dichloromethane (200 mL), and the combined organic layers were dried over anhydrous magnesium sulfate, filtered, and concentrated by evaporation of the volatiles. The residual oil, tert-butyl 2,4-bis((methylsulfonyloxy)methyl)pyrrolidine-1-carboxylate, was placed in 200 mL pressure tubes (˜10 mmol maximum in each tube). Concentrated aqueous ammonium hydroxide (150 mL) and CuI (190 mg, 10 mol %) were added to each pressure tube. The tubes were sealed and heated at 100° C. for 16 h. The tubes were cooled to ambient temperature, and the reaction mixture was concentrated by rotary evaporation at 60° C. (bath temperature). The solid was dissolved in methanol and filtered through diatomaceous earth to remove copper salts. The solvent was removed by rotary evaporation, and the residue was purified using an Analogix IntelliFlash 280 system with a SF25-120g Si column, eluting with a methanol in chloroform gradient (0-50% methanol over 30 min). Evaporation of the solvent gave tert-butyl 3,6-diazabicyclo[3.2.1]octane-6-carboxylate as a viscous oil (4.1 g, 40%).

### Example 6

### Synthesis of 3,6-diazabicyclo[3.2.1]octane-3-carboxylates

The following procedures were used to synthesize both single enantiomer and racemic compounds.

### Methyl (1S,5S)-3,6-diazabicyclo[3.2.1]octane-3-carboxylate

A sample of tert-butyl (1R,5S)-3,6-diazabicyclo[3.2.1]octane-6-carboxylate (60 mg, 0.28 mmol) was dissolved in 5 mL of dichloromethane. Triethylamine (77□L, 0.56 mmol) was added and the reaction was cooled to 0° C., before adding methyl chloroformate (22□L, 0.28 mmol). The reaction mixture was stirred for 1 h at ambient temperature, and the volatiles were removed in vacuo. The residue was partitioned between dichloromethane (20 mL) and aqueous sodium acetate (10 mL of 50 mM), and the organic layer was dried over anhydrous magnesium sulfate. Filtration and concentration of the filtrate by rotary evaporation gave 6-tert-butyl 3-methyl (1R,5S)-3,6-diazabicyclo[3.2.1]octane-3,6-dicarboxylate (75 mg, 100%). The entire sample (0.28 mmol) was dissolved in ethyl acetate (3 mL), and 3N HCl/ethyl acetate (3 mL) was added. The reaction mixture was stirred for 2 h at ambient temperature before removing solvent by rotary evaporation at 60° C. The residue was mixed with saturated aqueous potassium carbonate solution (2 mL). Concentration by rotary evaporation at 60° C. left a solid which was triturated with CMA 90 (chloroform:methanol:aqueous ammonium hydroxide (90:9:1)). The solvent was removed in vacuo, and the residue was purified using an Analogix IntelliFlash 280 system with a SF10-4-g Si column eluting with a chloroform to CMA 90 gradient over 21 min, to give methyl (1S,5S)-3,6-diazabicyclo[3.2.1]octane-3-carboxylate (41 mg, 90%) as a yellow oil.

### 3-Trifluoroacetyl-3,6-diazabicyclo[3.2.1]octane

A sample of tert-butyl 3,6-diazabicyclo[3.2.1]octane-6-carboxylate (500 mg, 2.36 mmol) was dissolved in dichloromethane (20 mL). Triethylamine (325 μL, 2.36 mmol) was added at 0° C., followed by trifluoroacetic anhydride (328 μL, 2.36 mmol). The reaction mixture was warmed to ambient temperature, quenched with aqueous sodium acetate (50 mM solution, 20 mL) and extracted with dichloromethane (2×20 mL). The combined organic extracts were dried over anhydrous magnesium sulfate, filtered and concentrated by rotary evaporation. The residue was purified using an Analogix IntelliFlash 280 system with a SF15-12g Si column, eluting with an ethyl acetate in dichloromethane gradient (0-50% ethyl acetate) over 24 min to give tert-butyl 3-trifluoroacetyl-3,6-diazabicyclo[3.2.1]octane-6-carboxylate (0.40 g, 92%) as an orange oil. The entire sample (1.3 mmol) was dissolved in dichloromethane and treated with a 25% trifluoroacetic acid/dichloromethane solution (0.5 mL). The reaction mixture was stirred for 2 h at ambient temperature, before partitioning between saturated sodium bicarbonate (20 mL), and dichloromethane (20 mL). The organic extracts were dried over anhydrous magnesium sulfate, concentrated, and purified using an Analogix IntelliFlash 280 system with a SF15-12g Si column, eluting with 100% chloroform to 100% (chloroform:methanol:ammonium hydroxide (90:9:1) gradient over 24 min. This gave 3-trifluoroacetyl-3,6-diazabicyclo[3.2.1]octane (0.25 g, 90%) as a yellow oil.

### Example 7

### Synthesis of (1R,5R)-3-(5-bromofuran-2-ylcarbonyl)-3,6-diazabicyclo[3.2.1]octane

The following procedures are exemplary of those used to produce various 3-(heteroarylcarbonyl)-3,6-diazabicyclo[3.2.1]octanes and were used to synthesize both single enantiomer and racemic compounds.

5-Bromofuroic acid (45 mg, 0.24 mmol), PS-DCC (polystyrene bound N,N′-dicyclohexylcarbodiimide) (0.37 g, 1.29 mmol/g, 0.48 mmol), and HOBt (54 mg, 0.41 mmol) were stirred in a reaction vial with dry dichloromethane (5 mL). After 10 min, (1S,5R) tert-butyl 3,6-diazabicyclo[3.2.1]octane-6-carboxylate (50 mg, 0.24 mmol) in dichloromethane (2.5 mL) was added, and the reaction was stirred for 2 h. Filtration of the mixture through a sintered funnel and concentration of the filtrate left an orange oil. Purification of this concentrate, using an Analogix IntelliFlash 280 system with a SF10-4g Si column, eluting with an ethyl acetate in chloroform gradient (100% chloroform-100% ethyl acetate) over 24 min, gave tert-butyl (1S,5R)-3-(5-bromofuran-2-ylcarbonyl)-3,6-diazabicyclo[3.2.1]octane-6-carboxylate (25 mg, 27%) as a yellow oil. The entire sample (0.070 mmol) was dissolved in ethyl acetate (3 mL), and 3N HCl/ethyl acetate (3 mL) was added. The reaction mixture was stirred for 2 h at ambient temperature, before removing the solvent in vacuo at 60° C. The residue was treated with saturated aqueous potassium carbonate (2 mL), and the mixture was again concentrated in vacuo at 60° C. This residue was triturated with CMA 90 (chloroform:methanol:ammonium hydroxide (90:9:1)) (5 mL), and the triturates were concentrated in vacuo. The residue was purified using an Analogix IntelliFlash 280 system with a SF10-4g Si column, eluting with a chloroform to CMA 90 gradient (100% chloroform-100% CMA 90) over 21 min, to give (1R,5R)-3-(5-bromofuran-2-ylcarbonyl)-3,6-diazabicyclo[3.2.1]octane (18 mg, 100%) as a yellow oil.

### Example 8

### Synthesis of 6-(5-bromofuran-2-ylcarbonyl)-3,6-diazabicyclo[3.2.1]octane

The following procedures are exemplary of those used to produce various 6-(heteroarylcarbonyl)-3,6-diazabicyclo[3.2.1]octanes and were used to synthesize both single enantiomer and racemic compounds.

5-Bromofuroic acid (45 mg, 0.24 mmol), PS-DCC (0.37 g, 1.29 mmol/g, 0.48 mmol), and HOBt (54 mg, 0.41 mmol) were stirred in a reaction vial with dry dichloromethane (5 mL). After 10 min, 3-trifluoroacetyl-3,6-diazabicyclo[3.2.1]octane (50 mg, 0.24 mmol) in dichloromethane (2.5 mL) was added, and the reaction stirred for 2 h at ambient temperature. Filtration of the mixture through a sintered funnel, followed by concentration in vacuo and purification using an Analogix IntelliFlash 280 system with a SF10-4g Si column, eluting with a 100% chloroform to 100% ethyl acetate gradient over 24 min, gave 3-trifluoroacetyl-6-(5-bromofuran-2-ylcarbonyl)-3,6-diazabicyclo[3.2.1]octane (30 mg, 33%) as a yellow oil. The entire sample (0.08 mmol) was dissolved in methanol (5 mL), and saturated potassium carbonate solution (2 mL) was added. The reaction was heated at 60° C. for 2 h and then cooled to ambient temperature. The solvent was removed in vacuo at 60° C. (bath temperature), and the resultant solid was triturated with CMA 90 (5 mL). The solvent was removed in vacuo, and the residue was purified using an Analogix IntelliFlash 280 system with a SF10-4g Si column, eluting with a gradient of 100% chloroform to 100% CMA 90 (chloroform:methanol:ammonium hydroxide (90:9:1)) over 21 min, to give 6-(5-bromofuran-2-ylcarbonyl)-3,6-diazabicyclo[3.2.1]octane (20 mg, 90%) as a light brown oil.

### Example 9

### Synthesis of tert-butyl 3,7-diazabicyclo[4.2.0]octane-7-carboxylate

The following procedures are adapted from those found in Frost et al., J. Med. Chem. 49: 7843 (2006) for the synthesis of tert-butyl 3,8-diazabicyclo[4.2.0]octane-8-carboxylate. While the procedures support the synthesis of the single enantiomers (by separation of one of more of the various diastereomeric intermediates via either chromatography or fractional crystallization) of tert-butyl 3,7-diazabicyclo[4.2.0]octane-7-carboxylate, the procedures reported here are for the racemate.

A mixture of commercially available ethyl N-benzyl-3-oxo-4-piperidinecarboxylate hydrochloride (100 g, 0.336 mol), di-tert-butyl dicarbonate (80 g, 0.37 mol), triethylamine (43.5 g, 0.43 mol), and palladium hydroxide on carbon (60 g, 20% in H₂O) in ethanol (1.5 L) was put under 60 psi of hydrogen gas and was shaken for 5 h. The mixture was then filtered, and the filtrate was concentrated under reduced pressure to provide 1-tert-butyl 3-ethyl 4-oxopiperidine-1,3-dicarboxylate (85 g, 93% yield), which was used in the next step without further purification.

A mixture of 1-tert-butyl 3-ethyl 4-oxopiperidine-1,3-dicarboxylate (180 g, 0.670 mol) and (R)-α-methylbenzylamine (89 g, 0.73 mol) in toluene (1.8 L) was refluxed for 16 h, with azeotropic removal of water. After cooling to ambient temperature, the solution was concentrated and re-dissolved in ethyl acetate (500 mL). Filtration through silica gel and diatomaceous earth and concentration under reduced pressure gave the crude 1-tert-butyl 3-ethyl 4-(1-phenylethylamino)-5,6-dihydropyridine-1,3(2H)-dicarboxylate (203 g, 81% yield), which was used in the next step without further purification.

To a mixture of 1-tert-butyl 3-ethyl 4-(1-phenylethylamino)-5,6-dihydropyridine-1,3(2H)-dicarboxylate (136 g, 0.34 mol), sodium triacetoxyborohydride (360 g, 1.7 mol), and 256 g of 4 Å powered molecular sieves in toluene (1.5 L) at 0° C. was added acetic acid (408 g, 6.8 mol) dropwise, while keeping the internal temperature below 5° C. After addition was complete, the mixture was allowed to warm to ambient temperature and stirred for 16 h. The reaction mixture was filtered and concentrated under reduced pressure to remove most of acetic acid. The residue was dissolved in 1 L of water, and solid sodium carbonate (300 g) was added slowly to neutralize the residual acid and bring the pH to 9. The layers were separated, and the aqueous layer was extracted with ethyl acetate (4×300 mL). The combined organics were dried over anhydrous sodium sulfate and concentrated under reduced pressure to give 1-tert-butyl 3-ethyl 4-(1-phenylethylamino)piperidine-1,3-dicarboxylate (109 g, 85% yield), which was used in the next step without further purification.

To a solution of 1-tert-butyl 3-ethyl 4-(1-phenylethylamino)piperidine-1,3-dicarboxylate (121 g. 0.321 mol) in THF (2 L) was added lithium aluminum hydride (13.5 g, 0.354 mol) in portions over 1 h while keeping the internal temperature below 0° C. After the addition, the reaction mixture was stirred at 0° C. for 1 h, then warmed to ambient temperature and stirred for 16 h. The reaction mixture was quenched by slow addition of 5 M aqueous sodium hydroxide solution (81 mL). The mixture was filtered to remove aluminate salts, concentrated under reduced pressure, and purified by silica gel chromatography (petroleum ether/ethyl acetate, 3:1; silica gel, 200-300 mesh) to yield tert-butyl 3-(hydroxymethyl)-4-(1-phenylethylamino)piperidine-1-carboxylate as a light yellow oil (44 g, 41%).

To a solution of tert-butyl 3-(hydroxymethyl)-4-(1-phenylethylamino)piperidine-1-carboxylate (56 g. 0.17 mol) in THF (1 L) at 0° C. was added triethylamine (55 mL, 0.40 mol), followed by methanesulfonyl chloride (16.9 mL, 0.22 mol). The ice bath was removed after the addition, and the reaction was warmed to ambient temperature and stirred for 1 h. Cesium carbonate (74.3 g, 0.39 mol) was added, and the mixture was warmed to 60° C. and stirred for 16 h. The reaction was cooled to ambient temperature and filtered. The filtrate was concentrated under reduced pressure. The material was purified by silica gel column chromatography (petroleum ether/ethyl acetate, 5:1; silica gel, 200-300 mesh), to give tert-butyl 7-(1-phenylethyl)-3,7-diazabicyclo[4.2.0]octane-3-carboxylate as a mixture of stereoisomers (25 g, 47%).

To a solution of tert-butyl 7-(1-phenylethyl)-3,7-diazabicyclo[4.2.0]octane-3-carboxylate (25 g, 79.0 mmol) in dichloromethane (70 mL) at 0° C. was added trifluoroacetic acid (35 mL). The ice bath was removed, and the mixture was stirred at ambient temperature for 1 h. The mixture was then concentrated and filtered through a plug of diatomaceous earth and silica gel with 9:1:0.1 dichloromethane/methanol/concentrated ammonium hydroxide. To the resulting free-amine intermediate (79.0 mmol) in THF (550 mL) at −30° C. was added triethylamine (15 mL, 108 mmol), followed by trifluoroacetic anhydride (11.7 mL, 83.1 mmol). This mixture was stirred for 1.5 h as it was warmed from −30 to −10° C. The mixture was quenched with saturated aqueous sodium bicarbonate (50 mL) and was warmed to ambient temperature. The layers were separated, and the aqueous layer was extracted with ethyl acetate (3×800 mL). The combined organics were washed with brine (30 mL) and then dried over anhydrous sodium sulfate, filtered, and concentrated under reduced pressure. The crude material was dissolved in ethyl acetate (100 mL) and filtered through a plug of diatomaceous earth and silica gel with ethyl acetate (600 mL) to give 3-trifluoroacetyl-7-(1-phenylethyl)-3,7-diazabicyclo[4.2.0]octane (8.3 g, 56% yield).

A mixture of 3-trifluoroacetyl-7-(1-phenylethyl)-3,7-diazabicyclo[4.2.0]octane (8.30 g, 26.6 mmol), di-tert-butyl dicarbonate (7.0 g, 32 mmol), and wet palladium hydroxide on carbon (20 wt %, 1.0 g) in ethyl acetate (300 mL) was shaken under a 60 psi atmosphere of hydrogen gas for 16.5 h at 50° C. The mixture was filtered, and the filtrate was concentrated under reduced pressure. The residue was dissolved in methanol (150 mL) and water (30 mL), and potassium carbonate (4.4 g, 31.8 mmol) was added. This mixture was stirred at ambient temperature for 16 h and then concentrated under reduced pressure. The crude material was purified via column chromatography (9:1:0.1 dichloromethane/methanol/concentrated ammonium hydroxide; silica gel, 200-300 mesh) to give tert-butyl 3,7-diazabicyclo[4.2.0]octane-7-carboxylate (3.4 g, 60% yield). ¹H NMR (300 MHz, CD₃OD) δ 4.35 (m, 1H), 3.90 (m, 1H), 3.54 (m, 1H), 3.20 (m, 1H), 2.82 (m, 3H), 2.52 (m, 1H), 2.00 (m, 1H), 1.82 (m, 1H), 1.44 (m, 9H); LC-MS (M⁺) 312.

### Example 10

### Synthesis of 3-(furan-3-ylcarbonyl)-3,7-diazabicyclo[4.2.0]octane

The following procedures are exemplary of those used to produce various 3-(heteroarylcarbonyl)-3,7-diazabicyclo[4.2.0]octanes and were used to synthesize both single enantiomer and racemic compounds.

To 3,7-diazabicyclo[4.2.0]octane-7-carboxylic acid tert-butyl ester (0.10 g, 0.47 mmol) in dichloromethane (3 mL) were added triethylamine (0.20 mL, 1.4 mmol) and 3-furoyl chloride (0.085 g, 0.0.66 mmol), and the mixture was stirred for 1 h at ambient temperature. The solvent was evaporated, and the crude amide was purified by reverse phase HPLC using acetonitrile and 0.05% aqueous TFA (trifluoroacetic acid) as the mobile phase, to obtain 3-(furan-3-ylcarbonyl)-3,7-diazabicyclo[4.2.0]octane-7-carboxylic acid tert-butyl ester as oil. This was dissolved in dichloromethane (3 mL), combined with trifluoroacetic acid (2 mL), and stirred at ambient temperature for 1 h. The solvent was evaporated, and the residue was purified by HPLC, using acetonitrile and 0.05% aqueous trifluoroacetic acid as the mobile phase, to obtain 0.046 g of 3-(furan-3-ylcarbonyl)-3,7-diazabicyclo[4.2.0]octane as an oil. ¹H NMR (CD₃OD, 300 MHz): δ 8.04 (dd, J=1.83 and 0.85 Hz, 1H), 7.61 (m, 1H), 6.74 (dd, J=1.95 and 0.85 Hz, 1H), 4.89-4.79 (m, 1H), 4.55-4.43 (m, 1H), 4.27-4.21 (m, 1H), 3.52-3.42 (m, 1H), 3.40-3.22 (m, 3H), 3.11-3.05 (m, 1H), 2.41-2.18 (m, 2H); MS (m/z): 207 (M+1).

### Example 11

### Synthesis of 7-(4-methyloxazol-5-ylcarbonyl)-3,7-diazabicyclo[4.2.0]octane

The following procedures are exemplary of those used to produce various 7-(heteroarylcarbonyl)-3,7-diazabicyclo[4.2.0]octanes and were used to synthesize both single enantiomer and racemic compounds.

To a solution of 3,7-diazabicyclo[4.2.0]octane-7-carboxylic acid tert-butyl ester (1.15 g, 5.42 g) and triethylamine (2.0 mL) in dichloromethane (25 mL) was added trifluoroacetic anhydride (2.0 mL) at 0° C., and reaction was stirred for 1 h. The reaction mixture was concentrated and the residue was dissolved in dichloromethane (10 mL). Trifluoroacetic acid (10 mL) was added to the reaction and it was stirred for 2 h at ambient temperature. The reaction mixture was concentrated, and the crude product was dissolved in dichloromethane (18 mL). The solution was split into 6 vials of 3 mL each, for coupling with each of six different acids.

In a representative synthesis, the above amine solution was treated with triethylamine (1 mL), followed by 4-methyloxazole-5-carbonyl chloride (0.265 g , 1.82 mmol), and the reaction was stirred for 1 h at ambient temperature. The reaction mixture was concentrated, and the crude amide was purified by reverse phase HPLC using acetonitrile and 0.05% aqueous trifluoroacetic acid as the mobile phase, to obtain 7-(4-methyloxazol-5-ylcarbonyl)-3-(trifluoroacetyl)-3,7-diaza-bicyclo[4.2.0]octane. This was then dissolved in methanol-water (3 mL, 4:1) and solid potassium carbonate (0.15 g) was added. The reaction mixture was stirred for 2 h at ambient temperature. The solvent was evaporated and the product was extracted with 20% methanol in dichloromethane (2×5 mL). The solvent was evaporated, and the product was purified by reverse phase HPLC, using acetonitrile and 0.05% aqueous TFA as the mobile phase, to obtain 7-(4-methyloxazol-5-ylcarbonyl)-3,7-diazabicyclo[4.2.0]octane as white solid (0.022 g). ¹H NMR (CD₃OD, 300 MHz): δ 8.22 (s, 1H), 4.82-4.76 (m, 1H), 4.75-4.61 (m, 1H), 4.41-4.35 (m, 1H), 3.58-3.22 (m, 4H), 3.18-3.00 (m, 1H), 2.44 (s, 3H), 2.44-2.23 (m, 2H); MS (m/z): 222 (M+1).

### Example 12

### Synthesis of 2-(furan-2-ylcarbonyl)-2,7-diazabicyclo[3.3.0]octane

The following procedures are exemplary of those used to produce various 2-(heteroarylcarbonyl)-2,7-diazabicyclo[3.3.0]octanes.

To a solution of furan-2-carboxylic acid (0.028 g, 0.25 mmol) in anhydrous THF (2.5 mL) was added DCC (0.064 g, 0.31 mmol) and HOBt (0.041 g, 0.31 mmol). This mixture was stirred at ambient temperature for 10 minutes, and then a solution of racemic tert-butyl 2,7-diazabicyclo[3.3.0]octane-7-carboxylate (commercially available) in THF (1 mL) was added. The reaction mixture was stirred at ambient temperature for 16 h. The solids were removed by filtration, and the residue was purified by reverse phase HPLC to give tert-butyl 2-(furan-2-ylcarbonyl)-2,7-diazabicyclo[3.3.0]octane-7-carboxylate (0.024 g, 32% yield) as colorless syrup. This material was dissolved in 1:1 mixture of trifluoroacetic acid and dichloromethane (1 mL) and shaken at ambient temperature for 1 h. The volatiles were removed under reduced pressure, and the residue was dried overnight at high vacuum, to give 0.012 g of 2-(furan-2-ylcarbonyl)-2,7-diazabicyclo[3.3.0]octane as a solid (50% yield).

### Example 13

### Synthesis of 7-(furan-3-ylcarbonyl)-2,7-diazabicyclo[3.3.0]octane

The following procedures are exemplary of those used to produce various 7-(heteroarylcarbonyl)-2,7-diazabicyclo[3.3.0]octanes.

To a solution of furan-3-carboxylic acid (0.084 g, 0.75 mmol) in anhydrous THF (5 mL) was added HBTU (0.28 g, 0.75 mmol), followed by triethylamine (0.2 g, 2 mmol). After stirring at ambient temperature for 10 min, the mixture was treated with a solution of tert-butyl 2,7-diazabicyclo[3.3.0]octane-2-carboxylate (commercially available) (0.106 g, 0.500 mmol) in THF (2 mL). The reaction mixture was stirred for 16 h at ambient temperature. The solvent was removed by rotary evaporation, and the residue was partitioned between ethyl acetate (5 mL) and saturated sodium bicarbonate (2 mL). The organic layer was concentrated, and the residue was purified by reverse phase HPLC to give tert-butyl 7-(furan-3-ylcarbonyl)-2,7-diazabicyclo[3.3.0]octane-2-carboxylate. This was dissolved in 1:1 mixture of trifluoroacetic acid and dichloromethane (1 mL) and the mixture was shaken at ambient temperature for 1 h. The volatiles were removed under reduced pressure, and the residue was dried overnight at high vacuum, to give 0.050 g of 7-(furan-3-ylcarbonyl)-2,7-diazabicyclo[3.3.0]octane as a syrup (0.050 g, 32% yield).

### Example 14

### Synthesis of 8-(heteroarylcarbonyl)-3,8-diazabicyclo[4.3.0]nonanes

Tert-butyl 3,8-diazabicyclo[4.3.0]nonane-3-carboxylate is commercially available in both its racemate and R,R forms. These materials were coupled and subsequently de-protected, using procedures described in previous examples, to produce 8-(heteroarylcarbonyl)-3,8-diazabicyclo[4.3.0]nonanes.

### Example 15

### Synthesis of 3-(heteroarylcarbonyl)-3,6-diazabicyclo[3.2.0]heptanes

The key intermediate, tert-butyl 3,6-diazabicyclo[3.2.0]heptane-6-carboxylate, was synthesized using procedures described in US patent application 2006/0035937. This material was coupled and subsequently de-protected, using procedures described in previous examples, to produce 3-(heteroarylcarbonyl)-3,6-diazabicyclo[3.2.0]heptanes.

### Example 16

### Spectral and Binding Data

Among the N-(heteroarylcarbonyl)diazabicycloalkanes produced using the procedures exemplified in Examples 5 through 15 are those shown in Table 1.

TABLE 1

| | Rat | | | MS: | |
|---|---|---|---|---|---|
| | α4β2 | Human | | m/z | |
| Structure | Ki | α4β2 Ki | α7 Ki | (M + H) | ¹H NMR: CD₃OD, 300 MHz |
| 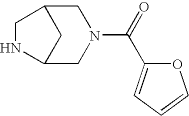 | 19.2 | 23.9 | ND; Failed HTS | 207 | 7.67-7.64 (m, 1H), 7.02-7.01 (m, 1H), 6.58-6.56 (m, 1H), 4.54-4.20 (m, 3H), 3.62-3.56 (m, 1H), 3.05- 2.90 (m, 3H), 2.55-2.45 (m, 1H), 1.98-1.80 (m, 2H) |
| 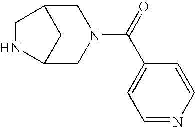 | ND | 29.7 | ND; Failed HTS | 218 | 8.62 (m, 2H), 7.55 (m, 1H), 7.45 (m, 1H), 4.45-4.38 (m, 1H), 3.65 (m, 1H), 3.35-3.25 (m, 3H), 3.15- 2.95 (m, 3H), 2.60-2.35 (2 br peaks, 1H), 1.98-1.90 (m, 2H) |
| 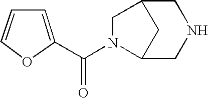 | ND | 217.4 | ND; Failed HTS | 207 | 7.75 (d, J = 16 Hz, 1H), 7.20 (dd, J = 10 Hz, 1H), 6.62 (m, 1H), 4.64 (m, 0.5H), 4.42 (m, 0.5H), 3.80- 3.60 (m, 1H), 3.15-3.05 (m, 1H), 2.90-3.62 (m, 3H), 2.24-2.38 (m, 1H), 2.05-2.00 (m, 1H) 1.98-1.82 (m, 2H) |
| 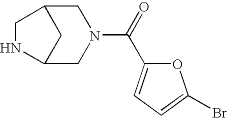 | 14.5 | 0.8 | ND; Failed HTS | 285, 287 | 7.02 (d, J = 4.5 Hz, 1H), 6.58 (d, J = 4.5 Hz, 1H), 4.45-4.10 (m, 2H, (3.60 (m, 2H), 3.15-2.91 (m, 3H), 2.54 (m, 1H), 1.95-1.84 (m, 2H) |
| 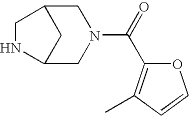 | ND | 3.4 | ND; Failed HTS | 221 | |
| 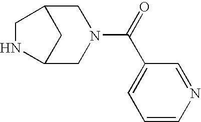 | ND | 359.1 | ND; Failed HTS | 218 | |
| 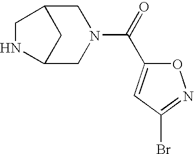 | 3.4 | 2.8 | ND; Failed HTS | 286, 288 | |
| 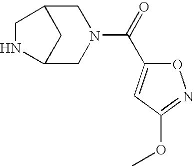 | 5 | 3.7 | ND; Failed HTS | 238 | |
| 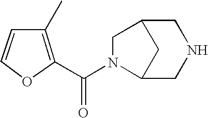 | 138.7 | 95.2 | ND; Failed HTS | 221 | 7.55 (m, 1H0, 6.48 (m, 1H), 4.55 (m, 0.5H0, 4.40 (m, 0.5H), 4.10- 3.95 (m, 1H), 3.80-3.70 (m, 0.5H), 3.68 (m, 0.5H), 3.20-3.08 (m, 1H), 2.95-2.68 (m, 3H), 2.46-2.30 (m, 4H), 2.10-1.80 (m, 2H) |
| 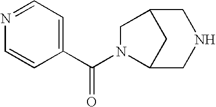 | 317.2 | 131.8 | ND; Failed HTS | 218 | |
| 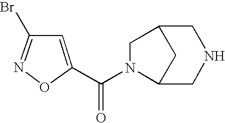 | 171.7 | 14.3 | ND; Failed HTS | 286, 288 | |
| 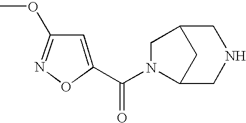 | 195.2 | 305.9 | ND; Failed HTS | 238 | |
| 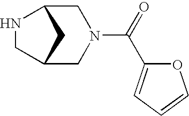 | 40.9 | 16.9 | 1018.7 | 207 | |
| 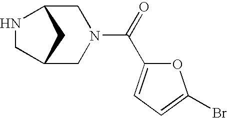 | 38.5 | 42.9 | ND; Failed HTS | 285, 287 | 7.05 (d, J = 4.5 Hz, 1H), 6.60 (d, J = 4.5 Hz, 1H), 4.55-4.40 (m, 2H), 4.15 (m, 1H), 3.60-3.20 (m, 4H), 2.70 (m, 1H), 2.10 (m, 2H) |
| 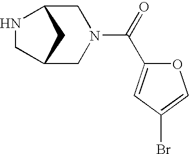 | 67.5 | 30.5 | ND; Failed HTS | 286, 288 | 7.00 (d, J = 14.3 Hz, 1H), 4.42 (m, 0.5H), 4.30 (m, 0.5H), 3.70 (m, 1H), 3.62-3.45 (m, 2H), 3.10-2.80 (m, 3H), 2.55-2.45 (m, 1H), 1.92- 1.80 (m, 2H) |
| 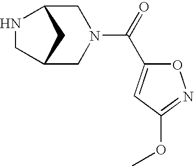 | 366.7 | 95.4 | ND; Failed HTS | 238 | |
| 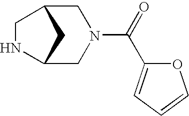 | 10.8 | 13.1 | ND; Failed HTS | 207 | 7.64 (s, 1H), 7.02 (m, 1H), 6.56 (m, 1H), 4.45-4.20 (m, 2H), 3.55 (m, 2H), 3.10-2.90 (m, 3H), 2.50 (m, 1H), 1.90-1.82 (m, 2H) |
| 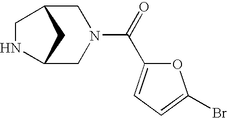 | 14.4 | 6.8 | ND; Failed HTS | 285, 287 | |
| 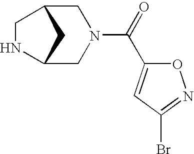 | 3.5 | 1.8 | ND; Failed HTS | 286, 288 | |
| 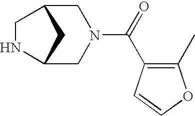 | 17.3 | 10.9 | ND; Failed HTS | 221 | |
| 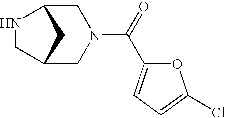 | 39.6 | 27.1 | ND; Failed HTS | 241 | 7.12 (m, 1H), 6.50 (m, 1H), 4.5- 4.38 (m, 2H), 4.15 (m, 1H), 3.45- 3.00 (m, 3H), 2.80 (m, 1H), 2.20- 2.05 (m, 2H) |
| 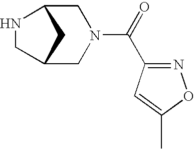 | 852.6 | 502.7 | ND; Failed HTS | 222 | |
| 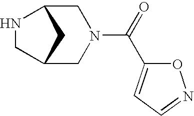 | 26.8 | 34.5 | ND; Failed HTS | 208 | 7.92 (brs, 1H), 6.90 (brs, 1H), 4.60-4.05 (m, 3H), 3.80-3.05 (m, 4H), 2.82 (m, 1H), 2.20-2.00 (m, 2H) |
| 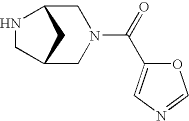 | 70.1 | 85.2 | ND; Failed HTS | 208 | |
| 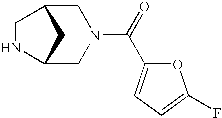 | 8.5 | 6.9 | ND; Failed HTS | 225 | |
| 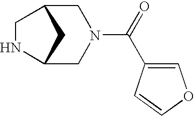 | 10.6 | 10.5 | ND; Failed HTS | 207 | |
| 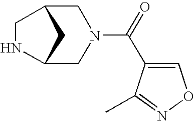 | 56.7 | 54.9 | ND; Failed HTS | 222 | |
| 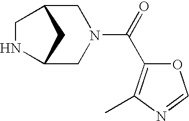 | 8.9 | 4.6 | ND; Failed HTS | 222 | |
| 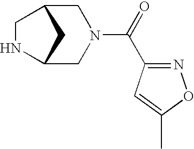 | 14.8 | 17.1 | ND; Failed HTS | 222 | |
| 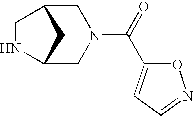 | 10.2 | 11.4 | ND; Failed HTS | 208 | |
| 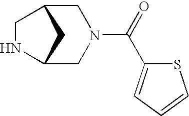 | 5.9 | 7.2 | ND; Failed HTS | 223 | |
| 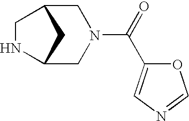 | 13 | 14 | ND; Failed HTS | 208 | |
| 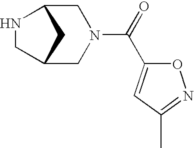 | 103.9 | 76.1 | ND; Failed HTS | 222 | |
| 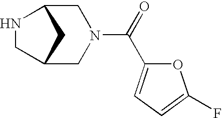 | 9.3 | 14.5 | ND; Failed HTS | 225 | |
| 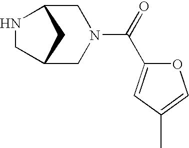 | 29.4 | 51.6 | ND; Failed HTS | 221 | |
| 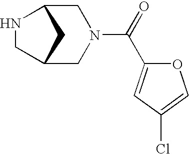 | 31.2 | 41.4 | ND; Failed HTS | 241 | 7.78 (s, 1H), 7.02 (s, 1H), 4.45- 4.05 (m, 2H), 3.60-3.40 (m, 2H), 3.15-2.85 (m, 3H), 2.50 (m, 1H), 1.96-1.81 (m, 2H) |
| 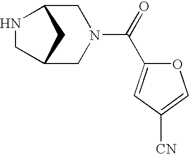 | 23.6 | 48.6 | ND; Failed HTS | 232 | |
| 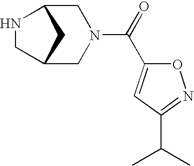 | 469.6 | 724.4 | ND; Failed HTS | 250 | |
| 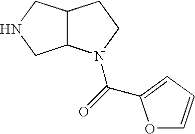 | 599.1 | 851 | ND; Failed HTS | 207 | d 7.74 (d, J = 0.97 Hz, 1H), 7.19 (d, J = 3.3 Hz, 1H), 6.62 (dd, J = 1.71 and 3.5 Hz, 1H), 4.68-4.80 (m, 1H), 3.95-4.20 (m, 2H), 3.80-3.45 (m, 3H), 3.40-3.15 (m, 2H), 2.38- 1.98 (m, 2H). |
| | 274.2 | 118.2 | ND; Failed HTS | 208 | |
| 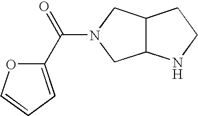 | 832 | 788.8 | ND; Failed HTS | 207 | |
| 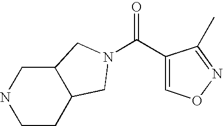 | 882.3 | 892.2 | ND; Failed HTS | 236 | |
| 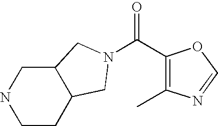 | 951.7 | 213.1 | ND; Failed HTS | 236 | 8.2 (s, 1H), 3.64-4.0 (m, 4H), 3.1-3.3 (m, 2H), 2.6-2.8 (m, 2H), 2.42 (s, 3H), 1.6-2.1 (m, 4H) |
| 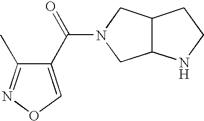 | 221.7 | 639 | ND; Failed HTS | 222 | |
| 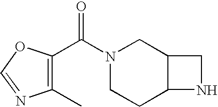 | ND | 791.7 | ND; Failed HTS | 222 | d 8.22 (s, 1H), 4.78-4.70 (m, 1H), 4.20-3.65 (m, 6H), 3.30-3.21 (m, 1H), 2.38 (s, 3H), 2.38-2.22 (m, 2H) |
| 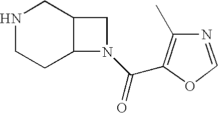 | ND | 514.9 | ND; Failed HTS | 222 | |

### Example 17

### Summary of Receptor Binding

Compounds of Table 1, representative of the present invention, exhibited inhibition constants (Ki values) at the rat and human α4β2 subtypes in the ranges of 3 nM to 1000 nM and 1 nM to 900 nM respectively, indicating high affinity for the α4β2 subtype. Ki values at the α7 subtype are greater than 1000 nM and many failed to bind sufficiently in high throughput screening (HTS) to warrant Ki determination, indicating low affinity for the α7 subtype.

The notation “failed HTS” as used herein for α7 subtype binding means that the compound failed to inhibit, at 5 μM concentration, the binding of 5 nM ³H-MLA (methyllycaconitine) by at least 50%.

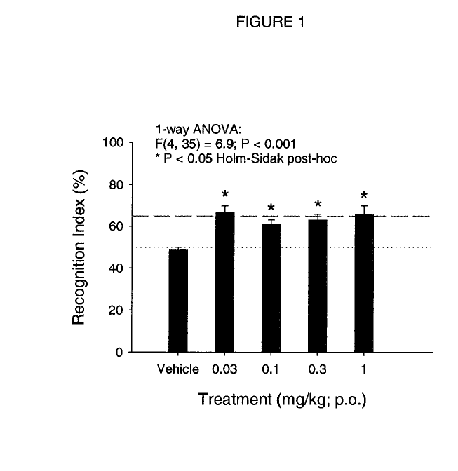

Certain exemplified compounds were assessed in the NOR (novel object recognition) task. Thus, (1S,5S)-3-(5-bromofuran-2-ylcarbonyl)-3,6-diazabicyclo[3.2.1]octane (FIG. 1) was active in NOR in rats, at 0.03 mg/kg. This provides evidence of the efficacy (and potency) of the compounds of the present invention in treating cognitive deficits, attentional disorders and dementias, and the potential of these compounds for human therapy.

Test compounds were employed in free or salt form.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula 1:
A-C(O)-Cy Formula 1
or a pharmaceutically acceptable salt thereof,
wherein A is a diazabicyclic core, containing 7, 8, or 9 ring atoms, and selected from the following: 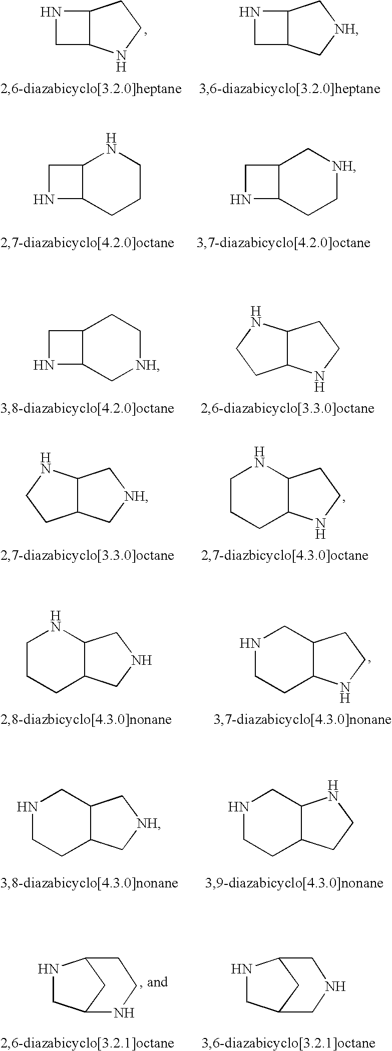
wherein the diazabicycle is attached as a radical to the depicted carbonyl via either one of the two ring nitrogen atoms, such that the carbonyl forms an amide bond with the ring nitrogen; Cy is a heteroaryl group selected from the group consisting of 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 1,3,4-oxadiazol-2-yl, 1,2,4-oxadiazol-3-yl, 1,2,4-oxadiazol-5-yl, 2-thiazolyl, 4-thiazolyl, 5-thiazolyl, 3-isothiazolyl, 4-isothiazolyl, 5-isothiazolyl, 1,3,4-thiadiazol-2-yl, 1,2,4-thiadiazol-3-yl, 1,2,4-thiadiazol-5-yl, 3-pyridinyl, and 4-pyridinyl, each of which may be optionally substituted with up to three non-hydrogen substituents selected from the group consisting of alkyl, alkenyl, heterocyclyl, cycloalkyl, aryl, heteroaryl, alkylaryl, arylalkyl, halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, and —NR′SO₂R″;
wherein each of alkyl, alkenyl, heterocyclyl, cycloalkyl, aryl, heteroaryl, alkylaryl, or arylalkyl may be substituted with one or more substituents selected from the group consisting of halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —C(═O)NR′R″, —NR′C(═O)O R″, —SO₂R′, —SO₂NR′R″, and —NR′SO₂R″ where R′ and R″ are individually selected from the group consisting of hydrogen, alkyl, cycloalkyl, heterocyclyl, aryl, heteroaryl, and arylalkyl, or R′ and R″ can combine with the atoms to which they are attached to form a 3- to 8-membered cyclic functionality.

2. The compound of claim 1 in isolated form.

3. The compound of claim 1, wherein A is selected from the group consisting of 3,7-diazabicyclo[4.2.0]octane, 2,7-diazabicyclo[4.2.0]octane, 3,8-diazabicyclo[4.2.0]octane, and 3,6-diazabicyclo[3.2.1]octane.

4. The compound of claim 1, wherein A is 3,6-diazabicyclo[3.2.1]octane.

5. The compound of claim 1, wherein Cy is selected from the group consisting of 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 3-pyridinyl, and 4-pyridinyl, each optionally substituted.

6. The compound of claim 5 wherein Cy is substituted with one or more of the group consisting of alkyl, aryl, heteroaryl, alkylaryl, arylalkyl, halogen, —CN, and —OR′, where R′ is selected from the group consisting of alkyl, aryl, and arylalkyl.

7. (canceled)

8. A method for treating central nervous system disorders, comprising administering a compound of Formula 1:
A-C(O)-Cy Formula 1
or a pharmaceutically acceptable salt thereof,
wherein A is a diazabicyclic core, containing 7, 8, or 9 ring atoms, and selected from the following: 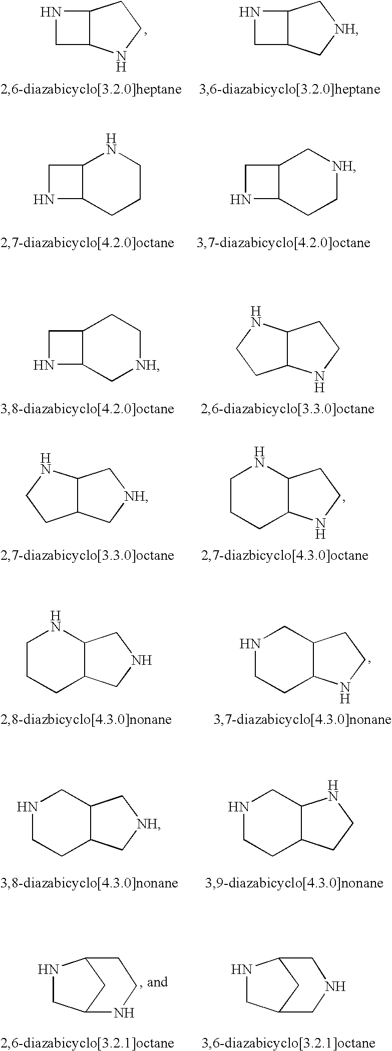
wherein the diazabicycle is attached as a radical to the depicted carbonyl via either one of the two ring nitrogen atoms, such that the carbonyl forms an amide bond with the ring nitrogen; Cy is a heteroaryl group selected from the group consisting of 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 1,3,4-oxadiazol-2-yl, 1,2,4-oxadiazol-3-yl, 1,2,4-oxadiazol-5-yl, 2-thiazolyl, 4-thiazolyl, 5-thiazolyl, 3-isothiazolyl, 4-isothiazolyl, 5-isothiazolyl, 1,3,4-thiadiazol-2-yl, 1,2,4-thiadiazol-3-yl, 1,2,4-thiadiazol-5-yl, 3-pyridinyl, and 4-pyridinyl, each of which may be optionally substituted with up to three non-hydrogen substituents selected from the group consisting of alkyl, alkenyl, heterocyclyl, cycloalkyl, aryl, heteroaryl, alkylaryl, arylalkyl, halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)R″, —SO₂R′, —SO₂NR′R″, and —NR′SO₂R″;
wherein each of alkyl, alkenyl, heterocyclyl, cycloalkyl, aryl, heteroaryl, alkylaryl, or arylalkyl may be substituted with one or more substituents selected from the group consisting of halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —C(═O)NR′R″, —NR′C(═O)OR″, —SO₂R′R″, and —NR′SO₂R″ where R′ and R″ are individually selected from the group consisting of hydrogen, alkyl, cycloalkyl, heterocyclyl, aryl, heteroaryl, and arylalkyl, or R′ and R″ can combine with the atoms to which they are attached to form a 3- to 8-membered cyclic functionality.

9. The method of claim 8, wherein the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder, and schizoaffective disorder.

10. A pharmaceutical composition comprising a compound of Formula 1:
A-C(O)-Cy Formula 1
or a pharmaceutically acceptable salt thereof,
wherein A is a diazabicyclic core, containing 7, 8, or 9 ring atoms, and selected from the following: 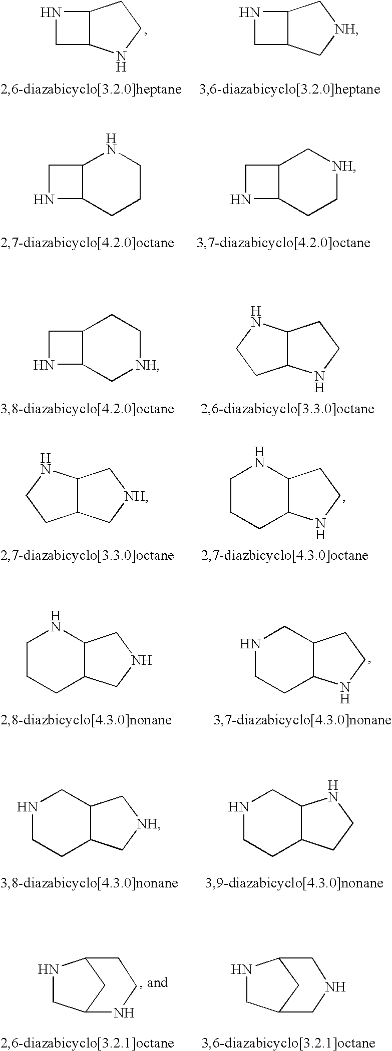
wherein the diazabicycle is attached as a radical to the depicted carbonyl via either one of the two ring nitrogen atoms, such that the carbonyl forms an amide bond with the ring nitrogen; Cy is a heteroaryl group selected from the group consisting of 2-furanyl, 3-furanyl, 2-thienyl, 3-thienyl, 2-oxazolyl, 4-oxazolyl, 5-oxazolyl, 3-isoxazolyl, 4-isoxazolyl, 5-isoxazolyl, 1,3,4-oxadiazol-2-yl, 1,2,4-oxadiazol-3-yl, 1,2,4-oxadiazol-5-yl, 2-thiazolyl, 4-thiazolyl, 5-thiazolyl, 3-isothiazolyl, 4-isothiazolyl, 5-isothiazolyl, 1,3,4-thiadiazol-2-yl, 1,2,4-thiadiazol-3-yl, 1,2,4-thiadiazol-5-yl, 3-pyridinyl, and 4-pyridinyl, each of which may be optionally substituted with up to three non-hydrogen substituents selected from the group consisting of alkyl, alkenyl, heterocyclyl, cycloalkyl, aryl, heteroaryl, alkylaryl, arylalkyl, halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, and —NR′SO₂R″;
wherein each of alkyl, alkenyl, heterocyclyl, cycloalkyl, aryl, heteroaryl, alkylaryl, or arylalkyl may be substituted with one or more substituents selected from the group consisting of halogen, —OR′, —NR′R″, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —C(═O)NR′R″, —NR′C(═O)O R″, —SO₂R′, —SO₂NR′R″, and —NR′SO₂R″ where R′ and R″ are individually selected from the group consisting of hydrogen, alkyl, cycloalkyl, heterocyclyl, aryl, heteroaryl, and arylalkyl, or R′ and R″ can combine with the atoms to which they are attached to form a 3- to 8-membered cyclic functionality.

11. The pharmaceutical composition according to claim 10 for treatment of central nervous system disorders.

12. A compound selected from the group consisting of:
2-(furan-2-ylcarbonyl)-2,6-diazabicyclo[3.2.0]heptane,
6-(furan-2-ylcarbonyl)-2,6-diazabicyclo[3.2.0]heptane,
2-(3-methylfuran-2-ylcarbonyl)-2,6-diazabicyclo[3.2.0]heptane,
6-(3-methylfuran-2-ylcarbonyl)-2,6-diazabicyclo[3.2.0]heptane,
2-(5-methylfuran-2-ylcarbonyl)-2,6-diazabicyclo[3.2.0]heptane,
6-(5-methylfuran-2-ylcarbonyl)-2,6-diazabicyclo[3.2.0]heptane,
2-(3-chlorofuran-2-ylcarbonyl)-2,6-diazabicyclo[3.2.0]heptane,
6-(3-chlorofuran-2-ylcarbonyl)-2,6-diazabicyclo[3.2.0]heptane,
2-(5-chlorofuran-2-ylcarbonyl)-2,6-diazabicyclo[3.2.0]heptane,
6-(5-chlorofuran-2-ylcarbonyl)-2,6-diazabicyclo[3.2.0]heptane,
2-(3-bromofuran-2-ylcarbonyl)-2,6-diazabicyclo[3.2.0]heptane,
6-(3-bromofuran-2-ylcarbonyl)-2,6-diazabicyclo[3.2.0]heptane,
2-(5-bromofuran-2-ylcarbonyl)-2,6-diazabicyclo[3.2.0]heptane,
6-(5-bromofuran-2-ylcarbonyl)-2,6-diazabicyclo[3.2.0]heptane,
2-(furan-3-ylcarbonyl)-2,6-diazabicyclo[3.2.0]heptane,
6-(furan-3-ylcarbonyl)-2,6-diazabicyclo[3.2.0]heptane,
2-(2-methylfuran-3-ylcarbonyl)-2,6-diazabicyclo[3.2.0]heptane,
6-(2-methylfuran-3-ylcarbonyl)-2,6-diazabicyclo[3.2.0]heptane,
2-(oxazol-2-ylcarbonyl)-2,6-diazabicyclo[3.2.0]heptane,
6-(oxazol-2-ylcarbonyl)-2,6-diazabicyclo[3.2.0]heptane,
2-(oxazol-5-ylcarbonyl)-2,6-diazabicyclo[3.2.0]heptane,
6-(oxazol-5-ylcarbonyl)-2,6-diazabicyclo[3.2.0]heptane,
2-(4-methyloxazol-5-ylcarbonyl)-2,6-diazabicyclo[3.2.0]heptane,
6-(4-methyloxazol-5-ylcarbonyl)-2,6-diazabicyclo[3.2.0]heptane,
2-(isoxazol-3-ylcarbonyl)-2,6-diazabicyclo[3.2.0]heptane,
6-(isoxazol-3-ylcarbonyl)-2,6-diazabicyclo[3.2.0]heptane,
2-(5-methylisoxazol-3-ylcarbonyl)-2,6-diazabicyclo[3.2.0]heptane,
6-(5-methylisoxazol-3-ylcarbonyl)-2,6-diazabicyclo[3.2.0]heptane,
2-(isoxazol-4-ylcarbonyl)-2,6-diazabicyclo[3.2.0]heptane,
6-(isoxazol-4-ylcarbonyl)-2,6-diazabicyclo[3.2.0]heptane,
2-(3-methylisoxazol-4-ylcarbonyl)-2,6-diazabicyclo[3.2.0]heptane,
6-(3-methylisoxazol-4-ylcarbonyl)-2,6-diazabicyclo[3.2.0]heptane,
2-(5-methylisoxazol-4-ylcarbonyl)-2,6-diazabicyclo[3.2.0]heptane,
6-(5-methylisoxazol-4-ylcarbonyl)-2,6-diazabicyclo[3.2.0]heptane,
2-(isoxazol-5-ylcarbonyl)-2,6-diazabicyclo[3.2.0]heptane,
6-(isoxazol-5-ylcarbonyl)-2,6-diazabicyclo[3.2.0]heptane,
2-(3-methylisoxazol-5-ylcarbonyl)-2,6-diazabicyclo[3.2.0]heptane,
6-(3-methylisoxazol-5-ylcarbonyl)-2,6-diazabicyclo[3.2.0]heptane,
2-(3-bromoisoxazol-5-ylcarbonyl)-2,6-diazabicyclo[3.2.0]heptane,
6-(3-bromoisoxazol-5-ylcarbonyl)-2,6-diazabicyclo[3.2.0]heptane,
2-(3-methoxyisoxazol-5-ylcarbonyl)-2,6-diazabicyclo[3.2.0]heptane,
6-(3-methoxyisoxazol-5-ylcarbonyl)-2,6-diazabicyclo[3.2.0]heptane,
2-(pyridin-4-ylcarbonyl)-2,6-diazabicyclo[3.2.0]heptane,
6-(pyridin-4-ylcarbonyl)-2,6-diazabicyclo[3.2.0]heptane,
3-(furan-2-ylcarbonyl)-3,6-diazabicyclo[3.2.0]heptane,
6-(furan-2-ylcarbonyl)-3,6-diazabicyclo[3.2.0]heptane,
3-(3-methylfuran-2-ylcarbonyl)-3,6-diazabicyclo[3.2.0]heptane,
6-(3-methylfuran-2-ylcarbonyl)-3,6-diazabicyclo[3.2.0]heptane,
3-(5-methylfuran-2-ylcarbonyl)-3,6-diazabicyclo[3.2.0]heptane,
6-(5-methylfuran-2-ylcarbonyl)-3,6-diazabicyclo[3.2.0]heptane,
3-(3-chlorofuran-2-ylcarbonyl)-3,6-diazabicyclo[3.2.0]heptane,
6-(3-chlorofuran-2-ylcarbonyl)-3,6-diazabicyclo[3.2.0]heptane,
3-(5-chlorofuran-2-ylcarbonyl)-3,6-diazabicyclo[3.2.0]heptane,
6-(5-chlorofuran-2-ylcarbonyl)-3,6-diazabicyclo[3.2.0]heptane,
3-(3-bromofuran-2-ylcarbonyl)-3,6-diazabicyclo[3.2.0]heptane,
6-(3-bromofuran-2-ylcarbonyl)-3,6-diazabicyclo[3.2.0]heptane,
3-(5-bromofuran-2-ylcarbonyl)-3,6-diazabicyclo[3.2.0]heptane,
6-(5-bromofuran-2-ylcarbonyl)-3,6-diazabicyclo[3.2.0]heptane,
3-(furan-3-ylcarbonyl)-3,6-diazabicyclo[3.2.0]heptane,
6-(furan-3-ylcarbonyl)-3,6-diazabicyclo[3.2.0]heptane,
3-(2-methylfuran-3-ylcarbonyl)-3,6-diazabicyclo[3.2.0]heptane,
6-(2-methylfuran-3-ylcarbonyl)-3,6-diazabicyclo[3.2.0]heptane,
3-(oxazol-2-ylcarbonyl)-3,6-diazabicyclo[3.2.0]heptane,
6-(oxazol-2-ylcarbonyl)-3,6-diazabicyclo[3.2.0]heptane,
3-(oxazol-5-ylcarbonyl)-3,6-diazabicyclo[3.2.0]heptane,
6-(oxazol-5-ylcarbonyl)-3,6-diazabicyclo[3.2.0]heptane,
3-(4-methyloxazol-5-ylcarbonyl)-3,6-diazabicyclo[3.2.0]heptane,
6-(4-methyloxazol-5-ylcarbonyl)-3,6-diazabicyclo[3.2.0]heptane,
3-(isoxazol-3-ylcarbonyl)-3,6-diazabicyclo[3.2.0]heptane,
6-(isoxazol-3-ylcarbonyl)-3,6-diazabicyclo[3.2.0]heptane,
3-(5-methylisoxazol-3-ylcarbonyl)-3,6-diazabicyclo[3.2.0]heptane,
6-(5-methylisoxazol-3-ylcarbonyl)-3,6-diazabicyclo[3.2.0]heptane,
3-(isoxazol-4-ylcarbonyl)-3,6-diazabicyclo[3.2.0]heptane,
6-(isoxazol-4-ylcarbonyl)-3,6-diazabicyclo[3.2.0]heptane,
3-(3-methylisoxazol-4-ylcarbonyl)-3,6-diazabicyclo[3.2.0]heptane,
6-(3-methylisoxazol-4-ylcarbonyl)-3,6-diazabicyclo[3.2.0]heptane,
3-(5-methylisoxazol-4-ylcarbonyl)-3,6-diazabicyclo[3.2.0]heptane,
6-(5-methylisoxazol-4-ylcarbonyl)-3,6-diazabicyclo[3.2.0]heptane,
3-(isoxazol-5-ylcarbonyl)-3,6-diazabicyclo[3.2.0]heptane,
6-(isoxazol-5-ylcarbonyl)-3,6-diazabicyclo[3.2.0]heptane,
3-(3-methylisoxazol-5-ylcarbonyl)-3,6-diazabicyclo[3.2.0]heptane,
6-(3-methylisoxazol-5-ylcarbonyl)-3,6-diazabicyclo[3.2.0]heptane,
3-(3-bromoisoxazol-5-ylcarbonyl)-3,6-diazabicyclo[3.2.0]heptane,
6-(3-bromoisoxazol-5-ylcarbonyl)-3,6-diazabicyclo[3.2.0]heptane,
3-(3-methoxyisoxazol-5-ylcarbonyl)-3,6-diazabicyclo[3.2.0]heptane,
6-(3-methoxyisoxazol-5-ylcarbonyl)-3,6-diazabicyclo[3.2.0]heptane,
3-(pyridin-4-ylcarbonyl)-3,6-diazabicyclo[3.2.0]heptane,
6-(pyridin-4-ylcarbonyl)-3,6-diazabicyclo[3.2.0]heptane,
2-(furan-2-ylcarbonyl)-2,7-diazabicyclo[4.2.0]octane,
7-(furan-2-ylcarbonyl)-2,7-diazabicyclo[4.2.0]octane,
2-(3-methylfuran-2-ylcarbonyl)-2,7-diazabicyclo[4.2.0]octane,
7-(3-methylfuran-2-ylcarbonyl)-2,7-diazabicyclo[4.2.0]octane,
2-(5-methylfuran-2-ylcarbonyl)-2,7-diazabicyclo[4.2.0]octane,
7-(5-methylfuran-2-ylcarbonyl)-2,7-diazabicyclo[4.2.0]octane,
2-(3-chlorofuran-2-ylcarbonyl)-2,7-diazabicyclo[4.2.0]octane,
7-(3-chlorofuran-2-ylcarbonyl)-2,7-diazabicyclo[4.2.0]octane,
2-(5-chlorofuran-2-ylcarbonyl)-2,7-diazabicyclo[4.2.0]octane,
7-(5-chlorofuran-2-ylcarbonyl)-2,7-diazabicyclo[4.2.0]octane,
2-(3-bromofuran-2-ylcarbonyl)-2,7-diazabicyclo[4.2.0]octane,
7-(3-bromofuran-2-ylcarbonyl)-2,7-diazabicyclo[4.2.0]octane,
2-(5-bromofuran-2-ylcarbonyl)-2,7-diazabicyclo[4.2.0]octane,
7-(5-bromofuran-2-ylcarbonyl)-2,7-diazabicyclo[4.2.0]octane,
2-(furan-3-ylcarbonyl)-2,7-diazabicyclo[4.2.0]octane,
7-(furan-3-ylcarbonyl)-2,7-diazabicyclo[4.2.0]octane,
2-(2-methylfuran-3-ylcarbonyl)-2,7-diazabicyclo[4.2.0]octane,
7-(2-methylfuran-3-ylcarbonyl)-2,7-diazabicyclo[4.2.0]octane,
2-(oxazol-2-ylcarbonyl)-2,7-diazabicyclo[4.2.0]octane,
7-(oxazol-2-ylcarbonyl)-2,7-diazabicyclo[4.2.0]octane,
2-(oxazol-5-ylcarbonyl)-2,7-diazabicyclo[4.2.0]octane,
7-(oxazol-5-ylcarbonyl)-2,7-diazabicyclo[4.2.0]octane,
2-(4-methyloxazol-5-ylcarbonyl)-2,7-diazabicyclo[4.2.0]octane,
7-(4-methyloxazol-5-ylcarbonyl)-2,7-diazabicyclo[4.2.0]octane,
2-(isoxazol-3-ylcarbonyl)-2,7-diazabicyclo[4.2.0]octane,
7-(isoxazol-3-ylcarbonyl)-2,7-diazabicyclo[4.2.0]octane,
2-(5-methylisoxazol-3-ylcarbonyl)-2,7-diazabicyclo[4.2.0]octane,
7-(5-methylisoxazol-3-ylcarbonyl)-2,7-diazabicyclo[4.2.0]octane,
2-(isoxazol-4-ylcarbonyl)-2,7-diazabicyclo[4.2.0]octane,
7-(isoxazol-4-ylcarbonyl)-2,7-diazabicyclo[4.2.0]octane,
2-(3-methylisoxazol-4-ylcarbonyl)-2,7-diazabicyclo[4.2.0]octane,
7-(3-methylisoxazol-4-ylcarbonyl)-2,7-diazabicyclo[4.2.0]octane,
2-(5-methylisoxazol-4-ylcarbonyl)-2,7-diazabicyclo[4.2.0]octane,
7-(5-methylisoxazol-4-ylcarbonyl)-2,7-diazabicyclo[4.2.0]octane,
2-(isoxazol-5-ylcarbonyl)-2,7-diazabicyclo[4.2.0]octane,
7-(isoxazol-5-ylcarbonyl)-2,7-diazabicyclo[4.2.0]octane,
2-(3-methylisoxazol-5-ylcarbonyl)-2,7-diazabicyclo[4.2.0]octane,
7-(3-methylisoxazol-5-ylcarbonyl)-2,7-diazabicyclo[4.2.0]octane,
2-(3-bromoisoxazol-5-ylcarbonyl)-2,7-diazabicyclo[4.2.0]octane,
7-(3-bromoisoxazol-5-ylcarbonyl)-2,7-diazabicyclo[4.2.0]octane,
2-(3-methoxyisoxazol-5-ylcarbonyl)-2,7-diazabicyclo[4.2.0]octane,
7-(3-methoxyisoxazol-5-ylcarbonyl)-2,7-diazabicyclo[4.2.0]octane,
2-(pyridin-4-ylcarbonyl)-2,7-diazabicyclo[4.2.0]octane,
7-(pyridin-4-ylcarbonyl)-2,7-diazabicyclo[4.2.0]octane,
3-(furan-2-ylcarbonyl)-3,7-diazabicyclo[4.2.0]octane,
7-(furan-2-ylcarbonyl)-3,7-diazabicyclo[4.2.0]octane,
3-(3-methylfuran-2-ylcarbonyl)-3,7-diazabicyclo[4.2.0]octane,
7-(3-methylfuran-2-ylcarbonyl)-3,7-diazabicyclo[4.2.0]octane,
3-(5-methylfuran-2-ylcarbonyl)-3,7-diazabicyclo[4.2.0]octane,
7-(5-methylfuran-2-ylcarbonyl)-3,7-diazabicyclo[4.2.0]octane,
3-(3-chlorofuran-2-ylcarbonyl)-3,7-diazabicyclo[4.2.0]octane,
7-(3-chlorofuran-2-ylcarbonyl)-3,7-diazabicyclo[4.2.0]octane,
3-(5-chlorofuran-2-ylcarbonyl)-3,7-diazabicyclo[4.2.0]octane,
7-(5-chlorofuran-2-ylcarbonyl)-3,7-diazabicyclo[4.2.0]octane,
3-(3-bromofuran-2-ylcarbonyl)-3,7-diazabicyclo[4.2.0]octane,
7-(3-bromofuran-2-ylcarbonyl)-3,7-diazabicyclo[4.2.0]octane,
3-(5-bromofuran-2-ylcarbonyl)-3,7-diazabicyclo[4.2.0]octane,
7-(5-bromofuran-2-ylcarbonyl)-3,7-diazabicyclo[4.2.0]octane,
3-(furan-3-ylcarbonyl)-3,7-diazabicyclo[4.2.0]octane,
7-(furan-3-ylcarbonyl)-3,7-diazabicyclo[4.2.0]octane,
3-(2-methylfuran-3-ylcarbonyl)-3,7-diazabicyclo[4.2.0]octane,
7-(2-methylfuran-3-ylcarbonyl)-3,7-diazabicyclo[4.2.0]octane,
3-(oxazol-2-ylcarbonyl)-3,7-diazabicyclo[4.2.0]octane,
7-(oxazol-2-ylcarbonyl)-3,7-diazabicyclo[4.2.0]octane,
3-(oxazol-5-ylcarbonyl)-3,7-diazabicyclo[4.2.0]octane,
7-(oxazol-5-ylcarbonyl)-3,7-diazabicyclo[4.2.0]octane,
3-(4-methyloxazol-5-ylcarbonyl)-3,7-diazabicyclo[4.2.0]octane,
7-(4-methyloxazol-5-ylcarbonyl)-3,7-diazabicyclo[4.2.0]octane,
3-(isoxazol-3-ylcarbonyl)-3,7-diazabicyclo[4.2.0]octane,
7-(isoxazol-3-ylcarbonyl)-3,7-diazabicyclo[4.2.0]octane,
3-(5-methylisoxazol-3-ylcarbonyl)-3,7-diazabicyclo[4.2.0]octane,
7-(5-methylisoxazol-3-ylcarbonyl)-3,7-diazabicyclo[4.2.0]octane,
3-(isoxazol-4-ylcarbonyl)-3,7-diazabicyclo[4.2.0]octane,
7-(isoxazol-4-ylcarbonyl)-3,7-diazabicyclo[4.2.0]octane,
3-(3-methylisoxazol-4-ylcarbonyl)-3,7-diazabicyclo[4.2.0]octane,
7-(3-methylisoxazol-4-ylcarbonyl)-3,7-diazabicyclo[4.2.0]octane,
3-(5-methylisoxazol-4-ylcarbonyl)-3,7-diazabicyclo[4.2.0]octane,
7-(5-methylisoxazol-4-ylcarbonyl)-3,7-diazabicyclo[4.2.0]octane,
3-(isoxazol-5-ylcarbonyl)-3,7-diazabicyclo[4.2.0]octane,
7-(isoxazol-5-ylcarbonyl)-3,7-diazabicyclo[4.2.0]octane,
3-(3-methylisoxazol-5-ylcarbonyl)-3,7-diazabicyclo[4.2.0]octane,
7-(3-methylisoxazol-5-ylcarbonyl)-3,7-diazabicyclo[4.2.0]octane,
3-(3-bromoisoxazol-5-ylcarbonyl)-3,7-diazabicyclo[4.2.0]octane,
7-(3-bromoisoxazol-5-ylcarbonyl)-3,7-diazabicyclo[4.2.0]octane,
3-(3-methoxyisoxazol-5-ylcarbonyl)-3,7-diazabicyclo[4.2.0]octane,
7-(3-methoxyisoxazol-5-ylcarbonyl)-3,7-diazabicyclo[4.2.0]octane,
3-(pyridin-4-ylcarbonyl)-3,7-diazabicyclo[4.2.0]octane,
7-(pyridin-4-ylcarbonyl)-3,7-diazabicyclo[4.2.0]octane,
3-(furan-2-ylcarbonyl)-3,8-diazabicyclo[4.2.0]octane,
8-(furan-2-ylcarbonyl)-3,8-diazabicyclo[4.2.0]octane,
3-(3-methylfuran-2-ylcarbonyl)-3,8-diazabicyclo[4.2.0]octane,
8-(3-methylfuran-2-ylcarbonyl)-3,8-diazabicyclo[4.2.0]octane,
3-(5-methylfuran-2-ylcarbonyl)-3,8-diazabicyclo[4.2.0]octane,
8-(5-methylfuran-2-ylcarbonyl)-3,8-diazabicyclo[4.2.0]octane,
3-(3-chlorofuran-2-ylcarbonyl)-3,8-diazabicyclo[4.2.0]octane,
8-(3-chlorofuran-2-ylcarbonyl)-3,8-diazabicyclo[4.2.0]octane,
3-(5-chlorofuran-2-ylcarbonyl)-3,8-diazabicyclo[4.2.0]octane,
8-(5-chlorofuran-2-ylcarbonyl)-3,8-diazabicyclo[4.2.0]octane,
3-(3-bromofuran-2-ylcarbonyl)-3,8-diazabicyclo[4.2.0]octane,
8-(3-bromofuran-2-ylcarbonyl)-3,8-diazabicyclo[4.2.0]octane,
3-(5-bromofuran-2-ylcarbonyl)-3,8-diazabicyclo[4.2.0]octane,
8-(5-bromofuran-2-ylcarbonyl)-3,8-diazabicyclo[4.2.0]octane,
3-(furan-3-ylcarbonyl)-3,8-diazabicyclo[4.2.0]octane,
8-(furan-3-ylcarbonyl)-3,8-diazabicyclo[4.2.0]octane,
3-(2-methylfuran-3-ylcarbonyl)-3,8-diazabicyclo[4.2.0]octane,
8-(2-methylfuran-3-ylcarbonyl)-3,8-diazabicyclo[4.2.0]octane,
3-(oxazol-2-ylcarbonyl)-3,8-diazabicyclo[4.2.0]octane,
8-(oxazol-2-ylcarbonyl)-3,8-diazabicyclo[4.2.0]octane,
3-(oxazol-5-ylcarbonyl)-3,8-diazabicyclo[4.2.0]octane,
8-(oxazol-5-ylcarbonyl)-3,8-diazabicyclo[4.2.0]octane,
3-(4-methyloxazol-5-ylcarbonyl)-3,8-diazabicyclo[4.2.0]octane,
8-(4-methyloxazol-5-ylcarbonyl)-3,8-diazabicyclo[4.2.0]octane,
3-(isoxazol-3-ylcarbonyl)-3,8-diazabicyclo[4.2.0]octane,
8-(isoxazol-3-ylcarbonyl)-3,8-diazabicyclo[4.2.0]octane,
3-(5-methylisoxazol-3-ylcarbonyl)-3,8-diazabicyclo[4.2.0]octane,
8-(5-methylisoxazol-3-ylcarbonyl)-3,8-diazabicyclo[4.2.0]octane,
3-(isoxazol-4-ylcarbonyl)-3,8-diazabicyclo[4.2.0]octane,
8-(isoxazol-4-ylcarbonyl)-3,8-diazabicyclo[4.2.0]octane,
3-(3-methylisoxazol-4-ylcarbonyl)-3,8-diazabicyclo[4.2.0]octane,
8-(3-methylisoxazol-4-ylcarbonyl)-3,8-diazabicyclo[4.2.0]octane,
3-(5-methylisoxazol-4-ylcarbonyl)-3,8-diazabicyclo[4.2.0]octane,
8-(5-methylisoxazol-4-ylcarbonyl)-3,8-diazabicyclo[4.2.0]octane,
3-(isoxazol-5-ylcarbonyl)-3,8-diazabicyclo[4.2.0]octane,
8-(isoxazol-5-ylcarbonyl)-3,8-diazabicyclo[4.2.0]octane,
3-(3-methylisoxazol-5-ylcarbonyl)-3,8-diazabicyclo[4.2.0]octane,
8-(3-methylisoxazol-5-ylcarbonyl)-3,8-diazabicyclo[4.2.0]octane,
3-(3-bromoisoxazol-5-ylcarbonyl)-3,8-diazabicyclo[4.2.0]octane,
8-(3-bromoisoxazol-5-ylcarbonyl)-3,8-diazabicyclo[4.2.0]octane,
3-(3-methoxyisoxazol-5-ylcarbonyl)-3,8-diazabicyclo[4.2.0]octane,
8-(3-methoxyisoxazol-5-ylcarbonyl)-3,8-diazabicyclo[4.2.0]octane,
3-(pyridin-4-ylcarbonyl)-3,8-diazabicyclo[4.2.0]octane,
8-(pyridin-4-ylcarbonyl)-3,8-diazabicyclo[4.2.0]octane,
2-(furan-2-ylcarbonyl)-2,6-diazabicyclo[3.3.0]octane,
2-(3-methylfuran-2-ylcarbonyl)-2,6-diazabicyclo[3.3.0]octane,
2-(5-methylfuran-2-ylcarbonyl)-2,6-diazabicyclo[3.3.0]octane,
2-(3-chlorofuran-2-ylcarbonyl)-2,6-diazabicyclo[3.3.0]octane,
2-(5-chlorofuran-2-ylcarbonyl)-2,6-diazabicyclo[3.3.0]octane,
2-(3-bromofuran-2-ylcarbonyl)-2,6-diazabicyclo[3.3.0]octane,
2-(5-bromofuran-2-ylcarbonyl)-2,6-diazabicyclo[3.3.0]octane,
2-(furan-3-ylcarbonyl)-2,6-diazabicyclo[3.3.0]octane,
2-(2-methylfuran-3-ylcarbonyl)-2,6-diazabicyclo[3.3.0]octane,
2-(oxazol-2-ylcarbonyl)-2,6-diazabicyclo[3.3.0]octane,
2-(oxazol-5-ylcarbonyl)-2,6-diazabicyclo[3.3.0]octane,
2-(4-methyloxazol-5-ylcarbonyl)-2,6-diazabicyclo[3.3.0]octane,
2-(isoxazol-3-ylcarbonyl)-2,6-diazabicyclo[3.3.0]octane,
2-(5-methylisoxazol-3-ylcarbonyl)-2,6-diazabicyclo[3.3.0]octane,
2-(isoxazol-4-ylcarbonyl)-2,6-diazabicyclo[3.3.0]octane,
2-(3-methylisoxazol-4-ylcarbonyl)-2,6-diazabicyclo[3.3.0]octane,
2-(5-methylisoxazol-4-ylcarbonyl)-2,6-diazabicyclo[3.3.0]octane,
2-(isoxazol-5-ylcarbonyl)-2,6-diazabicyclo[3.3.0]octane,
2-(3-methylisoxazol-5-ylcarbonyl)-2,6-diazabicyclo[3.3.0]octane,
2-(3-bromoisoxazol-5-ylcarbonyl)-2,6-diazabicyclo[3.3.0]octane,
2-(3-methoxyisoxazol-5-ylcarbonyl)-2,6-diazabicyclo[3.3.0]octane,
2-(pyridin-4-ylcarbonyl)-2,6-diazabicyclo[3.3.0]octane,
2-(furan-2-ylcarbonyl)-2,7-diazabicyclo[3.3.0]octane,
7-(furan-2-ylcarbonyl)-2,7-diazabicyclo[3.3.0]octane,
2-(3-methylfuran-2-ylcarbonyl)-2,7-diazabicyclo[3.3.0]octane,
7-(3-methylfuran-2-ylcarbonyl)-2,7-diazabicyclo[3.3.0]octane,
2-(5-methylfuran-2-ylcarbonyl)-2,7-diazabicyclo[3.3.0]octane,
7-(5-methylfuran-2-ylcarbonyl)-2,7-diazabicyclo[3.3.0]octane,
2-(3-chlorofuran-2-ylcarbonyl)-2,7-diazabicyclo[3.3.0]octane,
7-(3-chlorofuran-2-ylcarbonyl)-2,7-diazabicyclo[3.3.0]octane,
2-(5-chlorofuran-2-ylcarbonyl)-2,7-diazabicyclo[3.3.0]octane,
7-(5-chlorofuran-2-ylcarbonyl)-2,7-diazabicyclo[3.3.0]octane,
2-(3-bromofuran-2-ylcarbonyl)-2,7-diazabicyclo[3.3.0]octane,
7-(3-bromofuran-2-ylcarbonyl)-2,7-diazabicyclo[3.3.0]octane,
2-(5-bromofuran-2-ylcarbonyl)-2,7-diazabicyclo[3.3.0]octane,
7-(5-bromofuran-2-ylcarbonyl)-2,7-diazabicyclo[3.3.0]octane,
2-(furan-3-ylcarbonyl)-2,7-diazabicyclo[3.3.0]octane,
7-(furan-3-ylcarbonyl)-2,7-diazabicyclo[3.3.0]octane,
2-(2-methylfuran-3-ylcarbonyl)-2,7-diazabicyclo[3.3.0]octane,
7-(2-methylfuran-3-ylcarbonyl)-2,7-diazabicyclo[3.3.0]octane,
2-(oxazol-2-ylcarbonyl)-2,7-diazabicyclo[3.3.0]octane,
7-(oxazol-2-ylcarbonyl)-2,7-diazabicyclo[3.3.0]octane,
2-(oxazol-5-ylcarbonyl)-2,7-diazabicyclo[3.3.0]octane,
7-(oxazol-5-ylcarbonyl)-2,7-diazabicyclo[3.3.0]octane,
2-(4-methyloxazol-5-ylcarbonyl)-2,7-diazabicyclo[3.3.0]octane,
7-(4-methyloxazol-5-ylcarbonyl)-2,7-diazabicyclo[3.3.0]octane,
2-(isoxazol-3-ylcarbonyl)-2,7-diazabicyclo[3.3.0]octane,
7-(isoxazol-3-ylcarbonyl)-2,7-diazabicyclo[3.3.0]octane,
2-(5-methylisoxazol-3-ylcarbonyl)-2,7-diazabicyclo[3.3.0]octane,
7-(5-methylisoxazol-3-ylcarbonyl)-2,7-diazabicyclo[3.3.0]octane,
2-(isoxazol-4-ylcarbonyl)-2,7-diazabicyclo[3.3.0]octane,
7-(isoxazol-4-ylcarbonyl)-2,7-diazabicyclo[3.3.0]octane,
2-(3-methylisoxazol-4-ylcarbonyl)-2,7-diazabicyclo[3.3.0]octane,
7-(3-methylisoxazol-4-ylcarbonyl)-2,7-diazabicyclo[3.3.0]octane,
2-(5-methylisoxazol-4-ylcarbonyl)-2,7-diazabicyclo[3.3.0]octane,
7-(5-methylisoxazol-4-ylcarbonyl)-2,7-diazabicyclo[3.3.0]octane,
2-(isoxazol-5-ylcarbonyl)-2,7-diazabicyclo[3.3.0]octane,
7-(isoxazol-5-ylcarbonyl)-2,7-diazabicyclo[3.3.0]octane,
2-(3-methylisoxazol-5-ylcarbonyl)-2,7-diazabicyclo[3.3.0]octane,
7-(3-methylisoxazol-5-ylcarbonyl)-2,7-diazabicyclo[3.3.0]octane,
2-(3-bromoisoxazol-5-ylcarbonyl)-2,7-diazabicyclo[3.3.0]octane,
7-(3-bromoisoxazol-5-ylcarbonyl)-2,7-diazabicyclo[3.3.0]octane,
2-(3-methoxyisoxazol-5-ylcarbonyl)-2,7-diazabicyclo[3.3.0]octane,
7-(3-methoxyisoxazol-5-ylcarbonyl)-2,7-diazabicyclo[3.3.0]octane,
2-(pyridin-4-ylcarbonyl)-2,7-diazabicyclo[3.3.0]octane,
7-(pyridin-4-ylcarbonyl)-2,7-diazabicyclo[3.3.0]octane,
2-(furan-2-ylcarbonyl)-2,7-diazabicyclo[4.3.0]nonane,
7-(furan-2-ylcarbonyl)-2,7-diazabicyclo[4.3.0]nonane,
2-(3-methylfuran-2-ylcarbonyl)-2,7-diazabicyclo[4.3.0]nonane,
7-(3-methylfuran-2-ylcarbonyl)-2,7-diazabicyclo[4.3.0]nonane,
2-(5-methylfuran-2-ylcarbonyl)-2,7-diazabicyclo[4.3.0]nonane,
7-(5-methylfuran-2-ylcarbonyl)-2,7-diazabicyclo[4.3.0]nonane,
2-(3-chlorofuran-2-ylcarbonyl)-2,7-diazabicyclo[4.3.0]nonane,
7-(3-chlorofuran-2-ylcarbonyl)-2,7-diazabicyclo[4.3.0]nonane,
2-(5-chlorofuran-2-ylcarbonyl)-2,7-diazabicyclo[4.3.0]nonane,
7-(5-chlorofuran-2-ylcarbonyl)-2,7-diazabicyclo[4.3.0]nonane,
2-(3-bromofuran-2-ylcarbonyl)-2,7-diazabicyclo[4.3.0]nonane,
7-(3-bromofuran-2-ylcarbonyl)-2,7-diazabicyclo[4.3.0]nonane,
2-(5-bromofuran-2-ylcarbonyl)-2,7-diazabicyclo[4.3.0]nonane,
7-(5-bromofuran-2-ylcarbonyl)-2,7-diazabicyclo[4.3.0]nonane,
2-(furan-3-ylcarbonyl)-2,7-diazabicyclo[4.3.0]nonane,
7-(furan-3-ylcarbonyl)-2,7-diazabicyclo[4.3.0]nonane,
2-(2-methylfuran-3-ylcarbonyl)-2,7-diazabicyclo[4.3.0]nonane,
7-(2-methylfuran-3-ylcarbonyl)-2,7-diazabicyclo[4.3.0]nonane,
2-(oxazol-2-ylcarbonyl)-2,7-diazabicyclo[4.3.0]nonane,
7-(oxazol-2-ylcarbonyl)-2,7-diazabicyclo[4.3.0]nonane,
2-(oxazol-5-ylcarbonyl)-2,7-diazabicyclo[4.3.0]nonane,
7-(oxazol-5-ylcarbonyl)-2,7-diazabicyclo[4.3.0]nonane,
2-(4-methyloxazol-5-ylcarbonyl)-2,7-diazabicyclo[4.3.0]nonane,
7-(4-methyloxazol-5-ylcarbonyl)-2,7-diazabicyclo[4.3.0]nonane,
2-(isoxazol-3-ylcarbonyl)-2,7-diazabicyclo[4.3.0]nonane,
7-(isoxazol-3-ylcarbonyl)-2,7-diazabicyclo[4.3.0]nonane,
2-(5-methylisoxazol-3-ylcarbonyl)-2,7-diazabicyclo[4.3.0]nonane,
7-(5-methylisoxazol-3-ylcarbonyl)-2,7-diazabicyclo[4.3.0]nonane,
2-(isoxazol-4-ylcarbonyl)-2,7-diazabicyclo[4.3.0]nonane,
7-(isoxazol-4-ylcarbonyl)-2,7-diazabicyclo[4.3.0]nonane,
2-(3-methylisoxazol-4-ylcarbonyl)-2,7-diazabicyclo[4.3.0]nonane,
7-(3-methylisoxazol-4-ylcarbonyl)-2,7-diazabicyclo[4.3.0]nonane,
2-(5-methylisoxazol-4-ylcarbonyl)-2,7-diazabicyclo[4.3.0]nonane,
7-(5-methylisoxazol-4-ylcarbonyl)-2,7-diazabicyclo[4.3.0]nonane,
2-(isoxazol-5-ylcarbonyl)-2,7-diazabicyclo[4.3.0]nonane,
7-(isoxazol-5-ylcarbonyl)-2,7-diazabicyclo[4.3.0]nonane,
2-(3-methylisoxazol-5-ylcarbonyl)-2,7-diazabicyclo[4.3.0]nonane,
7-(3-methylisoxazol-5-ylcarbonyl)-2,7-diazabicyclo[4.3.0]nonane,
2-(3-bromoisoxazol-5-ylcarbonyl)-2,7-diazabicyclo[4.3.0]nonane,
7-(3-bromoisoxazol-5-ylcarbonyl)-2,7-diazabicyclo[4.3.0]nonane,
2-(3-methoxyisoxazol-5-ylcarbonyl)-2,7-diazabicyclo[4.3.0]nonane,
7-(3-methoxyisoxazol-5-ylcarbonyl)-2,7-diazabicyclo[4.3.0]nonane,
2-(pyridin-4-ylcarbonyl)-2,7-diazabicyclo[4.3.0]nonane,
7-(pyridin-4-ylcarbonyl)-2,7-diazabicyclo[4.3.0]nonane,
2-(furan-2-ylcarbonyl)-2,8-diazabicyclo[4.3.0]nonane,
8-(furan-2-ylcarbonyl)-2,8-diazabicyclo[4.3.0]nonane,
2-(3-methylfuran-2-ylcarbonyl)-2,8-diazabicyclo[4.3.0]nonane,
8-(3-methylfuran-2-ylcarbonyl)-2,8-diazabicyclo[4.3.0]nonane,
2-(5-methylfuran-2-ylcarbonyl)-2,8-diazabicyclo[4.3.0]nonane,
8-(5-methylfuran-2-ylcarbonyl)-2,8-diazabicyclo[4.3.0]nonane,
2-(3-chlorofuran-2-ylcarbonyl)-2,8-diazabicyclo[4.3.0]nonane,
8-(3-chlorofuran-2-ylcarbonyl)-2,8-diazabicyclo[4.3.0]nonane,
2-(5-chlorofuran-2-ylcarbonyl)-2,8-diazabicyclo[4.3.0]nonane,
8-(5-chlorofuran-2-ylcarbonyl)-2,8-diazabicyclo[4.3.0]nonane,
2-(3-bromofuran-2-ylcarbonyl)-2,8-diazabicyclo[4.3.0]nonane,
8-(3-bromofuran-2-ylcarbonyl)-2,8-diazabicyclo[4.3.0]nonane,
2-(5-bromofuran-2-ylcarbonyl)-2,8-diazabicyclo[4.3.0]nonane,
8-(5-bromofuran-2-ylcarbonyl)-2,8-diazabicyclo[4.3.0]nonane,
2-(furan-3-ylcarbonyl)-2,8-diazabicyclo[4.3.0]nonane,
8-(furan-3-ylcarbonyl)-2,8-diazabicyclo[4.3.0]nonane,
2-(2-methylfuran-3-ylcarbonyl)-2,8-diazabicyclo[4.3.0]nonane,
8-(2-methylfuran-3-ylcarbonyl)-2,8-diazabicyclo[4.3.0]nonane,
2-(oxazol-2-ylcarbonyl)-2,8-diazabicyclo[4.3.0]nonane,
8-(oxazol-2-ylcarbonyl)-2,8-diazabicyclo[4.3.0]nonane,
2-(oxazol-5-ylcarbonyl)-2,8-diazabicyclo[4.3.0]nonane,
8-(oxazol-5-ylcarbonyl)-2,8-diazabicyclo[4.3.0]nonane,
2-(4-methyloxazol-5-ylcarbonyl)-2,8-diazabicyclo[4.3.0]nonane,
8-(4-methyloxazol-5-ylcarbonyl)-2,8-diazabicyclo[4.3.0]nonane,
2-(isoxazol-3-ylcarbonyl)-2,8-diazabicyclo[4.3.0]nonane,
8-(isoxazol-3-ylcarbonyl)-2,8-diazabicyclo[4.3.0]nonane,
2-(5-methylisoxazol-3-ylcarbonyl)-2,8-diazabicyclo[4.3.0]nonane,
8-(5-methylisoxazol-3-ylcarbonyl)-2,8-diazabicyclo[4.3.0]nonane,
2-(isoxazol-4-ylcarbonyl)-2,8-diazabicyclo[4.3.0]nonane,
8-(isoxazol-4-ylcarbonyl)-2,8-diazabicyclo[4.3.0]nonane,
2-(3-methylisoxazol-4-ylcarbonyl)-2,8-diazabicyclo[4.3.0]nonane,
8-(3-methylisoxazol-4-ylcarbonyl)-2,8-diazabicyclo[4.3.0]nonane,
2-(5-methylisoxazol-4-ylcarbonyl)-2,8-diazabicyclo[4.3.0]nonane,
8-(5-methylisoxazol-4-ylcarbonyl)-2,8-diazabicyclo[4.3.0]nonane,
2-(isoxazol-5-ylcarbonyl)-2,8-diazabicyclo[4.3.0]nonane,
8-(isoxazol-5-ylcarbonyl)-2,8-diazabicyclo[4.3.0]nonane,
2-(3-methylisoxazol-5-ylcarbonyl)-2,8-diazabicyclo[4.3.0]nonane,
8-(3-methylisoxazol-5-ylcarbonyl)-2,8-diazabicyclo[4.3.0]nonane,
2-(3-bromoisoxazol-5-ylcarbonyl)-2,8-diazabicyclo[4.3.0]nonane,
8-(3-bromoisoxazol-5-ylcarbonyl)-2,8-diazabicyclo[4.3.0]nonane,
2-(3-methoxyisoxazol-5-ylcarbonyl)-2,8-diazabicyclo[4.3.0]nonane,
8-(3-methoxyisoxazol-5-ylcarbonyl)-2,8-diazabicyclo[4.3.0]nonane,
2-(pyridin-4-ylcarbonyl)-2,8-diazabicyclo[4.3.0]nonane,
8-(pyridin-4-ylcarbonyl)-2,8-diazabicyclo[4.3.0]nonane,
3-(furan-2-ylcarbonyl)-3,7-diazabicyclo[4.3.0]nonane,
7-(furan-2-ylcarbonyl)-3,7-diazabicyclo[4.3.0]nonane,
3-(3-methylfuran-2-ylcarbonyl)-3,7-diazabicyclo[4.3.0]nonane,
7-(3-methylfuran-2-ylcarbonyl)-3,7-diazabicyclo[4.3.0]nonane,
3-(5-methylfuran-2-ylcarbonyl)-3,7-diazabicyclo[4.3.0]nonane,
7-(5-methylfuran-2-ylcarbonyl)-3,7-diazabicyclo[4.3.0]nonane,
3-(3-chlorofuran-2-ylcarbonyl)-3,7-diazabicyclo[4.3.0]nonane,
7-(3-chlorofuran-2-ylcarbonyl)-3,7-diazabicyclo[4.3.0]nonane,
3-(5-chlorofuran-2-ylcarbonyl)-3,7-diazabicyclo[4.3.0]nonane,
7-(5-chlorofuran-2-ylcarbonyl)-3,7-diazabicyclo[4.3.0]nonane,
3-(3-bromofuran-2-ylcarbonyl)-3,7-diazabicyclo[4.3.0]nonane,
7-(3-bromofuran-2-ylcarbonyl)-3,7-diazabicyclo[4.3.0]nonane,
3-(5-bromofuran-2-ylcarbonyl)-3,7-diazabicyclo[4.3.0]nonane,
7-(5-bromofuran-2-ylcarbonyl)-3,7-diazabicyclo[4.3.0]nonane,
3-(furan-3-ylcarbonyl)-3,7-diazabicyclo[4.3.0]nonane,
7-(furan-3-ylcarbonyl)-3,7-diazabicyclo[4.3.0]nonane,
3-(2-methylfuran-3-ylcarbonyl)-3,7-diazabicyclo[4.3.0]nonane,
7-(2-methylfuran-3-ylcarbonyl)-3,7-diazabicyclo[4.3.0]nonane,
3-(oxazol-2-ylcarbonyl)-3,7-diazabicyclo[4.3.0]nonane,
7-(oxazol-2-ylcarbonyl)-3,7-diazabicyclo[4.3.0]nonane,
3-(oxazol-5-ylcarbonyl)-3,7-diazabicyclo[4.3.0]nonane,
7-(oxazol-5-ylcarbonyl)-3,7-diazabicyclo[4.3.0]nonane,
3-(4-methyloxazol-5-ylcarbonyl)-3,7-diazabicyclo[4.3.0]nonane,
7-(4-methyloxazol-5-ylcarbonyl)-3,7-diazabicyclo[4.3.0]nonane,
3-(isoxazol-3-ylcarbonyl)-3,7-diazabicyclo[4.3.0]nonane,
7-(isoxazol-3-ylcarbonyl)-3,7-diazabicyclo[4.3.0]nonane,
3-(5-methylisoxazol-3-ylcarbonyl)-3,7-diazabicyclo[4.3.0]nonane,
7-(5-methylisoxazol-3-ylcarbonyl)-3,7-diazabicyclo[4.3.0]nonane,
3-(isoxazol-4-ylcarbonyl)-3,7-diazabicyclo[4.3.0]nonane,
7-(isoxazol-4-ylcarbonyl)-3,7-diazabicyclo[4.3.0]nonane,
3-(3-methylisoxazol-4-ylcarbonyl)-3,7-diazabicyclo[4.3.0]nonane,
7-(3-methylisoxazol-4-ylcarbonyl)-3,7-diazabicyclo[4.3.0]nonane,
3-(5-methylisoxazol-4-ylcarbonyl)-3,7-diazabicyclo[4.3.0]nonane,
7-(5-methylisoxazol-4-ylcarbonyl)-3,7-diazabicyclo[4.3.0]nonane,
3-(isoxazol-5-ylcarbonyl)-3,7-diazabicyclo[4.3.0]nonane,
7-(isoxazol-5-ylcarbonyl)-3,7-diazabicyclo[4.3.0]nonane,
3-(3-methylisoxazol-5-ylcarbonyl)-3,7-diazabicyclo[4.3.0]nonane,
7-(3-methylisoxazol-5-ylcarbonyl)-3,7-diazabicyclo[4.3.0]nonane,
3-(3-bromoisoxazol-5-ylcarbonyl)-3,7-diazabicyclo[4.3.0]nonane,
7-(3-bromoisoxazol-5-ylcarbonyl)-3,7-diazabicyclo[4.3.0]nonane,
3-(3-methoxyisoxazol-5-ylcarbonyl)-3,7-diazabicyclo[4.3.0]nonane,
7-(3-methoxyisoxazol-5-ylcarbonyl)-3,7-diazabicyclo[4.3.0]nonane,
3-(pyridin-4-ylcarbonyl)-3,7-diazabicyclo[4.3.0]nonane,
7-(pyridin-4-ylcarbonyl)-3,7-diazabicyclo[4.3.0]nonane,
3-(furan-2-ylcarbonyl)-3,8-diazabicyclo[4.3.0]nonane,
8-(furan-2-ylcarbonyl)-3,8-diazabicyclo[4.3.0]nonane,
3-(3-methylfuran-2-ylcarbonyl)-3,8-diazabicyclo[4.3.0]nonane,
8-(3-methylfuran-2-ylcarbonyl)-3,8-diazabicyclo[4.3.0]nonane,
3-(5-methylfuran-2-ylcarbonyl)-3,8-diazabicyclo[4.3.0]nonane,
8-(5-methylfuran-2-ylcarbonyl)-3,8-diazabicyclo[4.3.0]nonane,
3-(3-chlorofuran-2-ylcarbonyl)-3,8-diazabicyclo[4.3.0]nonane,
8-(3-chlorofuran-2-ylcarbonyl)-3,8-diazabicyclo[4.3.0]nonane,
3-(5-chlorofuran-2-ylcarbonyl)-3,8-diazabicyclo[4.3.0]nonane,
8-(5-chlorofuran-2-ylcarbonyl)-3,8-diazabicyclo[4.3.0]nonane,
3-(3-bromofuran-2-ylcarbonyl)-3,8-diazabicyclo[4.3.0]nonane,
8-(3-bromofuran-2-ylcarbonyl)-3,8-diazabicyclo[4.3.0]nonane,
3-(5-bromofuran-2-ylcarbonyl)-3,8-diazabicyclo[4.3.0]nonane,
8-(5-bromofuran-2-ylcarbonyl)-3,8-diazabicyclo[4.3.0]nonane,
3-(furan-3-ylcarbonyl)-3,8-diazabicyclo[4.3.0]nonane,
8-(furan-3-ylcarbonyl)-3,8-diazabicyclo[4.3.0]nonane,
3-(2-methylfuran-3-ylcarbonyl)-3,8-diazabicyclo[4.3.0]nonane,
8-(2-methylfuran-3-ylcarbonyl)-3,8-diazabicyclo[4.3.0]nonane,
3-(oxazol-2-ylcarbonyl)-3,8-diazabicyclo[4.3.0]nonane,
8-(oxazol-2-ylcarbonyl)-3,8-diazabicyclo[4.3.0]nonane,
3-(oxazol-5-ylcarbonyl)-3,8-diazabicyclo[4.3.0]nonane,
8-(oxazol-5-ylcarbonyl)-3,8-diazabicyclo[4.3.0]nonane,
3-(4-methyloxazol-5-ylcarbonyl)-3,8-diazabicyclo[4.3.0]nonane,
8-(4-methyloxazol-5-ylcarbonyl)-3,8-diazabicyclo[4.3.0]nonane,
3-(isoxazol-3-ylcarbonyl)-3,8-diazabicyclo[4.3.0]nonane,
8-(isoxazol-3-ylcarbonyl)-3,8-diazabicyclo[4.3.0]nonane,
3-(5-methylisoxazol-3-ylcarbonyl)-3,8-diazabicyclo[4.3.0]nonane,
8-(5-methylisoxazol-3-ylcarbonyl)-3,8-diazabicyclo[4.3.0]nonane,
3-(isoxazol-4-ylcarbonyl)-3,8-diazabicyclo[4.3.0]nonane,
8-(isoxazol-4-ylcarbonyl)-3,8-diazabicyclo[4.3.0]nonane,
3-(3-methylisoxazol-4-ylcarbonyl)-3,8-diazabicyclo[4.3.0]nonane,
8-(3-methylisoxazol-4-ylcarbonyl)-3,8-diazabicyclo[4.3.0]nonane,
3-(5-methylisoxazol-4-ylcarbonyl)-3,8-diazabicyclo[4.3.0]nonane,
8-(5-methylisoxazol-4-ylcarbonyl)-3,8-diazabicyclo[4.3.0]nonane,
3-(isoxazol-5-ylcarbonyl)-3,8-diazabicyclo[4.3.0]nonane,
8-(isoxazol-5-ylcarbonyl)-3,8-diazabicyclo[4.3.0]nonane,
3-(3-methylisoxazol-5-ylcarbonyl)-3,8-diazabicyclo[4.3.0]nonane,
8-(3-methylisoxazol-5-ylcarbonyl)-3,8-diazabicyclo[4.3.0]nonane,
3-(3-bromoisoxazol-5-ylcarbonyl)-3,8-diazabicyclo[4.3.0]nonane,
8-(3-bromoisoxazol-5-ylcarbonyl)-3,8-diazabicyclo[4.3.0]nonane,
3-(3-methoxyisoxazol-5-ylcarbonyl)-3,8-diazabicyclo[4.3.0]nonane,
8-(3-methoxyisoxazol-5-ylcarbonyl)-3,8-diazabicyclo[4.3.0]nonane,
3-(pyridin-4-ylcarbonyl)-3,8-diazabicyclo[4.3.0]nonane,
8-(pyridin-4-ylcarbonyl)-3,8-diazabicyclo[4.3.0]nonane,
3-(furan-2-ylcarbonyl)-3,9-diazabicyclo[4.3.0]nonane,
9-(furan-2-ylcarbonyl)-3,9-diazabicyclo[4.3.0]nonane,
3-(3-methylfuran-2-ylcarbonyl)-3,9-diazabicyclo[4.3.0]nonane,
9-(3-methylfuran-2-ylcarbonyl)-3,9-diazabicyclo[4.3.0]nonane,
3-(5-methylfuran-2-ylcarbonyl)-3,9-diazabicyclo[4.3.0]nonane,
9-(5-methylfuran-2-ylcarbonyl)-3,9-diazabicyclo[4.3.0]nonane,
3-(3-chlorofuran-2-ylcarbonyl)-3,9-diazabicyclo[4.3.0]nonane,
9-(3-chlorofuran-2-ylcarbonyl)-3,9-diazabicyclo[4.3.0]nonane,
3-(5-chlorofuran-2-ylcarbonyl)-3,9-diazabicyclo[4.3.0]nonane,
9-(5-chlorofuran-2-ylcarbonyl)-3,9-diazabicyclo[4.3.0]nonane,
3-(3-bromofuran-2-ylcarbonyl)-3,9-diazabicyclo[4.3.0]nonane,
9-(3-bromofuran-2-ylcarbonyl)-3,9-diazabicyclo[4.3.0]nonane,
3-(5-bromofuran-2-ylcarbonyl)-3,9-diazabicyclo[4.3.0]nonane,
9-(5-bromofuran-2-ylcarbonyl)-3,9-diazabicyclo[4.3.0]nonane,
3-(furan-3-ylcarbonyl)-3,9-diazabicyclo[4.3.0]nonane,
9-(furan-3-ylcarbonyl)-3,9-diazabicyclo[4.3.0]nonane,
3-(2-methylfuran-3-ylcarbonyl)-3,9-diazabicyclo[4.3.0]nonane,
9-(2-methylfuran-3-ylcarbonyl)-3,9-diazabicyclo[4.3.0]nonane,
3-(oxazol-2-ylcarbonyl)-3,9-diazabicyclo[4.3.0]nonane,
9-(oxazol-2-ylcarbonyl)-3,9-diazabicyclo[4.3.0]nonane,
3-(oxazol-5-ylcarbonyl)-3,9-diazabicyclo[4.3.0]nonane,
9-(oxazol-5-ylcarbonyl)-3,9-diazabicyclo[4.3.0]nonane,
3-(4-methyloxazol-5-ylcarbonyl)-3,9-diazabicyclo[4.3.0]nonane,
9-(4-methyloxazol-5-ylcarbonyl)-3,9-diazabicyclo[4.3.0]nonane,
3-(isoxazol-3-ylcarbonyl)-3,9-diazabicyclo[4.3.0]nonane,
9-(isoxazol-3-ylcarbonyl)-3,9-diazabicyclo[4.3.0]nonane,
3-(5-methylisoxazol-3-ylcarbonyl)-3,9-diazabicyclo[4.3.0]nonane,
9-(5-methylisoxazol-3-ylcarbonyl)-3,9-diazabicyclo[4.3.0]nonane,
3-(isoxazol-4-ylcarbonyl)-3,9-diazabicyclo[4.3.0]nonane,
9-(isoxazol-4-ylcarbonyl)-3,9-diazabicyclo[4.3.0]nonane,
3-(3-methylisoxazol-4-ylcarbonyl)-3,9-diazabicyclo[4.3.0]nonane,
9-(3-methylisoxazol-4-ylcarbonyl)-3,9-diazabicyclo[4.3.0]nonane,
3-(5-methylisoxazol-4-ylcarbonyl)-3,9-diazabicyclo[4.3.0]nonane,
9-(5-methylisoxazol-4-ylcarbonyl)-3,9-diazabicyclo[4.3.0]nonane,
3-(isoxazol-5-ylcarbonyl)-3,9-diazabicyclo[4.3.0]nonane,
9-(isoxazol-5-ylcarbonyl)-3,9-diazabicyclo[4.3.0]nonane,
3-(3-methylisoxazol-5-ylcarbonyl)-3,9-diazabicyclo[4.3.0]nonane,
9-(3-methylisoxazol-5-ylcarbonyl)-3,9-diazabicyclo[4.3.0]nonane,
3-(3-bromoisoxazol-5-ylcarbonyl)-3,9-diazabicyclo[4.3.0]nonane,
9-(3-bromoisoxazol-5-ylcarbonyl)-3,9-diazabicyclo[4.3.0]nonane,
3-(3-methoxyisoxazol-5-ylcarbonyl)-3,9-diazabicyclo[4.3.0]nonane,
9-(3-methoxyisoxazol-5-ylcarbonyl)-3,9-diazabicyclo[4.3.0]nonane,
3-(pyridin-4-ylcarbonyl)-3,9-diazabicyclo[4.3.0]nonane,
9-(pyridin-4-ylcarbonyl)-3,9-diazabicyclo[4.3.0]nonane,
2-(furan-2-ylcarbonyl)-2,6-diazabicyclo[3.2.1]octane,
6-(furan-2-ylcarbonyl)-2,6-diazabicyclo[3.2.1]octane,
2-(3-methylfuran-2-ylcarbonyl)-2,6-diazabicyclo[3.2.1]octane,
6-(3-methylfuran-2-ylcarbonyl)-2,6-diazabicyclo[3.2.1]octane,
2-(5-methylfuran-2-ylcarbonyl)-2,6-diazabicyclo[3.2.1]octane,
6-(5-methylfuran-2-ylcarbonyl)-2,6-diazabicyclo[3.2.1]octane,
2-(3-chlorofuran-2-ylcarbonyl)-2,6-diazabicyclo[3.2.1]octane,
6-(3-chlorofuran-2-ylcarbonyl)-2,6-diazabicyclo[3.2.1]octane,
2-(5-chlorofuran-2-ylcarbonyl)-2,6-diazabicyclo[3.2.1]octane,
6-(5-chlorofuran-2-ylcarbonyl)-2,6-diazabicyclo[3.2.1]octane,
2-(3-bromofuran-2-ylcarbonyl)-2,6-diazabicyclo[3.2.1]octane,
6-(3-bromofuran-2-ylcarbonyl)-2,6-diazabicyclo[3.2.1]octane,
2-(5-bromofuran-2-ylcarbonyl)-2,6-diazabicyclo[3.2.1]octane,
6-(5-bromofuran-2-ylcarbonyl)-2,6-diazabicyclo[3.2.1]octane,
2-(furan-3-ylcarbonyl)-2,6-diazabicyclo[3.2.1]octane,
6-(furan-3-ylcarbonyl)-2,6-diazabicyclo[3.2.1]octane,
2-(2-methylfuran-3-ylcarbonyl)-2,6-diazabicyclo[3.2.1]octane,
6-(2-methylfuran-3-ylcarbonyl)-2,6-diazabicyclo[3.2.1]octane,
2-(oxazol-2-ylcarbonyl)-2,6-diazabicyclo[3.2.1]octane,
6-(oxazol-2-ylcarbonyl)-2,6-diazabicyclo[3.2.1]octane,
2-(oxazol-5-ylcarbonyl)-2,6-diazabicyclo[3.2.1]octane,
6-(oxazol-5-ylcarbonyl)-2,6-diazabicyclo[3.2.1]octane,
2-(4-methyloxazol-5-ylcarbonyl)-2,6-diazabicyclo[3.2.1]octane,
6-(4-methyloxazol-5-ylcarbonyl)-2,6-diazabicyclo[3.2.1]octane,
2-(isoxazol-3-ylcarbonyl)-2,6-diazabicyclo[3.2.1]octane,
6-(isoxazol-3-ylcarbonyl)-2,6-diazabicyclo[3.2.1]octane,
2-(5-methylisoxazol-3-ylcarbonyl)-2,6-diazabicyclo[3.2.1]octane,
6-(5-methylisoxazol-3-ylcarbonyl)-2,6-diazabicyclo[3.2.1]octane,
2-(isoxazol-4-ylcarbonyl)-2,6-diazabicyclo[3.2.1]octane,
6-(isoxazol-4-ylcarbonyl)-2,6-diazabicyclo[3.2.1]octane,
2-(3-methylisoxazol-4-ylcarbonyl)-2,6-diazabicyclo[3.2.1]octane,
6-(3-methylisoxazol-4-ylcarbonyl)-2,6-diazabicyclo[3.2.1]octane,
2-(5-methylisoxazol-4-ylcarbonyl)-2,6-diazabicyclo[3.2.1]octane,
6-(5-methylisoxazol-4-ylcarbonyl)-2,6-diazabicyclo[3.2.1]octane,
2-(isoxazol-5-ylcarbonyl)-2,6-diazabicyclo[3.2.1]octane,
6-(isoxazol-5-ylcarbonyl)-2,6-diazabicyclo[3.2.1]octane,
2-(3-methylisoxazol-5-ylcarbonyl)-2,6-diazabicyclo[3.2.1]octane,
6-(3-methylisoxazol-5-ylcarbonyl)-2,6-diazabicyclo[3.2.1]octane,
2-(3-bromoisoxazol-5-ylcarbonyl)-2,6-diazabicyclo[3.2.1]octane,
6-(3-bromoisoxazol-5-ylcarbonyl)-2,6-diazabicyclo[3.2.1]octane,
2-(3-methoxyisoxazol-5-ylcarbonyl)-2,6-diazabicyclo[3.2.1]octane,
6-(3-methoxyisoxazol-5-ylcarbonyl)-2,6-diazabicyclo[3.2.1]octane,
2-(pyridin-4-ylcarbonyl)-2,6-diazabicyclo[3.2.1]octane,
6-(pyridin-4-ylcarbonyl)-2,6-diazabicyclo[3.2.1]octane,
3-(furan-2-ylcarbonyl)-3,6-diazabicyclo[3.2.1]octane,
6-(furan-2-ylcarbonyl)-3,6-diazabicyclo[3.2.1]octane,
3-(3-methylfuran-2-ylcarbonyl)-3,6-diazabicyclo[3.2.1]octane,
6-(3-methylfuran-2-ylcarbonyl)-3,6-diazabicyclo[3.2.1]octane,
3-(5-methylfuran-2-ylcarbonyl)-3,6-diazabicyclo[3.2.1]octane,
6-(5-methylfuran-2-ylcarbonyl)-3,6-diazabicyclo[3.2.1]octane,
3-(3-chlorofuran-2-ylcarbonyl)-3,6-diazabicyclo[3.2.1]octane,
6-(3-chlorofuran-2-ylcarbonyl)-3,6-diazabicyclo[3.2.1]octane,
3-(5-chlorofuran-2-ylcarbonyl)-3,6-diazabicyclo[3.2.1]octane,
6-(5-chlorofuran-2-ylcarbonyl)-3,6-diazabicyclo[3.2.1]octane,
3-(3-bromofuran-2-ylcarbonyl)-3,6-diazabicyclo[3.2.1]octane,
6-(3-bromofuran-2-ylcarbonyl)-3,6-diazabicyclo[3.2.1]octane,
3-(5-bromofuran-2-ylcarbonyl)-3,6-diazabicyclo[3.2.1]octane,
6-(5-bromofuran-2-ylcarbonyl)-3,6-diazabicyclo[3.2.1]octane,
3-(furan-3-ylcarbonyl)-3,6-diazabicyclo[3.2.1]octane,
6-(furan-3-ylcarbonyl)-3,6-diazabicyclo[3.2.1]octane,
3-(2-methylfuran-3-ylcarbonyl)-3,6-diazabicyclo[3.2.1]octane,
6-(2-methylfuran-3-ylcarbonyl)-3,6-diazabicyclo[3.2.1]octane,
3-(oxazol-2-ylcarbonyl)-3,6-diazabicyclo[3.2.1]octane,
6-(oxazol-2-ylcarbonyl)-3,6-diazabicyclo[3.2.1]octane,
3-(oxazol-5-ylcarbonyl)-3,6-diazabicyclo[3.2.1]octane,
6-(oxazol-5-ylcarbonyl)-3,6-diazabicyclo[3.2.1]octane,
3-(4-methyloxazol-5-ylcarbonyl)-3,6-diazabicyclo[3.2.1]octane,
6-(4-methyloxazol-5-ylcarbonyl)-3,6-diazabicyclo[3.2.1]octane,
3-(isoxazol-3-ylcarbonyl)-3,6-diazabicyclo[3.2.1]octane,
6-(isoxazol-3-ylcarbonyl)-3,6-diazabicyclo[3.2.1]octane,
3-(5-methylisoxazol-3-ylcarbonyl)-3,6-diazabicyclo[3.2.1]octane,
6-(5-methylisoxazol-3-ylcarbonyl)-3,6-diazabicyclo[3.2.1]octane,
3-(isoxazol-4-ylcarbonyl)-3,6-diazabicyclo[3.2.1]octane,
6-(isoxazol-4-ylcarbonyl)-3,6-diazabicyclo[3.2.1]octane,
3-(3-methylisoxazol-4-ylcarbonyl)-3,6-diazabicyclo[3.2.1]octane,
6-(3-methylisoxazol-4-ylcarbonyl)-3,6-diazabicyclo[3.2.1]octane,
3-(5-methylisoxazol-4-ylcarbonyl)-3,6-diazabicyclo[3.2.1]octane,
6-(5-methylisoxazol-4-ylcarbonyl)-3,6-diazabicyclo[3.2.1]octane,
3-(isoxazol-5-ylcarbonyl)-3,6-diazabicyclo[3.2.1]octane,
6-(isoxazol-5-ylcarbonyl)-3,6-diazabicyclo[3.2.1]octane,
3-(3-methylisoxazol-5-ylcarbonyl)-3,6-diazabicyclo[3.2.1]octane,
6-(3-methylisoxazol-5-ylcarbonyl)-3,6-diazabicyclo[3.2.1]octane,
3-(3-bromoisoxazol-5-ylcarbonyl)-3,6-diazabicyclo[3.2.1]octane,
6-(3-bromoisoxazol-5-ylcarbonyl)-3,6-diazabicyclo[3.2.1]octane,
3-(3-methoxyisoxazol-5-ylcarbonyl)-3,6-diazabicyclo[3.2.1]octane,
6-(3-methoxyisoxazol-5-ylcarbonyl)-3,6-diazabicyclo[3.2.1]octane,
3-(pyridin-4-ylcarbonyl)-3,6-diazabicyclo[3.2.1]octane, and 6-(pyridin-4-ylcarbonyl)-3,6-diazabicyclo[3.2.1]octane,
or a pharmaceutically acceptable salt thereof.

13. The compound according to claim 12 in isolated form.

14. A method for treating central nervous system disorders, comprising administering a salt of a compound selected from the group consisting of:
2-(furan-2-ylcarbonyl)-2,6-diazabicyclo[3.2.0]heptane,
6-(furan-2-ylcarbonyl)-2,6-diazabicyclo[3.2.0]heptane,
2-(3-methylfuran-2-ylcarbonyl)-2,6-diazabicyclo[3.2.0]heptane,
6-(3-methylfuran-2-ylcarbonyl)-2,6-diazabicyclo[3.2.0]heptane,
2-(5-methylfuran-2-ylcarbonyl)-2,6-diazabicyclo[3.2.0]heptane,
6-(5-methylfuran-2-ylcarbonyl)-2,6-diazabicyclo[3.2.0]heptane,
2-(3-chlorofuran-2-ylcarbonyl)-2,6-diazabicyclo[3.2.0]heptane,
6-(3-chlorofuran-2-ylcarbonyl)-2,6-diazabicyclo[3.2.0]heptane,
2-(5-chlorofuran-2-ylcarbonyl)-2,6-diazabicyclo[3.2.0]heptane,
6-(5-chlorofuran-2-ylcarbonyl)-2,6-diazabicyclo[3.2.0]heptane,
2-(3-bromofuran-2-ylcarbonyl)-2,6-diazabicyclo[3.2.0]heptane,
6-(3-bromofuran-2-ylcarbonyl)-2,6-diazabicyclo[3.2.0]heptane,
2-(5-bromofuran-2-ylcarbonyl)-2,6-diazabicyclo[3.2.0]heptane,
6-(5-bromofuran-2-ylcarbonyl)-2,6-diazabicyclo[3.2.0]heptane,
2-(furan-3-ylcarbonyl)-2,6-diazabicyclo[3.2.0]heptane,
6-(furan-3-ylcarbonyl)-2,6-diazabicyclo[3.2.0]heptane,
2-(2-methylfuran-3-ylcarbonyl)-2,6-diazabicyclo[3.2.0]heptane,
6-(2-methylfuran-3-ylcarbonyl)-2,6-diazabicyclo[3.2.0]heptane,
2-(oxazol-2-ylcarbonyl)-2,6-diazabicyclo[3.2.0]heptane,
6-(oxazol-2-ylcarbonyl)-2,6-diazabicyclo[3.2.0]heptane,
2-(oxazol-5-ylcarbonyl)-2,6-diazabicyclo[3.2.0]heptane,
6-(oxazol-5-ylcarbonyl)-2,6-diazabicyclo[3.2.0]heptane,
2-(4-methyloxazol-5-ylcarbonyl)-2,6-diazabicyclo[3.2.0]heptane,
6-(4-methyloxazol-5-ylcarbonyl)-2,6-diazabicyclo[3.2.0]heptane,
2-(isoxazol-3-ylcarbonyl)-2,6-diazabicyclo[3.2.0]heptane,
6-(isoxazol-3-ylcarbonyl)-2,6-diazabicyclo[3.2.0]heptane,
2-(5-methylisoxazol-3-ylcarbonyl)-2,6-diazabicyclo[3.2.0]heptane,
6-(5-methylisoxazol-3-ylcarbonyl)-2,6-diazabicyclo[3.2.0]heptane,
2-(isoxazol-4-ylcarbonyl)-2,6-diazabicyclo[3.2.0]heptane,
6-(isoxazol-4-ylcarbonyl)-2,6-diazabicyclo[3.2.0]heptane,
2-(3-methylisoxazol-4-ylcarbonyl)-2,6-diazabicyclo[3.2.0]heptane,
6-(3-methylisoxazol-4-ylcarbonyl)-2,6-diazabicyclo[3.2.0]heptane,
2-(5-methylisoxazol-4-ylcarbonyl)-2,6-diazabicyclo[3.2.0]heptane,
6-(5-methylisoxazol-4-ylcarbonyl)-2,6-diazabicyclo[3.2.0]heptane,
2-(isoxazol-5-ylcarbonyl)-2,6-diazabicyclo[3.2.0]heptane,
6-(isoxazol-5-ylcarbonyl)-2,6-diazabicyclo[3.2.0]heptane,
2-(3-methylisoxazol-5-ylcarbonyl)-2,6-diazabicyclo[3.2.0]heptane,
6-(3-methylisoxazol-5-ylcarbonyl)-2,6-diazabicyclo[3.2.0]heptane,
2-(3-bromoisoxazol-5-ylcarbonyl)-2,6-diazabicyclo[3.2.0]heptane,
6-(3-bromoisoxazol-5-ylcarbonyl)-2,6-diazabicyclo[3.2.0]heptane,
2-(3-methoxyisoxazol-5-ylcarbonyl)-2,6-diazabicyclo[3.2.0]heptane,
6-(3-methoxyisoxazol-5-ylcarbonyl)-2,6-diazabicyclo[3.2.0]heptane,
2-(pyridin-4-ylcarbonyl)-2,6-diazabicyclo[3.2.0]heptane,
6-(pyridin-4-ylcarbonyl)-2,6-diazabicyclo[3.2.0]heptane,
3-(furan-2-ylcarbonyl)-3,6-diazabicyclo[3.2.0]heptane,
6-(furan-2-ylcarbonyl)-3,6-diazabicyclo[3.2.0]heptane,
3-(3-methylfuran-2-ylcarbonyl)-3,6-diazabicyclo[3.2.0]heptane,
6-(3-methylfuran-2-ylcarbonyl)-3,6-diazabicyclo[3.2.0]heptane,
3-(5-methylfuran-2-ylcarbonyl)-3,6-diazabicyclo[3.2.0]heptane,
6-(5-methylfuran-2-ylcarbonyl)-3,6-diazabicyclo[3.2.0]heptane,
3-(3-chlorofuran-2-ylcarbonyl)-3,6-diazabicyclo[3.2.0]heptane,
6-(3-chlorofuran-2-ylcarbonyl)-3,6-diazabicyclo[3.2.0]heptane,
3-(5-chlorofuran-2-ylcarbonyl)-3,6-diazabicyclo[3.2.0]heptane,
6-(5-chlorofuran-2-ylcarbonyl)-3,6-diazabicyclo[3.2.0]heptane,
3-(3-bromofuran-2-ylcarbonyl)-3,6-diazabicyclo[3.2.0]heptane,
6-(3-bromofuran-2-ylcarbonyl)-3,6-diazabicyclo[3.2.0]heptane,
3-(5-bromofuran-2-ylcarbonyl)-3,6-diazabicyclo[3.2.0]heptane,
6-(5-bromofuran-2-ylcarbonyl)-3,6-diazabicyclo[3.2.0]heptane,
3-(furan-3-ylcarbonyl)-3,6-diazabicyclo[3.2.0]heptane,
6-(furan-3-ylcarbonyl)-3,6-diazabicyclo[3.2.0]heptane,
3-(2-methylfuran-3-ylcarbonyl)-3,6-diazabicyclo[3.2.0]heptane,
6-(2-methylfuran-3-ylcarbonyl)-3,6-diazabicyclo[3.2.0]heptane,
3-(oxazol-2-ylcarbonyl)-3,6-diazabicyclo[3.2.0]heptane,
6-(oxazol-2-ylcarbonyl)-3,6-diazabicyclo[3.2.0]heptane,
3-(oxazol-5-ylcarbonyl)-3,6-diazabicyclo[3.2.0]heptane,
6-(oxazol-5-ylcarbonyl)-3,6-diazabicyclo[3.2.0]heptane,
3-(4-methyloxazol-5-ylcarbonyl)-3,6-diazabicyclo[3.2.0]heptane,
6-(4-methyloxazol-5-ylcarbonyl)-3,6-diazabicyclo[3.2.0]heptane,
3-(isoxazol-3-ylcarbonyl)-3,6-diazabicyclo[3.2.0]heptane,
6-(isoxazol-3-ylcarbonyl)-3,6-diazabicyclo[3.2.0]heptane,
3-(5-methylisoxazol-3-ylcarbonyl)-3,6-diazabicyclo[3.2.0]heptane,
6-(5-methylisoxazol-3-ylcarbonyl)-3,6-diazabicyclo[3.2.0]heptane,
3-(isoxazol-4-ylcarbonyl)-3,6-diazabicyclo[3.2.0]heptane,
6-(isoxazol-4-ylcarbonyl)-3,6-diazabicyclo[3.2.0]heptane,
3-(3-methylisoxazol-4-ylcarbonyl)-3,6-diazabicyclo[3.2.0]heptane,
6-(3-methylisoxazol-4-ylcarbonyl)-3,6-diazabicyclo[3.2.0]heptane,
3-(5-methylisoxazol-4-ylcarbonyl)-3,6-diazabicyclo[3.2.0]heptane,
6-(5-methylisoxazol-4-ylcarbonyl)-3,6-diazabicyclo[3.2.0]heptane,
3-(isoxazol-5-ylcarbonyl)-3,6-diazabicyclo[3.2.0]heptane,
6-(isoxazol-5-ylcarbonyl)-3,6-diazabicyclo[3.2.0]heptane,
3-(3-methylisoxazol-5-ylcarbonyl)-3,6-diazabicyclo[3.2.0]heptane,
6-(3-methylisoxazol-5-ylcarbonyl)-3,6-diazabicyclo[3.2.0]heptane,
3-(3-bromoisoxazol-5-ylcarbonyl)-3,6-diazabicyclo[3.2.0]heptane,
6-(3-bromoisoxazol-5-ylcarbonyl)-3,6-diazabicyclo[3.2.0]heptane,
3-(3-methoxyisoxazol-5-ylcarbonyl)-3,6-diazabicyclo[3.2.0]heptane,
6-(3-methoxyisoxazol-5-ylcarbonyl)-3,6-diazabicyclo[3.2.0]heptane,
3-(Pyridin-4-ylcarbonyl)-3,6-diazabicyclo[3.2.0]heptane,
6-(pyridin-4-ylcarbonyl)-3,6-diazabicyclo[3.2.0]heptane,
2-(furan-2-ylcarbonyl)-2,7-diazabicyclo[4.2.0]octane,
7-(furan-2-ylcarbonyl)-2,7-diazabicyclo[4.2.0]octane,
2-(3-methylfuran-2-ylcarbonyl)-2,7-diazabicyclo[4.2.0]octane,
7-(3-methylfuran-2-ylcarbonyl)-2,7-diazabicyclo[4.2.0]octane,
2-(5-methylfuran-2-ylcarbonyl)-2,7-diazabicyclo[4.2.0]octane,
7-(5-methylfuran-2-ylcarbonyl)-2,7-diazabicyclo[4.2.0]octane,
2-(3-chlorofuran-2-ylcarbonyl)-2,7-diazabicyclo[4.2.0]octane,
7-(3-chlorofuran-2-ylcarbonyl)-2,7-diazabicyclo[4.2.0]octane,
2-(5-chlorofuran-2-ylcarbonyl)-2,7-diazabicyclo[4.2.0]octane,
7-(5-chlorofuran-2-ylcarbonyl)-2,7-diazabicyclo[4.2.0]octane,
2-(3-bromofuran-2-ylcarbonyl)-2,7-diazabicyclo[4.2.0]octane,
7-(3-bromofuran-2-ylcarbonyl)-2,7-diazabicyclo[4.2.0]octane,
2-(5-bromofuran-2-ylcarbonyl)-2,7-diazabicyclo[4.2.0]octane,
7-(5-bromofuran-2-ylcarbonyl)-2,7-diazabicyclo[4.2.0]octane,
2-(furan-3-ylcarbonyl)-2,7-diazabicyclo[4.2.0]octane,
7-(furan-3-ylcarbonyl)-2,7-diazabicyclo[4.2.0]octane,
2-(2-methylfuran-3-ylcarbonyl)-2,7-diazabicyclo[4.2.0]octane,
7-(2-methylfuran-3-ylcarbonyl)-2,7-diazabicyclo[4.2.0]octane,
2-(oxazol-2-ylcarbonyl)-2,7-diazabicyclo[4.2.0]octane,
7-(oxazol-2-ylcarbonyl)-2,7-diazabicyclo[4.2.0]octane,
2-(oxazol-5-ylcarbonyl)-2,7-diazabicyclo[4.2.0]octane,
7-(oxazol-5-ylcarbonyl)-2,7-diazabicyclo[4.2.0]octane,
2-(4-methyloxazol-5-ylcarbonyl)-2,7-diazabicyclo[4.2.0]octane,
7-(4-methyloxazol-5-ylcarbonyl)-2,7-diazabicyclo[4.2.0]octane,
2-(isoxazol-3-ylcarbonyl)-2,7-diazabicyclo[4.2.0]octane,
7-(isoxazol-3-ylcarbonyl)-2,7-diazabicyclo[4.2.0]octane,
2-(5-methylisoxazol-3-ylcarbonyl)-2,7-diazabicyclo[4.2.0]octane,
7-(5-methylisoxazol-3-ylcarbonyl)-2,7-diazabicyclo[4.2.0]octane,
2-(isoxazol-4-ylcarbonyl)-2,7-diazabicyclo[4.2.0]octane,
7-(isoxazol-4-ylcarbonyl)-2,7-diazabicyclo[4.2.0]octane,
2-(3-methylisoxazol-4-ylcarbonyl)-2,7-diazabicyclo[4.2.0]octane,
7-(3-methylisoxazol-4-ylcarbonyl)-2,7-diazabicyclo[4.2.0]octane,
2-(5-methylisoxazol-4-ylcarbonyl)-2,7-diazabicyclo[4.2.0]octane,
7-(5-methylisoxazol-4-ylcarbonyl)-2,7-diazabicyclo[4.2.0]octane,
2-(isoxazol-5-ylcarbonyl)-2,7-diazabicyclo[4.2.0]octane,
7-(isoxazol-5-ylcarbonyl)-2,7-diazabicyclo[4.2.0]octane,
2-(3-methylisoxazol-5-ylcarbonyl)-2,7-diazabicyclo[4.2.0]octane,
7-(3-methylisoxazol-5-ylcarbonyl)-2,7-diazabicyclo[4.2.0]octane,
2-(3-bromoisoxazol-5-ylcarbonyl)-2,7-diazabicyclo[4.2.0]octane,
7-(3-bromoisoxazol-5-ylcarbonyl)-2,7-diazabicyclo[4.2.0]octane,
2-(3-methoxyisoxazol-5-ylcarbonyl)-2,7-diazabicyclo[4.2.0]octane,
7-(3-methoxyisoxazol-5-ylcarbonyl)-2,7-diazabicyclo[4.2.0]octane,
2-(pyridin-4-ylcarbonyl)-2,7-diazabicyclo[4.2.0]octane,
7-(Pyridin-4-ylcarbonyl)-2,7-diazabicyclo[4.2.0]octane,
3-(furan-2-ylcarbonyl)-3,7-diazabicyclo[4.2.0]octane,
7-(furan-2-ylcarbonyl)-3,7-diazabicyclo[4.2.0]octane,
3-(3-methylfuran-2-ylcarbonyl)-3,7-diazabicyclo[4.2.0]octane,
7-(3-methylfuran-2-ylcarbonyl)-3,7-diazabicyclo[4.2.0]octane 3-(5-methylfuran-2-ylcarbonyl)-3,7-diazabicyclo[4.2.0]octane,
7-(5-methylfuran-2-ylcarbonyl)-3,7-diazabicyclo[4.2.0]octane,
3-(3-chlorofuran-2-ylcarbonyl)-3,7-diazabicyclo[4.2.0]octane,
7-(3-chlorofuran-2-ylcarbonyl)-3,7-diazabicyclo[4.2.0]octane,
3-(5-chlorofuran-2-ylcarbonyl)-3,7-diazabicyclo[4.2.0]octane,
7-(5-chlorofuran-2-ylcarbonyl)-3,7-diazabicyclo[4.2.0]octane,
3-(3-bromofuran-2-ylcarbonyl)-3,7-diazabicyclo[4.2.0]octane,
7-(3-bromofuran-2-ylcarbonyl)-3,7-diazabicyclo[4.2.0]octane,
3-(5-bromofuran-2-ylcarbonyl)-3,7-diazabicyclo[4.2.0]octane,
7-(5-bromofuran-2-ylcarbonyl)-3,7-diazabicyclo[4.2.0]octane,
3-(furan-3-ylcarbonyl)-3,7-diazabicyclo[4.2.0]octane,
7-(furan-3-ylcarbonyl)-3,7-diazabicyclo[4.2.0]octane,
3-(2-methylfuran-3-ylcarbonyl)-3,7-diazabicyclo[4.2.0]octane,
7-(2-methylfuran-3-ylcarbonyl)-3,7-diazabicyclo[4.2.0]octane,
3-(oxazol-2-ylcarbonyl)-3,7-diazabicyclo[4.2.0]octane,
7-(oxazol-2-ylcarbonyl)-3,7-diazabicyclo[4.2.0]octane,
3-(oxazol-5-ylcarbonyl)-3,7-diazabicyclo[4.2.0]octane,
7-(oxazol-5-ylcarbonyl)-3,7-diazabicyclo[4.2.0]octane,
3-(4-methyloxazol-5-ylcarbonyl)-3,7-diazabicyclo[4.2.0]octane,
7-(4-methyloxazol-5-ylcarbonyl)-3,7-diazabicyclo[4.2.0]octane,
3-(isoxazol-3-ylcarbonyl)-3,7-diazabicyclo[4.2.0]octane,
7-(isoxazol-3-ylcarbonyl)-3,7-diazabicyclo[4.2.0]octane,
3-(5-methylisoxazol-3-ylcarbonyl)-3,7-diazabicyclo[4.2.0]octane,
7-(5-methylisoxazol-3-ylcarbonyl)-3,7-diazabicyclo[4.2.0]octane,
3-(isoxazol-4-ylcarbonyl)-3,7-diazabicyclo[4.2.0]octane,
7-(isoxazol-4-ylcarbonyl)-3,7-diazabicyclo[4.2.0]octane,
3-(3-methylisoxazol-4-ylcarbonyl)-3,7-diazabicyclo[4.2.0]octane,
7-(3-methylisoxazol-4-ylcarbonyl)-3,7-diazabicyclo[4.2.0]octane,
3-(5-methylisoxazol-4-ylcarbonyl)-3,7-diazabicyclo[4.2.0]octane,
7-(5-methylisoxazol-4-ylcarbonyl)-3,7-diazabicyclo[4.2.0]octane,
3-(isoxazol-5-ylcarbonyl)-3,7-diazabicyclo[4.2.0]octane,
7-(isoxazol-5-ylcarbonyl)-3,7-diazabicyclo[4.2.0]octane,
3-(3-methylisoxazol-5-ylcarbonyl)-3,7-diazabicyclo[4.2.0]octane,
7-(3-methylisoxazol-5-ylcarbonyl)-3,7-diazabicyclo[4.2.0]octane,
3-(3-bromoisoxazol-5-ylcarbonyl)-3,7-diazabicyclo[4.2.0]octane,
7-(3-bromoisoxazol-5-ylcarbonyl)-3,7-diazabicyclo[4.2.0]octane,
3-(3-methoxyisoxazol-5-ylcarbonyl)-3,7-diazabicyclo[4.2.0]octane,
7-(3-methoxyisoxazol-5-ylcarbonyl)-3,7-diazabicyclo[4.2.0]octane,
3-(pyridin-4-ylcarbonyl)-3,7-diazabicyclo[4.2.0]octane,
7-(pyridin-4-ylcarbonyl)-3,7-diazabicyclo[4.2.0]octane,
3-(furan-2-ylcarbonyl)-3,8-diazabicyclo[4.2.0]octane,
8-(furan-2-ylcarbonyl)-3,8-diazabicyclo[4.2.0]octane,
3-(3-methylfuran-2-ylcarbonyl)-3,8-diazabicyclo[4.2.0]octane,
8-(3-methylfuran-2-ylcarbonyl)-3,8-diazabicyclo[4.2.0]octane,
3-(5-methylfuran-2-ylcarbonyl)-3,8-diazabicyclo[4.2.0]octane,
8-(5-methylfuran-2-ylcarbonyl)-3,8-diazabicyclo[4.2.0]octane,
3-(3-chlorofuran-2-ylcarbonyl)-3,8-diazabicyclo[4.2.0]octane,
8-(3-chlorofuran-2-ylcarbonyl)-3,8-diazabicyclo[4.2.0]octane,
3-(5-chlorofuran-2-ylcarbonyl)-3,8-diazabicyclo[4.2.0]octane,
8-(5-chlorofuran-2-ylcarbonyl)-3,8-diazabicyclo[4.2.0]octane,
3-(3-bromofuran-2-ylcarbonyl)-3,8-diazabicyclo[4.2.0]octane,
8-(3-bromofuran-2-ylcarbonyl)-3,8-diazabicyclo[4.2.0]octane,
3-(5-bromofuran-2-ylcarbonyl)-3,8-diazabicyclo[4.2.0]octane,
8-(5-bromofuran-2-ylcarbonyl)-3,8-diazabicyclo[4.2.0]octane,
3-(furan-3-ylcarbonyl)-3,8-diazabicyclo[4.2.0]octane,
8-(furan-3-ylcarbonyl)-3,8-diazabicyclo[4.2.0]octane,
3-(2-methylfuran-3-ylcarbonyl)-3,8-diazabicyclo[4.2.0]octane,
8-(2-methylfuran-3-ylcarbonyl)-3,8-diazabicyclo[4.2.0]octane,
3-(oxazol-2-ylcarbonyl)-3,8-diazabicyclo[4.2.0]octane,
8-(oxazol-2-ylcarbonyl)-3,8-diazabicyclo[4.2.0]octane,
3-(oxazol-5-ylcarbonyl)-3,8-diazabicyclo[4.2.0]octane,
8-(oxazol-5-ylcarbonyl)-3,8-diazabicyclo[4.2.0]octane,
3-(4-methyloxazol-5-ylcarbonyl)-3,8-diazabicyclo[4.2.0]octane,
8-(4-methyloxazol-5-ylcarbonyl)-3,8-diazabicyclo[4.2.0]octane,
3-(isoxazol-3-ylcarbonyl)-3,8-diazabicyclo[4.2.0]octane,
8-(isoxazol-3-ylcarbonyl)-3,8-diazabicyclo[4.2.0]octane,
3-(5-methylisoxazol-3-ylcarbonyl)-3,8-diazabicyclo[4.2.0]octane,
8-(5-methylisoxazol-3-ylcarbonyl)-3,8-diazabicyclo[4.2.0]octane,
3-(isoxazol-4-ylcarbonyl)-3,8-diazabicyclo[4.2.0]octane,
8-(isoxazol-4-ylcarbonyl)-3,8-diazabicyclo[4.2.0]octane,
3-(3-methylisoxazol-4-ylcarbonyl)-3,8-diazabicyclo[4.2.0]octane,
8-(3-methylisoxazol-4-ylcarbonyl)-3,8-diazabicyclo[4.2.0]octane,
3-(5-methylisoxazol-4-ylcarbonyl)-3,8-diazabicyclo[4.2.0]octane,
8-(5-methylisoxazol-4-ylcarbonyl)-3,8-diazabicyclo[4.2.0]octane,
3-(isoxazol-5-ylcarbonyl)-3,8-diazabicyclo[4.2.0]octane,
8-(isoxazol-5-ylcarbonyl)-3,8-diazabicyclo[4.2.0]octane,
3-(3-methylisoxazol-5-ylcarbonyl)-3,8-diazabicyclo[4.2.0]octane,
8-(3-methylisoxazol-5-ylcarbonyl)-3,8-diazabicyclo[4.2.0]octane,
3-(3-bromoisoxazol-5-ylcarbonyl)-3,8-diazabicyclo[4.2.0]octane,
8-(3-bromoisoxazol-5-ylcarbonyl)-3,8-diazabicyclo[4.2.0]octane,
3-(3-methoxyisoxazol-5-ylcarbonyl)-3,8-diazabicyclo[4.2.0]octane,
8-(3-methoxyisoxazol-5-ylcarbonyl)-3,8-diazabicyclo[4.2.0]octane,
3-(pyridin-4-ylcarbonyl)-3,8-diazabicyclo[4.2.0]octane,
8-(pyridin-4-ylcarbonyl)-3,8-diazabicyclo[4.2.0]octane,
2-(furan-2-ylcarbonyl)-2,6-diazabicyclo[3.3.0]octane,
2-(3-methylfuran-2-ylcarbonyl)-2,6-diazabicyclo[3.3.0]octane,
2-(5-methylfuran-2-ylcarbonyl)-2,6-diazabicyclo[3.3.0]octane,
2-(3-chlorofuran-2-ylcarbonyl)-2,6-diazabicyclo[3.3.0]octane,
2-(5-chlorofuran-2-ylcarbonyl)-2,6-diazabicyclo[3.3.0]octane,
2-(3-bromofuran-2-ylcarbonyl)-2,6-diazabicyclo[3.3.0]octane,
2-(5-bromofuran-2-ylcarbonyl)-2,6-diazabicyclo[3.3.0]octane,
2-(furan-3-ylcarbonyl)-2,6-diazabicyclo[3.3.0]octane,
2-(2-methylfuran-3-ylcarbonyl)-2,6-diazabicyclo[3.3.0]octane,
2-(oxazol-2-ylcarbonyl)-2,6-diazabicyclo[3.3.0]octane,
2-(oxazol-5-ylcarbonyl)-2,6-diazabicyclo[3.3.0]octane,
2-(4-methyloxazol-5-ylcarbonyl)-2,6-diazabicyclo[3.3.0]octane,
2-(isoxazol-3-ylcarbonyl)-2,6-diazabicyclo[3.3.0]octane,
2-(5-methylisoxazol-3-ylcarbonyl)-2,6-diazabicyclo[3.3.0]octane,
2-(isoxazol-4-ylcarbonyl)-2,6-diazabicyclo[3.3.0]octane,
2-(3-methylisoxazol-4-ylcarbonyl)-2,6-diazabicyclo[3.3.0]octane,
2-(5-methylisoxazol-4-ylcarbonyl)-2,6-diazabicyclo[3.3.0]octane,
2-(isoxazol-5-ylcarbonyl)-2,6-diazabicyclo[3.3.0]octane,
2-(3-methylisoxazol-5-ylcarbonyl)-2,6-diazabicyclo[3.3.0]octane,
2-(3-bromoisoxazol-5-ylcarbonyl)-2,6-diazabicyclo[3.3.0]octane,
2-(3-methoxyisoxazol-5-ylcarbonyl)-2,6-diazabicyclo[3.3.0]octane,
2-(pyridin-4-ylcarbonyl)-2,6-diazabicyclo[3.3.0]octane,
2-(furan-2-ylcarbonyl)-2,7-diazabicyclo[3.3.0]octane,
7-(furan-2-ylcarbonyl)-2,7-diazabicyclo[3.3.0]octane,
2-(3-methylfuran-2-ylcarbonyl)-2,7-diazabicyclo[3.3.0]octane,
7-(3-methylfuran-2-ylcarbonyl)-2,7-diazabicyclo[3.3.0]octane,
2-(5-methylfuran-2-ylcarbonyl)-2,7-diazabicyclo[3.3.0]octane,
7-(5-methylfuran-2-ylcarbonyl)-2,7-diazabicyclo[3.3.0]octane,
2-(3-chlorofuran-2-ylcarbonyl)-2,7-diazabicyclo[3.3.0]octane,
7-(3-chlorofuran-2-ylcarbonyl)-2,7-diazabicyclo[3.3.0]octane,
2-(5-chlorofuran-2-ylcarbonyl)-2,7-diazabicyclo[3.3.0]octane,
7-(5-chlorofuran-2-ylcarbonyl)-2,7-diazabicyclo[3.3.0]octane,
2-(3-bromofuran-2-ylcarbonyl)-2,7-diazabicyclo[3.3.0]octane,
7-(3-bromofuran-2-ylcarbonyl)-2,7-diazabicyclo[3.3.0]octane,
2-(5-bromofuran-2-ylcarbonyl)-2,7-diazabicyclo[3.3.0]octane,
7-(5-bromofuran-2-ylcarbonyl)-2,7-diazabicyclo[3.3.0]octane,
2-(furan-3-ylcarbonyl)-2,7-diazabicyclo[3.3.0]octane,
7-(furan-3-ylcarbonyl)-2,7-diazabicyclo[3.3.0]octane,
2-(2-methylfuran-3-ylcarbonyl)-2,7-diazabicyclo[3.3.0]octane,
7-(2-methylfuran-3-ylcarbonyl)-2,7-diazabicyclo[3.3.0]octane,
2-(oxazol-2-ylcarbonyl)-2,7-diazabicyclo[3.0]octane,
7-(oxazol-2-ylcarbonyl)-2,7-diazabicyclo[3.3.0]octane,
2-(oxazol-5-ylcarbonyl)-2,7-diazabicyclo[3.3.0]octane,
7-(oxazol-5-ylcarbonyl)-2,7-diazabicyclo[3.3.0]octane,
2-(4-methyloxazol-5-ylcarbonyl)-2,7-diazabicyclo[3.3.0]octane,
7-(4-methyloxazol-5-ylcarbonyl)-2,7-diazabicyclo[3.3.0]octane,
2-(isoxazol-3-ylcarbonyl)-2,7-diazabicyclo[3.3.0]octane,
7-(isoxazol-3-ylcarbonyl)-2,7-diazabicyclo[3.3.0]octane,
2-(5-methylisoxazol-3-ylcarbonyl)-2,7-diazabicyclo[3.3.0]octane,
7-(5-methylisoxazol-3-ylcarbonyl)-2,7-diazabicyclo[3.3.0]octane,
2-(isoxazol-4-ylcarbonyl)-2,7-diazabicyclo[3.3.0]octane,
7-(isoxazol-4-ylcarbonyl)-2,7-diazabicyclo[3.3.0]octane,
2-(3-methylisoxazol-4-ylcarbonyl)-2,7-diazabicyclo[3.3.0]octane,
7-(3-methylisoxazol-4-ylcarbonyl)-2,7-diazabicyclo[3.3.0]octane,
2-(5-methylisoxazol-4-ylcarbonyl)-2,7-diazabicyclo[3.3.0]octane,
7-(5-methylisoxazol-4-ylcarbonyl)-2,7-diazabicyclo[3.3.0]octane,
2-(isoxazol-5-ylcarbonyl)-2,7-diazabicyclo[3.3.0]octane,
7-(isoxazol-5-ylcarbonyl)-2,7-diazabicyclo[3.3.0]octane,
2-(3-methylisoxazol-5-ylcarbonyl)-2,7-diazabicyclo[3.3.0]octane,
7-(3-methylisoxazol-5-ylcarbonyl)-2,7-diazabicyclo[3.3.0]octane,
2-(3-bromoisoxazol-5-ylcarbonyl)-2,7-diazabicyclo[3.3.0]octane,
7-(3-bromoisoxazol-5-ylcarbonyl)-2,7-diazabicyclo[3.3.0]octane,
2-(3-methoxyisoxazol-5-ylcarbonyl)-2,7-diazabicyclo[3.3.0]octane,
7-(3-methoxyisoxazol-5-ylcarbonyl)-2,7-diazabicyclo[3.3.0]octane,
2-(pyridin-4-ylcarbonyl)-2,7-diazabicyclo[3.3.0]octane,
7-(Pyridin-4-ylcarbonyl)-2,7-diazabicyclo[3.3.0]octane,
2-(furan-2-ylcarbonyl)-2,7-diazabicyclo[4.3.0]nonane,
7-(furan-2-ylcarbonyl)-2,7-diazabicyclo[4.3.0]nonane,
2-(3-methylfuran-2-ylcarbonyl)-2,7-diazabicyclo[4.3.0]nonane,
7-(3-methylfuran-2-ylcarbonyl)-2,7-diazabicyclo[4.3.0]nonane,
2-(5-methylfuran-2-ylcarbonyl)-2,7-diazabicyclo[4.3.0]nonane,
7-(5-methylfuran-2-ylcarbonyl)-2,7-diazabicyclo[4.3.0]nonane,
2-(3-chlorofuran-2-ylcarbonyl)-2,7-diazabicyclo[4.3.0]nonane,
7-(3-chlorofuran-2-ylcarbonyl)-2,7-diazabicyclo[4.3.0]nonane,
2-(5-chlorofuran-2-ylcarbonyl)-2,7-diazabicyclo[4.3.0]nonane,
7-(5-chlorofuran-2-ylcarbonyl)-2,7-diazabicyclo[4.3.0]nonane,
2-(3-bromofuran-2-ylcarbonyl)-2,7-diazabicyclo[4.3.0]nonane,
7-(3-bromofuran-2-ylcarbonyl)-2,7-diazabicyclo[4.3.0]nonane,
2-(5-bromofuran-2-ylcarbonyl)-2,7-diazabicyclo[4.3.0]nonane,
7-(5-bromofuran-2-ylcarbonyl)-2,7-diazabicyclo[4.3.0]nonane,
2-(furan-3-ylcarbonyl)-2,7-diazabicyclo[4.3.0]nonane,
7-(furan-3-ylcarbonyl)-2,7-diazabicyclo[4.3.0]nonane,
2-(2-methylfuran-3-ylcarbonyl)-2,7-diazabicyclo[4.3.0]nonane,
7-(2-methylfuran-3-ylcarbonyl)-2,7-diazabicyclo[4.3.0]nonane,
2-(oxazol-2-ylcarbonyl)-2,7-diazabicyclo[4.3.0]nonane,
7-(oxazol-2-ylcarbonyl)-2,7-diazabicyclo[4.3.0]nonane,
2-(oxazol-5-ylcarbonyl)-2,7-diazabicyclo[4.3.0]nonane,
7-(oxazol-5-ylcarbonyl)-2,7-diazabicyclo[4.3.0]nonane,
2-(4-methyloxazol-5-ylcarbonyl)-2,7-diazabicyclo[4.3.0]nonane,
7-(4-methyloxazol-5-ylcarbonyl)-2,7-diazabicyclo[4.3.0]nonane,
2-(isoxazol-3-ylcarbonyl)-2,7-diazabicyclo[4.3.0]nonane,
7-(isoxazol-3-ylcarbonyl)-2,7-diazabicyclo[4.3.0]nonane,
2-(5-methylisoxazol-3-ylcarbonyl)-2,7-diazabicyclo[4.3.0]nonane,
7-(5-methylisoxazol-3-ylcarbonyl)-2,7-diazabicyclo[4.3.0]nonane,
2-(isoxazol-4-ylcarbonyl)-2,7-diazabicyclo[4.3.0]nonane,
7-(isoxazol-4-ylcarbonyl)-2,7-diazabicyclo[4.3.0]nonane,
2-(3-methylisoxazol-4-ylcarbonyl)-2,7-diazabicyclo[4.3.0]nonane,
7-(3-methylisoxazol-4-ylcarbonyl)-2,7-diazabicyclo[4.3.0]nonane,
2-(5-methylisoxazol-4-ylcarbonyl)-2,7-diazabicyclo[4.3.0]nonane,
7-(5-methylisoxazol-4-ylcarbonyl)-2,7-diazabicyclo[4.3.0]nonane,
2-(isoxazol-5-ylcarbonyl)-2,7-diazabicyclo[4.3.0]nonane,
7-(isoxazol-5-ylcarbonyl)-2,7-diazabicyclo[4.3.0]nonane,
2-(3-methylisoxazol-5-ylcarbonyl)-2,7-diazabicyclo[4.3.0]nonane,
7-(3-methylisoxazol-5-ylcarbonyl)-2,7-diazabicyclo[4.3.0]nonane,
2-(3-bromoisoxazol-5-ylcarbonyl)-2,7-diazabicyclo[4.3.0]nonane,
7-(3-bromoisoxazol-5-ylcarbonyl)-2,7-diazabicyclo[4.3.0]nonane,
2-(3-methoxyisoxazol-5-ylcarbonyl)-2,7-diazabicyclo[4.3.0]nonane,
7-(3-methoxyisoxazol-5-ylcarbonyl)-2,7-diazabicyclo[4.3.0]nonane,
2-(pyridin-4-ylcarbonyl)-2,7-diazabicyclo[4.3.0]nonane,
7-(Pyridin-4-ylcarbonyl)-2,7-diazabicyclo[4.3.0]nonane,
2-(furan-2-ylcarbonyl)-2,8-diazabicyclo[4.3.0]nonane,
8-(furan-2-ylcarbonyl)-2,8-diazabicyclo[4.3.0]nonane,
2-(3-methylfuran-2-ylcarbonyl)-2,8-diazabicyclo[4.3.0]nonane,
8-(3-methylfuran-2-ylcarbonyl)-2,8-diazabicyclo[4.3.0]nonane,
2-(5-methylfuran-2-ylcarbonyl)-2,8-diazabicyclo[4.3.0]nonane,
8-(5-methylfuran-2-ylcarbonyl)-2,8-diazabicyclo[4.3.0]nonane,
2-(3-chlorofuran-2-ylcarbonyl)-2,8-diazabicyclo[4.3.0]nonane,
8-(3-chlorofuran-2-ylcarbonyl)-2,8-diazabicyclo[4.3.0]nonane,
2-(5-chlorofuran-2-ylcarbonyl)-2,8-diazabicyclo[4.3.0]nonane,
8-(5-chlorofuran-2-ylcarbonyl)-2,8-diazabicyclo[4.3.0]nonane,
2-(3-bromofuran-2-ylcarbonyl)-2,8-diazabicyclo[4.3.0]nonane,
8-(3-bromofuran-2-ylcarbonyl)-2,8-diazabicyclo[4.3.0]nonane,
2-(5-bromofuran-2-ylcarbonyl)-2,8-diazabicyclo[4.3.0]nonane,
8-(5-bromofuran-2-ylcarbonyl)-2,8-diazabicyclo[4.3.0]nonane,
2-(furan-3-ylcarbonyl)-2,8-diazabicyclo[4.3.0]nonane,
8-(furan-3-ylcarbonyl)-2,8-diazabicyclo[4.3.0]nonane,
2-(2-methylfuran-3-ylcarbonyl)-2,8-diazabicyclo[4.3.0]nonane,
8-(2-methylfuran-3-ylcarbonyl)-2,8-diazabicyclo[4.3.0]nonane,
2-(oxazol-2-ylcarbonyl)-2,8-diazabicyclo[4.3.0]nonane,
8-(oxazol-2-ylcarbonyl)-2,8-diazabicyclo[4.3.0]nonane,
2-(oxazol-5-ylcarbonyl)-2,8-diazabicyclo[4.3.0]nonane,
8-(oxazol-5-ylcarbonyl)-2,8-diazabicyclo[4.3.0]nonane,
2-(4-methyloxazol-5-ylcarbonyl)-2,8-diazabicyclo[4.3.0]nonane,
8-(4-methyloxazol-5-ylcarbonyl)-2,8-diazabicyclo[4.3.0]nonane,
2-(isoxazol-3-ylcarbonyl)-2,8-diazabicyclo[4.3.0]nonane,
8-(isoxazol-3-ylcarbonyl)-2,8-diazabicyclo[4.3.0]nonane,
2-(5-methylisoxazol-3-ylcarbonyl)-2,8-diazabicyclo[4.3.0]nonane,
8-(5-methylisoxazol-3-ylcarbonyl)-2,8-diazabicyclo[4.3.0]nonane,
2-(isoxazol-4-ylcarbonyl)-2,8-diazabicyclo[4.3.0]nonane,
8-(isoxazol-4-ylcarbonyl)-2,8-diazabicyclo[4.3.0]nonane,
2-(3-methylisoxazol-4-ylcarbonyl)-2,8-diazabicyclo[4.3.0]nonane,
8-(3-methylisoxazol-4-ylcarbonyl)-2,8-diazabicyclo[4.3.0]nonane,
2-(5-methylisoxazol-4-ylcarbonyl)-2,8-diazabicyclo[4.3.0]nonane,
8-(5-methylisoxazol-4-ylcarbonyl)-2,8-diazabicyclo[4.3.0]nonane,
2-(isoxazol-5-ylcarbonyl)-2,8-diazabicyclo[4.3.0]nonane,
8-(isoxazol-5-ylcarbonyl)-2,8-diazabicyclo[4.3.0]nonane,
2-(3-methylisoxazol-5-ylcarbonyl)-2,8-diazabicyclo[4.3.0]nonane,
8-(3-methylisoxazol-5-ylcarbonyl)-2,8-diazabicyclo[4.3.0]nonane,
2-(3-bromoisoxazol-5-ylcarbonyl)-2,8-diazabicyclo[4.3.0]nonane,
8-(3-bromoisoxazol-5-ylcarbonyl)-2,8-diazabicyclo[4.3.0]nonane,
2-(3-methoxyisoxazol-5-ylcarbonyl)-2,8-diazabicyclo[4.3.0]nonane,
8-(3-methoxyisoxazol-5-ylcarbonyl)-2,8-diazabicyclo[4.3.0]nonane,
2-(pyridin-4-ylcarbonyl)-2,8-diazabicyclo[4.3.0]nonane,
8-(pyridin-4-ylcarbonyl)-2,8-diazabicyclo[4.3.0]nonane,
3-(furan-2-ylcarbonyl)-3,7-diazabicyclo[4.3.0]nonane,
7-(furan-2-ylcarbonyl)-3,7-diazabicyclo[4.3.0]nonane,
3-(3-methylfuran-2-ylcarbonyl)-3,7-diazabicyclo[4.3.0]nonane,
7-(3-methylfuran-2-ylcarbonyl)-3,7-diazabicyclo[4.3.0]nonane,
3-(5-methylfuran-2-ylcarbonyl)-3,7-diazabicyclo[4.3.0]nonane,
7-(5-methylfuran-2-ylcarbonyl)-3,7-diazabicyclo[4.3.0]nonane,
3-(3-chlorofuran-2-ylcarbonyl)-3,7-diazabicyclo[4.3.0]nonane,
7-(3-chlorofuran-2-ylcarbonyl)-3,7-diazabicyclo[4.3.0]nonane,
3-(5-chlorofuran-2-ylcarbonyl)-3,7-diazabicyclo[4.3.0]nonane,
7-(5-chlorofuran-2-ylcarbonyl)-3,7-diazabicyclo[4.3.0]nonane,
3-(3-bromofuran-2-ylcarbonyl)-3,7-diazabicyclo[4.3.0]nonane,
7-(3-bromofuran-2-ylcarbonyl)-3,7-diazabicyclo[4.3.0]nonane,
3-(5-bromofuran-2-ylcarbonyl)-3,7-diazabicyclo[4.3.0]nonane,
7-(5-bromofuran-2-ylcarbonyl)-3,7-diazabicyclo[4.3.0]nonane,
3-(furan-3-ylcarbonyl)-3,7-diazabicyclo[4.3.0]nonane,
7-(furan-3-ylcarbonyl)-3,7-diazabicyclo[4.3.0]nonane,
3-(2-methylfuran-3-ylcarbonyl)-3,7-diazabicyclo[4.3.0]nonane,
7-(2-methylfuran-3-ylcarbonyl)-3,7-diazabicyclo[4.3.0]nonane,
3-(oxazol-2-ylcarbonyl)-3,7-diazabicyclo[4.3.0]nonane,
7-(oxazol-2-ylcarbonyl)-3,7-diazabicyclo[4.3.0]nonane,
3-(oxazol-5-ylcarbonyl)-3,7-diazabicyclo[4.3.0]nonane,
7-(oxazol-5-ylcarbonyl)-3,7-diazabicyclo[4.3.0]nonane,
3-(4-methyloxazol-5-ylcarbonyl)-3,7-diazabicyclo[4.3.0]nonane,
7-(4-methyloxazol-5-ylcarbonyl)-3,7-diazabicyclo[4.3.0]nonane,
3-(isoxazol-3-ylcarbonyl)-3,7-diazabicyclo[4.3.0]nonane,
7-(isoxazol-3-ylcarbonyl)-3,7-diazabicyclo[4.3.0]nonane,
3-(5-methylisoxazol-3-ylcarbonyl)-3,7-diazabicyclo[4.3.0]nonane,
7-(5-methylisoxazol-3-ylcarbonyl)-3,7-diazabicyclo[4.3.0]nonane,
3-(isoxazol-4-ylcarbonyl)-3,7-diazabicyclo[4.3.0]nonane,
7-(isoxazol-4-ylcarbonyl)-3,7-diazabicyclo[4.3.0]nonane,
3-(3-methylisoxazol-4-ylcarbonyl)-3,7-diazabicyclo[4.3.0]nonane,
7-(3-methylisoxazol-4-ylcarbonyl)-3,7-diazabicyclo[4.3.0]nonane,
3-(5-methylisoxazol-4-ylcarbonyl)-3,7-diazabicyclo[4.3.0]nonane,
7-(5-methylisoxazol-4-ylcarbonyl)-3,7-diazabicyclo[4.3.0]nonane,
3-(isoxazol-5-ylcarbonyl)-3,7-diazabicyclo[4.3.0]nonane,
7-(isoxazol-5-ylcarbonyl)-3,7-diazabicyclo[4.3.0]nonane,
3-(3-methylisoxazol-5-ylcarbonyl)-3,7-diazabicyclo[4.3.0]nonane,
7-(3-methylisoxazol-5-ylcarbonyl)-3,7-diazabicyclo[4.3.0]nonane,
3-(3-bromoisoxazol-5-ylcarbonyl)-3,7-diazabicyclo[4.3.0]nonane,
7-(3-bromoisoxazol-5-ylcarbonyl)-3,7-diazabicyclo[4.3.0]nonane,
3-(3-methoxyisoxazol-5-ylcarbonyl)-3,7-diazabicyclo[4.3.0]nonane,
7-(3-methoxyisoxazol-5-ylcarbonyl)-3,7-diazabicyclo[4.3.0]nonane,
3-(pyridin-4-ylcarbonyl)-3,7-diazabicyclo[4.3.0]nonane,
7-(Pyridin-4-ylcarbonyl)-3,7-diazabicyclo[4.3.0]nonane,
3-(furan-2-ylcarbonyl)-3,8-diazabicyclo[4.3.0]nonane,
8-(furan-2-ylcarbonyl)-3,8-diazabicyclo[4.3.0]nonane,
3-(3-methylfuran-2-ylcarbonyl)-3,8-diazabicyclo[4.3.0]nonane,
8-(3-methylfuran-2-ylcarbonyl)-3,8-diazabicyclo[4.3.0]nonane,
3-(5-methylfuran-2-ylcarbonyl)-3,8-diazabicyclo[4.3.0]nonane,
8-(5-methylfuran-2-ylcarbonyl)-3,8-diazabicyclo[4.3.0]nonane,
3-(3-chlorofuran-2-ylcarbonyl)-3,8-diazabicyclo[4.3.0]nonane,
8-(3-chlorofuran-2-ylcarbonyl)-3,8-diazabicyclo[4.3.0]nonane,
3-(5-chlorofuran-2-ylcarbonyl)-3,8-diazabicyclo[4.3.0]nonane,
8-(5-chlorofuran-2-ylcarbonyl)-3,8-diazabicyclo[4.3.0]nonane,
3-(3-bromofuran-2-ylcarbonyl)-3,8-diazabicyclo[4.3.0]nonane,
8-(3-bromofuran-2-ylcarbonyl)-3,8-diazabicyclo[4.3.0]nonane,
3-(5-bromofuran-2-ylcarbonyl)-3,8-diazabicyclo[4.3.0]nonane,
8-(5-bromofuran-2-ylcarbonyl)-3,8-diazabicyclo[4.3.0]nonane,
3-(furan-3-ylcarbonyl)-3,8-diazabicyclo[4.3.0]nonane,
8-(furan-3-ylcarbonyl)-3,8-diazabicyclo[4.3.0]nonane,
3-(2-methylfuran-3-ylcarbonyl)-3,8-diazabicyclo[4.3.0]nonane,
8-(2-methylfuran-3-ylcarbonyl)-3,8-diazabicyclo[4.3.0]nonane,
3-(oxazol-2-ylcarbonyl)-3,8-diazabicyclo[4.3.0]nonane,
8-(oxazol-2-ylcarbonyl)-3,8-diazabicyclo[4.3.0]nonane,
3-(oxazol-5-ylcarbonyl)-3,8-diazabicyclo[4.3.0]nonane,
8-(oxazol-5-ylcarbonyl)-3,8-diazabicyclo[4.3.0]nonane,
3-(4-methyloxazol-5-ylcarbonyl)-3,8-diazabicyclo[4.3.0]nonane,
8-(4-methyloxazol-5-ylcarbonyl)-3,8-diazabicyclo[4.3.0]nonane,
3-(isoxazol-3-ylcarbonyl)-3,8-diazabicyclo[4.3.0]nonane,
8-(isoxazol-3-ylcarbonyl)-3,8-diazabicyclo[4.3.0]nonane,
3-(5-methylisoxazol-3-ylcarbonyl)-3,8-diazabicyclo[4.3.0]nonane,
8-(5-methylisoxazol-3-ylcarbonyl)-3,8-diazabicyclo[4.3.0]nonane,
3-(isoxazol-4-ylcarbonyl)-3,8-diazabicyclo[4.3.0]nonane,
8-(isoxazol-4-ylcarbonyl)-3,8-diazabicyclo[4.3.0]nonane,
3-(3-methylisoxazol-4-ylcarbonyl)-3,8-diazabicyclo[4.3.0]nonane,
8-(3-methylisoxazol-4-ylcarbonyl)-3,8-diazabicyclo[4.3.0]nonane,
3-(5-methylisoxazol-4-ylcarbonyl)-3,8-diazabicyclo[4.3.0]nonane,
8-(5-methylisoxazol-4-ylcarbonyl)-3,8-diazabicyclo[4.3.0]nonane,
3-(isoxazol-5-ylcarbonyl)-3,8-diazabicyclo[4.3.0]nonane,
8-(isoxazol-5-ylcarbonyl)-3,8-diazabicyclo[4.3.0]nonane,
3-(3-methylisoxazol-5-ylcarbonyl)-3,8-diazabicyclo[4.3.0]nonane,
8-(3-methylisoxazol-5-ylcarbonyl)-3,8-diazabicyclo[4.3.0]nonane,
3-(3-bromoisoxazol-5-ylcarbonyl)-3,8-diazabicyclo[4.3.0]nonane,
8-(3-bromoisoxazol-5-ylcarbonyl)-3,8-diazabicyclo[4.3.0]nonane,
3-(3-methoxyisoxazol-5-ylcarbonyl)-3,8-diazabicyclo[4.3.0]nonane,
8-(3-methoxyisoxazol-5-ylcarbonyl)-3,8-diazabicyclo[4.3.0]nonane,
3-(pyridin-4-ylcarbonyl)-3,8-diazabicyclo[4.3.0]nonane,
8-(pyridin-4-ylcarbonyl)-3,8-diazabicyclo[4.3.0]nonane,
3-(furan-2-ylcarbonyl)-3,9-diazabicyclo[4.3.0]nonane,
9-(furan-2-ylcarbonyl)-3,9-diazabicyclo[4.3.0]nonane,
3-(3-methylfuran-2-ylcarbonyl)-3,9-diazabicyclo[4.3.0]nonane,
9-(3-methylfuran-2-ylcarbonyl)-3,9-diazabicyclo[4.3.0]nonane,
3-(5-methylfuran-2-ylcarbonyl)-3,9-diazabicyclo[4.3.0]nonane,
9-(5-methylfuran-2-ylcarbonyl)-3,9-diazabicyclo[4.3.0]nonane,
3-(3-chlorofuran-2-ylcarbonyl)-3,9-diazabicyclo[4.3.0]nonane,
9-(3-chlorofuran-2-ylcarbonyl)-3,9-diazabicyclo[4.3.0]nonane,
3-(5-chlorofuran-2-ylcarbonyl)-3,9-diazabicyclo[4.3.0]nonane,
9-(5-chlorofuran-2-ylcarbonyl)-3,9-diazabicyclo[4.3.0]nonane,
3-(3-bromofuran-2-ylcarbonyl)-3,9-diazabicyclo[4.3.0]nonane,
9-(3-bromofuran-2-ylcarbonyl)-3,9-diazabicyclo[4.3.0]nonane,
3-(5-bromofuran-2-ylcarbonyl)-3,9-diazabicyclo[4.3.0]nonane,
9-(5-bromofuran-2-ylcarbonyl)-3,9-diazabicyclo[4.3.0]nonane,
3-(furan-3-ylcarbonyl)-3,9-diazabicyclo[4.3.0]nonane,
9-(furan-3-ylcarbonyl)-3,9-diazabicyclo[4.3.0]nonane,
3-(2-methylfuran-3-ylcarbonyl)-3,9-diazabicyclo[4.3.0]nonane,
9-(2-methylfuran-3-ylcarbonyl)-3,9-diazabicyclo[4.3.0]nonane,
3-(oxazol-2-ylcarbonyl)-3,9-diazabicyclo[4.3.0]nonane,
9-(oxazol-2-ylcarbonyl)-3,9-diazabicyclo[4.3.0]nonane,
3-(oxazol-5-ylcarbonyl)-3,9-diazabicyclo[4.3.0]nonane,
9-(oxazol-5-ylcarbonyl)-3,9-diazabicyclo[4.3.0]nonane,
3-(4-methyloxazol-5-ylcarbonyl)-3,9-diazabicyclo[4.3.0]nonane,
9-(4-methyloxazol-5-ylcarbonyl)-3,9-diazabicyclo[4.3.0]nonane,
3-(isoxazol-3-ylcarbonyl)-3,9-diazabicyclo[4.3.0]nonane,
9-(isoxazol-3-ylcarbonyl)-3,9-diazabicyclo[4.3.0]nonane,
3-(5-methylisoxazol-3-ylcarbonyl)-3,9-diazabicyclo[4.3.0]nonane,
9-(5-methylisoxazol-3-ylcarbonyl)-3,9-diazabicyclo[4.3.0]nonane,
3-(isoxazol-4-ylcarbonyl)-3,9-diazabicyclo[4.3.0]nonane,
9-(isoxazol-4-ylcarbonyl)-3,9-diazabicyclo[4.3.0]nonane,
3-(3-methylisoxazol-4-ylcarbonyl)-3,9-diazabicyclo[4.3.0]nonane,
9-(3-methylisoxazol-4-ylcarbonyl)-3,9-diazabicyclo[4.3.0]nonane,
3-(5-methylisoxazol-4-ylcarbonyl)-3,9-diazabicyclo[4.3.0]nonane,
9-(5-methylisoxazol-4-ylcarbonyl)-3,9-diazabicyclo[4.3.0]nonane,
3-(isoxazol-5-ylcarbonyl)-3,9-diazabicyclo[4.3.0]nonane,
9-(isoxazol-5-ylcarbonyl)-3,9-diazabicyclo[4.3.0]nonane,
3-(3-methylisoxazol-5-ylcarbonyl)-3,9-diazabicyclo[4.3.0]nonane,
9-(3-methylisoxazol-5-ylcarbonyl)-3,9-diazabicyclo[4.3.0]nonane,
3-(3-bromoisoxazol-5-ylcarbonyl)-3,9-diazabicyclo[4.3.0]nonane,
9-(3-bromoisoxazol-5-ylcarbonyl)-3,9-diazabicyclo[4.3.0]nonane,
3-(3-methoxyisoxazol-5-ylcarbonyl)-3,9-diazabicyclo[4.3.0]nonane,
9-(3-methoxyisoxazol-5-ylcarbonyl)-3,9-diazabicyclo[4.3.0]nonane,
3-(pyridin-4-ylcarbonyl)-3,9-diazabicyclo[4.3.0]nonane,
9-(Pyridin-4-ylcarbonyl)-3,9-diazabicyclo[4.3.0]nonane,
2-(furan-2-ylcarbonyl)-2,6-diazabicyclo[3.2.1]octane,
6-(furan-2-ylcarbonyl)-2,6-diazabicyclo[3.2.1]octane,
2-(3-methylfuran-2-ylcarbonyl)-2,6-diazabicyclo[3.2.1]octane,
6-(3-methylfuran-2-ylcarbonyl)-2,6-diazabicyclo[3.2.1]octane,
2-(5-methylfuran-2-ylcarbonyl)-2,6-diazabicyclo[3.2.1]octane,
6-(5-methylfuran-2-ylcarbonyl)-2,6-diazabicyclo[3.2.1]octane,
2-(3-chlorofuran-2-ylcarbonyl)-2,6-diazabicyclo[3.2.1]octane,
6-(3-chlorofuran-2-ylcarbonyl)-2,6-diazabicyclo[3.2.1]octane,
2-(5-chlorofuran-2-ylcarbonyl)-2,6-diazabicyclo[3.2.1]octane,
6-(5-chlorofuran-2-ylcarbonyl)-2,6-diazabicyclo[3.2.1]octane,
2-(3-bromofuran-2-ylcarbonyl)-2,6-diazabicyclo[3.2.1]octane,
6-(3-bromofuran-2-ylcarbonyl)-2,6-diazabicyclo[3.2.1]octane,
2-(5-bromofuran-2-ylcarbonyl)-2,6-diazabicyclo[3.2.1]octane,
6-(5-bromofuran-2-ylcarbonyl)-2,6-diazabicyclo[3.2.1]octane,
2-(furan-3-ylcarbonyl)-2,6-diazabicyclo[3.2.1]octane,
6-(furan-3-ylcarbonyl)-2,6-diazabicyclo[3.2.1]octane,
2-(2-methylfuran-3-ylcarbonyl)-2,6-diazabicyclo[3.2.1]octane,
6-(2-methylfuran-3-ylcarbonyl)-2,6-diazabicyclo[3.2.1]octane,
2-(oxazol-2-ylcarbonyl)-2,6-diazabicyclo[3.2.1]octane,
6-(oxazol-2-ylcarbonyl)-2,6-diazabicyclo[3.2.1]octane,
2-(oxazol-5-ylcarbonyl)-2,6-diazabicyclo[3.2.1]octane,
6-(oxazol-5-ylcarbonyl)-2,6-diazabicyclo[3.2.1]octane,
2-(4-methyloxazol-5-ylcarbonyl)-2,6-diazabicyclo[3.2.1]octane,
6-(4-methyloxazol-5-ylcarbonyl)-2,6-diazabicyclo[3.2.1]octane,
2-(isoxazol-3-ylcarbonyl)-2,6-diazabicyclo[3.2.1]octane,
6-(isoxazol-3-ylcarbonyl)-2,6-diazabicyclo[3.2.1]octane,
2-(5-methylisoxazol-3-ylcarbonyl)-2,6-diazabicyclo[3.2.1]octane,
6-(5-methylisoxazol-3-ylcarbonyl)-2,6-diazabicyclo[3.2.1]octane,
2-(isoxazol-4-ylcarbonyl)-2,6-diazabicyclo[3.2.1]octane,
6-(isoxazol-4-ylcarbonyl)-2,6-diazabicyclo[3.2.1]octane,
2-(3-methylisoxazol-4-ylcarbonyl)-2,6-diazabicyclo[3.2.1]octane,
6-(3-methylisoxazol-4-ylcarbonyl)-2,6-diazabicyclo[3.2.1]octane,
2-(5-methylisoxazol-4-ylcarbonyl)-2,6-diazabicyclo[3.2.1]octane,
6-(5-methylisoxazol-4-ylcarbonyl)-2,6-diazabicyclo[3.2.1]octane,
2-(isoxazol-5-ylcarbonyl)-2,6-diazabicyclo[3.2.1]octane,
6-(isoxazol-5-ylcarbonyl)-2,6-diazabicyclo[3.2.1]octane,
2-(3-methylisoxazol-5-ylcarbonyl)-2,6-diazabicyclo[3.2.1]octane,
6-(3-methylisoxazol-5-ylcarbonyl)-2,6-diazabicyclo[3.2.1]octane,
2-(3-bromoisoxazol-5-ylcarbonyl)-2,6-diazabicyclo[3.2.1]octane,
6-(3-bromoisoxazol-5-ylcarbonyl)-2,6-diazabicyclo[3.2.1]octane,
2-(3-methoxyisoxazol-5-ylcarbonyl)-2,6-diazabicyclo[3.2.1]octane,
6-(3-methoxyisoxazol-5-ylcarbonyl)-2,6-diazabicyclo[3.2.1]octane,
2-(pyridin-4-ylcarbonyl)-2,6-diazabicyclo[3.2.1]octane,
6-(pyridin-4-ylcarbonyl)-2,6-diazabicyclo[3.2.1]octane,
3-(furan-2-ylcarbonyl)-3,6-diazabicyclo[3.2.1]octane,
6-(furan-2-ylcarbonyl)-3,6-diazabicyclo[3.2.1]octane,
3-(3-methylfuran-2-ylcarbonyl)-3,6-diazabicyclo[3.2.1]octane,
6-(3-methylfuran-2-ylcarbonyl)-3,6-diazabicyclo[3.2.1]octane,
3-(5-methylfuran-2-ylcarbonyl)-3,6-diazabicyclo[3.2.1]octane,
6-(5-methylfuran-2-ylcarbonyl)-3,6-diazabicyclo[3.2.1]octane,
3-(3-chlorofuran-2-ylcarbonyl)-3,6-diazabicyclo[3.2.1]octane,
6-(3-chlorofuran-2-ylcarbonyl)-3,6-diazabicyclo[3.2.1]octane,
3-(5-chlorofuran-2-ylcarbonyl)-3,6-diazabicyclo[3.2.1]octane,
6-(5-chlorofuran-2-ylcarbonyl)-3,6-diazabicyclo[3.2.1]octane,
3-(3-bromofuran-2-ylcarbonyl)-3,6-diazabicyclo[3.2.1]octane,
6-(3-bromofuran-2-ylcarbonyl)-3,6-diazabicyclo[3.2.1]octane,
3-(5-bromofuran-2-ylcarbonyl)-3,6-diazabicyclo[3.2.1]octane,
6-(5-bromofuran-2-ylcarbonyl)-3,6-diazabicyclo[3.2.1]octane,
3-(furan-3-ylcarbonyl)-3,6-diazabicyclo[3.2.1]octane,
6-(furan-3-ylcarbonyl)-3,6-diazabicyclo[3.2.1]octane,
3-(2-methylfuran-3-ylcarbonyl)-3,6-diazabicyclo[3.2.1]octane,
6-(2-methylfuran-3-ylcarbonyl)-3,6-diazabicyclo[3.2.1]octane,
3-(oxazol-2-ylcarbonyl)-3,6-diazabicyclo[3.2.1]octane,
6-(oxazol-2-ylcarbonyl)-3,6-diazabicyclo[3.2.1]octane,
3-(oxazol-5-ylcarbonyl)-3,6-diazabicyclo[3.2.1]octane,
6-(oxazol-5-ylcarbonyl)-3,6-diazabicyclo[3.2.1]octane,
3-(4-methyloxazol-5-ylcarbonyl)-3,6-diazabicyclo[3.2.1]octane,
6-(4-methyloxazol-5-ylcarbonyl)-3,6-diazabicyclo[3.2.1]octane,
3-(isoxazol-3-ylcarbonyl)-3,6-diazabicyclo[3.2.1]octane,
6-(isoxazol-3-ylcarbonyl)-3,6-diazabicyclo[3.2.1]octane,
3-(5-methylisoxazol-3-ylcarbonyl)-3,6-diazabicyclo[3.2.1]octane,
6-(5-methylisoxazol-3-ylcarbonyl)-3,6-diazabicyclo[3.2.1]octane,
3-(isoxazol-4-ylcarbonyl)-3,6-diazabicyclo[3.2.1]octane,
6-(isoxazol-4-ylcarbonyl)-3,6-diazabicyclo[3.2.1]octane,
3-(3-methylisoxazol-4-ylcarbonyl)-3,6-diazabicyclo[3.2.1]octane,
6-(3-methylisoxazol-4-ylcarbonyl)-3,6-diazabicyclo[3.2.1]octane,
3-(5-methylisoxazol-4-ylcarbonyl)-3,6-diazabicyclo[3.2.1]octane,
6-(5-methylisoxazol-4-ylcarbonyl)-3,6-diazabicyclo[3.2.1]octane,
3-(isoxazol-5-ylcarbonyl)-3,6-diazabicyclo[3.2.1]octane,
6-(isoxazol-5-ylcarbonyl)-3,6-diazabicyclo[3.2.1]octane,
3-(3-methylisoxazol-5-ylcarbonyl)-3,6-diazabicyclo[3.2.1]octane,
6-(3-methylisoxazol-5-ylcarbonyl)-3,6-diazabicyclo[3.2.1]octane,
3-(3-bromoisoxazol-5-ylcarbonyl)-3,6-diazabicyclo[3.2.1]octane,
6-(3-bromoisoxazol-5-ylcarbonyl)-3,6-diazabicyclo[3.2.1]octane,
3-(3-methoxyisoxazol-5-ylcarbonyl)-3,6-diazabicyclo[3.2.1]octane,
6-(3-methoxyisoxazol-5-ylcarbonyl)-3,6-diazabicyclo[3.2.1]octane,
3-(pyridin-4-ylcarbonyl)-3,6-diazabicyclo[3.2.1]octane, and 6-(pyridin-4-ylcarbonyl)-3,6-diazabicyclo[3.2.1]octane,
or a pharmaceutically acceptable salt thereof.

15. A method for treating central nervous system disorders, comprising administering a compound selected from the group consisting of:
2-(furan-2-ylcarbonyl)-2,6-diazabicyclo[3.2.0]heptane,
6-(furan-2-ylcarbonyl)-2,6-diazabicyclo[3.2.0]heptane,
2-(3-methylfuran-2-ylcarbonyl)-2,6-diazabicyclo[3.2.0]heptane,
6-(3-methylfuran-2-ylcarbonyl)-2,6-diazabicyclo[3.2.0]heptane,
2-(5-methylfuran-2-ylcarbonyl)-2,6-diazabicyclo[3.2.0]heptane,
6-(5-methylfuran-2-ylcarbonyl)-2,6-diazabicyclo[3.2.0]heptane,
2-(3-chlorofuran-2-ylcarbonyl)-2,6-diazabicyclo[3.2.0]heptane,
6-(3-chlorofuran-2-ylcarbonyl)-2,6-diazabicyclo[3.2.0]heptane,
2-(5-chlorofuran-2-ylcarbonyl)-2,6-diazabicyclo[3.2.0]heptane,
6-(5-chlorofuran-2-ylcarbonyl)-2,6-diazabicyclo[3.2.0]heptane,
2-(3-bromofuran-2-ylcarbonyl)-2,6-diazabicyclo[3.2.0]heptane,
6-(3-bromofuran-2-ylcarbonyl)-2,6-diazabicyclo[3.2.0]heptane,
2-(5-bromofuran-2-ylcarbonyl)-2,6-diazabicyclo[3.2.0]heptane,
6-(5-bromofuran-2-ylcarbonyl)-2,6-diazabicyclo[3.2.0]heptane,
2-(furan-3-ylcarbonyl)-2,6-diazabicyclo[3.2.0]heptane,
6-(furan-3-ylcarbonyl)-2,6-diazabicyclo[3.2.0]heptane,
2-(2-methylfuran-3-ylcarbonyl)-2,6-diazabicyclo[3.2.0]heptane,
6-(2-methylfuran-3-ylcarbonyl)-2,6-diazabicyclo[3.2.0]heptane,
2-(oxazol-2-ylcarbonyl)-2,6-diazabicyclo[3.2.0]heptane,
6-(oxazol-2-ylcarbonyl)-2,6-diazabicyclo[3.2.0]heptane,
2-(oxazol-5-ylcarbonyl)-2,6-diazabicyclo[3.2.0]heptane,
6-(oxazol-5-ylcarbonyl)-2,6-diazabicyclo[3.2.0]heptane,
2-(4-methyloxazol-5-ylcarbonyl)-2,6-diazabicyclo[3.2.0]heptane,
6-(4-methyloxazol-5-ylcarbonyl)-2,6-diazabicyclo[3.2.0]heptane,
2-(isoxazol-3-ylcarbonyl)-2,6-diazabicyclo[3.2.0]heptane,
6-(isoxazol-3-ylcarbonyl)-2,6-diazabicyclo[3.2.0]heptane,
2-(5-methylisoxazol-3-ylcarbonyl)-2,6-diazabicyclo[3.2.0]heptane,
6-(5-methylisoxazol-3-ylcarbonyl)-2,6-diazabicyclo[3.2.0]heptane,
2-(isoxazol-4-ylcarbonyl)-2,6-diazabicyclo[3.2.0]heptane,
6-(isoxazol-4-ylcarbonyl)-2,6-diazabicyclo[3.2.0]heptane,
2-(3-methylisoxazol-4-ylcarbonyl)-2,6-diazabicyclo[3.2.0]heptane,
6-(3-methylisoxazol-4-ylcarbonyl)-2,6-diazabicyclo[3.2.0]heptane,
2-(5-methylisoxazol-4-ylcarbonyl)-2,6-diazabicyclo[3.2.0]heptane,
6-(5-methylisoxazol-4-ylcarbonyl)-2,6-diazabicyclo[3.2.0]heptane,
2-(isoxazol-5-ylcarbonyl)-2,6-diazabicyclo[3.2.0]heptane,
6-(isoxazol-5-ylcarbonyl)-2,6-diazabicyclo[3.2.0]heptane,
2-(3-methylisoxazol-5-ylcarbonyl)-2,6-diazabicyclo[3.2.0]heptane,
6-(3-methylisoxazol-5-ylcarbonyl)-2,6-diazabicyclo[3.2.0]heptane,
2-(3-bromoisoxazol-5-ylcarbonyl)-2,6-diazabicyclo[3.2.0]heptane,
6-(3-bromoisoxazol-5-ylcarbonyl)-2,6-diazabicyclo[3.2.0]heptane,
2-(3-methoxyisoxazol-5-ylcarbonyl)-2,6-diazabicyclo[3.2.0]heptane,
6-(3-methoxyisoxazol-5-ylcarbonyl)-2,6-diazabicyclo[3.2.0]heptane,
2-(pyridin-4-ylcarbonyl)-2,6-diazabicyclo[3.2.0]heptane,
6-(pyridin-4-ylcarbonyl)-2,6-diazabicyclo[3.2.0]heptane,
3-(furan-2-ylcarbonyl)-3,6-diazabicyclo[3.2.0]heptane,
6-(furan-2-ylcarbonyl)-3,6-diazabicyclo[3.2.0]heptane,
3-(3-methylfuran-2-ylcarbonyl)-3,6-diazabicyclo[3.2.0]heptane,
6-(3-methylfuran-2-ylcarbonyl)-3,6-diazabicyclo[3.2.0]heptane,
3-(5-methylfuran-2-ylcarbonyl)-3,6-diazabicyclo[3.2.0]heptane,
6-(5-methylfuran-2-ylcarbonyl)-3,6-diazabicyclo[3.2.0]heptane,
3-(3-chlorofuran-2-ylcarbonyl)-3,6-diazabicyclo[3.2.0]heptane,
6-(3-chlorofuran-2-ylcarbonyl)-3,6-diazabicyclo[3.2.0]heptane,
3-(5-chlorofuran-2-ylcarbonyl)-3,6-diazabicyclo[3.2.0]heptane,
6-(5-chlorofuran-2-ylcarbonyl)-3,6-diazabicyclo[3.2.0]heptane,
3-(3-bromofuran-2-ylcarbonyl)-3,6-diazabicyclo[3.2.0]heptane,
6-(3-bromofuran-2-ylcarbonyl)-3,6-diazabicyclo[3.2.0]heptane,
3-(5-bromofuran-2-ylcarbonyl)-3,6-diazabicyclo[3.2.0]heptane,
6-(5-bromofuran-2-ylcarbonyl)-3,6-diazabicyclo[3.2.0]heptane,
3-(furan-3-ylcarbonyl)-3,6-diazabicyclo[3.2.0]heptane,
6-(furan-3-ylcarbonyl)-3,6-diazabicyclo[3.2.0]heptane,
3-(2-methylfuran-3-ylcarbonyl)-3,6-diazabicyclo[3.2.0]heptane,
6-(2-methylfuran-3-ylcarbonyl)-3,6-diazabicyclo[3.2.0]heptane,
3-(oxazol-2-ylcarbonyl)-3,6-diazabicyclo[3.2.0]heptane,
6-(oxazol-2-ylcarbonyl)-3,6-diazabicyclo[3.2.0]heptane,
3-(oxazol-5-ylcarbonyl)-3,6-diazabicyclo[3.2.0]heptane,
6-(oxazol-5-ylcarbonyl)-3,6-diazabicyclo[3.2.0]heptane,
3-(4-methyloxazol-5-ylcarbonyl)-3,6-diazabicyclo[3.2.0]heptane,
6-(4-methyloxazol-5-ylcarbonyl)-3,6-diazabicyclo[3.2.0]heptane,
3-(isoxazol-3-ylcarbonyl)-3,6-diazabicyclo[3.2.0]heptane,
6-(isoxazol-3-ylcarbonyl)-3,6-diazabicyclo[3.2.0]heptane,
3-(5-methylisoxazol-3-ylcarbonyl)-3,6-diazabicyclo[3.2.0]heptane,
6-(5-methylisoxazol-3-ylcarbonyl)-3,6-diazabicyclo[3.2.0]heptane,
3-(isoxazol-4-ylcarbonyl)-3,6-diazabicyclo[3.2.0]heptane,
6-(isoxazol-4-ylcarbonyl)-3,6-diazabicyclo[3.2.0]heptane,
3-(3-methylisoxazol-4-ylcarbonyl)-3,6-diazabicyclo[3.2.0]heptane,
6-(3-methylisoxazol-4-ylcarbonyl)-3,6-diazabicyclo[3.2.0]heptane,
3-(5-methylisoxazol-4-ylcarbonyl)-3,6-diazabicyclo[3.2.0]heptane,
6-(5-methylisoxazol-4-ylcarbonyl)-3,6-diazabicyclo[3.2.0]heptane,
3-(isoxazol-5-ylcarbonyl)-3,6-diazabicyclo[3.2.0]heptane,
6-(isoxazol-5-ylcarbonyl)-3,6-diazabicyclo[3.2.0]heptane,
3-(3-methylisoxazol-5-ylcarbonyl)-3,6-diazabicyclo[3.2.0]heptane,
6-(3-methylisoxazol-5-ylcarbonyl)-3,6-diazabicyclo[3.2.0]heptane,
3-(3-bromoisoxazol-5-ylcarbonyl)-3,6-diazabicyclo[3.2.0]heptane,
6-(3-bromoisoxazol-5-ylcarbonyl)-3,6-diazabicyclo[3.2.0]heptane,
3-(3-methoxyisoxazol-5-ylcarbonyl)-3,6-diazabicyclo[3.2.0]heptane,
6-(3-methoxyisoxazol-5-ylcarbonyl)-3,6-diazabicyclo[3.2.0]heptane,
3-(pyridin-4-ylcarbonyl)-3,6-diazabicyclo[3.2.0]heptane,
6-(pyridin-4-ylcarbonyl)-3,6-diazabicyclo[3.2.0]heptane,
2-(furan-2-ylcarbonyl)-2,7-diazabicyclo[4.2.0]octane,
7-(furan-2-ylcarbonyl)-2,7-diazabicyclo[4.2.0]octane,
2-(3-methylfuran-2-ylcarbonyl)-2,7-diazabicyclo[4.2.0]octane,
7-(3-methylfuran-2-ylcarbonyl)-2,7-diazabicyclo[4.2.0]octane,
2-(5-methylfuran-2-ylcarbonyl)-2,7-diazabicyclo[4.2.0]octane,
7-(5-methylfuran-2-ylcarbonyl)-2,7-diazabicyclo[4.2.0]octane,
2-(3-chlorofuran-2-ylcarbonyl)-2,7-diazabicyclo[4.2.0]octane,
7-(3-chlorofuran-2-ylcarbonyl)-2,7-diazabicyclo[4.2.0]octane,
2-(5-chlorofuran-2-ylcarbonyl)-2,7-diazabicyclo[4.2.0]octane,
7-(5-chlorofuran-2-ylcarbonyl)-2,7-diazabicyclo[4.2.0]octane,
2-(3-bromofuran-2-ylcarbonyl)-2,7-diazabicyclo[4.2.0]octane,
7-(3-bromofuran-2-ylcarbonyl)-2,7-diazabicyclo[4.2.0]octane,
2-(5-bromofuran-2-ylcarbonyl)-2,7-diazabicyclo[4.2.0]octane,
7-(5-bromofuran-2-ylcarbonyl)-2,7-diazabicyclo[4.2.0]octane,
2-(furan-3-ylcarbonyl)-2,7-diazabicyclo[4.2.0]octane,
7-(furan-3-ylcarbonyl)-2,7-diazabicyclo[4.2.0]octane,
2-(2-methylfuran-3-ylcarbonyl)-2,7-diazabicyclo[4.2.0]octane,
7-(2-methylfuran-3-ylcarbonyl)-2,7-diazabicyclo[4.2.0]octane,
2-(oxazol-2-ylcarbonyl)-2,7-diazabicyclo[4.2.0]octane,
7-(oxazol-2-ylcarbonyl)-2,7-diazabicyclo[4.2.0]octane,
2-(oxazol-5-ylcarbonyl)-2,7-diazabicyclo[4.2.0]octane,
7-(oxazol-5-ylcarbonyl)-2,7-diazabicyclo[4.2.0]octane,
2-(4-methyloxazol-5-ylcarbonyl)-2,7-diazabicyclo[4.2.0]octane,
7-(4-methyloxazol-5-ylcarbonyl)-2,7-diazabicyclo[4.2.0]octane,
2-(isoxazol-3-ylcarbonyl)-2,7-diazabicyclo[4.2.0]octane,
7-(isoxazol-3-ylcarbonyl)-2,7-diazabicyclo[4.2.0]octane,
2-(5-methylisoxazol-3-ylcarbonyl)-2,7-diazabicyclo[4.2.0]octane,
7-(5-methylisoxazol-3-ylcarbonyl)-2,7-diazabicyclo[4.2.0]octane,
2-(isoxazol-4-ylcarbonyl)-2,7-diazabicyclo[4.2.0]octane,
7-(isoxazol-4-ylcarbonyl)-2,7-diazabicyclo[4.2.0]octane,
2-(3-methylisoxazol-4-ylcarbonyl)-2,7-diazabicyclo[4.2.0]octane,
7-(3-methylisoxazol-4-ylcarbonyl)-2,7-diazabicyclo[4.2.0]octane,
2-(5-methylisoxazol-4-ylcarbonyl)-2,7-diazabicyclo[4.2.0]octane,
7-(5-methylisoxazol-4-ylcarbonyl)-2,7-diazabicyclo[4.2.0]octane,
2-(isoxazol-5-ylcarbonyl)-2,7-diazabicyclo[4.2.0]octane,
7-(isoxazol-5-ylcarbonyl)-2,7-diazabicyclo[4.2.0]octane,
2-(3-methylisoxazol-5-ylcarbonyl)-2,7-diazabicyclo[4.2.0]octane,
7-(3-methylisoxazol-5-ylcarbonyl)-2,7-diazabicyclo[4.2.0]octane,
2-(3-bromoisoxazol-5-ylcarbonyl)-2,7-diazabicyclo[4.2.0]octane,
7-(3-bromoisoxazol-5-ylcarbonyl)-2,7-diazabicyclo[4.2.0]octane,
2-(3-methoxyisoxazol-5-ylcarbonyl)-2,7-diazabicyclo[4.2.0]octane,
7-(3-methoxyisoxazol-5-ylcarbonyl)-2,7-diazabicyclo[4.2.0]octane,
2-(pyridin-4-ylcarbonyl)-2,7-diazabicyclo[4.2.0]octane,
7-(pyridin-4-ylcarbonyl)-2,7-diazabicyclo[4.2.0]octane,
3-(furan-2-ylcarbonyl)-3,7-diazabicyclo[4.2.0]octane,
7-(furan-2-ylcarbonyl)-3,7-diazabicyclo[4.2.0]octane,
3-(3-methylfuran-2-ylcarbonyl)-3,7-diazabicyclo[4.2.0]octane,
7-(3-methylfuran-2-ylcarbonyl)-3,7-diazabicyclo[4.2.0]octane,
3-(5-methylfuran-2-ylcarbonyl)-3,7-diazabicyclo[4.2.0]octane,
7-(5-methylfuran-2-ylcarbonyl)-3,7-diazabicyclo[4.2.0]octane,
3-(3-chlorofuran-2-ylcarbonyl)-3,7-diazabicyclo[4.2.0]octane,
7-(3-chlorofuran-2-ylcarbonyl)-3,7-diazabicyclo[4.2.0]octane,
3-(5-chlorofuran-2-ylcarbonyl)-3,7-diazabicyclo[4.2.0]octane,
7-(5-chlorofuran-2-ylcarbonyl)-3,7-diazabicyclo[4.2.0]octane,
3-(3-bromofuran-2-ylcarbonyl)-3,7-diazabicyclo[4.2.0]octane,
7-(3-bromofuran-2-ylcarbonyl)-3,7-diazabicyclo[4.2.0]octane,
3-(5-bromofuran-2-ylcarbonyl)-3,7-diazabicyclo[4.2.0]octane,
7-(5-bromofuran-2-ylcarbonyl)-3,7-diazabicyclo[4.2.0]octane,
3-(furan-3-ylcarbonyl)-3,7-diazabicyclo[4.2.0]octane,
7-(furan-3-ylcarbonyl)-3,7-diazabicyclo[4.2.0]octane,
3-(2-methylfuran-3-ylcarbonyl)-3,7-diazabicyclo[4.2.0]octane,
7-(2-methylfuran-3-ylcarbonyl)-3,7-diazabicyclo[4.2.0]octane,
3-(oxazol-2-ylcarbonyl)-3,7-diazabicyclo[4.2.0]octane,
7-(oxazol-2-ylcarbonyl)-3,7-diazabicyclo[4.2.0]octane,
3-(oxazol-5-ylcarbonyl)-3,7-diazabicyclo[4.2.0]octane,
7-(oxazol-5-ylcarbonyl)-3,7-diazabicyclo[4.2.0]octane,
3-(4-methyloxazol-5-ylcarbonyl)-3,7-diazabicyclo[4.2.0]octane,
7-(4-methyloxazol-5-ylcarbonyl)-3,7-diazabicyclo[4.2.0]octane,
3-(isoxazol-3-ylcarbonyl)-3,7-diazabicyclo[4.2.0]octane,
7-(isoxazol-3-ylcarbonyl)-3,7-diazabicyclo[4.2.0]octane,
3-(5-methylisoxazol-3-ylcarbonyl)-3,7-diazabicyclo[4.2.0]octane,
7-(5-methylisoxazol-3-ylcarbonyl)-3,7-diazabicyclo[4.2.0]octane,
3-(isoxazol-4-ylcarbonyl)-3,7-diazabicyclo[4.2.0]octane,
7-(isoxazol-4-ylcarbonyl)-3,7-diazabicyclo[4.2.0]octane,
3-(3-methylisoxazol-4-ylcarbonyl)-3,7-diazabicyclo[4.2.0]octane,
7-(3-methylisoxazol-4-ylcarbonyl)-3,7-diazabicyclo[4.2.0]octane,
3-(5-methylisoxazol-4-ylcarbonyl)-3,7-diazabicyclo[4.2.0]octane,
7-(5-methylisoxazol-4-ylcarbonyl)-3,7-diazabicyclo[4.2.0]octane,
3-(isoxazol-5-ylcarbonyl)-3,7-diazabicyclo[4.2.0]octane,
7-(isoxazol-5-ylcarbonyl)-3,7-diazabicyclo[4.2.0]octane,
3-(3-methylisoxazol-5-ylcarbonyl)-3,7-diazabicyclo[4.2.0]octane,
7-(3-methylisoxazol-5-ylcarbonyl)-3,7-diazabicyclo[4.2.0]octane,
3-(3-bromoisoxazol-5-ylcarbonyl)-3,7-diazabicyclo[4.2.0]octane,
7-(3-bromoisoxazol-5-ylcarbonyl)-3,7-diazabicyclo[4.2.0]octane,
3-(3-methoxyisoxazol-5-ylcarbonyl)-3,7-diazabicyclo[4.2.0]octane,
7-(3-methoxyisoxazol-5-ylcarbonyl)-3,7-diazabicyclo[4.2.0]octane,
3-(pyridin-4-ylcarbonyl)-3,7-diazabicyclo[4.2.0]octane,
7-(Pyridin-4-ylcarbonyl)-3,7-diazabicyclo[4.2.0]octane,
3-(furan-2-ylcarbonyl)-3,8-diazabicyclo[4.2.0]octane,
8-(furan-2-ylcarbonyl)-3,8-diazabicyclo[4.2.0]octane,
3-(3-methylfuran-2-ylcarbonyl)-3,8-diazabicyclo[4.2.0]octane,
8-(3-methylfuran-2-ylcarbonyl)-3,8-diazabicyclo[4.2.0]octane,
3-(5-methylfuran-2-ylcarbonyl)-3,8-diazabicyclo[4.2.0]octane,
8-(5-methylfuran-2-ylcarbonyl)-3,8-diazabicyclo[4.2.0]octane,
3-(3-chlorofuran-2-ylcarbonyl)-3,8-diazabicyclo[4.2.0]octane,
8-(3-chlorofuran-2-ylcarbonyl)-3,8-diazabicyclo[4.2.0]octane,
3-(5-chlorofuran-2-ylcarbonyl)-3,8-diazabicyclo[4.2.0]octane,
8-(5-chlorofuran-2-ylcarbonyl)-3,8-diazabicyclo[4.2.0]octane,
3-(3-bromofuran-2-ylcarbonyl)-3,8-diazabicyclo[4.2.0]octane,
8-(3-bromofuran-2-ylcarbonyl)-3,8-diazabicyclo[4.2.0]octane,
3-(5-bromofuran-2-ylcarbonyl)-3,8-diazabicyclo[4.2.0]octane,
8-(5-bromofuran-2-ylcarbonyl)-3,8-diazabicyclo[4.2.0]octane,
3-(furan-3-ylcarbonyl)-3,8-diazabicyclo[4.2.0]octane,
8-(furan-3-ylcarbonyl)-3,8-diazabicyclo[4.2.0]octane,
3-(2-methylfuran-3-ylcarbonyl)-3,8-diazabicyclo[4.2.0]octane,
8-(2-methylfuran-3-ylcarbonyl)-3,8-diazabicyclo[4.2.0]octane,
3-(oxazol-2-ylcarbonyl)-3,8-diazabicyclo[4.2.0]octane,
8-(oxazol-2-ylcarbonyl)-3,8-diazabicyclo[4.2.0]octane,
3-(oxazol-5-ylcarbonyl)-3,8-diazabicyclo[4.2.0]octane,
8-(oxazol-5-ylcarbonyl)-3,8-diazabicyclo[4.2.0]octane,
3-(4-methyloxazol-5-ylcarbonyl)-3,8-diazabicyclo[4.2.0]octane,
8-(4-methyloxazol-5-ylcarbonyl)-3,8-diazabicyclo[4.2.0]octane,
3-(isoxazol-3-ylcarbonyl)-3,8-diazabicyclo[4.2.0]octane,
8-(isoxazol-3-ylcarbonyl)-3,8-diazabicyclo[4.2.0]octane,
3-(5-methylisoxazol-3-ylcarbonyl)-3,8-diazabicyclo[4.2.0]octane,
8-(5-methylisoxazol-3-ylcarbonyl)-3,8-diazabicyclo[4.2.0]octane,
3-(isoxazol-4-ylcarbonyl)-3,8-diazabicyclo[4.2.0]octane,
8-(isoxazol-4-ylcarbonyl)-3,8-diazabicyclo[4.2.0]octane,
3-(3-methylisoxazol-4-ylcarbonyl)-3,8-diazabicyclo[4.2.0]octane,
8-(3-methylisoxazol-4-ylcarbonyl)-3,8-diazabicyclo[4.2.0]octane,
3-(5-methylisoxazol-4-ylcarbonyl)-3,8-diazabicyclo[4.2.0]octane,
8-(5-methylisoxazol-4-ylcarbonyl)-3,8-diazabicyclo[4.2.0]octane,
3-(isoxazol-5-ylcarbonyl)-3,8-diazabicyclo[4.2.0]octane,
8-(isoxazol-5-ylcarbonyl)-3,8-diazabicyclo[4.2.0]octane,
3-(3-methylisoxazol-5-ylcarbonyl)-3,8-diazabicyclo[4.2.0]octane,
8-(3-methylisoxazol-5-ylcarbonyl)-3,8-diazabicyclo[4.2.0]octane,
3-(3-bromoisoxazol-5-ylcarbonyl)-3,8-diazabicyclo[4.2.0]octane,
8-(3-bromoisoxazol-5-ylcarbonyl)-3,8-diazabicyclo[4.2.0]octane,
3-(3-methoxyisoxazol-5-ylcarbonyl)-3,8-diazabicyclo[4.2.0]octane,
8-(3-methoxyisoxazol-5-ylcarbonyl)-3,8-diazabicyclo[4.2.0]octane,
3-(pyridin-4-ylcarbonyl)-3,8-diazabicyclo[4.2.0]octane,
8-(pyridin-4-ylcarbonyl)-3,8-diazabicyclo[4.2.0]octane,
2-(furan-2-ylcarbonyl)-2,6-diazabicyclo[3.3.0]octane,
2-(3-methylfuran-2-ylcarbonyl)-2,6-diazabicyclo[3.3.0]octane,
2-(5-methylfuran-2-ylcarbonyl)-2,6-diazabicyclo[3.3.0]octane,
2-(3-chlorofuran-2-ylcarbonyl)-2,6-diazabicyclo[3.3.0]octane,
2-(5-chlorofuran-2-ylcarbonyl)-2,6-diazabicyclo[3.3.0]octane,
2-(3-bromofuran-2-ylcarbonyl)-2,6-diazabicyclo[3.3.0]octane,
2-(5-bromofuran-2-ylcarbonyl)-2,6-diazabicyclo[3.3.0]octane,
2-(furan-3-ylcarbonyl)-2,6-diazabicyclo[3.3.0]octane,
2-(2-methylfuran-3-ylcarbonyl)-2,6-diazabicyclo[3.3.0]octane,
2-(oxazol-2-ylcarbonyl)-2,6-diazabicyclo[3.3.0]octane,
2-(oxazol-5-ylcarbonyl)-2,6-diazabicyclo[3.3.0]octane,
2-(4-methyloxazol-5-ylcarbonyl)-2,6-diazabicyclo[3.3.0]octane,
2-(isoxazol-3-ylcarbonyl)-2,6-diazabicyclo[3.3.0]octane,
2-(5-methylisoxazol-3-ylcarbonyl)-2,6-diazabicyclo[3.3.0]octane,
2-(isoxazol-4-ylcarbonyl)-2,6-diazabicyclo[3.3.0]octane,
2-(3-methylisoxazol-4-ylcarbonyl)-2,6-diazabicyclo[3.3.0]octane,
2-(5-methylisoxazol-4-ylcarbonyl)-2,6-diazabicyclo[3.3.0]octane,
2-(isoxazol-5-ylcarbonyl)-2,6-diazabicyclo[3.3.0]octane,
2-(3-methylisoxazol-5-ylcarbonyl)-2,6-diazabicyclo[3.3.0]octane,
2-(3-bromoisoxazol-5-ylcarbonyl)-2,6-diazabicyclo[3.3.0]octane,
2-(3-methoxyisoxazol-5-ylcarbonyl)-2,6-diazabicyclo[3.3.0]octane,
2-(pyridin-4-ylcarbonyl)-2,6-diazabicyclo[3.3.0]octane,
2-(furan-2-ylcarbonyl)-2,7-diazabicyclo[3.3.0]octane,
7-(furan-2-ylcarbonyl)-2,7-diazabicyclo[3.3.0]octane,
2-(3-methylfuran-2-ylcarbonyl)-2,7-diazabicyclo[3.3.0]octane,
7-(3-methylfuran-2-ylcarbonyl)-2,7-diazabicyclo[3.3.0]octane,
2-(5-methylfuran-2-ylcarbonyl)-2,7-diazabicyclo[3.3.0]octane,
7-(5-methylfuran-2-ylcarbonyl)-2,7-diazabicyclo[3.3.0]octane,
2-(3-chlorofuran-2-ylcarbonyl)-2,7-diazabicyclo[3.3.0]octane,
7-(3-chlorofuran-2-ylcarbonyl)-2,7-diazabicyclo[3.3.0]octane,
2-(5-chlorofuran-2-ylcarbonyl)-2,7-diazabicyclo[3.3.0]octane,
7-(5-chlorofuran-2-ylcarbonyl)-2,7-diazabicyclo[3.3.0]octane,
2-(3-bromofuran-2-ylcarbonyl)-2,7-diazabicyclo[3.3.0]octane,
7-(3-bromofuran-2-ylcarbonyl)-2,7-diazabicyclo[3.3.0]octane,
2-(5-bromofuran-2-ylcarbonyl)-2,7-diazabicyclo[3.3.0]octane,
7-(5-bromofuran-2-ylcarbonyl)-2,7-diazabicyclo[3.3.0]octane,
2-(furan-3-ylcarbonyl)-2,7-diazabicyclo[3.3.0]octane,
7-(furan-3-ylcarbonyl)-2,7-diazabicyclo[3.3.0]octane,
2-(2-methylfuran-3-ylcarbonyl)-2,7-diazabicyclo[3.3.0]octane,
7-(2-methylfuran-3-ylcarbonyl)-2,7-diazabicyclo[3.3.0]octane,
2-(oxazol-2-ylcarbonyl)-2,7-diazabicyclo[3.3.0]octane,
7-(oxazol-2-ylcarbonyl)-2,7-diazabicyclo[3.3.0]octane,
2-(oxazol-5-ylcarbonyl)-2,7-diazabicyclo[3.3.0]octane,
7-(oxazol-5-ylcarbonyl)-2,7-diazabicyclo[3.3.0]octane,
2-(4-methyloxazol-5-ylcarbonyl)-2,7-diazabicyclo[3.3.0]octane,
7-(4-methyloxazol-5-ylcarbonyl)-2,7-diazabicyclo[3.3.0]octane,
2-(isoxazol-3-ylcarbonyl)-2,7-diazabicyclo[3.3.0]octane,
7-(isoxazol-3-ylcarbonyl)-2,7-diazabicyclo[3.3.0]octane,
2-(5-methylisoxazol-3-ylcarbonyl)-2,7-diazabicyclo[3.3.0]octane,
7-(5-methylisoxazol-3-ylcarbonyl)-2,7-diazabicyclo[3.3.0]octane,
2-(isoxazol-4-ylcarbonyl)-2,7-diazabicyclo[3.3.0]octane,
7-(isoxazol-4-ylcarbonyl)-2,7-diazabicyclo[3.3.0]octane,
2-(3-methylisoxazol-4-ylcarbonyl)-2,7-diazabicyclo[3.3.0]octane,
7-(3-methylisoxazol-4-ylcarbonyl)-2,7-diazabicyclo[3.3.0]octane,
2-(5-methylisoxazol-4-ylcarbonyl)-2,7-diazabicyclo[3.3.0]octane,
7-(5-methylisoxazol-4-ylcarbonyl)-2,7-diazabicyclo[3.3.0]octane,
2-(isoxazol-5-ylcarbonyl)-2,7-diazabicyclo[3.3.0]octane,
7-(isoxazol-5-ylcarbonyl)-2,7-diazabicyclo[3.3.0]octane,
2-(3-methylisoxazol-5-ylcarbonyl)-2,7-diazabicyclo[3.3.0]octane,
7-(3-methylisoxazol-5-ylcarbonyl)-2,7-diazabicyclo[3.3.0]octane,
2-(3-bromoisoxazol-5-ylcarbonyl)-2,7-diazabicyclo[3.3.0]octane,
7-(3-bromoisoxazol-5-ylcarbonyl)-2,7-diazabicyclo[3.3.0]octane,
2-(3-methoxyisoxazol-5-ylcarbonyl)-2,7-diazabicyclo[3.3.0]octane,
7-(3-methoxyisoxazol-5-ylcarbonyl)-2,7-diazabicyclo[3.3.0]octane,
2-(pyridin-4-ylcarbonyl)-2,7-diazabicyclo[3.3.0]octane,
7-(pyridin-4-ylcarbonyl)-2,7-diazabicyclo[3.3.0]octane,
2-(furan-2-ylcarbonyl)-2,7-diazabicyclo[4.3.0]nonane,
7-(furan-2-ylcarbonyl)-2,7-diazabicyclo[4.3.0]nonane,
2-(3-methylfuran-2-ylcarbonyl)-2,7-diazabicyclo[4.3.0]nonane,
7-(3-methylfuran-2-ylcarbonyl)-2,7-diazabicyclo[4.3.0]nonane,
2-(5-methylfuran-2-ylcarbonyl)-2,7-diazabicyclo[4.3.0]nonane,
7-(5-methylfuran-2-ylcarbonyl)-2,7-diazabicyclo[4.3.0]nonane,
2-(3-chlorofuran-2-ylcarbonyl)-2,7-diazabicyclo[4.3.0]nonane,
7-(3-chlorofuran-2-ylcarbonyl)-2,7-diazabicyclo[4.3.0]nonane,
2-(5-chlorofuran-2-ylcarbonyl)-2,7-diazabicyclo[4.3.0]nonane,
7-(5-chlorofuran-2-ylcarbonyl)-2,7-diazabicyclo[4.3.0]nonane,
2-(3-bromofuran-2-ylcarbonyl)-2,7-diazabicyclo[4.3.0]nonane,
7-(3-bromofuran-2-ylcarbonyl)-2,7-diazabicyclo[4.3.0]nonane,
2-(5-bromofuran-2-ylcarbonyl)-2,7-diazabicyclo[4.3.0]nonane,
7-(5-bromofuran-2-ylcarbonyl)-2,7-diazabicyclo[4.3.0]nonane,
2-(furan-3-ylcarbonyl)-2,7-diazabicyclo[4.3.0]nonane,
7-(furan-3-ylcarbonyl)-2,7-diazabicyclo[4.3.0]nonane,
2-(2-methylfuran-3-ylcarbonyl)-2,7-diazabicyclo[4.3.0]nonane,
7-(2-methylfuran-3-ylcarbonyl)-2,7-diazabicyclo[4.3.0]nonane,
2-(oxazol-2-ylcarbonyl)-2,7-diazabicyclo[4.3.0]nonane,
7-(oxazol-2-ylcarbonyl)-2,7-diazabicyclo[4.3.0]nonane,
2-(oxazol-5-ylcarbonyl)-2,7-diazabicyclo[4.3.0]nonane,
7-(oxazol-5-ylcarbonyl)-2,7-diazabicyclo[4.3.0]nonane,
2-(4-methyloxazol-5-ylcarbonyl)-2,7-diazabicyclo[4.3.0]nonane,
7-(4-methyloxazol-5-ylcarbonyl)-2,7-diazabicyclo[4.3.0]nonane,
2-(isoxazol-3-ylcarbonyl)-2,7-diazabicyclo[4.3.0]nonane,
7-(isoxazol-3-ylcarbonyl)-2,7-diazabicyclo[4.3.0]nonane,
2-(5-methylisoxazol-3-ylcarbonyl)-2,7-diazabicyclo[4.3.0]nonane,
7-(5-methylisoxazol-3-ylcarbonyl)-2,7-diazabicyclo[4.3.0]nonane,
2-(isoxazol-4-ylcarbonyl)-2,7-diazabicyclo[4.3.0]nonane,
7-(isoxazol-4-ylcarbonyl)-2,7-diazabicyclo[4.3.0]nonane,
2-(3-methylisoxazol-4-ylcarbonyl)-2,7-diazabicyclo[4.3.0]nonane,
7-(3-methylisoxazol-4-ylcarbonyl)-2,7-diazabicyclo[4.3.0]nonane,
2-(5-methylisoxazol-4-ylcarbonyl)-2,7-diazabicyclo[4.3.0]nonane,
7-(5-methylisoxazol-4-ylcarbonyl)-2,7-diazabicyclo[4.3.0]nonane,
2-(isoxazol-5-ylcarbonyl)-2,7-diazabicyclo[4.3.0]nonane,
7-(isoxazol-5-ylcarbonyl)-2,7-diazabicyclo[4.3.0]nonane,
2-(3-methylisoxazol-5-ylcarbonyl)-2,7-diazabicyclo[4.3.0]nonane,
7-(3-methylisoxazol-5-ylcarbonyl)-2,7-diazabicyclo[4.3.0]nonane,
2-(3-bromoisoxazol-5-ylcarbonyl)-2,7-diazabicyclo[4.3.0]nonane,
7-(3-bromoisoxazol-5-ylcarbonyl)-2,7-diazabicyclo[4.3.0]nonane,
2-(3-methoxyisoxazol-5-ylcarbonyl)-2,7-diazabicyclo[4.3.0]nonane,
7-(3-methoxyisoxazol-5-ylcarbonyl)-2,7-diazabicyclo[4.3.0]nonane,
2-(pyridin-4-ylcarbonyl)-2,7-diazabicyclo[4.3.0]nonane,
7-(pyridin-4-ylcarbonyl)-2,7-diazabicyclo[4.3.0]nonane,
2-(furan-2-ylcarbonyl)-2,8-diazabicyclo[4.3.0]nonane,
8-(furan-2-ylcarbonyl)-2,8-diazabicyclo[4.3.0]nonane,
2-(3-methylfuran-2-ylcarbonyl)-2,8-diazabicyclo[4.3.0]nonane,
8-(3-methylfuran-2-ylcarbonyl)-2,8-diazabicyclo[4.3.0]nonane,
2-(5-methylfuran-2-ylcarbonyl)-2,8-diazabicyclo[4.3.0]nonane,
8-(5-methylfuran-2-ylcarbonyl)-2,8-diazabicyclo[4.3.0]nonane,
2-(3-chlorofuran-2-ylcarbonyl)-2,8-diazabicyclo[4.3.0]nonane,
8-(3-chlorofuran-2-ylcarbonyl)-2,8-diazabicyclo[4.3.0]nonane,
2-(5-chlorofuran-2-ylcarbonyl)-2,8-diazabicyclo[4.3.0]nonane,
8-(5-chlorofuran-2-ylcarbonyl)-2,8-diazabicyclo[4.3.0]nonane,
2-(3-bromofuran-2-ylcarbonyl)-2,8-diazabicyclo[4.3.0]nonane,
8-(3-bromofuran-2-ylcarbonyl)-2,8-diazabicyclo[4.3.0]nonane,
2-(5-bromofuran-2-ylcarbonyl)-2,8-diazabicyclo[4.3.0]nonane,
8-(5-bromofuran-2-ylcarbonyl)-2,8-diazabicyclo[4.3.0]nonane,
2-(furan-3-ylcarbonyl)-2,8-diazabicyclo[4.3.0]nonane,
8-(furan-3-ylcarbonyl)-2,8-diazabicyclo[4.3.0]nonane,
2-(2-methylfuran-3-ylcarbonyl)-2,8-diazabicyclo[4.3.0]nonane,
8-(2-methylfuran-3-ylcarbonyl)-2,8-diazabicyclo[4.3.0]nonane,
2-(oxazol-2-ylcarbonyl)-2,8-diazabicyclo[4.3.0]nonane,
8-(oxazol-2-ylcarbonyl)-2,8-diazabicyclo[4.3.0]nonane,
2-(oxazol-5-ylcarbonyl)-2,8-diazabicyclo[4.3.0]nonane,
8-(oxazol-5-ylcarbonyl)-2,8-diazabicyclo[4.3.0]nonane,
2-(4-methyloxazol-5-ylcarbonyl)-2,8-diazabicyclo[4.3.0]nonane,
8-(4-methyloxazol-5-ylcarbonyl)-2,8-diazabicyclo[4.3.0]nonane,
2-(isoxazol-3-ylcarbonyl)-2,8-diazabicyclo[4.3.0]nonane,
8-(isoxazol-3-ylcarbonyl)-2,8-diazabicyclo[4.3.0]nonane,
2-(5-methylisoxazol-3-ylcarbonyl)-2,8-diazabicyclo[4.3.0]nonane,
8-(5-methylisoxazol-3-ylcarbonyl)-2,8-diazabicyclo[4.3.0]nonane,
2-(isoxazol-4-ylcarbonyl)-2,8-diazabicyclo[4.3.0]nonane,
8-(isoxazol-4-ylcarbonyl)-2,8-diazabicyclo[4.3.0]nonane,
2-(3-methylisoxazol-4-ylcarbonyl)-2,8-diazabicyclo[4.3.0]nonane,
8-(3-methylisoxazol-4-ylcarbonyl)-2,8-diazabicyclo[4.3.0]nonane,
2-(5-methylisoxazol-4-ylcarbonyl)-2,8-diazabicyclo[4.3.0]nonane,
8-(5-methylisoxazol-4-ylcarbonyl)-2,8-diazabicyclo[4.3.0]nonane,
2-(isoxazol-5-ylcarbonyl)-2,8-diazabicyclo[4.3.0]nonane,
8-(isoxazol-5-ylcarbonyl)-2,8-diazabicyclo[4.3.0]nonane,
2-(3-methylisoxazol-5-ylcarbonyl)-2,8-diazabicyclo[4.3.0]nonane,
8-(3-methylisoxazol-5-ylcarbonyl)-2,8-diazabicyclo[4.3.0]nonane,
2-(3-bromoisoxazol-5-ylcarbonyl)-2,8-diazabicyclo[4.3.0]nonane,
8-(3-bromoisoxazol-5-ylcarbonyl)-2,8-diazabicyclo[4.3.0]nonane,
2-(3-methoxyisoxazol-5-ylcarbonyl)-2,8-diazabicyclo[4.3.0]nonane,
8-(3-methoxyisoxazol-5-ylcarbonyl)-2,8-diazabicyclo[4.3.0]nonane,
2-(pyridin-4-ylcarbonyl)-2,8-diazabicyclo[4.3.0]nonane,
8-(pyridin-4-ylcarbonyl)-2,8-diazabicyclo[4.3.0]nonane,
3-(furan-2-ylcarbonyl)-3,7-diazabicyclo[4.3.0]nonane,
7-(furan-2-ylcarbonyl)-3,7-diazabicyclo[4.3.0]nonane,
3-(3-methylfuran-2-ylcarbonyl)-3,7-diazabicyclo[4.3.0]nonane,
7-(3-methylfuran-2-ylcarbonyl)-3,7-diazabicyclo[4.3.0]nonane,
3-(5-methylfuran-2-ylcarbonyl)-3,7-diazabicyclo[4.3.0]nonane,
7-(5-methylfuran-2-ylcarbonyl)-3,7-diazabicyclo[4.3.0]nonane,
3-(3-chlorofuran-2-ylcarbonyl)-3,7-diazabicyclo[4.3.0]nonane,
7-(3-chlorofuran-2-ylcarbonyl)-3,7-diazabicyclo[4.3.0]nonane,
3-(5-chlorofuran-2-ylcarbonyl)-3,7-diazabicyclo[4.3.0]nonane,
7-(5-chlorofuran-2-ylcarbonyl)-3,7-diazabicyclo[4.3.0]nonane,
3-(3-bromofuran-2-ylcarbonyl)-3,7-diazabicyclo[4.3.0]nonane,
7-(3-bromofuran-2-ylcarbonyl)-3,7-diazabicyclo[4.3.0]nonane,
3-(5-bromofuran-2-ylcarbonyl)-3,7-diazabicyclo[4.3.0]nonane,
7-(5-bromofuran-2-ylcarbonyl)-3,7-diazabicyclo[4.3.0]nonane,
3-(furan-3-ylcarbonyl)-3,7-diazabicyclo[4.3.0]nonane,
7-(furan-3-ylcarbonyl)-3,7-diazabicyclo[4.3.0]nonane,
3-(2-methylfuran-3-ylcarbonyl)-3,7-diazabicyclo[4.3.0]nonane,
7-(2-methylfuran-3-ylcarbonyl)-3,7-diazabicyclo[4.3.0]nonane,
3-(oxazol-2-ylcarbonyl)-3,7-diazabicyclo[4.3.0]nonane,
7-(oxazol-2-ylcarbonyl)-3,7-diazabicyclo[4.3.0]nonane,
3-(oxazol-5-ylcarbonyl)-3,7-diazabicyclo[4.3.0]nonane,
7-(oxazol-5-ylcarbonyl)-3,7-diazabicyclo[4.3.0]nonane,
3-(4-methyloxazol-5-ylcarbonyl)-3,7-diazabicyclo[4.3.0]nonane,
7-(4-methyloxazol-5-ylcarbonyl)-3,7-diazabicyclo[4.3.0]nonane,
3-(isoxazol-3-ylcarbonyl)-3,7-diazabicyclo[4.3.0]nonane,
7-(isoxazol-3-ylcarbonyl)-3,7-diazabicyclo[4.3.0]nonane,
3-(5-methylisoxazol-3-ylcarbonyl)-3,7-diazabicyclo[4.3.0]nonane,
7-(5-methylisoxazol-3-ylcarbonyl)-3,7-diazabicyclo[4.3.0]nonane,
3-(isoxazol-4-ylcarbonyl)-3,7-diazabicyclo[4.3.0]nonane,
7-(isoxazol-4-ylcarbonyl)-3,7-diazabicyclo[4.3.0]nonane,
3-(3-methylisoxazol-4-ylcarbonyl)-3,7-diazabicyclo[4.3.0]nonane,
7-(3-methylisoxazol-4-ylcarbonyl)-3,7-diazabicyclo[4.3.0]nonane,
3-(5-methylisoxazol-4-ylcarbonyl)-3,7-diazabicyclo[4.3.0]nonane,
7-(5-methylisoxazol-4-ylcarbonyl)-3,7-diazabicyclo[4.3.0]nonane,
3-(isoxazol-5-ylcarbonyl)-3,7-diazabicyclo[4.3.0]nonane,
7-(isoxazol-5-ylcarbonyl)-3,7-diazabicyclo[4.3.0]nonane,
3-(3-methylisoxazol-5-ylcarbonyl)-3,7-diazabicyclo[4.3.0]nonane,
7-(3-methylisoxazol-5-ylcarbonyl)-3,7-diazabicyclo[4.3.0]nonane,
3-(3-bromoisoxazol-5-ylcarbonyl)-3,7-diazabicyclo[4.3.0]nonane,
7-(3-bromoisoxazol-5-ylcarbonyl)-3,7-diazabicyclo[4.3.0]nonane,
3-(3-methoxyisoxazol-5-ylcarbonyl)-3,7-diazabicyclo[4.3.0]nonane,
7-(3-methoxyisoxazol-5-ylcarbonyl)-3,7-diazabicyclo[4.3.0]nonane,
3-(pyridin-4-ylcarbonyl)-3,7-diazabicyclo[4.3.0]nonane,
7-(pyridin-4-ylcarbonyl)-3,7-diazabicyclo[4.3.0]nonane,
3-(furan-2-ylcarbonyl)-3,8-diazabicyclo[4.3.0]nonane,
8-(furan-2-ylcarbonyl)-3,8-diazabicyclo[4.3.0]nonane,
3-(3-methylfuran-2-ylcarbonyl)-3,8-diazabicyclo[4.3.0]nonane,
8-(3-methylfuran-2-ylcarbonyl)-3,8-diazabicyclo[4.3.0]nonane,
3-(5-methylfuran-2-ylcarbonyl)-3,8-diazabicyclo[4.3.0]nonane,
8-(5-methylfuran-2-ylcarbonyl)-3,8-diazabicyclo[4.3.0]nonane,
3-(3-chlorofuran-2-ylcarbonyl)-3,8-diazabicyclo[4.3.0]nonane,
8-(3-chlorofuran-2-ylcarbonyl)-3,8-diazabicyclo[4.3.0]nonane,
3-(5-chlorofuran-2-ylcarbonyl)-3,8-diazabicyclo[4.3.0]nonane,
8-(5-chlorofuran-2-ylcarbonyl)-3,8-diazabicyclo[4.3.0]nonane,
3-(3-bromofuran-2-ylcarbonyl)-3,8-diazabicyclo[4.3.0]nonane,
8-(3-bromofuran-2-ylcarbonyl)-3,8-diazabicyclo[4.3.0]nonane,
3-(5-bromofuran-2-ylcarbonyl)-3,8-diazabicyclo[4.3.0]nonane,
8-(5-bromofuran-2-ylcarbonyl)-3,8-diazabicyclo[4.3.0]nonane,
3-(furan-3-ylcarbonyl)-3,8-diazabicyclo[4.3.0]nonane,
8-(furan-3-ylcarbonyl)-3,8-diazabicyclo[4.3.0]nonane,
3-(2-methylfuran-3-ylcarbonyl)-3,8-diazabicyclo[4.3.0]nonane,
8-(2-methylfuran-3-ylcarbonyl)-3,8-diazabicyclo[4.3.0]nonane,
3-(oxazol-2-ylcarbonyl)-3,8-diazabicyclo[4.3.0]nonane,
8-(oxazol-2-ylcarbonyl)-3,8-diazabicyclo[4.3.0]nonane,
3-(oxazol-5-ylcarbonyl)-3,8-diazabicyclo[4.3.0]nonane,
8-(oxazol-5-ylcarbonyl)-3,8-diazabicyclo[4.3.0]nonane,
3-(4-methyloxazol-5-ylcarbonyl)-3,8-diazabicyclo[4.3.0]nonane,
8-(4-methyloxazol-5-ylcarbonyl)-3,8-diazabicyclo[4.3.0]nonane,
3-(isoxazol-3-ylcarbonyl)-3,8-diazabicyclo[4.3.0]nonane,
8-(isoxazol-3-ylcarbonyl)-3,8-diazabicyclo[4.3.0]nonane,
3-(5-methylisoxazol-3-ylcarbonyl)-3,8-diazabicyclo[4.3.0]nonane,
8-(5-methylisoxazol-3-ylcarbonyl)-3,8-diazabicyclo[4.3.0]nonane,
3-(isoxazol-4-ylcarbonyl)-3,8-diazabicyclo[4.3.0]nonane,
8-(isoxazol-4-ylcarbonyl)-3,8-diazabicyclo[4.3.0]nonane,
3-(3-methylisoxazol-4-ylcarbonyl)-3,8-diazabicyclo[4.3.0]nonane,
8-(3-methylisoxazol-4-ylcarbonyl)-3,8-diazabicyclo[4.3.0]nonane,
3-(5-methylisoxazol-4-ylcarbonyl)-3,8-diazabicyclo[4.3.0]nonane,
8-(5-methylisoxazol-4-ylcarbonyl)-3,8-diazabicyclo[4.3.0]nonane,
3-(isoxazol-5-ylcarbonyl)-3,8-diazabicyclo[4.3.0]nonane,
8-(isoxazol-5-ylcarbonyl)-3,8-diazabicyclo[4.3.0]nonane,
3-(3-methylisoxazol-5-ylcarbonyl)-3,8-diazabicyclo[4.3.0]nonane,
8-(3-methylisoxazol-5-ylcarbonyl)-3,8-diazabicyclo[4.3.0]nonane,
3-(3-bromoisoxazol-5-ylcarbonyl)-3,8-diazabicyclo[4.3.0]nonane,
8-(3-bromoisoxazol-5-ylcarbonyl)-3,8-diazabicyclo[4.3.0]nonane,
3-(3-methoxyisoxazol-5-ylcarbonyl)-3,8-diazabicyclo[4.3.0]nonane,
8-(3-methoxyisoxazol-5-ylcarbonyl)-3,8-diazabicyclo[4.3.0]nonane,
3-(pyridin-4-ylcarbonyl)-3,8-diazabicyclo[4.3.0]nonane,
8-(pyridin-4-ylcarbonyl)-3,8-diazabicyclo[4.3.0]nonane,
3-(furan-2-ylcarbonyl)-3,9-diazabicyclo[4.3.0]nonane,
9-(furan-2-ylcarbonyl)-3,9-diazabicyclo[4.3.0]nonane,
3-(3-methylfuran-2-ylcarbonyl)-3,9-diazabicyclo[4.3.0]nonane,
9-(3-methylfuran-2-ylcarbonyl)-3,9-diazabicyclo[4.3.0]nonane,
3-(5-methylfuran-2-ylcarbonyl)-3,9-diazabicyclo[4.3.0]nonane,
9-(5-methylfuran-2-ylcarbonyl)-3,9-diazabicyclo[4.3.0]nonane,
3-(3-chlorofuran-2-ylcarbonyl)-3,9-diazabicyclo[4.3.0]nonane,
9-(3-chlorofuran-2-ylcarbonyl)-3,9-diazabicyclo[4.3.0]nonane,
3-(5-chlorofuran-2-ylcarbonyl)-3,9-diazabicyclo[4.3.0]nonane,
9-(5-chlorofuran-2-ylcarbonyl)-3,9-diazabicyclo[4.3.0]nonane,
3-(3-bromofuran-2-ylcarbonyl)-3,9-diazabicyclo[4.3.0]nonane,
9-(3-bromofuran-2-ylcarbonyl)-3,9-diazabicyclo[4.3.0]nonane,
3-(5-bromofuran-2-ylcarbonyl)-3,9-diazabicyclo[4.3.0]nonane,
9-(5-bromofuran-2-ylcarbonyl)-3,9-diazabicyclo[4.3.0]nonane,
3-(furan-3-ylcarbonyl)-3,9-diazabicyclo[4.3.0]nonane,
9-(furan-3-ylcarbonyl)-3,9-diazabicyclo[4.3.0]nonane,
3-(2-methylfuran-3-ylcarbonyl)-3,9-diazabicyclo[4.3.0]nonane,
9-(2-methylfuran-3-ylcarbonyl)-3,9-diazabicyclo[4.3.0]nonane,
3-(oxazol-2-ylcarbonyl)-3,9-diazabicyclo[4.3.0]nonane,
9-(oxazol-2-ylcarbonyl)-3,9-diazabicyclo[4.3.0]nonane,
3-(oxazol-5-ylcarbonyl)-3,9-diazabicyclo[4.3.0]nonane,
9-(oxazol-5-ylcarbonyl)-3,9-diazabicyclo[4.3.0]nonane,
3-(4-methyloxazol-5-ylcarbonyl)-3,9-diazabicyclo[4.3.0]nonane,
9-(4-methyloxazol-5-ylcarbonyl)-3,9-diazabicyclo[4.3.0]nonane,
3-(isoxazol-3-ylcarbonyl)-3,9-diazabicyclo[4.3.0]nonane,
9-(isoxazol-3-ylcarbonyl)-3,9-diazabicyclo[4.3.0]nonane,
3-(5-methylisoxazol-3-ylcarbonyl)-3,9-diazabicyclo[4.3.0]nonane,
9-(5-methylisoxazol-3-ylcarbonyl)-3,9-diazabicyclo[4.3.0]nonane,
3-(isoxazol-4-ylcarbonyl)-3,9-diazabicyclo[4.3.0]nonane,
9-(isoxazol-4-ylcarbonyl)-3,9-diazabicyclo[4.3.0]nonane,
3-(3-methylisoxazol-4-ylcarbonyl)-3,9-diazabicyclo[4.3.0]nonane,
9-(3-methylisoxazol-4-ylcarbonyl)-3,9-diazabicyclo[4.3.0]nonane,
3-(5-methylisoxazol-4-ylcarbonyl)-3,9-diazabicyclo[4.3.0]nonane,
9-(5-methylisoxazol-4-ylcarbonyl)-3,9-diazabicyclo[4.3.0]nonane,
3-(isoxazol-5-ylcarbonyl)-3,9-diazabicyclo[4.3.0]nonane,
9-(isoxazol-5-ylcarbonyl)-3,9-diazabicyclo[4.3.0]nonane,
3-(3-methylisoxazol-5-ylcarbonyl)-3,9-diazabicyclo[4.3.0]nonane,
9-(3-methylisoxazol-5-ylcarbonyl)-3,9-diazabicyclo[4.3.0]nonane,
3-(3-bromoisoxazol-5-ylcarbonyl)-3,9-diazabicyclo[4.3.0]nonane,
9-(3-bromoisoxazol-5-ylcarbonyl)-3,9-diazabicyclo[4.3.0]nonane,
3-(3-methoxyisoxazol-5-ylcarbonyl)-3,9-diazabicyclo[4.3.0]nonane,
9-(3-methoxyisoxazol-5-ylcarbonyl)-3,9-diazabicyclo[4.3.0]nonane,
3-(pyridin-4-ylcarbonyl)-3,9-diazabicyclo[4.3.0]nonane,
9-(pyridin-4-ylcarbonyl)-3,9-diazabicyclo[4.3.0]nonane,
2-(furan-2-ylcarbonyl)-2,6-diazabicyclo[3.2.1]octane,
6-(furan-2-ylcarbonyl)-2,6-diazabicyclo[3.2.1]octane,
2-(3-methylfuran-2-ylcarbonyl)-2,6-diazabicyclo[3.2.1]octane,
6-(3-methylfuran-2-ylcarbonyl)-2,6-diazabicyclo[3.2.1]octane,
2-(5-methylfuran-2-ylcarbonyl)-2,6-diazabicyclo[3.2.1]octane,
6-(5-methylfuran-2-ylcarbonyl)-2,6-diazabicyclo[3.2.1]octane,
2-(3-chlorofuran-2-ylcarbonyl)-2,6-diazabicyclo[3.2.1]octane,
6-(3-chlorofuran-2-ylcarbonyl)-2,6-diazabicyclo[3.2.1]octane,
2-(5-chlorofuran-2-ylcarbonyl)-2,6-diazabicyclo[3.2.1]octane,
6-(5-chlorofuran-2-ylcarbonyl)-2,6-diazabicyclo[3.2.1]octane,
2-(3-bromofuran-2-ylcarbonyl)-2,6-diazabicyclo[3.2.1]octane,
6-(3-bromofuran-2-ylcarbonyl)-2,6-diazabicyclo[3.2.1]octane,
2-(5-bromofuran-2-ylcarbonyl)-2,6-diazabicyclo[3.2.1]octane,
6-(5-bromofuran-2-ylcarbonyl)-2,6-diazabicyclo[3.2.1]octane,
2-(furan-3-ylcarbonyl)-2,6-diazabicyclo[3.2.1]octane,
6-(furan-3-ylcarbonyl)-2,6-diazabicyclo[3.2.1]octane,
2-(2-methylfuran-3-ylcarbonyl)-2,6-diazabicyclo[3.2.1]octane,
6-(2-methylfuran-3-ylcarbonyl)-2,6-diazabicyclo[3.2.1]octane,
2-(oxazol-2-ylcarbonyl)-2,6-diazabicyclo[3.2.1]octane,
6-(oxazol-2-ylcarbonyl)-2,6-diazabicyclo[3.2.1]octane,
2-(oxazol-5-ylcarbonyl)-2,6-diazabicyclo[3.2.1]octane,
6-(oxazol-5-ylcarbonyl)-2,6-diazabicyclo[3.2.1]octane,
2-(4-methyloxazol-5-ylcarbonyl)-2,6-diazabicyclo[3.2.1]octane,
6-(4-methyloxazol-5-ylcarbonyl)-2,6-diazabicyclo[3.2.1]octane,
2-(isoxazol-3-ylcarbonyl)-2,6-diazabicyclo[3.2.1]octane,
6-(isoxazol-3-ylcarbonyl)-2,6-diazabicyclo[3.2.1]octane,
2-(5-methylisoxazol-3-ylcarbonyl)-2,6-diazabicyclo[3.2.1]octane,
6-(5-methylisoxazol-3-ylcarbonyl)-2,6-diazabicyclo[3.2.1]octane,
2-(isoxazol-4-ylcarbonyl)-2,6-diazabicyclo[3.2.1]octane,
6-(isoxazol-4-ylcarbonyl)-2,6-diazabicyclo[3.2.1]octane,
2-(3-methylisoxazol-4-ylcarbonyl)-2,6-diazabicyclo[3.2.1]octane,
6-(3-methylisoxazol-4-ylcarbonyl)-2,6-diazabicyclo[3.2.1]octane,
2-(5-methylisoxazol-4-ylcarbonyl)-2,6-diazabicyclo[3.2.1]octane,
6-(5-methylisoxazol-4-ylcarbonyl)-2,6-diazabicyclo[3.2.1]octane,
2-(isoxazol-5-ylcarbonyl)-2,6-diazabicyclo[3.2.1]octane,
6-(isoxazol-5-ylcarbonyl)-2,6-diazabicyclo[3.2.1]octane,
2-(3-methylisoxazol-5-ylcarbonyl)-2,6-diazabicyclo[3.2.1]octane,
6-(3-methylisoxazol-5-ylcarbonyl)-2,6-diazabicyclo[3.2.1]octane,
2-(3-bromoisoxazol-5-ylcarbonyl)-2,6-diazabicyclo[3.2.1]octane,
6-(3-bromoisoxazol-5-ylcarbonyl)-2,6-diazabicyclo[3.2.1]octane,
2-(3-methoxyisoxazol-5-ylcarbonyl)-2,6-diazabicyclo[3.2.1]octane,
6-(3-methoxyisoxazol-5-ylcarbonyl)-2,6-diazabicyclo[3.2.1]octane,
2-(pyridin-4-ylcarbonyl)-2,6-diazabicyclo[3.2.1]octane,
6-(pyridin-4-ylcarbonyl)-2,6-diazabicyclo[3.2.1]octane,
3-(furan-2-ylcarbonyl)-3,6-diazabicyclo[3.2.1]octane,
6-(furan-2-ylcarbonyl)-3,6-diazabicyclo[3.2.1]octane,
3-(3-methylfuran-2-ylcarbonyl)-3,6-diazabicyclo[3.2.1]octane,
6-(3-methylfuran-2-ylcarbonyl)-3,6-diazabicyclo[3.2.1]octane,
3-(5-methylfuran-2-ylcarbonyl)-3,6-diazabicyclo[3.2.1]octane,
6-(5-methylfuran-2-ylcarbonyl)-3,6-diazabicyclo[3.2.1]octane,
3-(3-chlorofuran-2-ylcarbonyl)-3,6-diazabicyclo[3.2.1]octane,
6-(3-chlorofuran-2-ylcarbonyl)-3,6-diazabicyclo[3.2.1]octane,
3-(5-chlorofuran-2-ylcarbonyl)-3,6-diazabicyclo[3.2.1]octane,
6-(5-chlorofuran-2-ylcarbonyl)-3,6-diazabicyclo[3.2.1]octane,
3-(3-bromofuran-2-ylcarbonyl)-3,6-diazabicyclo[3.2.1]octane,
6-(3-bromofuran-2-ylcarbonyl)-3,6-diazabicyclo[3.2.1]octane,
3-(5-bromofuran-2-ylcarbonyl)-3,6-diazabicyclo[3.2.1]octane,
6-(5-bromofuran-2-ylcarbonyl)-3,6-diazabicyclo[3.2.1]octane,
3-(furan-3-ylcarbonyl)-3,6-diazabicyclo[3.2.1]octane,
6-(furan-3-ylcarbonyl)-3,6-diazabicyclo[3.2.1]octane,
3-(2-methylfuran-3-ylcarbonyl)-3,6-diazabicyclo[3.2.1]octane,
6-(2-methylfuran-3-ylcarbonyl)-3,6-diazabicyclo[3.2.1]octane,
3-(oxazol-2-ylcarbonyl)-3,6-diazabicyclo[3.2.1]octane,
6-(oxazol-2-ylcarbonyl)-3,6-diazabicyclo[3.2.1]octane,
3-(oxazol-5-ylcarbonyl)-3,6-diazabicyclo[3.2.1]octane,
6-(oxazol-5-ylcarbonyl)-3,6-diazabicyclo[3.2.1]octane,
3-(4-methyloxazol-5-ylcarbonyl)-3,6-diazabicyclo[3.2.1]octane,
6-(4-methyloxazol-5-ylcarbonyl)-3,6-diazabicyclo[3.2.1]octane,
3-(isoxazol-3-ylcarbonyl)-3,6-diazabicyclo[3.2.1]octane,
6-(isoxazol-3-ylcarbonyl)-3,6-diazabicyclo[3.2.1]octane,
3-(5-methylisoxazol-3-ylcarbonyl)-3,6-diazabicyclo[3.2.1]octane,
6-(5-methylisoxazol-3-ylcarbonyl)-3,6-diazabicyclo[3.2.1]octane,
3-(isoxazol-4-ylcarbonyl)-3,6-diazabicyclo[3.2.1]octane,
6-(isoxazol-4-ylcarbonyl)-3,6-diazabicyclo[3.2.1]octane,
3-(3-methylisoxazol-4-ylcarbonyl)-3,6-diazabicyclo[3.2.1]octane,
6-(3-methylisoxazol-4-ylcarbonyl)-3,6-diazabicyclo[3.2.1]octane,
3-(5-methylisoxazol-4-ylcarbonyl)-3,6-diazabicyclo[3.2.1]octane,
6-(5-methylisoxazol-4-ylcarbonyl)-3,6-diazabicyclo[3.2.1]octane,
3-(isoxazol-5-ylcarbonyl)-3,6-diazabicyclo[3.2.1]octane,
6-(isoxazol-5-ylcarbonyl)-3,6-diazabicyclo[3.2.1]octane,
3-(3-methylisoxazol-5-ylcarbonyl)-3,6-diazabicyclo[3.2.1]octane,
6-(3-methylisoxazol-5-ylcarbonyl)-3,6-diazabicyclo[3.2.1]octane,
3-(3-bromoisoxazol-5-ylcarbonyl)-3,6-diazabicyclo[3.2.1]octane,
6-(3-bromoisoxazol-5-ylcarbonyl)-3,6-diazabicyclo[3.2.1]octane,
3-(3-methoxyisoxazol-5-ylcarbonyl)-3,6-diazabicyclo[3.2.1]octane,
6-(3-methoxyisoxazol-5-ylcarbonyl)-3,6-diazabicyclo[3.2.1]octane,
3-(pyridin-4-ylcarbonyl)-3,6-diazabicyclo[3.2.1]octane, and
6-(pyridin-4-ylcarbonyl)-3,6-diazabicyclo[3.2.1]octane,
or a pharmaceutically acceptable salt thereof.

16. The method of claim 15, wherein the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, cognitive dysfunction in schizophrenia, schizophreniform disorder, and schizoaffective disorder.

17. The method of claim 15, wherein the disorder is selected from the group consisting of mild to moderate dementia of the Alzheimer's type, attention deficit disorder, mild cognitive impairment, and age associated memory impairment.

18. (1S,5S)-3-(5-bromofuran-2-ylcarbonyl)-3,6-diazabicyclo[3.2.1]octane or a pharmaceutically acceptable salt thereof.
